SEQUENCE LISTING (33 sequences)

SEQ ID NO: 1 - protein, 120 aa, Homo sapiens
EVQLVESGGGLMKPGGSLKLSCAASGFAFGSYDMSWVRQTPEKRLEWVAYISSGGGITFYPDSVRGRFTISRDNAKNSLYMEMSSLRSEDTAMYYCARWDWDLFAYWGQGTLVTVSAAAS

SEQ ID NO: 2 - protein, 107 aa, Homo sapiens
DIKMTQSPSSMYTSLGERVTITCKASQDINSCLSWFQQKPGKSPKALIFRANILVDGVPSRFSGSGSGQDYSLTISSLEYEDLGIYYCLQYDEFPWTFGGGTRLEIK

SEQ ID NO: 3 - protein, 119 aa, Homo sapiens
QVQLQQPGAELVKPGASVRLSCKAGGYTFTSYWLHWVKQRPGQGLEWIGMIHPNSGSYDYSETFRTKATLTVDKSSDTAYMQLTSLTSEDSAVYYCARGGSNYDIFAYWGQGTTLTVSS

SEQ ID NO: 4 - protein, 106 aa, Homo sapiens
NIVMTQSPKSMSMSVGERVTFNCRASENVGTYVFWYQQKPEQSPRLLIYGASNRYTGVPDRFTGSGSATDFTLTISGVQAEDLADYHCGQSYRHLTFGGGTKLEIK

SEQ ID NO: 5 - protein, 119 aa, Homo sapiens
QVQLQQPGTELVKPGASVKLSCKASGYTFTNYWMHWVKQRPGQGLEWVGNINPSNGGTNYNEKFKSKATLTVDKSSSTAYMQLSSLTSEDSAFYYCARGRGPPYYSDYWGQGSTLTVSS

SEQ ID NO: 6 - protein, 107 aa, Homo sapiens
NIQMTQSPASLSASVGETVTITCRASGNIHNYLAWYQQKQGKSPQLLVYNAKTLADGVPSRFSGSGSGTQYSLKINSLQPEDFGSYYCQHFWSTPFTFGSGTKLEIK

SEQ ID NO: 7 - protein, 121 aa, Homo sapiens
QVQLQQSGPELVKPGASVRISCKASGFTFTSYHVNWVKQRPGQGLEWIGWIYPGNVNTEYNEKFKGKATLTADKSSSTAYMQLSSLTSEDSAVYFCASHEYYGSDWYFDVWGAGTTVTVSS

SEQ ID NO: 8 - protein, 112 aa, Homo sapiens
DAVMTQTPLSLPVSLGDQASISCRSSQSLENRNGNTYLNWYLQKPGQSPQLLIYRVSNRFSGVLDRFSGSGSGTDFTLKISRVEAEDLGVYFCLQVTHVPYTFGGGTKLEIK

SEQ ID NO: 9 - protein, 469 aa, Homo sapiens
MEFGLSWLFLVAILKGVQCQVQLQQSGPELVKPGASVRISCKASGFTFSYHVNWVKQRPGQGLEWIGWIYPGNVNTEYNEKFKGKATLTADKSSSTAYMQLSSLTSEDSAVYFCASHEYYGSDWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 28 bp, Artificial Sequence
  Primer
caccatgaagttgcctgttaggctgttg

SEQ ID NO: 11 - DNA, 30 bp, Artificial Sequence
  Primer
gaagatctagacttactatgcagcatcagc

SEQ ID NO: 12 - DNA, 35 bp, Artificial Sequence
  Primer
atatccaccatggratgsagctgkgtmatsctctt

SEQ ID NO: 13 - DNA, 27 bp, Artificial Sequence
  Primer
ggcagcactagtaggggccagtggata

SEQ ID NO: 14 - protein, 2710 aa, Clostridium difficile
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNDSTLEFNSKDFIAEDINVFMKDDINTITGKYYVDNNTDKSIDFSISLVSKNQVKVNGLYLNESVYSSYLDFVKNSDGHHNTSNFMNLFLDNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIEFNLVTGWQTINGKKYYFDINTGAALTSYKIINGKHFYFNNDGVMQLGVFKGPDGFEYFAPANTQNNNIEGQAIVYQSKFLTLNGKKYYFDNNSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 15 - protein, 20 aa, Streptococcus pneumoniae
GTVTFNGQRLYFKPNGVQAK

SEQ ID NO: 16 - protein, 20 aa, Streptococcus pneumoniae
GARTINGQLLYFRANGVQVK

SEQ ID NO: 17 - protein, 20 aa, Streptococcus pneumoniae
GAQTIKGQKLYFKANGQQVK

SEQ ID NO: 18 - protein, 20 aa, Streptococcus pneumoniae
GWVKIGDGWYYFDNSGAMAT

SEQ ID NO: 19 - protein, 20 aa, Streptococcus pneumoniae
GWIKIADGWYYFDSDGAMAT

SEQ ID NO: 20 - protein, 20 aa, Streptococcus pneumoniae
GWQTINGKKYYFNTNTAAAA

SEQ ID NO: 21 - protein, 18 aa, Streptococcus pneumoniae
GLVIDDKKYYFDDDGIMQ

SEQ ID NO: 22 - protein, 20 aa, Streptococcus pneumoniae
GLITIDDKKYYFDDNGIMQV

SEQ ID NO: 23 - protein, 30 aa, Streptococcus pneumoniae
GVFKGPNGFEYFAPANTDNNNIEGQAIVYI

SEQ ID NO: 24 - protein, 30 aa, Streptococcus pneumoniae
GVFNTEDGFKYFAPANTLDENLEGEAINYI

SEQ ID NO: 25 - protein, 28 aa, Streptococcus pneumoniae
GVFNTPDGFKYFAPNTLDENEGESVNYT

SEQ ID NO: 26 - protein, 445 aa, Homo sapiens
EVQLVESGGGLMKPGGSLKLSCAASGFAFGSYDMSWVRQTPEKRLEWVAYISSGGGITFYPDSVRGRFTISRDNAKNSLYMEMSSLRSEDTAMYYCARWDWDLFAYWGQGTLVTVSATKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 214 aa, Homo sapiens
DIKMTQSPSSMYTSLGERVTITCKASQDINSCLSWFQQKPGKSPKALIFRANILVDGVPSRFSGSGSGQDYSLTISSLEYEDLGIYYCLQYDEFPWTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 447 aa, Homo sapiens
QVQLQQPGAELVKPGASVRLSCKAGGYTFTSYWLHWVKQRPGQGLEWIGMIHPNSGSYDYSETFRTKATLTVDKSSDTAYMQLTSLTSEDSAVYYCARGGSNYDIFAYWGQGTTLTVSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 213 aa, Homo sapiens
NIVMTQSPKSMSMSVGERVTFNCRASENVGTYVFWYQQKPEQSPRLLIYGASNRYTGVPDRFTGSGSATDFTLTISGVQAEDLADYHCGQSYRHLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 447 aa, Homo sapiens
QVQLQQPGTELVKPGASVKLSCKASGYTFTNYWMHWVKQRPGQGLEWVGNINPSNGGTNYNEKFKSKATLTVDKSSSTAYMQLSSLTSEDSAFYYCARGRGPPYYSDYWGQGSTLTVSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 214 aa, Homo sapiens
NIQMTQSPASLSASVGETVTITCRASGNIHNYLAWYQQKQGKSPQLLVYNAKTLADGVPSRFSGSGSGTQYSLKINSLQPEDFGSYYCQHFWSTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 7 aa, Homo sapiens
HAIYPRH

SEQ ID NO: 33 - protein, 12 aa, Homo sapiens
THRPPMWSPVWP

(19) United States
(12) Patent Application Publication
(10) Pub. No.: US 2009/0087478 A1
(43) Pub. Date: Apr. 2, 2009

(54) Orally Deliverable and Anti-Toxin Antibodies and Methods for Making and Using Them

(75) Inventors: Genevieve Hansen, San Diego, CA (US); Stephen J. Demarest, San Diego, CA (US)

Correspondence Address: KELLEY DRYE & WARREN LLP, 400 ALTLANTIC STREET , 13TH FLOOR, STAMFORD, CT, 06901, US

(73) Assignee: Progenics Pharmaceuticals (Nevada), Inc., Tarrytown, NY (US)

(21) Appl. No.: 11/794,491

(22) PCT Filed: Dec. 22, 2005
(86) PCT No.: PCT/US05/47100

§ 371 Date: Apr. 3, 2008

Related U.S. Application Data
(60) Provisional application No. 60/639,827, filed on Dec. 27, 2004.

(51) Int. Cl. A61K 9/127; C07K 16/18; C12N 15/11; A61K 9/48; C12P 21/04; C12N 15/00; A61K 39/395

(52) U.S. Cl. 424/450; 530/387.3; 536/23.53; 435/320.1; 424/133.1; 424/456; 435/69.6

(57) ABSTRACT

The invention provides antibodies with superior therapeutic efficacy and related methods of engineering such antibodies to increase their stability and resistance to proteases, e.g., in the digestive tract. Protease cleavage motifs are identified and subsequently modified to reduce or eliminate cleavage at that site. Methods of employing these orally deliverable antibodies as therapeutic compositions, particularly against gastrointestinal pathogens are also provided herein. In one aspect, the invention provides combinations of monoclonal antibodies, e.g., “synthetic polyclonals,” that work synergistically to neutralize bacterial toxins, particularly enteric bacterial toxins such as Clostridium difficile toxin A.

### CROSS-REFERENCE TO RELATED APPLICATIONS

This application claims benefit of priority to U.S. provisional patent application 60/639,827, filed Dec. 27, 2004. The contents of this document are expressly incorporated herein by reference in its entirety for all purposes.

### FIELD OF THE INVENTION

This invention generally relates to medicine, infectious disease and the use of recombinant antibodies in the treatment of bacterial disease, e.g., those caused by enteric bacterial toxins. In one aspect, the invention provides antibodies modified for increased resistance to proteolysis and/or acidic conditions to improve therapeutic efficacy, e.g., for oral administration, and methods for making and using these antibodies. In one aspect, the invention provides combinations of monoclonal antibodies, e.g., “synthetic polyclonals,” that work synergistically to neutralize bacterial toxins, e.g., enteric bacterial toxins such as Clostridium difficile toxin A.

### BACKGROUND

Antibodies (Abs) are ideal therapeutic agents for their specificity and flexibility. The antibody (Ab) targets a cell or an organism through its binding of a specific epitope on an antigen mediated as dictated by the variable region of the antibody molecule. The antibody's specificity is complemented by its ability to mediate and/or initiate a variety of biological activities. For example, antibodies can modulate receptor-ligand interactions as agonists or antagonists. Antibody binding can initiate intracellular signaling to stimulate cell growth, cytokine production, or apoptosis. Antibodies deliver agents bound to the Fc region to specific sites. Antibodies also elicit antibody-mediated cytotoxicity (ADCC), complement-mediated cytotoxicity (CDC), and phagocytosis. Thus, antibodies are increasingly being used as therapeutic agents to treat various diseases.

While the properties of antibodies make them very attractive therapeutic agents, there are a number of limitations. One of the primary limitations is the stability of the antibody following administration to a subject. Antibodies are proteins and thus are susceptible to degradation by proteolytic enzymes present in, for example, the blood and digestive tract. Complete or partial degradation of the antibody prevents a therapeutically effective amount from reaching a distant target site when the antibody is administered systemically.

Administration of antibodies to combat pathogens and diseases in the digestive tract also face an additional hurdle—the low pH environment. The stomach manufactures pepsin and contains hydrochloric acid (pH range between 1.5 and 3). The low pH denatures the proteins, resulting in an increased vulnerability to pepsin degradation. The average person secretes about 400 mL of gastric fluid per meal, containing 50 to 300 μg pepsin/mL. The transit time in the stomach varies from 0.5 to 4.5 h. Protein digestion continues in the duodenum and jejunum, where proteolytic enzymes of the pancreas (trypsin, trypsinogen, chymo-trypsinogen, pro-carboxy-peptidase, and pro-elastase) attack the remaining breakdown products. The pH of the small intestine ranges from 6.3 to 7.5 with a transit time in the order of 1 to 4 h. In the colon, the pH is between 7.5 and 8 with a transit time of 8-16 h, creating a harsh environment for any orally administered protein.

Like most proteins, antibodies are degraded after oral administration. Antibodies are initially degraded into F(ab′)₂, Fab and Fc fragments. Even after this initial degradation, the F(ab′)₂ and Fab fragments retain some of their biological activity. For example, stool samples from adults receiving bovine milk immunoglobulin orally contained detectable amounts of antibody with neutralizing activity. However, degradation in these harsh conditions significantly limits the usefulness of orally administered antibodies.

An application for orally administered therapeutic antibodies of the invention (e.g., made by a method of the invention) includes the prevention, treatment and/or diagnosis of gastrointestinal infections and diseases. For example, one enteric pathogen, Clostridium difficile, a common gram-positive, spore-forming, anaerobic bacillus, is the leading cause of nosocomial diarrhea associated with antibiotic therapy. C. difficile infection results from a disruption of the normal bacterial flora of the colon, followed by colonization of C. difficile, and the release of destructive toxins that lead to mucosal damage and inflammation. Antibiotic therapy is the key factor that is responsible for altering the colonic flora and allowing C. difficile to flourish. After colonization, the organism releases two toxins, A and B, which are responsible for causing diarrhea and colitis. While antibiotics are available against C. difficile, these treatments have significant relapse rates. Moreover, the growing incidence of antibiotic-resistant organism makes such treatments increasing likely to be unsuccessful. Therefore, there is a need for the development of effective prophylactic and therapeutic treatments that specifically targets organisms like C. difficile that can neutralize virulence factors and suppress antibiotic-resistant organisms while simultaneously avoiding normal microflora disruption. Thus, antibodies that retain biological activity following systemic, e.g., oral, administration offer a significant improvement over the current prophylactic and therapeutic options currently available.

### SUMMARY

In one aspect, the invention provides antibodies that are sequence modified, e.g., recombinantly engineered or sequence modified as synthetic proteins, to increase their stability in harsh conditions, e.g., conditions comprising acidic pH or the presence of proteases, and methods of making and using them. By having selective mutations for increased resistance to these harsh conditions, antibodies of the invention are useful as therapeutic antibodies that retain biological activity systemically and at the target site, e.g., in the gut (e.g., stomach, intestine) even after oral delivery. In one aspect, antibodies of the invention can be delivered orally and retain biological activity in the presence of low pH and/or proteolytic enzymes for efficacy in a digestive tract environment. In one aspect, antibodies of the invention have protease resistance (e.g., greater protease resistance than unaltered, or wildtype antibody), increased thermotolerance and/or reduced sensitivity to the negative effects of extreme pH, such as those in the stomach environment, in comparison the a starting, or unaltered (e.g., wildtype) antibody sequence. The amount of protease resistance added to the antibody by practicing the invention can be complete or partial, or even only involve the modification of one site. In one aspect, antibodies of the invention are therapeutic antibodies retaining biological activity systemically and at a target site.

The invention provides isolated or recombinant antibodies having resistance to proteolysis (e.g., an increased resistance to proteolysis in comparison the a starting, or unaltered (e.g., wildtype) antibody sequence) made by a method comprising: (a) providing an antibody having at least one protease cleavage site: and (b) engineering (e.g., genetic engineering a nucleic acid coding sequence) at least one amino acid residue modification in the antibody, wherein the at least one amino acid residue modification(s) results in a resistance to (e.g., an increased resistance to) proteolysis, and the at least one amino acid residue modification comprises:

(i) at least one amino acid substitution at any one or more of amino acid positions T155, L179, L235, F241, Y296, L309, Y349, L365, L398, F404, Y407 or Y436 of an IgG heavy chain;

(ii) at least one amino acid substitution at any one or more of amino acid positions L234, L242, F243, F275, Y278, Y300, L306, W313, L314, Y319, L351, L368, Y391, F405, L406, L410, F423, L432, or Y436 of an IgG heavy chain;

(iii) at least one amino acid substitution at any one or more of amino acid positions F116, K126, R143, K169 or K183 of a kappa chain;

(iv) at least one amino acid substitution at any one or more of amino acid positions K133, K205, K210, K274, K326, K340, R355, K360 or K392 of an IgG heavy chain;

(v) at least one amino acid residue modification comprising at least one amino acid substitution at a P1 or P1′ site of cleavage in a trypsin cleavage motif, wherein the substituted amino acid is K or R;

(vi) at least one amino acid substitution at a P1 or P1′ site of cleavage in a pepsin cleavage motif, wherein the substituted amino acid is L, F, Y, W, I, or T;

(vii) at least one amino acid substitution at a P1 or P1′ site of cleavage in a chymotrypsin cleavage motif, wherein the substituted amino acid is F, Y, or W;

(viii) at least one amino acid substitution selected from the group of amino acid substitutions of L235P, L398Q, F404Y, L179I, and T155S in an IgG₁ heavy chain;

(ix) at least one amino acid substitution selected from the group of amino acid substitutions of F116S and K126A in a kappa light chain;

(x) at least one amino acid substitution selected from the group of amino acid substitutions of K133G and K274Q in a IgG heavy chain; or (xi) a combination of any of the modifications of steps (a) to (h),

wherein the numbering of the residues in the variant amino acid sequence is that of the EU index in the Kabat numbering system (see discussion, below, including Example 1). In one aspect, any one or combination of modifications of steps (i) to (x) are in a variable antibody region, a constant antibody region, or in both the variable antibody region and the constant antibody region. In one aspect, the antibody comprises human antibody sequence in the constant region, human antibody sequence in the variable region or human antibody sequence in the constant and the variable region.

In one aspect, the invention provides an isolated or recombinant antibody comprising a “variant portion” (e.g., a modified amino acid sequence, a modified motif, a modified protease cleavage site, and the like) comprising at least one amino acid modification, wherein said variant portion results in resistance to (e.g., an increased resistance to) proteolysis, and methods for making and using these modified antibodies. In some embodiments, the modification is in a protease cleavage site or at a site flanking the protease cleavage site. In alternative embodiments, the modification is at the P1, P1′, P2, P3, P4, P2′, P3′, or P4 residue of the protease cleavage site. In one aspect, the modification to the amino acid sequence generates a protease resistance motif, rendering the protease cleavage site non-cleavable or less susceptible to protease cleavage. In alternative aspects, the modifications are in a variable antibody region, a constant antibody region, or in both the variable antibody region (e.g., a CDR region) and the constant antibody region.

In one aspect, the variant portion comprises any number of modifications including one, two, three, four, five, six, seven, eight, nine, ten, eleven, or more amino acid residue modifications. In some embodiments, the modifications are made to the same protease cleavage motif throughout the antibody. In other embodiment, the modifications are made to different protease cleavage motifs. The modifications can be made in a protease cleavage site that is not flanked by an amino acid residue known to inhibit or attenuate protease cleavage. Such amino acids include, for example, Pro, Lys, Arg and His.

In one aspect, the variant portion of the antibody modified comprises any portion of the antibody including the heavy chain, a light chain, or both, or variable region or constant region or both. In some embodiments, the variant portion (modified part of the antibody sequence) is the Fc region, the hinge region, the CH_{L} domain, the CH₁ domain, the CH₂ domain, the CH₃ domain, the Fab region, or any combination thereof. In alternative embodiments, the variant portion is a V_{H} or V_{L} domain, provided the cleavage site does not have a negative effect on the desired antibody function.

In one aspect, the modifications in an antibody of the invention comprise at least one mutation in the amino acid sequence of the antibody. The mutation can be introduced by modifications, additions or deletions to a nucleic acid encoding the antibody. The modifications, additions or deletions to a nucleic acid encoding the antibody can be introduced by any method, including for example error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturation Mutagenesis (GSSM), synthetic ligation reassembly (SLR) or a combination thereof. The modifications, additions or deletions to a nucleic acid encoding the antibody can also be introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, or a combination thereof.

In one embodiment, the variant portion of an antibody of the invention comprises at least one amino acid substitution at any one or more of amino acid positions T155, L179, L235, F241, Y296, L309, Y349, L365, L398, F404, Y407, and Y436 of an IgG heavy chain, e.g., SEQ ID NO:1, SEQ ID NO:3 and/or SEQ ID NO:5, wherein the numbering of the residues in the modified amino acid residues (i.e., the variant portion) is that of the EU index as in Kabat, whereby the amino acid substitution confers increased resistance to pepsin proteolysis. In another embodiment, the variant portion comprises at least one amino acid substitution at any one or more of amino acid positions L234, L242, F243, F275, Y278, Y300, L306, W313, L314, Y319, L351, L368, Y391, F405, L406, L410, F423, L432, or Y436 of an IgG heavy chain, e.g., SEQ ID NO:1, SEQ ID NO:3 and/or SEQ ID NO:5, wherein the numbering of the residues in the variant portion is that of the EU index as in Kabat, whereby the amino acid substitution confers increased resistance to pepsin proteolysis.

In one embodiment, the variant portion comprises at least one amino acid substitution at any one or more of amino acid positions F116, K126, R143, K169 or K183 of a kappa (light) chain, e.g., SEQ ID NO:2, SEQ ID NO:4 and/or SEQ ID NO:6, wherein the numbering of the residues in the variant portion is that of the EU index as in Kabat, whereby the amino acid substitution confers increased resistance to pancreatin proteolysis.

In another embodiment, the variant portion comprises at least one amino acid substitution at any one or more of amino acid positions K133, K205, K210, K274, K326, K340, R355, K360 or K392 of an IgG heavy chain, e.g., SEQ ID NO:1, SEQ ID NO:3 and/or SEQ ID NO:5, wherein the numbering of the residues in the variant portion is that of the EU index as in Kabat, whereby the amino acid substitution confers increased resistance to pancreatin proteolysis.

In one aspect, the modified amino acid residues (i.e., the variant portion) of an antibody of the invention comprises at least one amino acid substitution at the P1 or P1′ site of cleavage in a trypsin cleavage motif, wherein the substituted amino acid is K or R, whereby the amino acid substitution confers increased resistance to trypsin proteolysis. In one embodiment, the variant portion comprises at least one amino acid substitution, at the P1 or P1′ site of cleavage in a pepsin cleavage motif, wherein the substituted amino acid is L, F, Y, W, I, or T, whereby the amino acid substitution confers increased resistance to pepsin proteolysis. In some embodiments, the variant portion comprises at least one amino acid substitution at the P1 or P1′ site of cleavage in a chymotrypsin cleavage motif, wherein the substituted amino acid is F, Y, or W, whereby the amino acid substitution confers increased resistance to chymotrypsin proteolysis.

In one embodiment, antibodies of the invention comprise at least one amino acid substitution, or all of the combination of amino acid substitutions, as set forth in Tables 3A or 3B (Example 1), Table 4 (Example 1), or Table 5 (Example 1), below.

In one embodiment, the modified amino acid residues (i.e., the variant portion) of an antibody of the invention comprises at least one amino acid substitution selected from the group of amino acid substitutions of L235P, L398Q, F404Y, L179I, and T155S in the IgG₁ heavy chain, wherein the numbering of the residues in the variant portion is that of the EU index as in Kabat, whereby the amino acid substitution confers increased resistance to pepsin proteolysis. In another specific embodiment, the variant portion comprises at least one amino acid substitution selected from the group of amino acid substitutions of F116S and K126A in the kappa light chain, wherein the numbering of the residues in the variant portion is that of the EU index as in Kabat, whereby the amino acid substitution confers increased resistance to pepsin proteolysis. In yet another specific embodiment, the variant portion comprises at least one amino acid substitution selected from the group of amino acid substitutions of K133G and K274Q in the IgG heavy chain, wherein the numbering of the residues in the variant portion is that of the EU index as in Kabat, whereby the amino acid substitution confers increased resistance to pepsin proteolysis.

In some embodiments, the resistance to proteolysis of an antibody of the invention, or an antibody used in a method of the invention, comprises a greater resistance to proteolysis relative to a corresponding unmodified, or “wildtype,” antibody. The increased resistance to proteolysis can be at least about 1%, 2%, 3%, 4%, 5%, 10%, 15%, 20%, 25%, 30%, 40%, 50%, 60%, 70%, 80% or more than that of the unmodified antibody. The modified antibody can be partially or completely resistant to cleavage by more than one protease.

In one aspect, an antibody of the invention, or an antibody used in a method of the invention, comprises an IgG, IgM, IgD, IgE, or IgA antibody. In alternative embodiments, the antibody is an IgG antibody of a particular isotype, e.g., an IgG₁, IgG₂, IgG₃, or IgG₄ antibody. The antibody can be a human, murine, rat, rabbit, bovine, camel, llama, dromedary, or simian antibody. The antibody can be a chimeric antibody (e.g., a humanized antibody, for example, a mixture of mouse and human sequence, such as SEQ ID NO:1 and SEQ ID NO:2 as variable regions, with human sequence completing the sequence for a complete antibody), a bispecific antibody, a fusion protein, or a biologically active (e.g., antigen binding) fragment thereof.

In one aspect, the humanized antibody comprises a variable region comprising a mouse sequence or a sequence derived from a mouse and a constant region comprising a human sequence. For example, in one aspect, an antibody of the invention comprises the heavy chain variable region sequence encoded in SEQ ID NO:1 and the light chain variable region sequence encoded in SEQ ID NO:2 and the remainder of the antibody (e.g., constant region) comprising human sequence, thus making a “humanized” chimeric antibody (similarly, in alternative aspects, the “humanized” chimeric antibody comprises the variable region sequence combinations SEQ ID NO:3 and SEQ ID NO:4, or, SEQ ID NO:5 and SEQ ID NO:6, or, SEQ ID NO:7 and SEQ ID NO:8).

An antibody of the invention or an antibody used in a method of the invention, can be modified in any suitable manner. In some embodiments, the modification comprises the addition of a post-translational modification site, an N-glycosylation site, an O-glycosylation site, an alkyl chain, or a small molecule. At times, the modification comprises covalent or non-covalent addition of a second molecule, e.g., to the Fc chain of the antibody.

In one aspect, the second molecule comprises an antibody secretory component, a carbohydrate, a disulfide bond site, or a salt bridge site. In one aspect, the second molecule or addition sequence comprises a moiety that serves to “shuttle” the protein from the gut into a cell and/or into the bloodstream (e.g., acting as a “transport” or “carrier” moiety to shuttle an orally administered protein into the blood or plasma). For example, in one aspect, a transferrin polypeptide moiety, a cell wall binding domain (CWB) domain of Clostridium difficile toxin A, or an equivalent protein, serves as a “shuttle”, “transport” or “carrier” moiety or domain to allow an antibody of the invention (or an antibody used in a method of the invention) enter cells (e.g., those lining the gut) or to allow an orally administered antibody of the invention enter into the bloodstream from the gut. The “shuttle”, “transport” or “carrier” moiety or domain can be sequence spliced into an antibody sequence or be covalently or non-covalently joined to or linked to an antibody sequence. In one aspect, the antibody and the “shuttle” domain are linked by a cleavable domain that is cleaved after entry into a cell and/or the bloodstream (thus “liberating” the antibody from the “shuttle” domain).

In some embodiment, the Fc region of an antibody of the invention is further modified to alter an activity of the Fc region, e.g., to abrogate, diminish or enhance an Fc-mediated antibody-mediated cytotoxicity (ADCC), a complement-mediated cytotoxicity (CDC), complement activation, Fc receptor activation and/or binding or phagocytosis. The Fc region of the antibody can also be further modified to increase or decrease binding affinity to the Fc receptor (FcR). In one embodiment, the antibody is further modified to have a) an antigen binding activity comparable to, less than, or superior to the unmodified antibody; b) a chemical stability comparable to, less than, or superior to the unmodified antibody; c) a thermostability or thermotolerance comparable to, less than, or superior to the unmodified antibody; d) a pH tolerance comparable to, less than, or superior to the unmodified antibody; e) a reduced immunogenicity; f) a reduced aggregation; g) an increased half-life relative to the unmodified antibody; h) an increased expression in a host cell; i) a stability in pharmaceutical formulation comparable or superior to that of the unmodified antibody; j) an enhanced or diminished dimerization of Fc regions; or k) any combination thereof.

In some embodiments, an antibody of the invention has a) an antigen binding activity comparable to, less than, or superior to the unmodified antibody; b) a chemical stability comparable to, less than, or superior to the unmodified antibody; c) a thermostability or thermotolerance comparable to, less than, or superior to the unmodified antibody; d) a pH tolerance comparable to, less than, or superior to the unmodified antibody; e) a reduced immunogenicity; f) a reduced aggregation; g) an increased half-life relative to the unmodified antibody; h) an increased expression in a host cell; i) a stability in pharmaceutical formulation comparable or superior to that of the unmodified antibody; j) an enhanced or diminished dimerization of Fc regions; or k) any combination thereof.

In one embodiment, the antibody of the invention maintains its native conformation at about pH 6.5, pH 6, pH 5.5, pH 5, pH 4.5, pH 4 or pH 3 or more acidic (a lower pH) or is further modified to do so. In another specific embodiment, the antibody retains biological activity (e.g., antigen binding) at about pH 6.5, pH 6, pH 5.5, pH 5, pH 4.5, pH 4 or pH 3 or more acidic (a lower pH) or is further modified to do so. In some embodiments, the antibody can further comprise additional mutations that render the antibody more, or less, resistant to pH dependent unfolding.

In some embodiments, the proteolysis inhibited by the antibody modifications of the invention includes digestion mediated by proteases from the gastrointestinal track, the blood, or the bile. In alternative embodiments, the proteolysis is mediated by pepsin, pancreatin, trypsin, trypsinogen, chymo-trypsinogen, carboxy-peptidase, pro-carboxy-peptidase, elastase, pro-elastase, or any combination thereof. The protease can be selected from a group of proteases released or produced by an exogenous organism or any organism within the digestive tract or released or produced within the digestive tract, e.g., by cells within the tract. In some embodiments, the proteases inhibited by the antibody modifications of the invention include proteases released or produced by an abnormal, infected, cancerous or otherwise diseased tissue.

In some embodiments, an antibody of the invention, or an antibody used in the methods of the invention, specifically binds to a pathogen. The pathogen can be a bacterium, a virus and a fungus. In some cases, the pathogen is an intestinal pathogen, including but not limited to enterotoxigenic E. coli, rotavirus, Cryptosporidium parvum, Clostridium difficile, Shigella flexneri, Enterococcus faecalis, Enterococcus faecium, Campylobacter jejuni, Staphylococcus aureus, E. coli O157:H7, Helicobacter pylori, Pseudomonas aeruginosa, Shigella dysenteriae, Salmonella enteritidis, Salmonella typhi, Clostridium perfringens, Aeromonas hydrophila, and Aeromanas aerolysin. In some embodiments, the pathogen is Streptococcus mutans.

In one aspect, an antibody of the invention, or an antibody used in the methods of the invention, specifically binds to a toxin. The toxin can be a bacterial toxin, a chemical toxin or an environmental toxin. In some embodiments the bacterial toxin is a cholera toxin, an Escherichia coli toxin, a Streptococcus toxin, a Bordetella pertussis toxin, and a Clostridium toxin. The Clostridium toxin can comprise a botulinum toxin or a Clostridium difficile toxin. The botulinum toxin or Clostridium difficile toxin can comprise botulinum neurotoxin, C. difficile toxin A, or C. difficile toxin B.

An antibody of the invention, or an antibody used in the methods of the invention, can specifically bind a virulence factor. The virulence factor can be an adherence factor, a coat protein, an invasion factor, a capsule, an exotoxin, or an endotoxin. An antibody of the invention can specifically bind to a dietary enzyme. The dietary enzyme can be a lipase, an esterase, a urease, a lyase, a protease, an isomerase, a ligase or a synthetase.

In another aspect, the invention provides an isolated or recombinant nucleic acid comprising a sequence encoding an antibody of the invention, a vector comprising the encoding nucleic acid, and a cell comprising the encoding nucleic acid or the vector comprising the encoding nucleic acid.

In one aspect, the invention provides a pharmaceutical composition comprising an antibody of the invention, or an antibody used in or made by a method of the invention, and a suitable excipient. In some embodiments, the composition is formulated as a suspension, a liquid, a capsule, a tablet, a gel, a powder, a microsphere, a liposome, a multiparticulate core particle or a spray. In one embodiment, the antibody comprises from about 1%, 5%, 10%, 20%, 30%, 40%, 50%, 60%, 70%, 80%, 90% or 95% or more, or from about 50% to about 95% of the batch size (weight/weight) of the pharmaceutical composition. In some embodiments, the composition is formulated for enteric delivery. In one embodiment, the pharmaceutical composition further comprises an enteric coating.

In another aspect, the invention provides a method of ameliorating, treating or preventing gastrointestinal infections or other disorders caused by a pathogen or a toxin comprising administering orally a pharmaceutically effective amount of the antibody of invention, or the pharmaceutical composition comprising an antibody of the invention, or an antibody used in or made by a method of the invention, to a subject in need thereof, whereby the infection or other disorders is treated or prevented.

In yet another aspect, the invention provides a kit for ameliorating or preventing one or more symptoms of virulence factor-associated symptom or disease, comprising a) a pharmaceutical composition comprising an antibody of the invention, or an antibody used in or made by a method of the invention; and b) instruction for administering the pharmaceutical composition.

In one aspect, the invention also provides a method of identifying a protease cleavage site in an antibody, which method comprises the steps of: a) determining putative sites of protease cleavage in the antibody; b) prioritizing the protease cleavage sites based on the likely exposure of the site to proteases; and c) identifying a site as the protease cleavage site as one whose position results in an exposure to proteases in the three-dimensional antibody structure. In some embodiments, the putative sites of protease cleavage are determined in step (a) by identifying protease cleavage motifs using N-terminal sequencing, gel electrophoresis analysis, or mass spectral analysis of peptide fragments derived from an antibody digested by protease. The putative sites of protease cleavage can also be determined in step (a) by identifying known protease motifs. In some embodiments, the protease cleavage sites are prioritized in step (b) based on the surface exposure on the folded form of the antibody solved by x-ray crystallography or NMR spectroscopy. The protease cleavage sites can also be prioritized in step (b) based on the surface exposure determined using a probe of 1.4 angstroms.

In alternative aspects, one, several or all steps of this method are carried out as a computer implemented method. Thus, the invention also provides a computer program product having embedded thereon code for a computer implemented method for identifying a protease cleavage site in an antibody; where in one aspect the method comprises the steps of: a) determining putative sites of protease cleavage in the antibody; b) prioritizing the protease cleavage sites based on the likely exposure of the site to proteases; and c) identifying a site as the protease cleavage site as one whose position results in an exposure to proteases in the three-dimensional antibody structure. Also provided is an article (e.g., a product of manufacture, e.g., a computer) comprising a machine-readable medium including machine-executable instructions, the instructions being operative to cause a machine to practice a method of this invention. A computer comprising this computer program product is also provided.

In some embodiments, the identified protease cleavage site has 20% surface area exposure to the probe, wherein the protease cleavage site comprises hydrophobic and aromatic amino acids. In other embodiments, the identified protease cleavage site has 35% surface area exposure to the probe, wherein the protease cleavage site comprises basic amino acids.

Any number of protease sites can be identified by the method of the invention. In one aspect, at least one protease cleavage site is identified. In some embodiments, the protease cleavage sites comprise the same protease cleavage motif. In other embodiments, the protease cleavage sites comprise two or more different protease cleavage motifs. The protease cleavage sites can be identified in the Fc region, the Fab region, the hinge region, CL, CH₁, CH₂, CH₃, V_{L}, V_{H}, or a combination thereof. The identified protease cleavage motifs include, but are not limited to, a protease selected from the group consisting of pepsin, pancreatin, trypsin, trypsinogen, chymo-trypsin, pro-carboxy-peptidase and pro-elastase.

In one aspect, the invention provides a method of engineering a protease-resistant antibody, which method comprises the steps of: a) providing an antibody or an amino acid sequence of the antibody; b) identifying at least one protease cleavage site in the amino acid sequence of the antibody; and c) introducing at least one modification in the amino acid sequence of the antibody, whereby the modification results in a variant portion that has an increased resistance to proteolysis.

In another aspect, the invention provides a method of generating an engineered antibody that is orally deliverable, which method comprises the steps of: a) providing a nucleic acid encoding a wildtype antibody; b) introducing at least one modification into the coding sequence of the wildtype antibody to generate a modified antibody coding sequence, wherein the modification of the coding sequence is in or proximate to the coding sequence of at least one protease cleavage site and the modification results in expression of an antibody that is partially or completely resistant to digestion by the protease; and c) expressing the modified antibody coding sequence of step b) to generate an engineered antibody, wherein the engineered antibody retains its ability to specifically bind to antigen in the digestive system following oral administration, thereby rendering the engineered antibody orally deliverable.

In some embodiments, the modification is in a protease cleavage site or at a site flanking the protease cleavage site. In alternative embodiments, the modification is at the P1, P1′, P2, P3, P4, P2′, P3′, or P4 residue of the protease cleavage site. The modification to the amino acid sequence generates a protease resistance motif, rendering the protease cleavage site non-cleavable or less susceptible to protease cleavage.

An engineered antibody of the invention can comprise any number of modifications, including but not limited to, two, three, four, five, six, seven, eight, nine, ten, eleven, or more amino acid modifications. The modifications can be in a protease cleavage site or at a site flanking the protease cleavage site. The modification can be made to the same protease cleavage motif within the antibody or to different protease cleavage motifs. In some embodiments, the modification is made in a protease cleavage site that is not flanked by an amino acid residue known to inhibit or attenuate protease cleavage. Such amino acids include Pro, Lys, Arg and His.

An engineered antibody of the invention can comprise an IgG, IgM, IgD, IgE, or IgA antibody. In some embodiments, the antibody is an IgG antibody. The antibody can be an IgG₁, IgG₂, IgG₃, or IgG₄ antibody. The antibody can be a human, murine, rat, rabbit, bovine, camel, llama, dromedary, or simian antibody. The antibody can be a humanized antibody, a chimeric antibody, a bispecific antibody, a fusion protein, or a biologically active (e.g., antigen binding) fragment thereof.

An engineered antibody of the invention can be modified in any portion of the antibody including the heavy chain, a light chain, or both. In some embodiments, the modified portion is the Fc region, the hinge region, the CH_{L} domain, the CH₁ domain, the CH₂ domain, the CH₃ domain, the Fab region, or any combination thereof. In alternative embodiments, the modified portion is a V_{H} or V_{L} domain, provided the cleavage site does not have a negative effect on the desired antibody function.

The modifications in the antibody of the invention comprise at least one mutation in the amino acid sequence of the antibody. The mutation is introduced by modifications, additions or deletions to a nucleic acid encoding the antibody. The modifications, additions or deletions to a nucleic acid encoding the antibody can be introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturation Mutagenesis (GSSM), synthetic ligation reassembly (SLR) or a combination thereof. The modifications, additions or deletions to a nucleic acid encoding the antibody can also be introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, or a combination thereof.

In one embodiment, an engineered antibody of the invention comprises at least one amino acid substitution at any one or more of amino acid positions T155, L179, L235, F241, Y296, L309, Y349, L365, L398, F404, Y407, and Y436 of an IgG heavy chain, e.g., SEQ ID NO:1, SEQ ID NO:3 and/or SEQ ID NO:5, wherein the numbering of the residues in the variant portion is that of the EU index as in Kabat, whereby the amino acid substitution confers increased resistance to pepsin proteolysis. In another embodiment, the variant portion comprises at least one amino acid substitution at any one or more of amino acid positions L234, L242, F243, F275, Y278, Y300, L306, W313, L314, Y319, L351, L368, Y391, F405, L406, L410, F423, L432, or Y436 of, e.g., SEQ ID NO:1, SEQ ID NO:3 and/or SEQ ID NO:5, wherein the numbering of the residues in the variant portion is that of the EU index as in Kabat, whereby the amino acid substitution confers increased resistance to pepsin proteolysis.

In one embodiment, the variant portion comprises at least one amino acid substitution at any one or more of amino acid positions F116, K126, R143, K169 or K183 of a kappa chain, e.g., SEQ ID NO:2, SEQ ID NO:4 and/or SEQ ID NO:6, wherein the numbering of the residues in the variant portion is that of the EU index as in Kabat, whereby the amino acid substitution confers increased resistance to pancreatin proteolysis.

In another embodiment, the variant portion comprises at least one amino acid substitution at any one or more of amino acid positions K133, K205, K210, K274, K326, K340, R355, K360 or K392 of, e.g., SEQ ID NO:1, SEQ ID NO:3 and/or SEQ ID NO:5, wherein the numbering of the residues in the variant portion is that of the EU index as in Kabat, whereby the amino acid substitution confers increased resistance to pancreatin proteolysis.

In one aspect, an engineered antibody of the invention comprises at least one amino acid substitution at the P1 or P1′ site of cleavage in a trypsin cleavage motif, wherein the substituted amino acid is K or R, whereby the amino acid substitution confers increased resistance to trypsin proteolysis. In one embodiment, an engineered antibody comprises at least one amino acid substitution, at the P1 or P1′ site of cleavage in a pepsin cleavage motif, wherein the substituted amino acid is L, F, Y, W, I, or T, whereby the amino acid substitution confers increased resistance to pepsin proteolysis. In some embodiments, the engineered antibody comprises at least one amino acid substitution at the P1 or P1′ site of cleavage in a chymotrypsin cleavage motif, wherein the substituted amino acid is F, Y, or W, whereby the amino acid substitution confers increased resistance to chymotrypsin proteolysis.

In one embodiment, an engineered antibody of the invention comprises at least one amino acid substitution selected from the group of amino acid substitutions of L235P, L398Q, F404Y, L179I, and T155S in an IgG₁ heavy chain, wherein the numbering of the residues in an engineered antibody is that of the EU index as in Kabat, whereby the amino acid substitution(s) confer increased resistance to pepsin proteolysis. In another specific embodiment, an engineered antibody comprises at least one amino acid substitution selected from the group of amino acid substitutions of F116S and K126A in a kappa light chain, wherein the numbering of the residues in an engineered antibody is that of the EU index as in Kabat, whereby the amino acid substitution confers increased resistance to pepsin proteolysis. In yet another specific embodiment, an engineered antibody comprises at least one amino acid substitution selected from the group of amino acid substitutions of K133G and K274Q in an IgG heavy chain, wherein the numbering of the residues in the engineered antibody is that of the EU index as in Kabat, whereby the amino acid substitution confers increased resistance to pepsin proteolysis.

In some embodiments, an engineered antibody of the invention (including any antibody made by a method of the invention, in addition to those disclosed herein) has greater resistance to proteolysis relative to the wildtype antibody. In one aspect, the increased resistance to proteolysis is at least 1%, 2%, 3%, 4%, 5%, 10%, 15%, 20%, 25%, 30%, 40%, 50%, 60%, 70%, 80% or more than that of the unmodified (e.g., unaltered, or “wildtype”) antibody. An engineered antibody can be partially or completely resistant to cleavage by more than one protease.

An engineered antibody of the invention (including any antibody made by a method of the invention, in addition to those disclosed herein) can be modified (including further modified) in any suitable manner. In some embodiments, the modification comprises the addition of a post-translational modification site, an N-glycosylation site, an O-glycosylation site, an alkyl chain, or a small molecule. At times, the modification comprises covalent or non-covalent addition of a second molecule to the Fc chain of the antibody. The second molecule comprises an antibody secretory component, a carbohydrate, a disulfide bond site, or a salt bridge site.

In some embodiment, the Fc region of an engineered antibody of the invention is further modified to elliance antibody-dependent cellular cytotoxicity (ADCC), complement-deperident cytotoxicity (CDC) and/or phagocytosis. The Fc region of the antibody can also be further modified to increase binding affinity to the Fc receptor (FcR). In one embodiment, an engineered antibody is further modified to have a) an antigen binding activity comparable to or superior to the unmodified antibody; b) a chemical stability comparable to or superior to the unmodified antibody; c) a thermostability or thermotolerance comparable to or superior to the unmodified antibody; d) a pH tolerance comparable to or superior to the unmodified antibody; e) a reduced immunogenicity; f) a reduced aggregation; g) an increased half-life relative to the unmodified antibody; h) an increased expression in a host cell; i) a stability in pharmaceutical formulation comparable or superior to that of the unmodified antibody; j) an enhanced dimerization of Fc regions; or k) any combination thereof.

In some embodiments, an antibody of the invention has a) an antigen binding activity comparable to or superior to the unmodified antibody; b) a chemical stability comparable to or superior to the unmodified antibody; c) a thermostability or thermotolerance comparable to or superior to the unmodified antibody; d) a pH tolerance comparable to or superior to the unmodified antibody; e) a reduced immunogenicity; f) a reduced aggregation; g) an increased half-life relative to the unmodified antibody; h) an increased expression in a host cell; i) a stability in pharmaceutical formulation comparable or superior to that of the unmodified antibody; j) an enhanced dimerization of Fc regions; or k) any combination thereof.

In one embodiment, an engineered antibody of the invention is modified to maintain (is modified such that it maintains) its native, or a least a functional (antigen-binding), conformation at about pH 6.5, pH 6, pH 5.5, pH 5, pH 4.5, pH 4 or pH 3 or more acidic (a lower pH). In another specific embodiment, the antibody retains at least some biological activity (antigen-binding) at pH 6.5, pH 6, pH 5.5, pH 5, pH 4.5, pH 4 or pH 3 or more acidic conditions. In some embodiments, the antibody can further comprise additional mutations that render the antibody more resistant to pH dependent unfolding. In one embodiment, an engineered antibody of the invention is modified to maintain (is modified such that it maintains) its native, or a least a functional (antigen-binding), conformation at alkaline conditions, e.g., pH 7.5, pH 8.0, pH 8.5, pH 9, pH 9.5, pH 10, pH 10.5 or pH 11. In one aspect, non-natural amino acids are incorporated into an antibody of the invention to farther increase resistance to pH dependent unfolding or resistance to proteases, e.g., see U.S. Patent App. No. 20050260711.

In some embodiments, the proteolysis is the digestion mediated by proteases from the gastrointestinal tract, the blood, or the bile. In alternative embodiments, the proteolysis is mediated by pepsin, pancreatin, trypsin, trypsinogen, chymo-trypsinogen, carboxy-peptidase, pro-carboxy-peptidase, elastase, pro-elastase, or any combination thereof. The protease can be selected from a group of proteases released or produced by an exogenous organism or any organism within the digestive tract, or released or produced in the digestive tract. In some embodiments, the protease can be selected from a group of proteases released by an abnormal, infected, cancerous or otherwise diseased tissue.

In some embodiments, an engineered antibody of the invention specifically binds to a pathogen. The pathogen can be a bacterium, a virus and a fungus. In some cases, the pathogen is an intestinal pathogen, including but not limited to enterotoxigenic E. coli, rotavirus, Cryptosporidium parvum, Clostridium difficile, Shigella flexneri, Enterococcus faecalis, Enterococcus faecium, Campylobacter jejuni, Staphylococcus aureus, E. coli O157:H7, Helicobacter pylori, Pseudomonas aeruginosa, Shigella dysenteriae, Salmonella enteritidis, Salmonella typhi, Clostridium perfringens, Aeromonas hydrophila, and Aeromanas aerolysin. In some embodiments, the pathogen is Streptococcus mutans.

In one aspect, an engineered antibody of the invention specifically binds to a toxin. The toxin can be selected from the group consisting of a bacterial toxin, a chemical toxin and an environmental toxin. In some embodiments the bacterial toxin is a cholera toxin, an Escherichia coli toxin, a Streptococcus toxin, a Bordetella pertussis toxin, and a Clostridium toxin. The Clostridium toxin can comprise a botulinum toxin or a Clostridium difficile toxin. The botulinum toxin or Clostridium difficile toxin can comprise botulinum neurotoxin, C. difficile toxin A (see below), or C. difficile toxin B (see, e.g., U.S. Patent App. No. 20040028705).

An engineered antibody of the invention can specifically bind a virulence factor. The virulence factor can be an adherence factor, a coat protein, an invasion factor, a capsule, an exotoxin, or an endotoxin. An engineered antibody of the invention can specifically bind to a dietary enzyme. The dietary enzyme can be a lipase, an esterase, a urease, a lyase, a protease, an isomerase, a ligase or a synthetase.

In another aspect, the invention provides an isolated or recombinant nucleic acid comprising a sequence encoding an engineered antibody of the invention (including the antibodies disclosed herein), a vector comprising the encoding nucleic acid, and a cell comprising the encoding nucleic acid or the vector comprising the encoding nucleic acid.

In one aspect, the invention provides a pharmaceutical composition comprising an engineered antibody of the invention (including the antibodies disclosed herein, and antibodies used in or made by a method of the invention), and a suitable excipient. In some embodiments, the composition is formulated as a suspension, a liquid, a capsule, a tablet, a gel, a microsphere, a liposome, a multiparticulate core particle or a spray. In one embodiment, the antibody comprises from about 1%, 5%, 10%, 20%, 30%, 40%, 50%, 60%, 70%, 80%, 90% or 95% or more, or from about 50% to about 95%, of the batch size (weight/weight) of the pharmaceutical composition. In some embodiments, the composition is formulated for enteric delivery. In one embodiment, the pharmaceutical composition further comprises an enteric coating.

In another aspect, the invention provides a method of ameliorating, treating or preventing gastrointestinal infections or other disorders caused by a pathogen or a toxin comprising administering orally a pharmaceutically effective amount of an engineered antibody of invention, or antibodies made by a method of the invention, or the pharmaceutical composition comprising an antibody of the invention, to a subject in need thereof, whereby the infection or other disorders is treated or prevented.

In yet another aspect, the invention provides a kit for ameliorating or preventing one or more symptoms of toxin-associated or virulence factor-associated symptom or disease, comprising a) the pharmaceutical composition comprising an engineered antibody of the invention (including antibodies made by a method of the invention, and the exemplary antibodies disclosed herein); and b) instruction for administering the pharmaceutical composition.

The invention provides methods for ameliorating and/or preventing toxicity associated with Clostridium difficile, comprising administering to a subject in need thereof: a) a therapeutically effective amount of a first monoclonal antibody (or equivalent synthetic Abs), wherein the first monoclonal antibody comprises the heavy chain variable region sequence of SEQ ID NO:1 and the light chain variable region sequence of SEQ ID NO:2; and in one aspect, the remainder of the antibody (e.g., the constant region) comprises human Ab sequence; and b) a therapeutically effective amount of a second monoclonal antibody (or equivalent synthetic Ab), wherein the second monoclonal antibody comprising the heavy chain variable region sequence of SEQ ID NO:3 and the light chain variable region sequence of SEQ ID NO:4 and in one aspect, the remainder of the antibody (e.g., the constant region) comprises human Ab sequence, whereby these antibodies ameliorate or prevent the toxicity associated with Clostridium difficile toxin A. In one embodiment, the method further comprises administering a third monoclonal antibody (or equivalent synthetic Ab), wherein the third antibody is a monoclonal antibody comprising the heavy chain variable region sequence of SEQ ID NO:5 and the light chain variable region sequence of SEQ ID NO:6, and in one aspect the remainder of the antibody (e.g., the constant region) comprises human Ab sequence, whereby the antibodies ameliorate or prevent the toxicity associated with Clostridium difficile toxin B.

In another aspect, the invention provides a method of ameliorating or preventing toxicity associated with Clostridium difficile, comprising administering to a subject in need thereof: a) a first antibody that partially or completely inhibits binding of a Clostridium difficile toxin A to a cell; and b) a second antibody that partially or completely inhibits intracellular internalization of the Clostridium difficile toxin A, wherein the first antibody and the second antibody bind to the Clostridium difficile toxin A at non-overlapping epitopes. In one embodiment, the method farther comprises administering a therapeutically effective amount of at least a third antibody that partially or completely neutralizes Clostridium difficile toxin B. In one embodiment, the second antibody is not the monoclonal antibody PCG-4 (see discussion below; Lyerly (1986) Infect Immun. 54:70-76). The invention also provides pharmaceutical compositions comprising these combinations of antibodies which are, in one aspect, formulated for oral administration.

In some embodiments, the first and second antibodies synergize to neutralize the toxin (e.g., virulence factor) at an antibody concentration lower than the antibody concentration necessary to observe partial neutralization by each antibody alone. In one embodiment, the first monoclonal antibody and the second monoclonal antibody bind to a Clostridium difficile toxin A at ToxA:1800-2710. In some embodiments, the third antibody is a monoclonal antibody that binds to a Clostridium difficile toxin B at ToxB:1807-2366. In one aspect, the first monoclonal antibody and the second monoclonal antibody do not bind Clostridium difficile toxin B, and the third monoclonal antibody does not bind Clostridium difficile toxin A.

The invention provides methods for ameliorating or preventing toxicity associated with a bacterial toxin, comprising administering to a subject in need thereof (a) a first antibody that partially or completely inhibits binding of the bacterial toxin to a cell; and (b) a second antibody that partially or completely inhibits intracellular internalization of the toxin, wherein the first antibody and the second antibody bind to the toxin at non-overlapping epitopes. In one aspect, the bacterial toxin comprises a Clostridium difficile toxin A or a Clostridium difficile toxin B. In one aspect, the first and the second antibodies are formulated together in a pharmaceutical composition. The first and the second antibodies can be formulated for oral administration.

The invention provides pharmaceutical compositions comprising (a) a first antibody that partially or completely inhibits binding of the bacterial toxin to a cell; and (b) a second antibody that partially or completely inhibits intracellular internalization of the toxin, wherein the first antibody and the second antibody bind to the toxin at non-overlapping epitopes. In one aspect, the bacterial toxin comprises a Clostridium difficile toxin A or a Clostridium difficile toxin B. In one aspect, the first and the second antibodies are formulated together in a pharmaceutical composition. In one aspect, the first and the second antibodies are formulated for oral administration.

The methods of the invention can be useful in the treatment of the Clostridium toxin-related toxicity in a subject, wherein the toxicity comprises Clostridium-associated diarrhea, colitis or a related condition, whereby one or more symptoms of the Clostridium-induced diarrhea, colitis, or related condition are ameliorated or prevented following administration of a pharmaceutical composition of the invention, e.g., a composition comprising one or more monoclonal antibodies of the invention, or an antibody modified by a method of the invention.

In one embodiment, the methods of the invention employ monoclonal antibodies comprising recombinant or synthetic antibodies. One or more of the antibodies can be rendered partially or completely resistant to proteolysis and/or orally deliverable using the antibody engineering methods of the invention.

In another aspect, the invention provides a monoclonal antibody, or a biologically active (e.g., antigen binding) fragment thereof (or equivalent synthetic Abs), that binds to Clostridium difficile toxin A, wherein the variable region sequences of the antibody comprise SEQ ID NO:1 and/or SEQ ID NO:2; and/or SEQ ID NO:3 and/or SEQ ID NO:4 (and in alternative aspects, the remainder of the antibody—such as the constant region—comprises human Ab sequence). The invention also provides an isolated or recombinant nucleic acid comprising a sequence encoding the antibody, a vector comprising the nucleic acid, and a cell comprising the nucleic acid or the vector. Pharmaceutical compositions and kits comprising the antibody are also provided.

The invention provides a monoclonal antibody, or a biologically active (e.g., antigen binding) fragment thereof (or equivalent synthetic Abs), that binds to Clostridium difficile toxin B, wherein the variable region sequences of the antibody comprise SEQ ID NO:5 and/or SEQ ID NO:6 (and in alternative aspects, the remainder of the antibody—such as the constant region—comprises human Ab sequence). The invention also provides an isolated or recombinant nucleic acid comprising a sequence encoding the antibody, a vector comprising the nucleic acid, and a cell comprising the nucleic acid or the vector. Pharmaceutical compositions and kits comprising the antibody are also provided.

The antibodies of the invention can comprise an IgG antibody or fragments thereof. In one embodiment, the antibody comprises a human, murine, rat, rabbit, camel, bovine, llama, dromedary, or simian antibody. In some embodiments, the antibody comprises a humanized antibody, chimeric antibody, bispecific antibody, fusion antibody, a minibody or nanobody, a bivalent scFv (i.e., a diabody, having two chains and two binding sites, and may be monospecific or bispecific), a triabody (three single chain antibodies), scFv, or biologically active (e.g., antigen binding) fragments thereof (see, e.g., U.S. Patent App. Pub. No. 20050234225).

An antibody can be modified to increase resistance to proteolysis using the methods of the invention. The antibody can be modified to be orally deliverable, using, for example, the methods of the invention. The antibody can be modified to abrogate, diminish or enhance antibody-mediated cytotoxicity (ADCC), a complement-mediated cytotoxicity (CDC), or phagocytosis. In some embodiments, the Fc region of the antibody is modified to abrogate, diminish or increase (enhance) binding affinity to the Fc receptor (FcR). In one embodiment, the antibody is modified to have: a) an antigen binding activity comparable to, less than or superior to the unmodified antibody; b) a chemical stability comparable to, less than or superior to the unmodified antibody; c) a thermostability or thermotolerance comparable to, less than or superior to the unmodified antibody; d) a pH tolerance comparable to or superior to the unmodified antibody; e) a reduced immunogenicity; f) a reduced aggregation; g) an increased half-life relative to the unmodified antibody; h) an increased expression in a host cell; i) a stability in pharmaceutical formulation comparable or superior to that of the unmodified antibody; j) an enhanced dimerization of Fc regions; or k) any combination thereof. In another embodiment, the antibody has: a) an antigen binding activity comparable to or superior to the unmodified antibody; b) a chemical stability comparable to or superior to the unmodified antibody; c) a thermostability or thermotolerance comparable to or superior to the unmodified antibody; d) a pH tolerance comparable to or superior to the unmodified antibody; e) a reduced immunogenicity; f) a reduced aggregation; g) an increased half-life relative to the unmodified antibody; h) an increased expression in a host cell; i) a stability in pharmaceutical formulation comparable or superior to that of the unmodified antibody; j) an enhanced dimerization of Fc regions; or k) any combination thereof.

In one aspect, the invention provides a monoclonal antibody produced by or isolated from a hybridoma selected from the group consisting of ATCC Accession No. ______ (Ab designated 227 or 3359), ATCC Accession No. ______ (Ab designated 543 or 3358), ATCC Accession No. ______ (Ab designated F85), ATCC Accession No. ______ (Ab designated F2), and ATCC Accession No. ______ (Ab designated F87) (or equivalent synthetic Abs). Also provided herein is a synthetic, isolated or recombinant antibody, wherein the antibody has the same antigen binding specificity (e.g., binds to the same epitope) as a monoclonal antibody of the invention, or antibodies made by a method of the invention, including, e.g., antibodies having the same antigen binding specificity as the Ab designated 227 or 3359, the Ab designated 543 or 3358, the Ab designated F85, the Ab designated F2 and/or the Ab designated F87, or, a chimeric (e.g., “humanized”) antibody having the same sequence or binding specificity as an antibody comprising the (light/heavy) variable region pairs SEQ ID NO:1 and SEQ ID NO:2, or SEQ ID NO:3 and SEQ ID NO:4, or SEQ ID NO:5 and SEQ ID NO:6.

The methods and compositions of the invention greatly increase the therapeutic efficacy of antibodies by increasing the stability of the antibody following administration to the patient.

The details of one or more aspects of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### BRIEF DESCRIPTION OF DRAWINGS

The following drawings are illustrative of aspects of the invention and are not meant to limit the scope of the invention as encompassed by the claims.

FIG. 1 shows the pepsin digestion profile of IgG₁, IgG₂, IgG₃, and IgG₄, as discussed in Examples 1 and 3, below.

FIG. 2 depicts the pepsin digestion profile of IgG₁ on a reducing gel, as discussed in Example 1, below.

FIG. 3 shows the chimeric antibody rPBA3 after it was expressed in mammalian cells, purified, and dialyzed, as discussed in detail in Example 1, below.

FIG. 4 depicts the pH dependence of IgG₁ structure demonstrated by Circular Dichroism (CD) experiments, as discussed in Example 1, below.

FIGS. 5A and 5B show the degradation of rPBA-3 by acid, pepsin and high temperature, as discussed in detail in Example 1, below.

FIGS. 6A and 6B shows the pepsin digestion profile of wildtype (unaltered) and mutant antibodies at pH 1.2, as discussed in detail in Example 1, below.

FIGS. 7A and 7B illustrates pepsin digestion profile of wildtype and mutant antibodies at pH 3.0, as discussed in detail in Example 1, below.

FIG. 8A illustrates the CLUSTALW alignment of repeat domains of several CWBs, as discussed in detail in Example 2, below. FIG. 8B illustrates spectra of toxin A and toxin B repeat domains, as discussed in detail in Example 2, below. FIG. 8C illustrates the temperature dependence of CWB-domain structure, as discussed in detail in Example 2, below.

FIG. 9 illustrates photographs of adherent CHO cells cultured in the absence (media only) and presence of 20 ng (100 μL total volume) toxin A with and without anti-toxin A antibodies present, as discussed in detail in Example 2, below.

FIGS. 10A, 10B, 10C, 10D and 10E illustrates the antibody competition for toxin binding sites using static concentrations of toxin and titrating the amount of antibody in solution, as discussed in detail in Example 2, below.

FIG. 11A illustrates the thermal denaturation of ToxA:2459-2710 in the absence and presence of CWB-binding ligands monitored by the CD signal of the protein at 230 nm, as illustrated in FIG. 11B-E, as discussed in detail in Example 2, below.

FIG. 12 depicts the attenuation of cell surface binding of ToxA: 2459-2710 by anti-toxin A antibodies as determined by flow cytometry, as discussed in detail in Example 2, below.

FIG. 13 illustrates the ileal loop model, as discussed in detail in Examples 2 and 4, below.

FIGS. 14A to D show the activity of anti-Clostridium difficile toxin A antibodies in the ileal loop model, as discussed in detail in Examples 2 and 4, below.

FIGS. 15A to F illustrate the histology of rat intestinal mucosa, as discussed in detail in Example 4, below.

FIG. 16 shows weight versus length measurement for rat ileal loops incubated with saline, 5 μg toxin A independently and in the presence of various concentrations of 3359 (or 227) and 543 antibodies (alone and in combination), as discussed in detail in Example 4, below.

FIG. 17(A) illustrates data showing the titration of antibody 3358 in the presence of fixed amounts of the 3359 antibody; FIG. 17(B) illustrates the titration of antibody 3359 in the presence of fixed amounts of the 3358 antibody, as discussed in detail in Example 5, below.

FIGS. 18A to D illustrate data for antibody competition for toxin binding site experiments, studied by surface plasmon resonance, as discussed in detail in Example 5, below.

FIGS. 19A to D illustrate data showing CHO cell surface binding of ToxA:11R, as determined by flow cytometry, as discussed in detail in Example 5, below.

FIGS. 20A to F illustrate SSC and fluorescence profile data showing the effect of anti-toxin A antibodies on ToxA:11R cell surface association; FIGS. 20A and 20C illustrate data showing that both the 3358 and rPCG-4 antibodies significantly increased the amount of CWB-domain detected at the cell surface; FIG. 20B illustrates data showing that the 3359 antibody inhibited cell surface association of ToxA:11R; FIG. 20D illustrates data showing that the combination of the 3359 and 3358 antibody inhibits ToxA:11R binding the CHO cell surface similar to the behavior of 3359 alone; as discussed in detail in Example 5, below.

FIGS. 21A to F illustrate photomicrographs of the histology of rat intestinal mucosa after treatment with toxin A with or without anti-toxin A antibodies, as discussed in detail in Example 5, below.

FIG. 22 graphically illustrates data from a rat ileal loop assay showing that antibodies 3359 and 3358 prevent toxin A-induced intestinal fluid secretion in rat ileal loops, as discussed in detail in Example 5, below.

FIG. 23 graphically illustrates data showing the efficacy of systemic dosing with anti-toxin A and anti-toxin B antibodies in C. difficile in hamsters, as discussed in detail in Example 5, below.

FIGS. 24A to F illustrate photomicrographs of the histology of hamster intestinal mucosa after C. difficile challenge, as discussed in detail in Example 5, below.

FIG. 25 (both panels) illustrates the results of the pepsin digestion profiles of IgG1, IgG2, IgG3 and IgG, as discussed in detail in Example 6, below.

FIGS. 26A and 26B illustrate data showing that acidic conditions alone in the absence of pepsin led to decreases in functional antibody, as discussed in detail in Example 6, below.

FIGS. 27A to D illustrate a graphic summary of data showing the pH dependence of IgG1 structure demonstrated by Circular Dichroism (CD) experiments, as discussed in detail in Example 6, below.

FIGS. 28A and B illustrate the pepsin digestibility of the wildtype antibody and the mutant combinations at acidic conditions where the molecule remained folded and the pepsin is still active; examples of pepsin digestions are shown in FIG. 28A; data summarized in FIG. 28B, as discussed in detail in Example 6, below.

FIG. 29 illustrates pictures of cells cultured in the presence or absence of toxin and toxin-neutralizing antibody after pepsin digestion, as discussed in detail in Example 6, below.

FIG. 30 illustrates pictures of gels showing pancreatin digestion profiles of IgG1, IgG2, IgG3 and IgG4, as discussed in detail in Example 6, below.

FIG. 31 in table form summarizes combinations of antibody mutations identified to confer resistance to pancreatin digestion, as discussed in detail in Example 6, below.

FIG. 32 illustrates data from a time course of IgG recovery from the stomach, cecum and distal colon after oral administration of antibody, as discussed in detail in Example 6, below.

FIG. 33 illustrates data from a time course of antibody recovery from mouse feces after oral administration of 1 mg of the optimized antibody and a control antibody, as discussed in detail in Example 6, below.

FIG. 34 depicts a time course of antibody recovery from mouse feces after oral administration of 2.5 mg of the optimized antibody and a control antibody, as discussed in detail in Example 6, below.

FIG. 35 illustrates a photograph of a Western blot analysis of samples described in FIG. 34, as discussed in detail in Example 6, below.

Like reference symbols in the various drawings indicate like elements.

### DETAILED DESCRIPTION

The invention provides antibodies modified to improve their therapeutic efficacy upon administration to a subject, e.g., after oral administration, and methods for making and using them. In one aspect, amino acid residues in the antibody are modified to improve the stability of the antibody. In one aspect, an antibody of the invention is modified or engineered to increase protease resistance, thereby reducing or eliminating sensitivity to proteolysis. In one aspect, the antibodies are further modified to alter other characteristics, such as thermotolerance, pH stability, binding affinity, immunogenicity, half-life, host cell expression and stability in pharmaceutical formulations that also contribute to therapeutic efficacy. For example, an antibody of the invention also can be modified to include post-translation modification sites, second molecules, disulfide bond sites, or salt bridges that enhance antibody stability upon administration, particularly by the oral route. The invention also provides methods for engineering such antibodies, e.g., by modifying the nucleic acid sequence that encodes the antibody.

Antibodies of the invention, including antibodies made by methods of the invention, and antibodies described herein, are useful in methods of ameliorating, treating or preventing a disease, infection, or other disorder caused by an abnormal cell, pathogen, or toxin comprising administering orally a pharmaceutically effective amount of the antibody. The antibody of the invention can be in the form of a pharmaceutical composition for administration to a subject in need thereof, e.g., to treat, prevent or ameliorate a disease, infection or other disorder.

An antibody of the invention can be co-administered with at least one bioactive agent or drug that can include, but are not limited to an antibiotic, a second antibody, a radionuclide, a chemotherapeutic agent, or a biologically active (e.g., antigen binding or toxic) protein. In some embodiments, the biologically active protein is a toxin-degrading or inactivating protease. Treatment of certain infectious diseases, such as Clostridium difficile, is particularly amenable to treatment with oral antibodies, including the antibodies of the invention, such as antibodies made by methods of the invention.

In one aspect, an antibody of the invention is co-administered with an agent that facilitates protease resistance or stability to harsh conditions, e.g., extremes in pH, such as the acidic conditions of the stomach and/or the alkaline conditions of the intestine.

Unless defined otherwise, all technical and scientific terms used herein have the same meaning as is commonly understood by one of ordinary skill in the art to which this invention belongs. All patents, applications, published applications and other publications and sequences from GenBank and other databases referred to herein are incorporated by reference in their entirety. If a definition set forth in this section is contrary to or otherwise inconsistent with a definition set forth in applications, published applications and other publications and sequences from GenBank and other data bases that are herein incorporated by reference, the definition set forth in this section prevails over the definition that is incorporated herein by reference.

As used herein, “a” or “an” means “at least one” or “one or more.”

“Antibody-dependent cell-mediated cytotoxicity” and “ADCC” refer to a cell-mediated reaction in which nonspecific cytotoxic cells that express FcRs; natural killer (NK) cells, neutrophils, and macrophages can recognize bound antibody on a target cell and subsequently lyse the target cell. See e.g., Ravetch (1991) Annu. Rev. Immunol. 9:457-92.

“Amino acid” or “amino acid sequence” include an oligopeptide, peptide, polypeptide, peptidomimetic or protein sequence, or to a fragment, portion, or subunit of any of these, and to naturally occurring or synthetic molecules that encodes an antibody of the invention, or biologically active (e.g., antigen binding) fragment thereof. The terms “polypeptide” and “protein” include amino acids joined to each other by peptide bonds or modified peptide bonds, i.e., peptide isosteres, and may contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms. The term “polypeptide” also includes peptides and polypeptide comprising non-natural residues.

The term “engineered protease site” refers to a protease site that has been modified from the naturally existing sequence by at least one amino acid substitution. The term “protease resistance motif” refers to a protease site that has been modified from the naturally existing sequence to generate a motif that is less susceptible or resistant to protease cleavage.

As used herein, the term “protease” refers to all polypeptides, e.g., enzymes, which catalyze the hydrolysis of peptide bonds. Protease activity includes hydrolyzing peptide bonds at high temperatures, low temperatures, alkaline pHs and at acidic pHs. The proteases can be naturally occurring, recombinantly generated, and/or synthetic. Exemplary proteases include pepsin, trypsin, trypsinogen, chymo-trypsin, pro-carboxy-peptidase, and pro-elastase.

The “nucleic acids” and “nucleic acid sequences” of the invention include oligonucleotides, nucleotides, polynucleotides or fragments of any of these, to e.g., DNA or RNA (e.g., mRNA, rRNA, tRNA) of genomic or synthetic origin which may be single-stranded or double-stranded which encodes an antibody of the invention, or a biologically active (e.g., antigen binding) fragment thereof. The term encompasses nucleic acids, i.e., oligonucleotides, containing known analogues of natural nucleotides. The term also encompasses nucleic-acid-like structures with synthetic backbones. See e.g., Mata, Toxicol. Appl. Pharmacol. 144:189-97 (1997); Strauss-Soukup, Biochemistry 36:8692-98 (1997); and Samstag, Antisense Nucleic Acid Drug Dev 6:153-56 (1996).

The term “isolated” includes a material (e.g., an antibody used to practice the invention) removed from its original environment, e.g., the natural environment if it is naturally occurring. For example, a naturally occurring polynucleotide or polypeptide present in a living animal is not isolated, but the same polynucleotide or polypeptide, separated from some or all of the coexisting materials in the natural system, is isolated. Such polynucleotides can be part of a vector and/or such polynucleotides or polypeptides can be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity.

The invention comprises isolated, recombinant or synthetic Ab light or variable region polypeptides that are “substantially identical” to an exemplary sequence of the invention, e.g., having a sequence identity to SEQ ID NO:1, SEQ ID NO:2, SEQ ID NO:3, SEQ ID NO:4, SEQ ID NO:5, SEQ ID NO:6, SEQ ID NO:7 and/or SEQ ID NO:8, and having the same (or substantially the same) antigen binding specificity, as discussed below.

A “substantially identical” amino acid sequence also can include a sequence that differs from a reference sequence (e.g., an exemplary sequence of the invention, e.g., an Ab sequence of the invention comprising the variable regions SEQ ID NO:1, SEQ ID NO:2, SEQ ID NO:3, SEQ ID NO:4, SEQ ID NO:5, SEQ ID NO:6, SEQ ID NO:7 and/or SEQ ID NO:8,) by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from an antibody, resulting in modification of the structure of the polypeptide, without significantly altering its biological activity. For example, amino- or carboxyl-terminal amino acids that are not required for antibody activity can be removed.

A “substantially identical” amino acid sequence also can include a sequence that hybridizes under stringent conditions to a reference sequence (e.g., an exemplary sequence of the invention, e.g., an Ab sequence of the invention comprising the variable regions SEQ ID NO:1, SEQ ID NO:2, SEQ ID NO:3, SEQ ID NO:4, SEQ ID NO:5, SEQ ID NO:6, SEQ ID NO:7 and/or SEQ ID NO:8), as discussed, below.

As used herein, the term “synergize” refers to the ability of one agent to increase the anti-pathogenic or neutralizing effect of a second agent. Synergistic activity, thus, includes but is not limited to an increased biological effect (e.g., more potent or longer lasting) using the two agents together that is not observed when the agents are used separately, a more effective biological effect, e.g., elimination of multiple types of toxicity not achievable with the administration of a single agent, or a reduction in the amount of agents necessary for administration to achieve the biological effect observed with a single agent.

The term “pathogen” refers to any organism that induces or elicits a undesired symptom or disease state. A pathogen may be a bacteria, virus, or fungus. The pathogen can be an organism residing at a site that has gained antibiotic resistance or has overgrown other flora.

As employed herein, the term “subject” embraces human as well as other animal species, such as, for example, canine, feline, bovine, porcine, rodent, and the like. It will be understood by the skilled practitioner that the subject having a pathogen or disease targeted by the antibody of the invention.

As used herein the term “ameliorating, treating or preventing” include a postponement of one or more symptoms associated with the gastrointestinal infection or other disorder, a reduction in the severity of such symptoms that will or are expected to develop, or a complete elimination of such symptoms. These terms further include ameliorating existing pathogen-related symptoms, reducing duration of disease, preventing additional symptoms, ameliorating or preventing serious sequelae, preventing or reversing mortality, reducing or preventing fecal shedding, and reducing or preventing pathogen transmission. Thus, the terms denote that a beneficial result has been conferred on a subject with a pathogen, or with the potential of exposure to such a pathogen. If the gastrointestinal infection or disorder is elicited or induced by a toxin, the term “ameliorating, treating or preventing” further includes inhibiting the activity of a toxin which is associated with the development of a particular disease state or medical condition. The microbial toxin can be an endotoxin or exotoxin produced by a microorganism, such as a bacterium, a fungus or a protozoan. The toxin can be inhibited by any mechanism, including, but not limited to, binding of the toxin by the antibody.

As used herein, a “therapeutically effective amount” or a “pharmaceutically effective amount” is an amount sufficient to inhibit or prevent, partially or totally, tissue damage or other symptoms associated with the action of the virulence factor within or on the body of the subject or to prevent or reduce the further progression of such symptoms. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

As used herein, the term “bioactive agent” refers to any synthetic or naturally occurring compound that binds the antigen and/or enhances or mediates a desired biological effect. Bioactive agents include, for example, a pharmaceutical agent, such as a chemotherapeutic drug, a toxin, a cytokine, a ligand, another antibody, regulatory moieties such as zinc fingers and leucine zippers, or any combination thereof. In one embodiment, the agent in an antitumor agent. As used herein, the term “antitumor agent” refers to agent that inhibits tumor growth through the induction of an immune response, stasis, cell death, senescence, apoptosis, ankoisis (constitutive epithelial cell apoptosis resulting from detachment from basement membrane) or necrosis.

### Antibody Compositions and Related Methods

The invention provides antibodies with improved therapeutic efficacy. In one aspect, the invention provides isolated, recombinant or synthetic antibodies comprising a variant portion and/or a constant region comprising at least one amino acid modification, wherein said variant portion results in an increased resistance to proteolysis. In one aspect, an antibody of the invention comprises the heavy chain variable region sequence encoded in SEQ ID NO:1 and the light chain variable region sequence encoded in SEQ ID NO:2, and the remainder of the antibody (e.g., constant region) comprises human sequence, thus making a “humanized” chimeric antibody. In alternative aspects, the “humanized” chimeric antibody comprises the variable region sequence combinations SEQ ID NO:3 and SEQ ID NO:4, or, SEQ ID NO:5 and SEQ ID NO:6, or, SEQ ID NO:7 and SEQ ID NO:8. The invention also provides novel combinations of monoclonal antibodies that when administered together have a synergistic anti-toxin effect, as described herein.

Any suitable method of generating an antibody to be modified using the methods of the invention, can be employed. Methods of immunization, producing and isolating antibodies (polyclonal and monoclonal) are known to those of skill in the art and described in the scientific and patent literature. See, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom Trends Biotechnol. 15:62-70 (1997); Katz (1997) Annu. Rev. Biophys. Biomol. Struct. 26:27-45 (1997).

In practicing the methods of the invention, in one aspect, any isolated or recombinant antibody or biologically active (e.g., antigen binding) fragment thereof, can be modified to increase the resistance to proteolysis. Antibodies can be isolated from natural sources, be synthetic, or be recombinantly generated polypeptides. The antibodies can be recombinantly expressed in vitro or in vivo. The antibodies of the invention can be made and isolated using any method known in the art. Antibodies of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., THERAPEUTIC PEPTIDES AND PROTEINS, FORMULATION, PROCESSING AND DELIVERY SYSTEMS (1995) Technomic Publishing Co., Lancaster, Pa. For example, antibody synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer. Exemplary descriptions of recombinant means of antibody generation and production include Delves, ANTIBODY PRODUCTION: ESSENTIAL TECHNIQUES (Wiley, 1997); Shephard, et al., MONOCLONAL ANTIBODIES (Oxford University Press, 2000); Goding, MONOCLONAL ANTIBODIES: PRINCIPLES AND PRACTICE (Academic Press, 1993); CURRENT PROTOCOLS IN IMMUNOLOGY (John Wiley & Sons, most recent edition).

Any form of the antigen can be used to generate the antibody that is sufficient to generate a specific antibody for a virulence factor or other antigen. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. In one embodiment, the DNA encoding the antigen may genomic or non-genomic (e.g., cDNA) and encodes at least one epitope in the extracellular domain of the antigen. Any genetic vectors suitable for transformation of the cells of interest may be employed, including but not limited to adenoviral vectors, plasmids, and non-viral vectors, such as cationic lipids. In one embodiment, the antibody of the methods and compositions herein specifically bind at least one epitope of the extracellular domain of the virulence factor of interest.

As used herein, the term “antibody” (“Ab”) refers to any form of a peptide, polypeptide or peptidomimetic derived from, modeled after or substantially encoded by, an immunoglobulin gene or immunoglobulin genes, or fragments thereof, capable of specifically binding an antigen or epitope. See, e.g., FUNDAMENTAL IMMUNOLOGY, Fifth Edition, W. E. Paul, ed., Lipincott, Williams & Wilkins (2003); Wilson (1994) J. Immunol. Methods 175:267-273; Yarmush (1992) J. Biochem. Biophys. Methods 25:85-97. Examples of antibody fragments are those that retain antigen-binding and include Fab, Fab′, F(ab′)₂, Fd, and Fv fragments; diabodies; triabodies; linear antibodies; single-chain antibody molecules, e.g., sc-Fv; minibodies, nanobodies, minibodies and multispecific antibodies formed from antibody fragments. In alternative aspects, an Ab binding fragment or derivative retains at least 5%, 10%, 20%, 30%, 40%, 50%, 60%, 70%, 80%, 90%, 95%, 99% or 100% of its biological activity.

An antigen-binding fragment of the invention (encompassed by the term “antibody of the invention”) can include conservative amino acid substitutions or non-natural residues that do not substantially alter its binding and/or biologic activity. Antibodies of the invention also encompass monoclonal (including full length monoclonal antibodies), polyclonal, multispecific (e.g., bispecific), minibody, heteroconjugate, diabody, triabody, chimeric, humanized, human, murine, and synthetic antibodies as well as antibody fragments that specifically bind a desired antigen and exhibit a desired binding property and/or biological activity.

As used herein, the term “variant portion” refers to an amino acid sequence which differs from the native amino acid sequence of an antibody by virtue of at least one amino acid residue modification. A native (or “wildtype” or unaltered) amino acid sequence refers to the amino acid sequence of an antibody found in nature. A “variant portion” of the antibody includes any domain or region of the antibody that has an amino acid modification, or any subdomain or subregion thereof. “Variant portions” of the antibody include, but are not limited to the Fc region, the Fab region, the CH₁ domain, the CH₂ domain, the CH₃ domain, the hinge region, the variable region, the constant region, the light chain and/or the heavy chain.

As used herein, the term “specific” can refer to the selective binding of the antibody to the target antigen epitope. Antibodies can be tested for specificity of binding by comparing binding to appropriate antigen to binding to irrelevant antigen or antigen mixture under a given set of conditions. In one aspect, the antibody lacks significant binding to unrelated antigens.

The term “antigen” refers to a molecule which is specifically recognized and bound by an antibody. An antigen which elicits an immune response in an organism, as evidenced by production of specific antibodies within the organism is termed an “immunogen.” The specific portion of the antigen or immunogen which is bound by the antibody is termed the “binding epitope” or “epitope.”

An “amino acid modification” refers to a change in the amino acid sequence of a predetermined amino acid sequence. Exemplary modifications include an amino acid substitution, insertion and/or deletion. In one aspect of the methods of the invention, an amino acid modification comprises an amino acid residue substitution. An amino acid modification at a specified position, e.g. of the Fc region, refers to the substitution, deletion, or deletion of the specified residue where the numbering of the residues is that of the EU index in Kabat. See, e.g., Kabat, et al., Sequences of Proteins of Immunological Interest, Fifth Edition. NIH Publication No. 91-3242 (1991). Thus, a designation of F116S indicates that the phenylalanine (F) at position 116 is substituted with a serine (S) at position 116. See Example 1, below.

The term “chimeric” antibody refers to an antibody in which a portion of the heavy and/or light chain is identical with or homologous to corresponding sequences in an antibody derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity. See, e.g., U.S. Pat. No. 4,816,567; and Morrison et al., Proc. Natl. Acad. Sci. USA 81: 6851-6855 (1984). As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and in one aspect two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), or that of a human immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1; Queen et al. (1989) Proc. Nat'l Acad. Sci. USA 86:10029-10033; and ANTIBODY ENGINEERING: A PRACTICAL APPROACH (Oxford University Press 1996).

In one aspect, an antibody of the invention, or an antibody used in a method of the invention, comprises a heterologous moiety that serves as a “shuttle”, “transport” or “carrier” moiety or domain to allow an antibody of the invention (or an antibody used in a method of the invention) enter cells (e.g., those lining the gut) or to allow an orally administered antibody of the invention enter into the bloodstream from the gut. In one aspect, a “shuttle”, “transport” or “carrier” moiety or domain comprises a transferrin polypeptide moiety (or active binding-internalization fragment thereof), Pseudomonas exotoxin (or active binding-internalization fragment thereof), a cell wall binding domain (CWB) domain of Clostridium difficile toxin A (or active binding-internalization fragment thereof), or an equivalent protein. For example, see Bai (2005) Proc. Natl. Acad. Sci. USA 102:7292-7296, describing use of transferrin as a “shuttle”, “transport” or “carrier” moiety, and that transferrin is a natural transport protein well known in the art; see also U.S. Pat. Nos. 6,891,028; 6,825,037; 6,743,893; 6,361,779. For example, the antibodies of the invention, or an antibody used in a method of the invention, can comprise a transferrin fragment (e.g., a human transferrin fragment), a peptide capable of binding to a transferrin receptor (e.g., a human transferrin receptor), thereby internalizing (into a cell); the sequences comprising HAIYPRH (SEQ ID NO:32) and THRPPMWSPVWP (SEQ ID NO:33); see, e.g., U.S. Pat. No. 6,743,893. When these peptides are fused with antibodies of the invention (e.g., as a recombinant chimeric polypeptide), the fusion product is internalized in cells expressing a transferrin receptor (e.g., a human transferrin receptor (hTfR)). The antibodies of the invention, or an antibody used in a method of the invention, also comprise recombinant fusions or heteromolecules (the Ab does not have to be recombinant, as the “shuttle”, “transport” or “carrier” moiety can be joined to the Ab by chemical or other means, too) with other known peptides or proteins that can effect the internalization of the chimeric polypeptide into a cell, e.g., examples of exemplary naturally occurring ligands that can be used as “shuttle”, “transport” or “carrier” moieties with Abs of the invention (and the receptors to which they bind) include: bombesin, gastrin, low density lipoprotein (LDL), epidermal growth factor (EGF), tumor necrosis factor (TNF), tumor growth factor (TGF), catecholamines (beta adrenergic receptors), asialofetuin (asialoglycoprotein receptor), somatostatin, N-formyl peptide, insulin, angiotensin, urokinase, carbachol (muscarinic receptors), folate, and/or insulin-like growth factor (IGF), or active binding-internalization fragments thereof, and the like; see U.S. Pat. No. 6,511,967.

In one aspect, a “shuttle”, “transport” or “carrier” moiety or domain comprises the translocation domain of a bacterial toxin, e.g., Clostridium difficile toxin A or toxin B, Pseudomonas exotoxin (e.g., Pseudomonas exotoxin A) (Trinity Biosystems, Menlo Park, Calif.), cholera toxin, ricin toxin or Shiga-like toxin, or active binding-internalization fragments thereof, or equivalent proteins, all of which are well known in the art; see, e.g., U.S. Pat. Nos. 6,022,950; 5,328,984; 5,080,898; 4,675,382; 4,666,837; 4,594,336.

In one embodiment, the antibody provided herein is a human antibody. In one aspect, the term “human antibody” refers to an antibody in which essentially the entire, or substantially all of, sequences of the light chain and heavy chain sequences, including the complementary determining regions (CDRs), are derived from human genes. However, human antibodies of the invention can also include non-natural or synthetic residues or peptidomimetic residues.

In one embodiment, human monoclonal antibodies are prepared by the trioma technique, the human B-cell technique (see, e.g., Kozbor, et al., Immunol. Today 4: 72 (1983), EBV transformation technique (see, e.g., Cole et al. MONOCLONAL ANTIBODIES AND CANCER THERAPY 77-96 (1985)), or using phage display (see, e.g., Marks et al., J. Mol. Biol. 222:581 (1991)). In one embodiment, the human antibody is generated in a transgenic mouse. Techniques for making such partially to fully human antibodies are known in the art and any such techniques can be used. In one aspect, fully human antibody sequences are made in a transgenic mouse engineered to express human heavy and light chain antibody genes. An exemplary description of preparing transgenic mice that produce human antibodies found in Application No. WO 02/43478. B cells from transgenic mice that produce the desired antibody can then be fused to make hybridoma cell lines for continuous production of the antibody. See, e.g., U.S. Pat. Nos. 5,569,825; 5,625,126; 5,633,425; 5,661,016; and 5,545,806; and Jakobovits, Adv. Drug Del. Rev. 31:33-42 (1998); Green, et al., J. Exp. Med. 188:483-95 (1998).

As used herein, the term “bispecific antibody” refers to an antibody, or a monoclonal antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al., Nature 305:537-39 (1983). Alternatively, bispecific antibodies can be prepared using chemical linkage. See, e.g., Brennan, et al., Science 229:81 (1985). Bispecific antibodies include bispecific antibody fragments. See, e.g., Hollinger, et al., Proc. Natl. Acad. Sci. U.S.A. 90:6444-48 (1993), Gruber, et al., J. Immunol. 152:5368 (1994).

As used herein, the term “heteroconjugate antibody” refers to two covalently joined antibodies. Such antibodies can be prepared using known methods in synthetic protein chemistry, including using crosslinking agents. See, e.g., U.S. Pat. No. 4,676,980.

As used herein, the term “single-chain Fv” or “scFv” antibody refers to antibody fragments comprising the V_{H} and V_{L} domains of antibody, wherein these domains are present in a single polypeptide chain. The Fv polypeptide can further comprises a polypeptide linker between the V_{H} and V_{L} domains, e.g., in one aspect this enables the sFv to form the desired structure for antigen binding. Designing and making scFVs and Fvs are well known in the art, see, e.g., Pluckthun, THE PHARMACOLOGY OF MONOCLONAL ANTIBODIES, vol. 113, Rosenburg and Moore eds. Springer-Verlag, New York, pp. 269-315 (1994). Techniques for the production of single chain antibodies (U.S. Pat. No. 4,946,778) also can be adapted to produce single chain antibodies of the invention, including fragments of exemplary antibodies or binding fragments comprising at least 5, 10, 15, 20,25, 30, 35, 40, 50, 75, 100, or 150 consecutive amino acids thereof. Alternatively, transgenic mice may be used to express antibodies of the invention, e.g., humanized antibodies of the invention.

As used herein, the term “diabodies” can refer to antibody fragments (e.g., small antibody fragments) with two antigen-binding sites; the fragments can comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Hollinger (1993) Proc. Natl. Acad. Sci. USA 90:6444-48. The term “triabodies” refers to antibody fragments with three antigen-binding sites.

As used herein, the term “minibody” refers to a scFv joined to a CH3 domain may also be made using an antibody of the invention. See, e.g., U.S. Pat. No. 5,837,821; Hu et al., Cancer Res. 56:3055-61 (1996). The term “nanobody” refers to a single variable region (VHH) domain, originally characterized in camels and llamas can also be employed. See, e.g., Davies et al., BioTechnology 13:475-79 (1995); Cortez-Retamozo, et al., Cancer Res. 64:2853-57 (2004).

As used herein, the term “fusion protein” refers to an antibody or an antigen binding fragment thereof that is fused to a heterologous protein or protein fragment. Such fusion proteins include N-terminal identification peptides which impart desired characteristics, such as increased stability or simplified purification. Detection and purification facilitating domains include, e.g., metal chelating peptides such as polyhistidine tracts, histidine-tryptophan modules, FLAG tags, cleavable linker sequences (e.g., Factor Xa or enterokinase). See, e.g., Williams, Biochemistry 34:1787-97 (1995); Dobeli, Protein Expr. Purif. 12:404-14 (1998); Kroll, DNA Cell Biol. 12:441-53 (1993); PROTEIN PURIFICATION: A PRACTICAL APPROACH (Roe, ed., Oxford University Press 2001); MONOCLONAL ANTIBODIES: A PRACTICAL APPROACH (Shephard et al., eds., Oxford University Press 2000).

As used herein, the term “biologically active” refers to an antibody or antibody fragment that is capable of binding the desired the antigenic epitope and directly or indirectly exerting a biologic effect. Direct effects include, but are not limited to the modulation of a growth signal, the modulation of an anti-apoptotic signal, the modulation of an apoptotic or necrotic signal, the modulation of the ADCC cascade, the modulation of the CDC cascade, inhibition of ligand-receptor interactions, modulation of internalization, and eliciting phagocytosis. Modulation of an activity can include the inhibition or stimulation of a particular activity. Indirect effects include, but are not limited to toxicity due to conjugate delivery (e.g., radionuclide) or sensitization to secondary agents (e.g., phototoxic agent).

The term “protease cleavage site” refers to residues on the antibody sequence recognized and cleaved by a particular protease when accessible to the protease. To cleave a peptide bond within the antibody, the protease recognizes and binds a region of the polypeptide that brackets the scissile peptide bond, i.e., the bond that is to be cleaved. Most proteases bind several amino acid residues in their active sites. Using the nomenclature of Schechter and Berger (Biochem. Biophys. Res. Commun. 27:157 (1967)), the bond to be hydrolyzed is formed between the P1 residue (N-terminal side of the cleaved bond) and the P1′ residue (C-terminal side of the cleaved bond) of the substrate. The residues adjacent to P1 on the N-terminal side of the sessile bond are labeled P2-Pn, and the residue adjacent to the P1′ site on the C-terminal side are labeled P2′-Pn′. The protease has corresponding “subsites” where the residues of the substrate fit, identified as S1, S1′, etc. The protease cleavage sites of the invention can consist on two, three, four, five, six, or more residues.

In alternative aspect, an antibody of the invention specifically binds to a pathogen, a virulence factor, a dietary enzyme or a toxin, such as a bacterial toxin, e.g., Clostridium difficile toxin. While the term “specifically binds” in reference to an antibody binding to an antigen is well known in the art, in alternative aspects the term “specifically binds” means that an Ab is binding to an Ab at a binding constant of at least 10⁻⁴, 10⁻⁵, 10⁻⁶, 10^{31 7}, 10⁻⁸ or 10⁻⁹, or anywhere within the range of between 10⁻⁴ and 10⁻⁹.

### Introducing Sequence Variations

The invention provides modified antibodies, and methods (both stochastic and nonstochastic) for modifying antibody sequences for, e.g., generating a protease resistant antibodies, e.g., for oral administration. In one aspect, modifications in an antibody of the invention comprise at least one mutation in the amino acid sequence of the antibody. The variant portion in the antibody sequence can comprise any number of modifications including two, three, four, five, six, seven, eight, nine, ten, eleven, or more amino acid modifications.

In some embodiments, the modification of the antibody is in a protease cleavage site or at a site flanking the protease cleavage site. A protease cleavage site can be identified by any suitable method. In some embodiments, sites of protease cleavage are identified using known protease cleavage motifs. In other embodiments, sites of protease cleavage are identified by characterizing the fragments that result from protease digestion. Such methods include, but are not limited to well known methods that characterize sequences following protease digestion, e.g., N-terminal sequencing, gel electrophoresis analysis, mass spectral analysis, and crystallographic studies. See e.g., CURRENT PROTOCOLS IN MOLECULAR BIOLOGY (John Wiley & Sons, most recent edition); Perona (1995) Protein Sci. 4:337.

In alternative embodiments, the modification is at the P1, P1′, P2, P3, P4, P2′, P3′, or P4 residue of the protease cleavage site. The modification to the amino acid sequence generates a protease resistance motif, rendering the protease cleavage site non-cleavable or less susceptible to protease cleavage.

In some embodiments, the modifications are made to the same protease cleavage motif throughout the antibody. In other embodiment, the modifications are made to different protease cleavage motifs. The modifications can be made in a protease cleavage site that is not flanked by an amino acid residue known to inhibit or attenuate protease cleavage. Such amino acids include Pro, Lys, Arg and His. An inhibitory or attenuating residue is any residue that interferes with the formation of the catalytic triad or two catalytic diads that acts as a proton shuttle or reduces the availability of the catalytic site.

The variant portion of the antibody (the amino acid residue modifications) can include any portion of the antibody, e.g., including the heavy chain, a light chain, or both. In some embodiments, the amino acid residue modifications are in (the variant portion is in) the Fc region, the hinge region, the CH_{L} domain, the CH₁ domain, the CH₂ domain, the CH₃ domain, the Fab region, or any combination thereof. In alternative embodiments, the variant portion is a V_{H} or V_{L} domain, provided the cleavage site does not have a negative effect on the desired antibody function. In one aspect, a mutation does not have a negative impact on antibody function if the antibody at least retains some of its ability to specifically bind its antigen (e.g., in some aspects, with less specific binding affinity). In alternative aspects, the antibody retains at least one of its biological activities (e.g., Fc receptor function) in addition to its ability to specifically bind the antigen.

The mutation is introduced by modifications, additions or deletions to a nucleic acid encoding the antibody. Thus, a nucleic acid encoding the antibody modified by the method of the invention can be altered by any suitable means. For example, site-directed mutagenesis may be employed. See, e.g., Ling et al. (1997) Anal Biochem. 254(2): 157-178; Dale et al. (1996) Methods Mol. Biol. 57:369-374; Smith (1985) Ann. Rev. Genet. 19:423-462; Botstein & Shortle (1985) Science 229:1193-1201; Carter (1986) Biochem. J. 237:1-7; and Kunkel (1987) Nucleic Acids & Molecular Biology (Eckstein, F. and Lilley, D. M. J. eds., Springer Verlag, Berlin); Kunkel (1985) Proc. Natl. Acad. Sci. USA 82:488-492; Kunkel et al. (1987) Methods in Enzymol. 154, 367-382; Bass et al. (1988) Science 242:240-245); Methods in Enzymol. 100: 468-500 (1983); Methods in Enzymol. 154: 329-350 (1987); Zoller & Smith (1982) Nucleic Acids Res. 10:6487-6500; Zoller & Smith (1983) Methods in Enzymol. 100:468-500; Zoller & Smith (1987) Methods in Enzymol. 154:329-350); Taylor et al. (1985) Nucl. Acids Res. 13: 8749-8764; Taylor et al. (1985) Nucl. Acids Res. 13: 8765-8787 (1985); Nakamaye (1986) Nucl. Acids Res. 14: 9679-9698; Sayers et al. (1988) Nucl. Acids Res. 16:791-802; and Sayers et al. (1988) Nucl. Acids Res. 16: 803-814); Kramer et al. (1984) Nucl. Acids Res. 12: 9441-9456; Kramer & Fritz (1987) Methods in Enzymol. 154:350-367; Kramer et al. (1988) Nucl. Acids Res. 16: 7207; and Fritz et al. (1988) Nucl. Acids Res. 16: 6987-6999).

Additional protocols that can be used to practice the invention (e.g., to modify antibody sequences to generate protease resistant Abs for oral administration) include point mismatch repair (Kramer (1984) Cell 38:879-887), mutagenesis using repair-deficient host strains (Carter et al. (1985) Nucl. Acids Res. 13: 4431-4443; and Carter (1987) Methods in Enzymol. 154: 382-403), deletion mutagenesis (Eghtedarzadeh (1986) Nucl. Acids Res. 14: 5115), restriction-selection and restriction-selection and restriction-purification (Wells et al. (1986) Phil. Trans. R. Soc. Lond. A 317: 415-423), mutagenesis by total gene synthesis (Nambiar et al. (1984) Science 223: 1299-1301; Sakamar and Khorana (1988) Nucl. Acids Res. 14: 6361-6372; Wells et al. (1985) Gene 34:315-323; and Grundstrom et al. (1985) Nucl. Acids Res. 13: 3305-3316), double-strand break repair (Mandecki (1986); Arnold (1993) Current Opinion in Biotechnology 4:450-455; and Proc. Natl. Acad. Sci. USA, 83:7177-7181). Additional details on many of the above methods can be found in Methods in Enzymology Volume 154, which also describes useful controls for trouble-shooting problems with various mutagenesis methods.

Other exemplary protocols for modifying antibody sequences, e.g., to generate protease resistant Ab sequences for oral administration, include those found in, e.g., in U.S. Pat. No. 5,605,793, U.S. Pat. No. 5,811,238, U.S. Pat. No. 5,830,721, U.S. Pat. No. 5,834,252, U.S. Pat. No. 5,837,458, WO 95/22625, WO 96/33207, WO 97/20078, WO 97/3596, WO 99/4140, WO 99/41383, WO 99/41369, WO 99/41368, EP 752008, EP 0932670, WO 99/23107, WO 99/21979, WO 98/31837, WO 98/27230, WO 98/27230, WO 00/00632, WO 00/09679, WO 98/42832, WO 99/29902, WO 98/41653, WO 98/41622, and WO 98/42727.

Protocols that can be used to practice the invention (e.g., to modify antibody sequences to generate protease resistant Abs for oral administration) and provide details regarding various diversity generating methods are described, e.g., in U.S. patent application Ser. No. 09/407,800, filed Sep. 28, 1999; U.S. Pat. No. 6,379,964; U.S. Pat. Nos. 6,319,714; 6,368,861; 6,376,246; 6,423,542; 6,426,224 and PCT/US00/01203; U.S. Pat. No. 6,436,675; PCT/US00/01202, filed Jan. 18, 2000, and, e.g. U.S. Ser. No. 09/618,579, filed Jul. 18, 2000; PCT/US00/01138, filed Jan. 18, 2000; and U.S. Ser. No. 09/656,549, filed Sep. 6, 2000; and U.S. Pat. Nos. 6,177,263; 6,153,410.

Non-stochastic, or “directed evolution,” methods useful in generating an antibody of the invention include, e.g., “gene site saturation mutagenesis” (GSSM) or “saturation mutagenesis”, synthetic ligation reassembly (SLR), or a combination thereof are used to modify the nucleic acids of the invention to generate antibodies with new or altered properties (e.g., activity under highly acidic or alkaline conditions, high temperatures, and the like). Polypeptides encoded by the modified nucleic acids can be screened for an activity before testing for proteolytic or other activity. Any testing modality or protocol can be used, e.g., using a capillary array platform. See, e.g., U.S. Pat. Nos. 6,361,974; 6,280,926; 5,939,250.

The modifications, additions or deletions to a nucleic acid encoding the antibody can be introduced by any suitable method including, but not limited to error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturation Mutagenesis (GSSM), synthetic ligation reassembly (SLR) or a combination thereof. The modifications, additions or deletions to a nucleic acid encoding the antibody can also be introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, or a combination thereof.

Gene Site Saturation Mutagenesis, or, GSSM

Antibodies of the invention, including protease resistant antibodies for oral administration, can be generated by non-stochastic mutation of the nucleic acids that encode them by, e.g., Gene Site Saturation Mutagenesis, or, GSSM, as described, e.g., in U.S. Pat. No. 6,171,820, Nos. 6,562,594, 6,764,835, and U.S. Patent Publication No. 2004 0018607.

In GSSM, codon primers containing a degenerate N,N,G/T sequence are used to introduce point mutations into a polynucleotide, e.g., an antibody of the invention, so as to generate a set of progeny polypeptides in which a full range of single amino acid substitutions is represented at each amino acid position, e.g., an amino acid residue in an enzyme active site or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids. In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used—either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position X 100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide molecules (e.g., anti-toxin antibodies of the invention) such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual progeny polypeptide is identified by screening to display a favorable change in property (when compared to the parental polypeptide, such as increased glucan hydrolysis activity under alkaline or acidic conditions), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if two specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined—6 single point mutations (i.e. 2 at each of three positions) and no change at any position.

In yet another aspect, site-saturation mutagenesis can be used together with shuffling, chimerization, recombination and other mutagenizing processes, along with screening. This invention provides for the use of any mutagenizing process(es), including saturation mutagenesis, in an iterative manner. In one exemplification, the iterative use of any mutagenizing process(es) is used in combination with screening.

In one aspect, the GSSM comprises use of codon primers (containing a degenerate N,N,N sequence) to introduce point mutations into a polynucleotide, so as to generate a set of progeny polypeptides in which a full range of single amino acid substitutions is represented at each amino acid position. The oligos used are comprised contiguously of a first homologous sequence, a degenerate N,N,N sequence and in one aspect but not necessarily a second homologous sequence. The downstream progeny translational products from the use of such oligos include all possible amino acid changes at each amino acid site along the polypeptide, because the degeneracy of the N,N,N sequence includes codons for all 20 amino acids. In one aspect, one such degenerate oligo (comprised of one degenerate N,N,N cassette) is used for subjecting each original codon in a parental polynucleotide template to a full range of codon substitutions. In another aspect, at least two degenerate N,N,N cassettes are used—either in the same oligo or not, for subjecting at least two original codons in a parental polynucleotide template to a full range of codon substitutions. Thus, more than one N,N,N sequence can be contained in one oligo to introduce amino acid mutations at more than one site. This plurality of N,N,N sequences can be directly contiguous, or separated by one or more additional nucleotide sequence(s). In another aspect, oligos serviceable for introducing additions and deletions can be used either alone or in combination with the codons containing an N,N,N sequence, to introduce any combination or permutation of amino acid additions, deletions and/or substitutions.

In one aspect, it is possible to simultaneously mutagenize two or more contiguous amino acid positions using an oligo that contains contiguous N,N,N triplets, i.e. a degenerate (N,N,N)ₙ sequence. In another aspect, the invention provides for the use of degenerate cassettes having less degeneracy than the N,N,N sequence. For example, it may be desirable in some instances to use (e.g. in an oligo) a degenerate triplet sequence comprised of only one N, where the N can be in the first second or third position of the triplet. Any other bases including any combinations and permutations thereof can be used in the remaining two positions of the triplet. Alternatively, it may be desirable in some instances to use (e.g., in an oligo) a degenerate N,N,N triplet sequence, N,N,G/T, or an N,N,G/C triplet sequence.

It is appreciated, however, that the use of a degenerate triplet (such as N,N,G/T or an N,N, G/C triplet sequence) as disclosed in the instant invention is advantageous for several reasons. In one aspect, this invention provides a means to systematically and fairly easily generate the substitution of the full range of possible amino acids (for a total of 20 amino acids) into each and every amino acid position in a polypeptide. Thus, for a 100 amino acid polypeptide, the invention provides a way to systematically and fairly easily generate 2000 distinct species (i.e., 20 possible amino acids per position times 100 amino acid positions). It is appreciated that there is provided, through the use of an oligo containing a degenerate N,N,G/T or an N,N, G/C triplet sequence, 32 individual sequences that code for 20 possible amino acids. Thus, in a reaction vessel in which a parental polynucleotide sequence is subjected to saturation mutagenesis using one such oligo, there are generated 32 distinct progeny polynucleotides encoding 20 distinct polypeptides. In contrast, the use of a non-degenerate oligo in site-directed mutagenesis leads to only one progeny polypeptide product per reaction vessel.

This invention also provides for the use of nondegenerate oligos, which can optionally be used in combination with degenerate primers disclosed. It is appreciated that in some situations, it is advantageous to use nondegenerate oligos to generate specific point mutations in a working polynucleotide. This provides a means to generate specific silent point mutations, point mutations leading to corresponding amino acid changes and point mutations that cause the generation of stop codons and the corresponding expression of polypeptide fragments.

Thus, in one aspect of this invention, each saturation mutagenesis reaction vessel contains polynucleotides encoding at least 20 progeny polypeptide molecules such that all 20 amino acids are represented at the one specific amino acid position corresponding to the codon position mutagenized in the parental polynucleotide. The 32-fold degenerate progeny polypeptides generated from each saturation mutagenesis reaction vessel can be subjected to clonal amplification (e.g., cloned into a suitable E. coli host using an expression vector) and subjected to expression screening. When an individual progeny polypeptide is identified by screening to display a favorable change in property (when compared to the parental polypeptide), it can be sequenced to identify the correspondingly favorable amino acid substitution contained therein.

It is appreciated that upon mutagenizing each and every amino acid position in a parental polypeptide (e.g., an antibody to be modified for protease resistance) using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid and each of two favorable changes) and 3 positions. Thus, there are 3×3×3 or 27 total possibilities, including 7 that were previously examined—6 single point mutations (i.e., 2 at each of three positions) and no change at any position.

Thus, in a non-limiting exemplification, this invention provides for the use of saturation mutagenesis in combination with additional mutagenization processes, such as process where two or more related polynucleotides are introduced into a suitable host cell such that a hybrid polynucleotide is generated by recombination and reductive reassortment.

In addition to performing mutagenesis along the entire sequence of a gene, the instant invention provides that mutagenesis can be use to replace each of any number of bases in a polynucleotide sequence, wherein the number of bases to be mutagenized is in one aspect every integer from 15 to 100,000. Thus, instead of mutagenizing every position along a molecule, one can subject every or a discrete number of bases (in one aspect a subset totaling from 15 to 100,000) to mutagenesis. In one aspect, a separate nucleotide is used for mutagenizing each position or group of positions along a polynucleotide sequence. A group of 3 positions to be mutagenized may be a codon. The mutations can be introduced using a mutagenic primer, containing a heterologous cassette, or a mutagenic cassette. Exemplary cassettes can have from 1 to 500 bases. Each nucleotide position in such heterologous cassettes be N, A, C, G, T, A/C, A/G, A/T, C/G, C/T, G/T, C/G/T, A/G/T, A/C/T, A/C/G, or E, where E is any base that is not A, C, G, or T (E can be referred to as a designer oligo).

Saturation mutagenesis can comprise mutagenizing a complete set of mutagenic cassettes (wherein each cassette is in one aspect about 1-500 bases in length) in defined polynucleotide sequence to be mutagenized (wherein the sequence to be mutagenized is in one aspect from about 15 to 100,000 bases in length). Thus, a group of mutations (ranging from 1 to 100 mutations) is introduced into each cassette to be mutagenized. A grouping of mutations to be introduced into one cassette can be different or the same from a second grouping of mutations to be introduced into a second cassette during the application of one round of saturation mutagenesis. Such groupings are exemplified by deletions, additions, groupings of particular codons and groupings of particular nucleotide cassettes.

Defined sequences to be mutagenized include a whole gene, pathway, cDNA, an entire open reading frame (ORF) and entire promoter, enhancer, repressor/transactivator, origin of replication, intron, operator, or any polynucleotide functional group. Generally, a “defined sequences” for this purpose may be any polynucleotide that a 15 base-polynucleotide sequence and polynucleotide sequences of lengths between 15 bases and 15,000 bases (this invention specifically names every integer in between). Considerations in choosing groupings of codons include types of amino acids encoded by a degenerate mutagenic cassette.

In one exemplification a grouping of mutations that can be introduced into a mutagenic cassette, this invention specifically provides for degenerate codon substitutions (using degenerate oligos) that code for 2, 3, 4, 5, 6, 7, 8, 9, 10, 11, 12, 13, 14, 15, 16, 17, 18, 19 and 20 amino acids at each position and a library of polypeptides encoded thereby.

Synthetic Ligation Reassembly (SLR)

A non-stochastic gene modification system termed “synthetic ligation reassembly,” or simply “SLR,” a “directed evolution process,” can also be used to generate protease resistant antibodies for oral administration. SLR is described, e.g., in U.S. Pat. Nos. 6,537,776 and 6,605,449.

SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10¹⁰⁰ different chimeras. SLR can be used to generate libraries comprised of over 10¹⁰⁰⁰ different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus, the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g. T4 DNA ligase), to achieve covalent bonding of the building pieces.

In one aspect, the design of the oligonucleotide building blocks is obtained by analyzing a set of progenitor nucleic acid sequence templates (e.g., anti-toxin antibodies) that serve as a basis for producing a progeny set of finalized chimeric polynucleotides (e.g., nucleic acids encoding protease resistant Abs). These parental oligonucleotide templates thus serve as a source of sequence information that aids in the design of the nucleic acid building blocks that are to be mutagenized, e.g., chimerized or shuffled. In one aspect of this method, the sequences of a plurality of parental nucleic acid templates are aligned in order to select one or more demarcation points. The demarcation points can be located at an area of homology, and are comprised of one or more nucleotides. These demarcation points are in one aspect shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (comprised of at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Even more in one aspect a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

In one aspect, a ligation reassembly process is performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another aspect, the assembly order (i.e. the order of assembly of each building block in the 5′ to 3 sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g. one by one. In other words this invention provides that, through the selective and judicious use of specific nucleic acid building blocks, coupled with the selective and judicious use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups. Because of its ability to perform chimerizations in a manner that is highly flexible yet exhaustive and systematic as well, particularly when there is a low level of homology among the progenitor molecules, these methods provide for the generation of a library (or set) comprised of a large number of progeny molecules. Because of the non-stochastic nature of the instant ligation reassembly invention, the progeny molecules generated in one aspect comprise a library of finalized chimeric nucleic acid molecules having an overall assembly order that is chosen by design. The saturation mutagenesis and optimized directed evolution methods also can be used to generate different progeny molecular species. It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the operability of this invention. In fact, demarcation points can even be chosen in areas of little or no intermolecular homology. For example, because of codon wobble, i.e. the degeneracy of codons, nucleotide substitutions can be introduced into nucleic acid building blocks without altering the amino acid originally encoded in the corresponding progenitor template. Alternatively, a codon can be altered such that the coding for an originally amino acid is altered. This invention provides that such substitutions can be introduced into the nucleic acid building block in order to increase the incidence of intermolecular homologous demarcation points and thus to allow an increased number of couplings to be achieved among the building blocks, which in turn allows a greater number of progeny chimeric molecules to be generated.

In one aspect, the present invention provides a non-stochastic method termed synthetic gene reassembly, that is somewhat related to stochastic shuffling, save that the nucleic acid building blocks are not shuffled or concatenated or chimerized randomly, but rather are assembled non-stochastically.

The synthetic gene reassembly method does not depend on the presence of a high level of homology between polynucleotides to be shuffled. The invention can be used to non-stochastically generate libraries (or sets) of progeny molecules comprised of over 10¹⁰⁰ different chimeras. Conceivably, synthetic gene reassembly can even be used to generate libraries comprised of over 10¹⁰⁰⁰ different progeny chimeras.

Thus, in one aspect, the invention provides a non-stochastic method of producing a set of finalized chimeric nucleic acid molecules having an overall assembly order that is chosen by design, which method is comprised of the steps of generating by design a plurality of specific nucleic acid building blocks having serviceable mutually compatible ligatable ends and assembling these nucleic acid building blocks, such that a designed overall assembly order is achieved.

In another aspect, the design of nucleic acid building blocks is obtained upon analysis of the sequences of a set of progenitor nucleic acid templates that serve as a basis for producing a progeny set of finalized chimeric nucleic acid molecules. These progenitor nucleic acid templates thus serve as a source of sequence information that aids in the design of the nucleic acid building blocks that are to be mutagenized, i.e. chimerized or shuffled.

Optimized Directed Evolution System

A non-stochastic gene modification system termed “optimized directed evolution system” can also be used to generate antibodies of the invention, or used in methods of the invention to modify antibody-encoding sequences, e.g., to generate protease resistant Abs. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events.

A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

One method for creating a chimeric progeny polynucleotide sequence is to create oligonucleotides corresponding to fragments or portions of each parental sequence. Each oligonucleotide in one aspect includes a unique region of overlap so that mixing the oligonucleotides together results in a new variant that has each oligonucleotide fragment assembled in the correct order. Additional information can also be found, e.g., in U.S. Pat. Nos. 6,537,776, and 6,361,974.

In vivo Shuffling

In vivo shuffling of nucleic acids can also be used to generate antibodies of the invention, or used in methods of the invention to modify antibody-encoding sequences, e.g., to generate protease resistant Abs. In vivo shuffling can be performed utilizing the natural property of cells to recombine multimers. While recombination in vivo has provided the major natural route to molecular diversity, genetic recombination remains a relatively complex process that involves 1) the recognition of homologies; 2) strand cleavage, strand invasion, and metabolic steps leading to the production of recombinant chiasma; and finally 3) the resolution of chiasma into discrete recombined molecules. The formation of the chiasma requires the recognition of homologous sequences.

In vivo reassortment is focused on “inter-molecular” processes collectively referred to as “recombination” which in bacteria, is generally viewed as a “RecA-dependent” phenomenon. The invention can use recombination processes of a host cell to recombine and re-assort (e.g., antibody) sequences, or the cells' ability to mediate reductive processes to decrease the complexity of quasi-repeated sequences in the cell by deletion. This process of “reductive reassortment” occurs by an “intra-molecular”, RecA-independent process. Thus, in another aspect of the invention, novel polynucleotides can be generated by the process of reductive reassortment. The method involves the generation of constructs containing consecutive sequences (original encoding sequences), their insertion into an appropriate vector and their subsequent introduction into an appropriate host cell. The reassortment of the individual molecular identities occurs by combinatorial processes between the consecutive sequences in the construct possessing regions of homology, or between quasi-repeated units. The reassortment process recombines and/or reduces the complexity and extent of the repeated sequences and results in the production of novel molecular species. Various treatments may be applied to enhance the rate of reassortment. These can include treatment with ultra-violet light, or DNA damaging chemicals and/or the use of host cell lines displaying enhanced levels of “genetic instability”. Thus the reassortment process may involve homologous recombination or the natural property of quasi-repeated sequences to direct their own evolution.

### Kabat Index and Numbering Scheme

The invention provides antibodies having modified sequences based on the Kabat numbering system, i.e., based on the EU index as in Kabat (the Kabat numbering scheme is a widely adopted standard for numbering the residues in an antibody in a consistent manner). Where the EU index in Kabat is not mentioned, a specific position or mutation may refer to the absolute position (residue number) in an antibody sequence or subsequence, as will be clear from context, for example in Tables 2, 3A, 3B, 4, 5, and 9 (Examples 1 to 3), and Example 6, Tables 1 and 2. Equivalents between absolute positions and Kabat/EU designations are given in Example 1, Table 1 and Example 3, Table 8.

In one aspect, the variant portion of the antibody of the invention comprises at least one amino acid substitution at any one or more of amino acid positions T155, L179, L235, F241, Y296, L309, Y349, L365, L398, F404, Y407, and Y436 of an IgG heavy chain, e.g., SEQ ID NO:1, SEQ ID NO:3 and/or SEQ ID NO:5, wherein the numbering of the residues in the variant portion is that of the EU index as in Kabat, whereby the amino acid substitution confers increased resistance to pepsin proteolysis. In another embodiment, the variant portion comprises at least one amino acid substitution at any one or more of amino acid positions L234, L242, F243, F275, Y278, Y300, L306, W313, L314, Y319, L351, L368, Y391, F405, L406, L410, F423, L432, or Y436 of a IgG heavy chain, e.g., SEQ ID NO:1, SEQ ID NO:3 and/or SEQ ID NO:5, wherein the numbering of the residues in the variant portion is that of the EU index as in Kabat, whereby the amino acid substitution confers increased resistance to pepsin proteolysis.

Example 1 describes, inter alia, an exemplary method using the Kabat numbering system (“wherein the numbering of the residues in the variant portion is that of the EU index as in Kabat”) to design/ make an antibody within the scope of the invention, see also Table 1 of Example 1, below.

In one embodiment, the variant portion comprises at least one amino acid substitution at any one or more of amino acid positions F116, K126, R143, K169 or K183 of a kappa (light) chain, e.g., SEQ ID NO:2, SEQ ID NO:4 and/or SEQ ID NO:6, wherein the numbering of the residues in the variant portion is that of the EU index as in Kabat, whereby the amino acid substitution confers increased resistance to pancreatin proteolysis.

In another embodiment, the variant portion comprises at least one amino acid substitution at any one or more of amino acid positions K133, K205, K210, K274, K326, K340, R355, K360 or K392 of a IgG heavy chain, e.g., SEQ ID NO:1, SEQ ID NO:3 and/or SEQ ID NO:5, wherein the numbering of the residues in the variant portion is that of the EU index as in Kabat, whereby the amino acid substitution confers increased resistance to pancreatin proteolysis.

In one aspect, the variant portion of an antibody of the invention comprises at least one amino acid substitution at the P1 or P1′ site of cleavage in a trypsin cleavage motif, wherein the substituted amino acid is K or R, whereby the amino acid substitution confers increased resistance to trypsin proteolysis. In one embodiment, the variant portion comprises at least one amino acid substitution, at the P1 or P1′ site of cleavage in a pepsin cleavage motif, wherein the substituted amino acid is L, F, Y, W, I, or T, whereby the amino acid substitution confers increased resistance to pepsin proteolysis. In some embodiments, the variant portion comprises at least one amino acid substitution at the P1 or P1′ site of cleavage in a chymotrypsin cleavage motif, wherein the substituted amino acid is F, Y, or W, whereby the amino acid substitution confers increased resistance to chymotrypsin proteolysis.

In one embodiment, the variant portion of an antibody of the invention comprises at least one amino acid substitution selected from the group of amino acid substitutions of L235P, L398Q, F404Y, L179I, and T155S, in an IgG₁ heavy chain, wherein the numbering of the residues in the variant portion is that of the EU index as in Kabat, whereby the amino acid substitution confers increased resistance to pepsin proteolysis. In another specific embodiment, the variant portion comprises at least one amino acid substitution selected from the group of amino acid substitutions of F116S and K126A in a kappa light chain, wherein the numbering of the residues in the variant portion is that of the EU index as in Kabat, whereby the amino acid substitution confers increased resistance to pepsin proteolysis. In another embodiment, the variant portion comprises at least one amino acid substitution selected from the group of amino acid substitutions of K133G and K274Q in a IgG heavy chain, e.g., SEQ ID NO:1, SEQ ID NO:3 and/or SEQ ID NO:5, wherein the numbering of the residues in the variant portion is that of the EU index as in Kabat, whereby the amino acid substitution confers increased resistance to pepsin proteolysis.

The Kabat numbering scheme is a widely adopted standard for numbering the residues in an antibody in a consistent manner. The Kabat numbering systems and database of aligned sequences are well known in the art, see, e.g., Kabat, et al. (1991) Sequences of Proteins of Immunological Interest, Fifth Edition. NIH Publication No. 91-3242. See also, e.g., Johnson (1997) Genetics 145:777-786; Johnson (1997) Immunol. Cell Biol. 75:580-583; Johnson (2000) Nucleic Acids Res. 28: 214-218; Johnson (2001) Nucleic Acids Res. 29(1):205-206; Johnson (2004) Methods Mol. Biol. 248:11-25; Ramirez-Benitez (2001) Biosystems 61(2-3):125-31. The Kabat Database of aligned sequences of proteins of immunological interest provides useful correlations between structure and function for, e.g., immunoglobulin nucleotide and amino acid sequences and their tertiary structures. The Kabat Database, initially started in 1970 to determine the combining site of antibodies based on the available amino acid sequences, allows precise delineation of complementarity determining regions (CDR) of both light and heavy chains, and can be used to align sequences to derive structural and functional information, and to construct artificial antibodies with prescribed specificities. Antibody sequences can be compared to and tested against the Kabat sequence database.

In one aspect, an antibody of the invention, e.g., a wildtype antibody modified using the Kabat database according to the methods of the invention, has greater resistance to proteolysis relative to its comparable unaltered or “wildtype” antibody form. The increased resistance to proteolysis can be at least 1%, 2%, 3%, 4%, 5%, 10%, 15%, 20%, 25%, 30%, 40%, 50%, 60%, 70%, 80% or more than that of the unmodified antibody. The modified antibody can be partially or completely resistant to cleavage by more than one protease. Any suitable to determine sensitivity to proteolysis may be employed. See, e.g., PROTEOLYTIC ENZYMES: A PRACTICAL APPROACH 2ND ED. (Benyon et al., ed., Oxford University Press (2001); U.S. Pat. No. 5,981,200. Such assays include, but are not limited to, continuous spectrophotometric reading, fluorometric, ninhydrin methods, HPLC, capillary electrophoresis, and ELISA methods. The time and conditions for proteolysis measurement can be modified as needed to simulate the native state proteolytic reaction.

An antibody of the invention, or an antibody modified by a method of the invention, can be an IgG, IgM, IgD, IgE, or IgA antibody. In some embodiments, the antibody is an IgG antibody. In one aspect, the antibody can be an IgG₁, IgG₂, IgG₃, or IgG₄ antibody. Any suitable source can be used for the antibody. For example, the antibody can be a human, murine, rat, rabbit, bovine, camel, llama, dromedary, or simian antibody. The antibody can be a humanized antibody, a chimeric antibody, a bispecific antibody, a fusion protein, or a biologically active fragment thereof. In some embodiments, the antibody (or biologically active fragment thereof) is a fusion protein. The fusion protein can encompass additional peptide sequence that simplifies purification or production. Fusion proteins also may include domains and/or whole polypeptides that are biologically active in a manner that complements the activity of the antibody. For example, the antibody can be fused to a cytokine, ligand, adhesion molecule, peptide, receptors, enzymes, therapeutic proteins, dyes, small organic molecules, or any biologically active portion thereof.

In some embodiments, the proteolysis is the digestion mediated by proteases from the gastrointestinal tract, the blood, or the bile. In alternative embodiments, the proteolysis is mediated by pepsin, pancreatin, trypsin, trypsinogen, chymo-trypsinogen, carboxy-peptidase, pro-carboxy-peptidase, elastase, pro-elastase, or any combination thereof. The protease can be one selected from a group of proteases released by an exogenous organism or any organism within the digestive tract, or released or produced in the digestive tract. In some embodiments, the protease can be selected from a group of proteases released or produced by an abnormal, infected, cancerous or otherwise diseased tissue.

An antibody of the invention can be modified in any suitable manner to confer or enhance a desirable effector function or physical characteristic. In some embodiment, the Fc region of an antibody of the invention is further modified to enhance ADCC, CDC, or phagocytosis. The Fc region of the antibody can also be further modified to increase binding affinity to the Fc receptor (FcR). See, e.g., U.S. Pat. No. 6,737,056; and US 2004/0132101. In one embodiment, the antibody is further modified to have a) an antigen binding activity comparable to or superior to the unmodified antibody; b) a chemical stability comparable to or superior to the unmodified antibody; c) a thermostability or thermotolerance comparable to or superior to the unmodified antibody; d) a pH tolerance comparable to or superior to the unmodified antibody; e) a reduced immunogenicity; f) a reduced aggregation; g) an increased half-life relative to the unmodified antibody; h) an increased expression in a host cell; i) a stability in pharmaceutical formulation comparable or superior to that of the unmodified antibody; j) an enhanced dimerization of Fc regions; or k) any combination thereof. In some embodiments, an antibody of the invention has a) an antigen binding activity comparable to or superior to the unmodified antibody; b) a chemical stability comparable to or superior to the unmodified antibody; c) a thermostability or thermotolerance comparable to or superior to the unmodified antibody; d) a pH tolerance comparable to or superior to the unmodified antibody; e) a reduced immunogenicity; f) a reduced aggregation; g) an increased half-life relative to the unmodified antibody; h) an increased expression in a host cell; i) a stability in pharmaceutical formulation comparable or superior to that of the unmodified antibody; j) an enhanced dimerization of Fc regions; or k) any combination thereof.

In some embodiments, the modification of the antibody comprises one or more additions of post-translational modification sites. An antibody of the invention can also be glycosylated. The modifications can also comprise the addition of one or more N-glycosylation site or an O-glycosylation site, an alkyl chain or a small molecule, an addition of a disulfide bond site or a salt bridge site, and/or a covalent or non-covalent addition of a second molecule to the Fc chain of the antibody. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

In some embodiment, the second molecule comprises an antibody secretory component. The invention also provides methods for modifying the polypeptides of the invention by either natural processes, such as post-translational processing (e.g., phosphorylation, acylation, etc), or by chemical modification techniques, and the resulting modified polypeptides.

Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., PROTEINS—STRUCTURE AND MOLECULAR PROPERTIES 2nd Ed., W. H. Freeman and Company, New York (1993); POSTTRANSLATIONAL COVALENT MODIFICATION OF PROTEINS, B. C. Johnson, Ed., Academic Press, New York, pp. 1-12 (1983).

Any suitable means can be used to determine the binding affinity of an antibody of the invention. In one example, the affinity is determined by surface plasmon resonance (Biacore). An antibody of the invention can also be modified further to increase binding affinity using methods known in the art. See, e.g., U.S. Pat. No. 6,350,861.

In one aspect, the modified antibody is more thermostable or thermotolerant than the wildtype antibody. Any suitable means may be employed to assess the thermostability of an antibody of the invention. Thus, the modified antibody retains at least its binding activity under conditions comprising a temperature range of between about 37° C. to about 95° C.; between about 55° C. to about 85° C., between about 70° C. to about 95° C., or, between about 90° C. to about 95° C. The increased thermostability confers a significant advantage for long-term storage for the antibodies of the invention, particularly when the antibodies require storage in places with little or no ability to control storage temperature, e.g., remote regions of third world countries. In some embodiments, an antibody of the invention retains its binding activity as well as at least one desirable biological activity.

An antibody of the invention can also be further modified to reduce the immunogenicity of the antibody upon administration to the subject. The immunogenicity of the antibody includes the elimination of one or several antigenic epitopes within the antibody (e.g., through amino acid substitution) as well as residues and/or motifs that triggers non-specific xenogenic or innate responses that interfere with or reduce the antibody's therapeutic efficacy. See, e.g., Schellekans, Clin. Ther. 24:1720-40 (2002); Graddis et al., Curr. Pharm. Biotech. 3:285-97 (2002).

In one embodiment, an antibody of the invention results in reduced aggregation with itself or other antibodies or can be further modified to reduce such aggregation. It is desirable to reduce the aggregation of the antibodies as this property can result in increased immunogenicity and/or increase clearance, i.e., reduced half-life, for the antibody. Any suitable methods can be used to determine the amount of aggregation for the antibody. See, e.g., Graddis et al., Curr. Pharm. Biotech. 3:285-97 (2002). Modifications can then be made using the molecular biology methods known in the art including those disclosed herein.

The determination of the half-life of the antibody can be determined by any suitable means. For example, the antibody half-life can be determined by detection of the presence of the antibody (T_{1/2}), examining the biological activity half-life, or any combination thereof.

In one embodiment, the modified or engineered antibody has an increased tolerance to acidic pH conditions (e.g., pH 6.5, pH 6, pH 5.5, pH 5, pH 4.5, pH 4 or pH 3 or more acidic conditions) relative to wildtype antibody. In another embodiment, the engineered antibody has increased tolerance to alkaline pH conditions (e.g., pH 7.5, pH 8.0, pH 8.5, pH 9, pH 9.5, pH 10, pH 10.5 or pH 11 or more) relative to wildtype antibody. Any suitable method can be employed to determine pH tolerance. In some embodiments, the engineered antibody is identified as pH tolerant when the antibody maintains sufficient native conformation at about pH 3 and above to maintain some biological activity. In some embodiments, the antibody maintains all its native conformation. In one embodiment, the engineered antibody is identified as having greater protease resistance when the digestibility of the engineered antibody by the protease is increased at pH 3 relative to that of the wildtype antibody. The protease can be pepsin, trypsin, trypsinogen, chymo-trypsinogen, pro-carboxy-peptidase and/or pro-elastase. In one aspect, an engineered antibody it selected to retains biological activity in (or survives structurally, e.g., being “tolerant to”, or is resistant to pH dependent unfolding) conditions comprising the conditions of the stomach, which approximate an acidity of at least pH 3. Thus the present method can further comprise introducing additional mutations into a “wildtype” amino acid sequence to render an antibody more resistant to pH dependent unfolding.

In one aspect, the antibodies of the invention, or antibodies used in the methods of the invention, are dimerized or trimerized (e.g., diabody or triabody Abs). Enhanced dimerization or other multimerization of Fc regions of antibodies can result in a greater biological efficacy for some targets. Such increased dimerization can be determined using any suitable means employing methods known in the art.

In one embodiment, the antibody is modified to improve solubility, e.g., improving solubility under conditions of alkaline or acidic conditions, e.g., as those in the stomach. Solubility of proteins can be determined using routine methods in the art.

An antibody of the invention also can contain amino acid modifications that increase expression of the antibody in the host cell. The modified or engineered antibody can be expressed in vitro or in vivo. Any suitable host cell can be employed including, but not limited to, prokaryotic cells and eukaryotic cells such as bacterial cells, fungal cells, yeast cells, mammalian cells, insect cells, or plant cells. Exemplary bacterial cells include E. coli, Streptomyces, Bacillus subtilis, Salmonella typhimurium, and various species within the genera Pseudomonas, Streptomyces, and Staphylococcus. Exemplary insect cells include Drosophila S2, and Spodoptera Sf9. Exemplary animal cells include CHO, COS, or Bowes melanoma, or any known mouse or human cell line. In one embodiment, the modifications permit or enhance antibody expression in a mammalian expression system or in a plant expression system. The modifications include mutation of the nucleic acid sequence encoding the antibody to provide codons in a nucleic acid to increase or decrease its expression in a host cell. Any suitable method can be used to identify the mutations that permit or enhance host cell expression of the recombinant antibody. For example, the method can comprise identifying a “non-preferred” or a “less preferred” codon in antibody-encoding nucleic acid and replacing one or more of these non-preferred or less preferred codons with a “preferred codon” encoding the same amino acid as the replaced codon and at least one non-preferred or less preferred codon in the nucleic acid has been replaced by a preferred codon encoding the same amino acid. A preferred codon is a codon over-represented in coding sequences in genes in the host cell and a non-preferred or less preferred codon is a codon under-represented in coding sequences in genes in the host cell. Techniques for transfecting such host cells are well known in the art. See, e.g., CURRENT PROTOCOLS IN MOLECULAR BIOLOGY (John Wiley & Sons 1998). Techniques for transforming a wide variety of higher plant species are well known and described in the technical and scientific literature. See, e.g., Weising et al., Ann. Rev. Genetics 22:421-77 (1988); U.S. Pat. No. 5,750,870. The term “plant” includes whole plants, plant parts (e.g., leaves, stems, flowers, roots, etc.), plant protoplasts, seed, and plant cells and progeny of same. The class of plants which can be used to produce the antibodies of the invention is generally as broad as the class of higher plants amenable to transformation techniques, including angiosperms (monocotyledous and dicotyledonous plants), as well as gymnosperms. It includes plants of a variety of ploidy levels, including polyploidy, diploid, or haploid cells.

The antibodies and biologically active fragments thereof, of the invention include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function (e.g., antigen binding) is not substantially altered.

Antibodies of the invention can partially or completely comprise polypeptide mimetics, and can contain any combination of non-natural structural components. In alternative aspect, antibody mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues in antibodies of the invention can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester. See, e.g., Spatola (1983) in CHEMISTRY AND BIOCHEMISTRY OF AMINO ACIDS, PEPTIDES AND PROTEINS, Vol. 7, pp 267-357, “Peptide Backbone Modifications,” Marcell Dekker, NY.

Antibodies of the invention can also be characterized as mimetics and can contain (comprise) all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1,-2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluorophenylalanine; D- or L-p-biphenylphenylalanine; D- or L-p-methoxy-biphenylphenylalanine; D-or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acids in antibodies of the invention include, e.g., thiazolyl, thiophenyl, pyrazolyl, benzimidazolyl, naphthyl, furanyl, pyrrolyl, and pyridyl aromatic rings.

Mimetics of acidic amino acids in antibodies of the invention can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl)carbodiimide or 1-ethyl-3(4-azonia-4,4-dimetholpentyl)carbodiimide. Aspartyl or glutamyl can also be converted to asparaginyl and glutaminyl residues by reaction with ammonium ions.

Mimetics of basic amino acids in antibodies of the invention can be generated by substitution with, e.g., (in addition to lysine and arginine) the amino acids omithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, in one aspect, under alkaline conditions.

Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively.

Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl)propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole.

Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate.

Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide.

Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

An amino acid substitution in antibodies of the invention can also include the substitution of an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but also can be referred to as the R- or S-form.

In some embodiments, an antibody of the invention specifically binds to a pathogen. Any pathogen can be targeted by an antibody of the invention. In some embodiments, the pathogen is selected from the group consisting of a bacteria, a virus and a fungus. More specifically, the pathogen can be an intestinal pathogen. In specific embodiments, the intestinal pathogen is selected from the group consisting of enterotoxigenic E. coli, rotavirus, Cryptosporidium parvum, Clostridium difficile, Shigella flexneri, Campylobacter jejuni, Staphylococcus aureus, E. coli O157:H7, Helicobacter pylori, Pseudomonas aeruginosa, Shigella dysenteriae, Salmonella enteritidis, Salmonella typhi, Clostridium perfringens, Aeromonas hydrophila, and Aeromanas aerolysin. In another specific embodiment, the pathogen is Streptococcus mutans. In some embodiments, the bacteria is a Helicobacter pylori, an Escherichia sp., a Cryptosporidium sp., a Clostridium sp. or a Shigella sp. In some embodiments, the fungal pathogen is Candida albicans or Aspergillus fumigatus. In some embodiments, the viral pathogen is a species in the genera of rotavirus, hepatitis, astrovirus, picornavirus, adenovirus, or parvovirus.

The anti-pathogenic effect of an antibody of the invention, or an antibody used in a method of the invention, can result from the specific binding of the antibody to a virulence factor. The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of an antibody to the sample may be detected using a secondary antibody having such a detectable label thereon. Alternative assays include ELISA assays, sandwich assays, radioimmunoassays, and Western Blots. See e.g., ANTIBODY ENGINEERING: A PRACTICAL APPROACH (Oxford University Press, 1996). These monoclonal antibodies can bind with at least a K_{d} of about 1 μM, or at least about 300 nM, or at least about 30 nM, or in one aspect, at least about 10 nM, in one aspect, at least about 3 nM or better, usually determined by ELISA.

Any suitable method may be employed to determine the biological activity of an antibody in the presence of the virulence factor. Such assays include binding assays, in vitro assays assessing morphology, viability, phagocytosis, cytotoxicity (e.g., ADCC and CDC), and/or proliferation, and in vivo models such as the ileal loop models and passive immunotherapy models. See, e.g., CURRENT PROTOCOLS IN IMMUNOLOGY (John Wiley & Sons, latest edition); Rafael et al., ADVANCED CURRENT PROTOCOLS IN CELLULAR IMMUNOLOGY (CRC Press 2000); and Gazzano-Santoro et al., J. Immunol. Methods 202:163 (1996). In one embodiment, an antibody has anti-virulence factor activity if the antibody reduces the pathogenicity of the organism and/or the toxicity of the virulence factor by at least 20%, at least 50%, 60%, 70%, 80%, 90%, or 100%. In another embodiment, an antibody ofthe invention has anti-virulence activity if the antibody reduces the pathogenicity of the organism and/or the toxicity of the virulence factor by at least 10%, 20%, 30%, 40%, 50%, 60%, 70%, 80%, 90% or 95% or 100% in presence of one or more other anti-virulence factors.

To determine the effectiveness of the antibody, a cell can be contacted with the antibody and the virulence factor, pathogenic organism, or cell in any suitable manner for any suitable length of time. The cells can be contacted with the antibody more than once during incubation or treatment. In one aspect, the dose required is in the range of about 1 μg/ml to 1000 μg/ml, or in the range of 100 μg/ml to 800 μg/ml. The exact dose can be readily determined from in vitro cultures of the cells and exposure of the cell to varying dosages of the antibody. In one aspect, the length of time the cell is contacted with the antibody is 1 hour to 3 days, or for 24 hours.

In one aspect, an antibody of the invention, or an antibody used in method of the invention, specifically binds to a toxin. The toxin can be selected from the group consisting of a bacterial toxin, a chemical toxin and an environmental toxin. In some embodiments the bacterial toxin is a cholera toxin, an Escherichia coli toxin, a Streptococcus toxin, a Bordetella pertussis toxin, and a Clostridium toxin. The Clostridium toxin can comprise a botulinum toxin or a Clostridium difficile toxin. The botulinum toxin or Clostridium difficile toxin can comprise botulinum neurotoxin, C. difficile toxin A, or C. difficile toxin B. In one embodiment, the toxin is Ricin toxin. The anti-pathogenic effect of the antibody can result from the antibody binding the toxin, clearance of the toxin, inactivation of the toxin, and the like.

An antibody of the invention, or an antibody used in method of the invention, can specifically bind a virulence factor. The virulence factor can be an adherence factor, a coat protein, an invasion factor, a capsule, an exotoxin, or an endotoxin. The anti-pathogenic effect of the antibody can result from the antibody binding a virulence factor, clearance of the factor, inactivation of the factor, and the like. Exemplary adherence factors include those found in Bordetella pertussis, Campylobacter jejuni, Corynebacterium diphtheriae, Eikenella histolytica, Escherichia coli, Helicobacter pylori, Salmonella enteriditis, Staphylococcus pyogenes, Streptococcus pyogenes, Vibrio cholerae, and Streptococcus viridans. Others include antiphagocytic components (Streptococcus pyogenes, Vibrio vunificans) and the toxins found in, e.g., Camplobacter jejuni, Cornebacterium diptheriae, Legionella pneumophila, Pseudomonas aeruginosa, Shigella dysenterie, Trichomonas vaginalis, Staphylococcus aureus, Bartonella spp., Francisella tularensis, Proteus mirabilis, Salmonella spp., Yersinia spp., and Bacillus cereus. Exemplary capsule components include those found in Bacillus anthracis, Bordetella pertussis, Escherichia coli, Neisseria meningitides, Pasturella multocida, Staphylcoccus epidermis, and Yersinia pestis. Invasion factors include those associated with Clostridium spp., Leptospira interrogans, Staphylococcus aureus, and Vibrio spp.

An antibody of the invention (e.g., Abs made by the methods of the invention, or described herein) is also suitable to modulate the activity of other proteins. For example, the antibody can bind a dietary enzyme, and thus modulate its activity. The dietary enzyme can be a lipase, an esterase, a urease, a lyase, a protease, an isomerase, a ligase or a synthetase. See, e.g., US 2004/0002583.

### Nucleic Acids Encoding and Expressing Abs of the Invention

The invention provides isolated, recombinant and synthetic nucleic acids comprising a sequence encoding an antibody of the invention, a vector comprising the encoding nucleic acid, and/or a cell comprising the encoding nucleic acid or the vector comprising the encoding nucleic acid. In one aspect, the vector comprises the antibody-encoding nucleic acid operably linked to a promoter suitable for expression in the designated host cell.

Host cells for expressing the nucleic acids, expression cassettes and vectors of the invention include bacteria, yeast, fungi, plant cells, insect cells and mammalian cells. Thus, the invention provides methods for optimizing codon usage in all of these cells, codon-altered nucleic acids and polypeptides made by the codon-altered nucleic acids. Exemplary host cells include gram negative bacteria, such as Escherichia coli and Pseudomonas fluorescens; gram positive bacteria, such as Streptomyces diversa, Lactobacillus gasseri, Lactococcus lactis, Lactococcus cremoris, and Bacillus subtilis. Exemplary host cells also include eukaryotic organisms, e.g., various yeast, such as Saccharomyces spp., including Saccharomyces cerevisiae, Schizosaccharomyces pombe, Pichia pastoris, and Kluyveromyces lactis, Hansenula polymorpha, Aspergillus niger, and mammalian cells and cell lines, and insect cells and cell lines. Thus, the invention also includes antibodies and their encoding nucleic acids optimized for expression in these organisms and species. See, e.g., U.S. Pat. No. 5,795,737; Baca (2000) Int. J. Parasitol. 30:113-118; Hale (1998) Protein Expr. Purif. 12:185-188; Narum (2001) Infect. Immun. 69:7250-7253; Narum (2001) Infect. Immun. 69:7250-7253; Outchkourov (2002) Protein Expr. Purif. 24:18-24; Feng (2000) Biochemistry 39:15399-15409; and Humphreys (2000) Protein Expr. Purif. 20:252-264.

The phrase “substantially identical” in the context of two nucleic acids or polypeptides, can refer to two or more sequences that have, e.g., at least about 50%, 55%, 60%, 65%, 70%, 75%, 80%, 85%, 90%, 95%, 98% or 99% or more nucleotide or amino acid residue (sequence) identity, when compared and aligned for maximum correspondence, as measured using one any known sequence comparison algorithm, or by visual inspection. For example, the invention comprises isolated, recombinant or synthetic Ab light or variable region polypeptides having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:1, SEQ ID NO:2, SEQ ID NO:3, SEQ ID NO:4, SEQ ID NO:5, SEQ ID NO:6, SEQ ID NO:7 and/or SEQ ID NO:8, and having the same (or substantially the same) antigen binding specificities as SEQ ID NO:1, SEQ ID NO:2, SEQ ID NO:3, SEQ ID NO:4, , SEQ ID NO:6, SEQ ID NO:7 and/or SEQ ID NO:8, respectively, or to any of the (deposited) monoclonal antibodies of the invention. For example, in alternative aspects, the invention provides nucleic acid and polypeptide sequences having substantial sequence identity to an exemplary sequence of the invention, e.g., SEQ ID NO:26 is the full length of the heavy chain of the Ab designated 227, or 3359; the full length of the light chain of the Ab designated 227, or 3359 (SEQ ID NO:27); the full length of the heavy chain of the Ab designated 543, or 3358 (SEQ ID NO:28); the full length of the light chain of the Ab designated 543, or 3358 (SEQ ID NO:29); the full length of the heavy chain of the Ab designated F87 (SEQ ID NO:30); the full length of the light chain of the Ab designated F87 (SEQ ID NO:31), or any of these sequences over a region of at least about 10, 20, 30, 40, 50, 100, 150, 200, 250, 300, 350, 400, 450, 500, 550, 600, 650, 700, 750, 800, 850, 900, 950, 1000 or more residues, or a region ranging from between about 10, 20, 30, 40, 50, 100, 150, 200, 250 or more residues to the full length of the nucleic acid or polypeptide, wherein these polypeptides (or, the polypeptides encoded by the nucleic acids) have the same antigen binding specificity as the exemplary Ab sequence from which they were derived (e.g., the Ab designated 227, or 3359; or, the Ab designated 543, or 3358; or, the Ab designated F87). Nucleic acid sequences of the invention can be substantially identical over the entire length of an exemplary polypeptide coding region.

A “substantially identical” amino acid sequence also can include a sequence that hybridizes under stringent conditions to a reference sequence (e.g., an exemplary sequence of the invention, e.g., an Ab sequence of the invention comprising the variable regions SEQ ID NO:1, SEQ ID NO:2, SEQ ID NO:3, SEQ ID NO:4, , SEQ ID NO:6, SEQ ID NO:7 and/or SEQ ID NO:8). “Hyblidization” includes the process by which a nucleic acid strand joins with a complementary strand through base pairing. Hybridization reactions can be sensitive and selective so that a particular sequence of interest can be identified even in samples in which it is present at low concentrations. Stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization, as described in detail, below. In alternative aspects, nucleic acids of the invention are defined by their ability to hybridize under various stringency conditions (e.g., high, medium, and low), as set forth herein. In one aspect, hybridization under stringent conditions comprises hybridization in a buffer (solution) comprising about 50% formamide at about 37° C. to 42° C.; or, hybridization under stringent conditions can occur at conditions comprising about 35% to 25% formamide at about 30° C. to 35° C., or, under conditions comprising about 42° C. in 50% formamide, 5×SSPE, 0.3% SDS and 200 n/ml sheared and denatured salmon sperm DNA. The temperature range corresponding to a particular level of stringency can be further narrowed by calculating the purine to pyrimidine ratio of the nucleic acid of interest and adjusting the temperature accordingly. Variations on the above ranges and conditions are well known in the art.

However, the selection of a hybridization format is not always critical—it is the stringency of the wash conditions that set forth the conditions which determine whether a nucleic acid is within the scope of the invention. Wash conditions used to identify nucleic acids within the scope of the invention include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

As used herein, the term “recombinant” can include nucleic acids (e.g., nucleic acids used to practice the invention) adjacent to a “backbone” nucleic acid to which it is not adjacent in its natural environment. In one aspect, nucleic acids represent 5% or more of the number of nucleic acid inserts in a population of nucleic acid “backbone molecules.” “Backbone molecules” according to the invention include nucleic acids such as expression vectors, self-replicating nucleic acids, viruses, integrating nucleic acids, and other vectors or nucleic acids used to maintain or manipulate a nucleic acid insert of interest. In one aspect, the enriched nucleic acids represent 5%, 10%, 15%, 20%, 30%, 40%, 50%, 60%, 70%, 80%, 90%, 95%, 98% or more of the number of nucleic acid inserts in the population of recombinant backbone molecules. “Recombinant” polypeptides or proteins can refer to polypeptides or proteins produced by recombinant DNA techniques; e.g., produced from cells transformed by an exogenous DNA construct encoding the desired polypeptide or protein. “Synthetic” polypeptides or protein are those prepared by chemical synthesis, also are described, below.

The term “expression cassette” as used herein refers to a nucleotide sequence which is capable of affecting expression of a structural gene (i.e., a protein coding sequence, such as an antibody of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression may also be used, e.g., enhancers. Thus, expression cassettes also include plasmids, expression vectors, recombinant viruses, any form of recombinant “naked DNA” vector, and the like.

“Operably linked” as used herein refers to a functional relationship between two or more nucleic acid (e.g., DNA) segments. In one aspect, it refers to the functional relationship of transcriptional regulatory sequence to a transcribed sequence. For example, a promoter is operably linked to a coding sequence, such as a nucleic acid of the invention, if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

As used herein, the term “promoter” includes all sequences capable of driving transcription of a coding sequence in a cell, e.g., a mammalian or plant cell. Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences can interact with proteins or other biomolecules to carry out (turn on/off, regulate, modulate, etc.) transcription. “Constitutive” promoters are those that drive expression continuously under most environmental conditions and states of development or cell differentiation. “Inducible” or “regulatable” promoters direct expression of the nucleic acid of the invention under the influence of environmental conditions or developmental conditions. Examples of environmental conditions that may affect transcription by inducible promoters include anaerobic conditions, elevated temperature, drought, or the presence of light.

“Plasmids” can be commercially available, publicly available on an unrestricted basis, or can be constructed from available plasmids in accord with published procedures. Equivalent plasmids to those described herein are known in the art and will be apparent to the ordinarily skilled artisan.

A promoter sequence can be “operably linked to” a coding sequence when RNA polymerase which initiates transcription at the promoter will transcribe the coding sequence into mRNA, as discussed further, below.

In another aspect, the invention provides a monoclonal antibody, or a biologically active fragment thereof, that binds to Clostridium difficile toxin A, wherein the variable region sequences of the antibody comprise SEQ ID NO:1 and SEQ ID NO:2; or SEQ ID NO:3 and SEQ ID NO:4. The invention also provides an isolated or recombinant nucleic acid comprising a sequence encoding the antibody, a vector comprising the nucleic acid, and a cell comprising the nucleic acid or the vector. Pharmaceutical compositions and kits comprising the antibody are also provided.

In yet another aspect, the invention provides a monoclonal antibody, or a biologically active fragment thereof, that binds to Clostridium difficile toxin B, wherein the variable region sequences of the antibody comprise SEQ ID NO:5 and SEQ ID NO:6. The invention also provides an isolated or recombinant nucleic acid comprising a sequence encoding the antibody, a vector comprising the nucleic acid, and a cell comprising the nucleic acid or the vector. Pharmaceutical compositions and kits comprising the antibody are also provided.

In one aspect, the invention provides a monoclonal antibody produced by or isolated from a hybridoma selected from the group consisting of ATCC Accession No. ______ (Ab designated 227 or 3359), ATCC Accession No. ______ (Ab designated 543 or 3358), ATCC Accession No. ______ (Ab designated F85), ATCC Accession No. ______ (Ab designated F2), and ATCC Accession No. ______ (Ab designated F87). The invention also provides hybridomas comprising ATCC Accession No. ______ (Ab designated 227 or 3359), ATCC Accession No. ______ (Ab designated 543 or 3358), ATCC Accession No. ______ (Ab designated F85), ATCC Accession No. ______ (Ab designated F2), and ATCC Accession No. ______ (Ab designated F87) (“hybridomas of the invention).

The invention provides isolated or recombinant Abs having the same antigen binding specificity as a monoclonal antibody of the invention, and the nucleic acids that encode them. In one aspect, the invention provides isolated or recombinant polypeptides having a sequence identity (e.g., at least about 50%, 55%, 60%, 65%, 70%, 75%, 80%, 85%, 90%, 95%, 96%, 97%, 98%, 99%, or more, or complete (100%) to a sequence of an antibody of the invention, e.g., an Ab produced by a hybridoma of the invention. The identity can be over the full length of the polypeptide, or, the identity can be over a region of at least about 30, 40, 50, 60, 70, 80, 90, 100, 150, 200, 250, 300, 350, 400, 450, 500, 550, 600, 650, 700 or more residues. A sequence that hybridizes to the disclosed sequences under high stringency conditions (see, e.g., Sambrook) is also provided by the invention

Antibodies of the invention can also be shorter than the full length of exemplary antibodies. In alternative aspects, the invention provides antibodies (peptides, fragments) ranging in size between about 5 and the full length of a polypeptide, e.g., as an antibody; exemplary sizes being of about 5, 10, 15, 20, 25, 30, 35, 40, 45, 50, 55, 60, 65, 70, 75, 80, 85, 90, 100, 125, 150, 175, 200, 250, 300, 350, 400, 450, 500, 550, 600, 650, 700, or more residues, e.g., contiguous residues of an exemplary antibody of the invention, where the antibody fragment at least retains the ability to bind the antigen of interest.

Further provided is an isolated or recombinant peptide comprising an epitope bound by a monoclonal antibody of the invention, or a monoclonal antibody generated by a hybridoma of the invention, e.g., a hybridoma selected from the group consisting of ATCC Accession No. ______ (Ab designated 227 or 3359), ATCC Accession No. ______ (Ab designated 543 or 3358), ATCC Accession No. ______ (Ab designated F85), ATCC Accession No. ______ (Ab designated F2), and ATCC Accession No. ______ (Ab designated F87). The invention also provides hybridomas comprising ATCC Accession No. ______ (Ab designated 227 or 3359), ATCC Accession No. ______ (Ab designated 543 or 3358), ATCC Accession No. ______ (Ab designated F85), ATCC Accession No. ______ (Ab designated F2), and ATCC Accession No. ______ (Ab designated F87).

### Methods of Identifying Protease Cleavage Sites and Engineering Oral Antibodies

In one aspect, the invention provides methods of identifying a protease cleavage site in an antibody, which method comprises the steps of: a) determining putative sites of protease cleavage in the antibody; b) prioritizing the protease cleavage sites based on the likely exposure of the site to proteases; and c) identifying a site as the protease cleavage site as one whose position results in an exposure to proteases in the three-dimensional antibody structure. In some embodiments, the putative sites of protease cleavage are determined in step (a) by identifying protease cleavage motifs using N-terminal sequencing, gel electrophoresis analysis, or mass spectral analysis of peptide fragments derived from an antibody digested by protease. The putative sites of protease cleavage can also be determined in step (a) by identifying known protease motifs.

Prioritization of the protease cleavage sites to be modified can be accomplished by any suitable methods. In one embodiment, the protease cleavage sites are prioritized based on the physical location in the antibody, e.g., the hinge region, and the relative exposure to proteases, e.g., sites available after solvent treatment. In some embodiments, the protease cleavage sites are prioritized based on the anticipated protease profile of the target microenvironment. In some embodiments, the protease cleavage sites are prioritized in step (b) based on the surface exposure on the folded form of the antibody solved by x-ray crystallography or NMR spectroscopy. The protease cleavage sites can also be prioritized in step (b) based on the surface exposure determined using a probe of 1.4 angstroms.

The antibodies can be prioritized using any suitable amount of surface exposure. For example, 5, 10, 15, 20, 25, 30, 35, 40, 45, 50, 55, 60, 65, 70, 75, 80, 85, 90, 95, 99 and/or 100% exposure can be used as standards for prioritization. In some embodiments, the identified protease cleavage site has 20% surface area exposure to the probe, wherein the protease cleavage site comprises hydrophobic and aromatic amino acids. In other embodiments, the identified protease cleavage site has 35% surface area exposure to the probe, wherein the protease cleavage site comprises basic amino acids.

Measurements of solvent accessibility can also be used to prioritize the protease cleavage motifs using the exposed van der waals surface or surface residues extrapolated from B-values (PRINCIPLES OF PROTEIN X-RAY CRYSTALLOGRAPHY, (Drenth, ed., Springer Verlag 1994)) or order parameters (Lipari et al., J. Amer. Chem. Soc. 104: 4546 (1982)) from X-ray or NMR structure determination methodologies, respectively. However, the ‘exposed residue’ cutoffs are comparable, even if they do not numerically provide the exact surface area in angstroms squared.

In one aspect, the putative protease cleavage sites within an antibody are prioritized by their surface exposure within the context of the folded form of the antibody. The contact surface area of every residue is calculated from the antibody structure using probe of 1.4 Angstroms (the approximate radius of a water molecule). The surface exposure of an IgG antibody was calculated from the deposited crystal structures of the human IgG1 Fc constant domain (Sonderman et al., Nature 406: 267 (2000)) and the Fab domain (Cho, et al., Nature 421: 756 (2003)) using the program MolMol (Koradi, et al., J. Mol. Graph. 14: 51 (1996)). A probe of 1.4 Angstroms is used to scan the surface of a protein and the area in square Angstroms that the probe is able to contact is defined as the solvent accessible surface area. A cutoff of 20% surface area exposure to solvent (i.e. the probe) was used for hydrophobic and aromatic amino acids (i.e. L, M, I, V, F, Y and W) and a cutoff of 35% was used for basic residues (K, R and H) to classify them as highly exposed and susceptible to potential proteolysis by proteases such as pepsin and those found in pancreatin.

Any number of protease sites can be identified by the method of the invention. In one aspect, at least one protease cleavage site is identified. In some embodiments, the protease cleavage sites comprise the same protease cleavage motif. In other embodiments, the protease cleavage sites comprise two or more different protease cleavage motifs. The protease cleavage sites can be identified in the Fc region, the Fab region, the hinge region, C_{L}, CH₁, CH₂, CH₃, V_{L}, V_{H}, or a combination thereof. The identified protease cleavage motifs include, but are not limited to, a protease selected from the group consisting of pepsin, pancreatin, trypsin, trypsinogen, chymo-trypsin, pro-carboxy-peptidase and pro-elastase.

In one aspect, the invention provides a method of engineering a protease-resistant antibody, which method comprises the steps of: a) providing an antibody or an amino acid sequence of the antibody; b) identifying at least one protease cleavage site in the amino acid sequence of the antibody; and c) introducing at least one modification in the amino acid sequence of the antibody, whereby the modification results in a variant portion that has an increased resistance to proteolysis.

In another aspect, the invention provides a method of generating an engineered antibody that is orally deliverable, which method comprises the steps of: a) providing a nucleic acid encoding a wildtype antibody; b) introducing at least one modification into the coding sequence of the wildtype antibody to generate a modified antibody coding sequence, wherein the modification of the coding sequence is in or proximate to the coding sequence of at least one protease cleavage site and the modification results in expression of an antibody that is partially or completely resistant to digestion by the protease; and c) expressing the modified antibody coding sequence of step b) to generate an engineered antibody, wherein an engineered antibody retains its ability to specifically bind to antigen in the digestive system following oral administration, thereby rendering the engineered antibody orally deliverable.

In some embodiments, the modification is in a protease cleavage site or at a site flanking the protease cleavage site. In alternative embodiments, the modification is at the P1, P1′, P2, P3, P4, P2′, P3′, or P4 residue of the protease cleavage site. One type of modification, therefore, that can be made is to modify a residue that is know to naturally occur within antibody sequences such as IgG, IgA, IgM, IgD, IgE, etc. Such substitutions can identifies through database analysis. See, e.g., Demerast et al., J. Mol. Biol. 335:41-48 (2004). The modification to the amino acid sequence generates a protease resistance motif, rendering the protease cleavage site non-cleavable or less susceptible to protease cleavage.

An engineered antibody of the invention, or an Ab used in a method the invention, can comprise any number of modifications, including but not limited to, two, three, four, five, six, seven, eight, nine, ten, eleven, or more amino acid modifications. The modifications can be in a protease cleavage site or at a site flanking the protease cleavage site. The modification can be made to the same protease cleavage motif within the antibody or to different protease cleavage motifs. In some embodiments, the modification is made in a protease cleavage site that is not flanked by an amino acid residue known to inhibit or attenuate protease cleavage. Such amino acids include Pro, Lys, Arg and His.

An engineered antibody of the invention, or an Ab used in a method the invention, can be an IgG, IgM, IgD, IgE, or IgA antibody. In some embodiments, the antibody is an IgG antibody. An antibody can be an IgG₁, IgG₂, IgG₃, or IgG₄ antibody. The antibody can be a human, murine, rat, rabbit, bovine, camel, llama, dromedary, or simian antibody. The antibody can be a humanized antibody, a chimeric antibody, a bispecific antibody, a fusion protein, or a biologically active fragment thereof.

An engineered antibody of the invention, or an Ab used in a method the invention, can be modified in any portion of the antibody including the heavy chain, a light chain, or both. In some embodiments, the modified portion is the Fc region, the hinge region, the CH_{L} domain, the CH₁ domain, the CH₂ domain, the CH₃ domain, the Fab region, or any combination thereof. In alternative embodiments, the modified portion is a V_{H} or V_{L} domain, provided the cleavage site does not have a negative effect on the desired antibody function.

In one aspect, modifications in the antibody of the invention, or an Ab used in a method the invention, comprise at least one mutation in the amino acid sequence of the antibody. The mutation is introduced by modifications, additions or deletions to a nucleic acid encoding the antibody. The modifications, additions or deletions to a nucleic acid encoding the antibody can be introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturation Mutagenesis (GSSM), synthetic ligation reassembly (SLR) or a combination thereof. The modifications, additions or deletions to a nucleic acid encoding the antibody can also be introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, or a combination thereof.

In one embodiment, an engineered antibody of the invention, or an Ab used in a method the invention, comprises at least one amino acid substitution at any one or more of amino acid positions T155, L179, L235, F241, Y296, L309, Y349, L365, L398, F404, Y407, and Y436 of a IgG heavy chain, wherein the numbering of the residues in the variant portion is that of the EU index as in Kabat, whereby the amino acid substitution confers increased resistance to pepsin proteolysis. In another embodiment, the variant portion comprises at least one amino acid substitution at any one or more of amino acid positions L234, L242, F243, F275, Y278, Y300, L306, W313, L314, Y319, L351, L368, Y391, F405, L406, L410, F423, L432, or Y436 of a IgG heavy chain, wherein the numbering of the residues in the variant portion is that of the EU index as in Kabat, whereby the amino acid substitution confers increased resistance to pepsin proteolysis. In one embodiment, the variant portion comprises at least one amino acid substitution at any one or more of amino acid positions F116, K126, R143, K169 or K183 of a kappa chain, wherein the numbering of the residues in the variant portion is that of the EU index as in Kabat, whereby the amino acid substitution confers increased resistance to pancreatin proteolysis. In another embodiment, the variant portion comprises at least one amino acid substitution at any one or more of amino acid positions K133, K205, K210, K274, K326, K340, R355, K360 or K392 of a IgG heavy chain, wherein the numbering of the residues in the variant portion is that of the EU index as in Kabat, whereby the amino acid substitution confers increased resistance to pancreatin proteolysis.

In one aspect, an engineered antibody of the invention comprises at least one amino acid substitution at the P1 or P1′ site of cleavage in a trypsin cleavage motif, wherein the substituted amino acid is K or R, whereby the amino acid substitution confers increased resistance to trypsin proteolysis. In one embodiment, an engineered antibody comprises at least one amino acid substitution, at the P1 or P1′ site of cleavage in a pepsin cleavage motif, wherein the substituted amino acid is L, F, Y, W, I, or T, whereby the amino acid substitution confers increased resistance to pepsin proteolysis. In some embodiments, the engineered antibody comprises at least one amino acid substitution at the P1 or P1′ site of cleavage in a chymotrypsin cleavage motif, wherein the substituted amino acid is F, Y, or W, whereby the amino acid substitution confers increased resistance to chymotrypsin proteolysis.

In one embodiment, an engineered antibody of the invention comprises at least one amino acid substitution selected from the group of amino acid substitutions of L235P, L398Q, F404Y, L179I, and T155S in an IgG₁ heavy chain, wherein the numbering of the residues in the engineered antibody is that of the EU index as in Kabat, whereby the amino acid substitution confers increased resistance to pepsin proteolysis. In another specific embodiment, the engineered antibody comprises at least one amino acid substitution selected from the group of amino acid substitutions of F116S and K126A in a kappa light chain, wherein the numbering of the residues in the engineered antibody is that of the EU index as in Kabat, whereby the amino acid substitution confers increased resistance to pepsin proteolysis. In yet another specific embodiment, the engineered antibody comprises at least one amino acid substitution selected from the group of amino acid substitutions of K133G and K274Q in an IgG heavy chain, wherein the numbering of the residues in the engineered antibody is that of the EU index as in Kabat, whereby the amino acid substitution confers increased resistance to pepsin proteolysis.

In some embodiments, an engineered antibody of the invention, or an Ab used in a method the invention, has greater resistance to proteolysis relative to the wildtype antibody. The increased resistance to proteolysis is at least 1%, 5%, 10%, 20%, 30%, 40%, 50%, 60%, 70%, 80%, 90% or 95% or more than that of the unmodified antibody. An engineered antibody can be partially or completely resistant to cleavage by more than one protease. An engineered antibody of the invention can be modified in any suitable manner. In some embodiments, the modification comprises the addition of a post-translational modification site, an N-glycosylation site, an O-glycosylation site, an alkyl chain, or a small molecule. At times, the modification comprises covalent or non-covalent addition of a second molecule to the Fc chain of the antibody. The second molecule comprises an antibody secretory component, a carbohydrate, a disulfide bond site, or a salt bridge site.

In some embodiment, the Fc region of an engineered antibody of the invention, or an Ab used in a method the invention, is further modified to enhance ADCC, CDC, or phagocytosis. The Fc region of the antibody can also be further modified to increase binding affinity to the Fc receptor (FcR). In one embodiment, an engineered antibody is further modified to have a) an antigen binding activity comparable to or superior to the unmodified antibody; b) a chemical stability comparable to or superior to the unmodified antibody; c) a thermostability or thermotolerance comparable to or superior to the unmodified antibody; d) a pH tolerance comparable to or superior to the unmodified antibody; e) a reduced immunogenicity; f) a reduced aggregation; g) an increased half-life relative to the unmodified antibody; h) an increased expression in a host cell; i) a stability in pharmaceutical formulation comparable or superior to that of the unmodified antibody; j) an enhanced dimerization of Fc regions; or k) any combination thereof. In some embodiments, an antibody of the invention has a) an antigen binding activity comparable to or superior to the unmodified antibody; b) a chemical stability comparable to or superior to the unmodified antibody; c) a thermostability or thermotolerance comparable to or superior to the unmodified antibody; d) a pH tolerance comparable to or superior to the unmodified antibody; e) a reduced immunogenicity; f) a reduced aggregation; g) an increased half-life relative to the unmodified antibody; h) an increased expression in a host cell; i) a stability in pharmaceutical formulation comparable or superior to that of the unmodified antibody; j) an enhanced dimerization of Fc regions; or k) any combination thereof.

In one embodiment, an engineered antibody of the invention, or an Ab used in a method the invention, maintains its native conformation at about pH 3 and above or is modified to do so. In another specific embodiment, the antibody retains biological activity at pH 3 or is further modified to do so. In some embodiments, the antibody further comprises additional mutations that render the antibody more resistant to pH dependent unfolding.

In some embodiments, the proteolysis is the digestion mediated by proteases from the gastrointestinal track, the blood, or the bile. In alternative embodiments, the proteolysis is mediated by pepsin, pancreatin, trypsin, trypsinogen, chymo-trypsinogen, carboxy-peptidase, pro-carboxy-peptidase, elastase, pro-elastase, or any combination thereof. The protease can be selected from a group of proteases released by an exogenous organism or any organism within the digestive tract, or released or produced in the digestive tract. In some embodiments, the protease can be selected from a group of proteases released or produced by an abnormal, infected, cancerous or otherwise diseased tissue.

In some embodiments, an engineered antibody of the invention specifically binds to a pathogen. The pathogen can be a bacteria, a virus and a fungus. In some cases, the pathogen is an intestinal pathogen, including but not limited to enterotoxigenic E. coli, rotavirus, Cryptosporidium parvum, Clostridium difficile, Shigella flexneri, Enterococcus faecalis, Enterococcus faecium, Campylobacter jejuni, Staphylococcus aureus, E. coli O157:H7, Helicobacter pylori, Pseudomonas aeruginosa, Shigella dysenteriae, Salmonella enteritidis, Salmonella typhi, Clostridium perfringens, Aeromonas hydrophila, and Aeromanas aerolysin. In some embodiments, the pathogen is Streptococcus mutans.

In one aspect, an engineered antibody of the invention specifically bind to a toxin. The toxin can be selected from the group consisting of a bacterial toxin, a chemical toxin and an environmental toxin. In some embodiments the bacterial toxin is a cholera toxin, an Escherichia coli toxin, a Streptococcus toxin, a Bordetella pertussis toxin, and a Clostridium toxin. The Clostridium toxin can comprise a botulinum toxin or a Clostridium difficile toxin. The botulinum toxin or Clostridium difficile toxin can comprise botulinum neurotoxin, C. difficile toxin A, or C. difficile toxin B.

An engineered antibody of the invention, or an Ab used in a method the invention, can specifically bind a virulence factor. The virulence factor can be an adherence factor, a coat protein, an invasion factor, a capsule, an exotoxin, or an endotoxin.

An engineered antibody of the invention, or an Ab used in a method the invention, can specifically binds to a dietary enzyme. The dietary enzyme can be a lipase, an esterase, a urease, a lyase, a protease, an isomerase, a ligase or a synthetase.

In another aspect, the invention provides an isolated or recombinant nucleic acid comprising a sequence encoding an engineered antibody of the invention, a vector comprising the encoding nucleic acid, and a cell comprising the encoding nucleic acid or the vector comprising the encoding nucleic acid.

In yet another aspect, the invention provides a method of stabilizing antibody activity in the presence of a protease comprising introducing at least one mutation into the amino acid sequence of the antibody that reduces or eliminates the loss of antibody structural integrity after protease digestion, thereby stabilizing antibody activity. The stabilized antibody maintains at least a portion of its biological activity. Some of the mutations of the invention result in greater antibody stability include an additional disulfide bond or glycosylation site.

In another aspect, the invention provides a method of stabilizing antibody activity in the presence of a protease comprising crosslinking linking one or more antibodies, wherein the antibody comprises an antibody made by a method of the invention or an antibody described herein, and crosslinking reduces or eliminates the loss of antibody structural integrity after protease digestion, thereby stabilizing antibody activity.

In yet another aspect of the invention, the invention provides a method of stabilizing antibody activity in the presence of a protease comprising introducing at least one mutation into the amino acid sequence of the antibody, wherein the mutation permits association of the antibody with a secretory component, wherein the association with the secretory component reduces or eliminates the loss of antibody structural integrity after protease digestion, thereby stabilizing antibody activity by maintaining the structural integrity of the antibody.

### Therapeutic Uses of Antibodies and Compositions Thereof

In another aspect, the invention provides methods of ameliorating, treating or preventing disease, infection, or other disorder caused by an abnormal cell, pathogen or toxin comprising administering orally a pharmaceutically effective amount of the antibody of invention, or the pharmaceutical composition comprising the antibody, to a subject in need thereof, whereby the disease, infection or other disorder is treated or prevented. Any subject can be treated using the antibody of the invention where the disease, infection, or disorder suggests the desirability of such treatment. Thus, any mammal can be treated, including but not limited to humans, cattle, horses, hogs, dogs, cats, and the like.

An antibody of the invention, or an antibody used in a method of the invention, can target any abnormal cell, e.g., a cancer cell. In one aspect, the antibody will bind at least one antigen expressed on the cell surface of the cell. The cancer treated by this method can be an adenocarcinoma, squamous carcinoma, leukemia, lymphoma, melanoma, sarcoma, or teratocarcinoma. In some embodiments, the tumor is a cancer of the adrenal gland, bladder, bone, bone marrow, brain, breast, cervix, colon, gall bladder, ganglia, gastrointestinal tract, head and neck, heart, kidney, liver, lung, muscle, ovary, pancreas, parathyroid, penis, prostate, rectum, salivary glands, skin, spleen, testis, thymus, thyroid, or uterus. In some embodiments, the cancer is colon cancer or gastrointestinal cancer.

The abnormal cell targeted by an antibody of the invention, or an antibody used in a method of the invention, can be an inflammatory cell or a chronically activated cell. Such cells often result in chronic inflammation, inflammatory sequelae, or autoimmunity. The antibody can also target a virulence factor on a pathogen, including a toxin such as those disclosed herein.

In one embodiment, the invention provides a method of ameliorating, treating or preventing gastrointestinal infections or other disorders caused by a pathogen or a toxin comprising administering orally a pharmaceutically effective amount of an engineered antibody of invention, or the pharmaceutical composition comprising the antibody, to a subject in need thereof, whereby the infection or other disorders is treated or prevented.

The present method further comprises the co-administration of at least one anti-infectious agent or drug. Any suitable anti-infectious agent or drug can be used. In some embodiments, the anti-infectious agent or drug is selected from the group consisting of an antibiotic, a second antibody, and a biologically active protein. Any suitable antibiotic can be used in the methods of the invention. Exemplary antibiotics include beta-lactams, aminoglycosides, vancomycin, linezolid, chloramphenicol, macrolide antibiotics, trimethoprim/sulfamethazole, clindamycin, metronidazole, rifampin, mucopirin, fluoroquinolones, as well as generational derivatives of known classes of antibiotics.

In one aspect, the second antibody comprises a second orally deliverable antibody produced by the method provided herein, wherein the second antibody is directed to a different target epitope or protein than the first antibody. The second antibody can also be targeted to a different virulence factor.

In one aspect, the invention provides a method to ameliorate or prevent toxicity associated with Clostridium difficile, comprising administering to a subject in need thereof: a) a therapeutically effective amount of a first monoclonal antibody, wherein the first monoclonal antibody comprises the heavy chain variable region sequence of SEQ ID NO:1 and the light chain variable region sequence of SEQ ID NO:2; and b) a therapeutically effective amount of a second monoclonal antibody, wherein the second monoclonal antibody comprising the heavy chain variable region sequence of SEQ ID NO:3 and the light chain variable region sequence of SEQ ID NO:4, whereby the antibodies ameliorate or prevent the toxicity associated with Clostridium difficile toxin A. In one embodiment, the method further comprises administering a third monoclonal antibody, wherein the third antibody is a monoclonal antibody comprising the heavy chain variable region sequence of SEQ ID NO:5 and the light chain variable region sequence of SEQ ID NO:6, whereby the antibodies ameliorate or prevent the toxicity associated with Clostridium difficile toxin B.

In another aspect, the invention provides a method of ameliorating or preventing toxicity associated with Clostridium difficile, comprising administering to a subject in need thereof: a) a first antibody that partially or completely inhibits binding of a Clostridium difficile toxin A to a cell; and b) a second antibody that partially or completely inhibits intracellular internalization of the Clostridium difficile toxin A, wherein the first antibody and the second antibody bind to the Clostridium difficile toxin A at non-overlapping epitopes. In one embodiment, the method further comprises administering a therapeutically effective amount of a third antibody that partially or completely neutralizes Clostridium difficile toxin B. In one embodiment, the second antibody is not the monoclonal antibody PCG-4.

In some embodiments, the first and second antibodies synergize to neutralize the virulence factor at an antibody concentration lower than the antibody concentration necessary to observe partial neutralization by each antibody alone. In one embodiment, the first monoclonal antibody and the second monoclonal antibody bind to a Clostridium difficile toxin A at ToxA:1800-2710. In some embodiments, the third antibody is a monoclonal antibody that binds to a Clostridium difficile toxin B at ToxB:1807-2366. In one aspect, the first monoclonal antibody and the second monoclonal antibody do not bind Clostridium difficile toxin B, and the third monoclonal antibody does not bind Clostridium difficile toxin A.

In one embodiment, the methods of the invention employ monoclonal antibodies comprising recombinant or synthetic antibodies. One or more of the antibodies can be rendered partially or completely resistant to proteolysis and/or orally deliverable using the antibody engineering methods of the invention.

The antibodies and methods of the invention can be useful in the treatment of the Clostridium toxin-related toxicity in a subject, wherein the toxicity comprises Clostridium-associated diarrhea, colitis or a related condition, whereby one or more symptoms of the Clostridium-induced diarrhea, colitis, or related condition are ameliorated or prevented following administration of the monoclonal antibodies. In one aspect, these antibodies can be an IgG antibody. In one embodiment, the antibody is a human, murine, rat, rabbit, bovine, camel, llama, dromedary, or simian antibody.

In some embodiments, the antibody of the invention, or the Ab used in a method of the invention, is a humanized antibody, chimeric antibody, bispecific antibody, fusion antibody, nanobody, diabody, scFv, or biologically active fragment thereof. An antibody of the invention, or an Ab used in a method of the invention, can be modified to increase resistance to proteolysis. The antibody can be modified to be orally deliverable, using, for example, when practicing the methods of the invention.

Any suitable biologically active protein may be employed in practicing the methods of the invention. In one embodiment, the biologically active protein is a toxin-degrading or toxin-inactivating protease. In one aspect, the protease is capable of partially or completely degrading or inactivating the targeted toxin. The toxin can come from any source, including but limited to a bacterial toxin, a chemical toxin and an environmental toxin.

When an antibody of the invention is co-administered with one or more biologically active agents, the antibody provided herein may be administered either simultaneously with the biologically active agent(s), or sequentially. If administered sequentially, the attending physician will decide on the appropriate sequence of administering protein of the invention in combination with the biologically active agent(s).

### Pharmaceutical Compositions and Formulations

The invention provides pharmaceutical compositions and formulations comprising an antibody of the invention, or the novel combination of antibodies of the invention, or an antibody made by method of the invention (e.g., an antibody modified to be resistant, completely or partially, to a protease). The invention provides pharmaceutical compositions comprising an Ab of the invention, or the novel combination of antibodies of the invention, or an antibody used in or made by a method of the invention, and a suitable excipient (e.g., a pharmaceutically acceptable excipient). In one aspect, the invention provides combinations of monoclonal and/or synthetic antibodies, e.g., “synthetic polyclonals,” that work synergistically to neutralize bacterial toxins, e.g., enteric bacterial toxins such as Clostridium difficile toxin A.

In some embodiments, the pharmaceutical composition is formulated as a suspension, a liquid, a capsule, a tablet, a gel, a microsphere, a liposome, a multiparticulate core particle or a spray. In one embodiment, the antibody comprises 1%, 5%, 10%, 20%, 30%, 40%, 50%, 60%, 70%, 80%, 90% or 95% or more, or from about 50% to about 95%, of the batch size (weight/weight) of the pharmaceutical composition. In some embodiments, the pharmaceutical composition is formulated for enteric or oral delivery. In one embodiment, the pharmaceutical composition further comprises an enteric coating or any coating for oral delivery, e.g., as gelatin capsules, liposomes or formulated as a pre-liposome formulation and then put into a capsule.

The antibodies of the invention may serve as diagnostic tools. In one aspect, antibodies are labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. In addition, the antibodies provided herein can be useful as the antigen-binding component of fluorobodies. See e.g., Zeytun et al., Nat. Biotechnol. 21:1473-79 (2003).

In one aspect, the invention provides a pharmaceutical composition comprising an engineered antibody of the invention, or an antibody used in or made by a method of the invention, and a suitable excipient. In some embodiments, the composition is formulated as a suspension, a liquid, a capsule, a tablet, a gel, a microsphere, a liposome, a multiparticulate core particle or a spray. In one embodiment, the antibody comprises from about 1%, 5%, 10%, 20%, 30%, 40%, 50%, 60%, 70%, 80%, 90% or 95% or more, or from about 50% to about 95%, of the batch size (weight/weight) of the pharmaceutical composition. In some embodiments, the composition is formulated for enteric or oral delivery. In one embodiment, the pharmaceutical composition further comprises an enteric coating or any coating for oral delivery, e.g., as gelatin capsules, liposomes or formulated as a pre-liposome formulation and then put into a capsule.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Antibodies exhibiting high therapeutic indices are used to practice the invention, e.g., are the antibodies modified by the methods of the invention.

The data obtained from these cell culture assays and animal studies can be used in formulating a range of dosage for use in human. The dosage of such compounds lies in one aspect within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl. et al., THE PHARMACOLOGICAL BASIS OF THERAPEUTICS 1 (latest edition). Dosage amount and interval may be adjusted individually to provide plasma levels of the active moiety sufficient to maintain the desired therapeutic effects, or minimal effective concentration (MEC). The MEC will vary for each compound but can be estimated from in vitro data; for example, the concentration necessary to achieve 50% neutralization of the targeted virulence factor activity.

In some embodiments, the subject is pretreated to lower the pH of the intestine (make more acidic) or increase (make more basic) the pH of the stomach. Such methods are well known in the art. In some embodiments, the subject is pretreated or co-treated with at least one antibiotic. In an alternative embodiment, the disorder is an ulcer.

Any enteric pathogen can be treated using an antibody of the invention, or using the methods of the invention. In one embodiment, the enteric pathogen is Clostridium difficile. In one aspect, the invention provides a method of ameliorating or preventing toxicity associated with a first virulence factor in a subject, comprising administering to the subject in need thereof a therapeutically effective amount of at least two monoclonal antibodies, wherein the antibodies synergize to neutralize the effects of the first virulence factor, thereby ameliorating or preventing the toxicity associated with the first virulence factor. As described above, any virulence factor can be targeted. In one embodiment, the first virulence factor is a toxin, alternatively a Clostridium sp. toxin, a toxin A or toxin B.

Thus, in one embodiment, the antibodies administered comprise a first antibody that partially or completely inhibits binding of a Clostridium difficile toxin to a cell; and a second antibody that partially or completely inhibits intracellular internalization of the Clostridium toxin, wherein the first antibody and the second antibody bind to the Clostridium toxin at non-overlapping epitopes. In one aspect, the monoclonal antibodies neutralize the first virulence factor to the same degree or greater than a polyclonal antiserum. In one embodiment, the Clostridium difficile toxin is a Clostridium difficile toxin A or a Clostridium difficile toxin B. The monoclonal antibodies of the invention, or an Ab used in a method of the invention, can be recombinant or synthetic antibodies as described above.

In some embodiment, a method of the invention comprises administering a therapeutically effective amount of a third antibody that partially or completely neutralizes a second virulence factor. In one embodiment, the second virulence factor is Clostridium difficile toxin B and the first virulence factor is Clostridium difficile toxin A. The Clostridium toxin-related toxicity in the subject treated by the methods of the invention comprises Clostridium-associated diarrhea, colitis or a related condition, and whereby one or more symptoms of the Clostridium-induced diarrhea, colitis, or related condition are ameliorated or prevented following administration of the monoclonal antibodies. In alternative embodiments, at least one of the antibodies produced by the method of invention is partially or completely protease-resistant. In one aspect, an Fe portion of the antibody is partially or completely protease-resistant. These antibodies can be administered orally, e.g., be formulated for oral delivery.

In some embodiments, the first and second antibodies synergize to neutralize the virulence factor at an antibody concentration lower than the antibody concentration necessary to observe partial neutralization by each antibody alone.

In one aspect, the virulence factor is Clostridium difficile toxin A. The first monoclonal antibody and the second monoclonal antibody bind to a Clostridium difficile toxin A at ToxA:1800-2710. In one embodiment, the second monoclonal antibody is not PCG-4. In some embodiments, the third antibody is a monoclonal antibody that binds to a Clostridium difficile toxin B at ToxB:1807-2366. In one aspect, the first monoclonal antibody and the second monoclonal antibody do not bind Clostridium difficile toxin B, and the third monoclonal antibody does not bind Clostridium difficile toxin A. In some embodiments, the antibodies each bind different virulence factors of the pathogen.

The therapeutic methods of the invention can further comprise administering a therapeutically effective amount of a protease that partially or completely neutralizes the toxin. The protease is administered in the same formulation as the first antibody, the same formulation as the second antibody, or in the same formulation as the first and the second antibody.

In one embodiment, the antibodies and any other bioactive agent, e.g., a protease, are administered in an enteral (enteric) formulation. Any suitable enteral formulation may be employed. See e.g., REMINGTON: THE SCIENCE AND PRACTICE OF PHARMACY (latest edition). Thus, the antibodies can be formulated as a suspension, a liquid, a capsule, a tablet, a gel, a plant matrix material, a microsphere, a liposome, a multiparticulate core particle or a spray.

In another aspect, the invention provides a method of ameliorating or preventing toxicity associated with a virulence factor in a cell, comprising administering to the cell a therapeutically effective amount of at least two monoclonal antibodies, wherein the antibodies synergize to neutralize the effects of the virulence factor, thereby ameliorating or preventing the toxicity associated with the virulence factor.

In yet another aspect, the invention provides a method of ameliorating or preventing toxicity associated with a virulence factor in a cell, comprising administering to the cell a) a first antibody that partially or completely inhibits binding of a Clostridium difficile toxin to a cell; and b) a second antibody that partially or completely inhibits intracellular internalization of the Clostridium difficile toxin, wherein the first antibody and the second antibody bind to the Clostridium difficile toxin at non-overlapping epitopes, and with the proviso that the second antibody is not the monoclonal Ab PCG-4.

Any suitable biologically active agent may be co-administered with an antibody of the invention. In some embodiments, the antibodies or antigen binding fragments thereof provided herein may be conjugated to a bioactive agent.

In practicing the methods of the invention, or when administering an antibody of the invention, e.g., for treating an infection or a disease or a condition, such as a cancer, or for a diagnostic purpose, the Ab or antibodies can be co-administered with at least one bioactive agent. These antibodies and/or agents can be administered sequentially or simultaneously. Co-administered agents include but are not limited to agents such as cytokines, such as IL-2, IL-12, interferon (IFN), Tumor Necrosis Factor (TNF); photosensitizers (for use in photodynamic therapy), including aluminum (III) phthalocyanine tetrasulfonate, hematoporphyrin, and phthalocyanine; radionuclides, such as indium-111 (¹¹¹In), iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re), and rhenium-188 (¹⁸⁸Re); antibiotics, such as doxorubicin, daunorubicin, methotrexate, neocarzinostatin, and carboplatin; bacterial, plant, and other toxins, such as diphtheria toxin, pseudomonas exotoxin A, mystatin, staphylococcal enterotoxin A, abrin-A toxin, ricin A (deglycosylated ricin A and native ricin A), TGF-α toxin, cytotoxin from Chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; 1y207702 (a difluorinated purine nucleoside); liposomes containing antitumor agents (e.g., antisense oligonucleotides, siRNA, plasmids encoding toxins, methotrexate, etc.); other antibodies or antibody fragments, such as F(ab); anti-angiogenic agents including protamine, heparin, steroids, thalidomide, TNP-470, carboxyamidotriazole (CAI), interferon alpha (IFN-α), angiostatin, endostatin, and Avastin™ (anti-VEGF); enzymes (e.g., asparaginase); catalytic nucleic acids, (e.g., hammerhead ribozymes), hormonal agents (e.g., tamoxifen and onapristone), and the like.

The invention further provides antibodies engineered by the methods of the invention and useful in the methods of treatment of pathogen-induced symptoms and diseases as described above. Thus, in one embodiment, the first monoclonal antibody is produced by the hybridoma ATCC Accession No. ______ (Ab designated 227 or 3359). The second monoclonal antibody is produced by ATCC Accession No. ______ (Ab designated 543 or 3358). The third antibody comprises a monoclonal antibody produced by a hybridoma selected from the group consisting of ATCC Accession No. ______ (Ab designated F85), ATCC Accession No. ______ (Ab designated F2), and ATCC Accession No. ______ (Ab designated F87).

The invention provides pharmaceutical compositions comprising at least one antibody of the invention, e.g., a monoclonal antibody (Mab) or a novel combination of Mabs of the invention, and a suitable excipient. Formulations and excipients useful in the pharmaceutical compositions are those well known in the art. An antibody of the invention, or any antibody used in the methods of the invention (from whatever source derived, including without limitation from recombinant sources), may be administered to a subject in need, by itself, or in pharmaceutical compositions where it is mixed with suitable carriers or excipient(s) at doses to treat or ameliorate a variety of disorders. See e.g., REMINGTON: THE SCIENCE AND PRACTICE OF PHARMACY (latest edition). Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain other anti-pathogen or anti-tumor agents such cytokines or chemotherapeutic agents as is desirable.

The precise dose will depend upon a number of factors, including whether the antibody is for diagnosis or for treatment, the size and location of the area to be treated, the precise nature of the antibody (e.g., whole antibody, fragment, diabody or triabody), and the nature of any other molecule attached to the antibody. The exact formulation, route of administration and dosage can be chosen by the individual physician in view of the patient's condition. See, e.g., THE PHARMACOLOGICAL BASIS OF THERAPEUTICS (Goodman et al., eds., McGraw-Hill Professionals, 9th Ed. 1996). Dosage amount and interval may be adjusted individually to provide plasma levels of the active moiety sufficient to maintain the desired therapeutic effects, or minimal effective concentration (MEC).

In one aspect, the antibody dose is in the range of between about 0.1, 0.5, 1.0, 5.0 or 10.0 μg to 50, 60, 70, 80, 90 or 100 μg, or alternatively, 100, 200, 300, 400 or 500 μg to about 600, 700, 800, 900 or 1,000 μg (1 mg), or from about 1, 5, 10, 50 100, 200, 300, 400 or 500 mg to about 600, 700, 800, 900 or 1,000 mg (1 gm) or more for oral applications. In one aspect, the antibody is a whole antibody, in one aspect an IgG isotype, e.g., the IgG₁ isotype. In one aspect, a dose for a single treatment of an adult patient, as described herein, is proportionally adjusted for children and infants, and also adjusted for other antibody formats in proportion to molecular weight. Treatments may be repeated at, e.g., hourly, every 2, 4, 6, or 12 hours, daily, twice-weekly, weekly, every 21 days, every 28 days, or monthly intervals, at the discretion of the physician. In one aspect, treatment is periodic, and the period between administrations is, e.g., hourly, every 2, 4, 6, or 12 hours daily (e.g., b.i.d., t.i.d.), or weekly, or about two weeks or more, or about three weeks or more, or about four weeks or more, or about once a month.

Pharmaceutical compositions for use in practicing the methods of the invention, or for formulating Abs of the invention, can be formulated in any conventional manner using one or more physiologically acceptable carriers comprising excipients and auxiliaries that facilitate processing of the active compounds into preparations that can be used pharmaceutically. These pharmaceutical compositions may be manufactured in a manner that is itself known, e.g., by means of conventional mixing, dissolving, granulating, dragee-making, levigating (e.g., to make a smooth, fine powder or paste, as by grinding when moist), emulsifying, encapsulating, entrapping or lyophilizing processes. Proper formulation is dependent upon the route of administration chosen. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the invention, and in one aspect from about 1 to 50% protein of the invention.

Antibodies of the invention, or antibodies used in the methods of the invention, can be encapsulated into gelatin capsules, liposomes or formulated as a pre-liposome formulation and then put into a capsule. The capsule can be a soft gel capsule capable of tolerating a certain amount of water, a two piece capsule capable of tolerating a certain amount of water or a two piece capsule where the liposomes are preformed then dehydrated. The liposomes used to practice the invention can comprise any bilayer forming lipid, e.g., phospholipids, sphingolipids, glycosphingolipids, and ceramides. When using gelatin capsules, e.g., a soft gel capsule can be 10% on the interior, or, the concentration of water in a liposome formulation can range from 5% to 90% water. Capsulation can protect the liposome-antibody complex from the low pH of the stomach, emulsification from bile salts and degradation by digestive enzymes. This protection can be further enhanced when the outer shell of the capsule is coated with a polymer like hydroxyethylmethyl cellulose propylethyl acetate or hydroxypropylmethylcellulose propylethyl thallate. See, e.g., U.S. Pat. No. 6,726,924.

In one aspect, the bile acid transport system is manipulated to provide sustained systemic concentrations of orally delivered antibody formulations of the invention,

### Kits

The invention provides kits comprising the compositions, e.g., nucleic acids, expression cassettes, vectors, cells, transgenic seeds or plants or plant parts, polypeptides (e.g. antibodies) and/or antibodies of the invention. The kits also can contain instructional material teaching the methodologies and industrial uses of the invention, as described herein.

In one aspect, the invention provides a kit for ameliorating or preventing one or more symptoms of virulence factor-associated symptom or disease, comprising a) a pharmaceutical composition comprising the monoclonal antibodies disclosed herein and a suitable excipient; and b) instruction for administering the pharmaceutical composition. In some embodiments, the pharmaceutical composition comprises a) a first monoclonal antibody that partially or completely inhibits binding of Clostridium difficile toxin A to a cell; b) a second monoclonal antibody that inhibits Clostridium difficile toxin A intracellular internalization, wherein the first monoclonal antibody and the second monoclonal antibody bind toxin A at non-overlapping epitopes, and with the proviso that the second monoclonal antibody is not PCG-4; c) a third monoclonal antibody that partially or completely neutralizes Clostridium difficile toxin B; d) an anti-Clostridium difficile toxin protease; e) a suitable excipient; and instructions for administering the pharmaceutical composition.

Specifically provided herein is a compartment kit comprising one or more containers, wherein a first container comprises one or more antibodies engineered by the methods of the invention, and one or more other containers comprising one or more of the following: wash reagents, reagents necessary for administration of the antibody or capable of detecting presence of a bound antibody. The containers can be glass, plastic, or strips of plastic or paper. Types of detection agents include labeled secondary antibodies, other labeled secondary binding agents, or in the alternative, if the primary antibody is labeled, the enzymatic, or antibody binding reagents that are capable of reacting with the labeled antibody. Ancillary materials to assist in or to enable performing such a method may be included within a kit of the invention.

The following examples are intended to illustrate but not to limit the invention

### EXAMPLES

### Example 1

### Engineering Antibodies Resistant to Intestinal Fluids

The invention provides antibodies for oral delivery, and methods for the development of antibodies that are stable in the digestive-tract environment. This example describes an exemplary method of the invention for developing antibodies that are stable in the digestive-tract environment, i.e., antibodies for oral delivery, and exemplary antibodies of the invention made by these methods. This example describes an exemplary method of the invention using the Kabat numbering system to design/make an antibody with the scope of the invention (see, e.g., Table 1, below).

To create an antibody molecule stable in gastric fluids, various antibody classes were tested in simulated gastric fluids. Antibody molecules were incubated with pepsin at pH 1.2 in order to simulate the gastric phase of digestion. Initial time course digestibility profiles were performed with pepsin on human IgG₁, IgG₂, IgG₃, and IgG₄. All antibody classes were rapidly proteolyzed into small fragments, see FIG. 1. In FIG. 1, antibodies were digested for 0, 2, 5, 10, 20 and 30 min with pepsin. The letter C denotes the test antibody without pepsin. Molecular weight markers (MW-kDa) are indicated between the gels.

IgG₂ and IgG₄ appeared to undergo extensive proteolysis at very early time points. However, IgG₁ and IgG₃ seemed to display superior “resistance” to pepsin digestion. On a reducing gel, IgG₁ exhibited a higher proportion of light chain maintained throughout time when compared to IgG₃, see FIG. 2. In FIG. 2, IgG₁ (10 μg/lane) was digested for 0, 2, 5, 10, 20 and 30 min with pepsin. Molecular weight marker (MW-kDa) is indicated.

Interestingly, the acidic conditions alone in the absence of pepsin led to decreases in functional antibody as tested by ELISA, see FIG. 3. In FIG. 3, 1 μg was digested for 0, 2, 5, 10 20, and 30 min with pepsin (×0.005) at pH 1.5. The molecular weight marker (MW-kDa) is indicated. Samples were either loaded on a 4-12% Bis-Tris gel and Coomassie stained or tested in ELISA. An AP-labeled mouse anti-human Fc antibody was used for detection in the ELISA. A1 and A2: pH 1.5 and pepsin. B1 and B2: pH 1.5. This decrease was not detectable by electrophoretic analysis of the digestion. Between pH 2 and pH 3, the antibody molecule exhibits a pH dependent unfolding event (see FIG. 4) leading to some irreversible degradation/aggregation. In FIG. 4, the spectra of IgG₁ at pH values of 3 and above were highly indicative of β-sheet-like structure with a single minimum at 217 nm. At pH 2 and below, the spectra changed radically to spectra highly indicative of random coil (unfolded) with the characteristic minimum at 197 nm.

Determination of Cleavage Sites to Mutate:

To identify residues that should be mutated to engineer an antibody molecule with increased resistance to pepsin, two approaches were employed: a proteomic approach and the calculation of surface exposure of potential pepsin cleavage sites within the antibody.

Approach 1: Digested IgG1 was assessed by mass spectral analysis to identify the pepsin cleavage sites. Because antibody fragments were still too large for analysis by tandem mass spectrometry (MS/MS), trypsin was used to generate smaller peptides in the presence of a 1:1 mixture of ¹⁶O/¹⁸O, so that peptides produced with pepsin should have a normal isotopic distribution (singlet) and peptides produced from trypsin should have a modified distribution (doublet). Four pepsin cleavage sites were identified using this approach.

Approach 2: The human IgG Fc structure was analyzed for exposed pepsin cleavage motifs as previously described in Delano, et al. 2000; Keil, 1992 (see below). 10 highly exposed sites were determined as potential candidates for directed mutagenesis. Interestingly, 2 of these sites were also found by mass spectral analysis to be pepsin cleavage sites. A potential cleavage site within the hinge region was also found by sequence analysis. This set of 10 mutants along with the cleavage sites identified by mass spectral analysis (12 total) comprised the list of residues deemed significant for pepsin resistance. See Table 1, below.

A secondary set of residues was also determined simply by identification of pepsin recognition motifs without consideration of surface exposure. Addition of this second set of residues provided a total of 31 potential sites for mutation within the constant domain of the heavy chain (Table 1).

Table 1 (below) shows the position of mutations engineered in the human IgG1 heavy chain. In red (or only bolded) are the highest priority based on surface exposure, sequence and proteomic analysis. In blue (or underlined) are the other potential pepsin cleavage sites based on sequence analysis only. All prioritized residues were >20% exposed to solvent based on an available crystal structure (Delano et al., 2000) and all de-prioritized residues were <20% exposed unless otherwise indicated. ^{a}Proteomically determined cleavage site; ^{b}proteolytic enhancing flanking motif; ^{c}proteolytic inhibitory flanking motif; ^{d}Carbohydrate interacting residue; ^{e}Greater than 20% exposed, de-prioritized; ^{f}Less than 20% exposed, prioritized; ^{g}Mutation derived from IgA sequence comparison; ^{h}Mutation derived from kappa chain sequence comparison; ⁱResidue within the hinge region.

Mutations Engineered in an Exemplary Human IgG1 Heavy Chain (SEQ ID NO:9).

(SEQ ID NO: 9)

MEFGLSWLFLVAILKGVQCQVQLQQSGPELVKPGASVRISCKASGFTFSY
HVNWVKQRPGQGLEWIGWIYPGNVNTEYNEKFKGKATLTADKSSSTAYMQ
LSSLTSEDSAVYFCASHEYYGSDWYFDVWGAGTTVTVSSASTKGPSVFPL
APSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVM
HEALHNHYTQKSLSLSPGK

TABLE 1

| Position | Kabat EU | % | Proposed | Position | Kabat EU | % | Proposed |
|---|---|---|---|---|---|---|---|
| (bolded) | Numbering | Exposure | mutation | (underlined) | Numbering | Exposure | mutation |
| T178^{a} | T155S | 36 | S | L257^{c,i} | L234I | 58 | I |
| N202 | L179I | 9 | I | L265 | L242I | 10 | I |
| L258ⁱ | L235P | 38 | P | F266^{c} | F243Y | 31 | Y |
| F264 | F241Y | 32 | Y | F298 | F275I | 9 | Y, I |
| Y319^{d} | Y296N | 71 | H, N | Y301 | Y278H | 19 | H |
| L332 | L309Q | 32 | I, Q | Y323^{d} | Y300H | 13 | H |
| Y372^{b,g} | Y349H | 28 | H | L329 | L306R | 2 | I, R |
| L388 | L365V | 0 | I, V | W336^{d} | W313Y | 5 | Y |
| L421^{a,b} | L398Q | 40 | I, Q | L337^{g} | L314M | | N, M |
| F427^{a,f} | F404Y | 12 | Y | Y342^{c} | Y319H | 2 | H |
| Y436^{g} | Y407T | 30 | T | L374^{c,e} | L351I | 23 | I |
| Y459 | Y436H | 25 | H | L391 | L368M | 10 | I, M |
| | | | | Y414 | Y391H | 20 | S, H |
| | | | | F428 | F405I | 16 | I |
| | | | | L429 | L406V | 2 | I, V |
| | | | | L433 | L410I | 0 | I |
| | | | | F446^{h} | F423Y | 0 | Y |
| | | | | L455 | L432I | 4 | I |
| | | | | Y459^{e} | Y436H | | S, H |

Selection of replacement residues for pepsin vulnerable sites: The selection of residues to replace the potential cleavage sites was based on information from an “unbiased” database of IgG Fc sequences (Demarest et al. 2004) (see below). Mutations were made to the next most frequently observed residue within the dataset of IgG sequences. In order to understand the mutational tolerance of each position, mutations were also made to Alanine. A few mutant combinations were also constructed prior to the original tolerance screening yielding a theoretical total of 72 library members. 66 of these were successfully cloned (Table 2) and screened for expression and thermotolerance (Table 3).

Table 2 shows the expression and thermotolerance results for the entire mutant library. A score was given to each variant to describe its expression. +: Expression was greater than wildtype; : Equivalent expression compared to wildtype; −: Less material was expressed than the wildtype; −: No expression. Each antibody variant was given a thermotolerance score according to the following criteria: +: A greater percentage of folded protein remaining at 75° C. and/or 80° C. compared to wildtype; : Equivalent percentage of folded protein remaining at each temperature point compared to wildtype; −: A lesser percentage of folded protein remaining at 75° C. than wildtype; −: Thermal unfolding observed at 70° C. Detection: *Detected using anti Fab-AP. Otherwise detected with anti Fc-AP.

TABLE 2

| Clone Name | Mutation | Expression | Thermotolerance |
|---|---|---|---|
| BD12611 | T178S | − | ? |
| BD12636 | L258P | | |
| BD12619 | L332Q | | |
| BD12625 | L388V | | − |
| BD12628 | L421Q | | |
| BD12629 | F427Y | | |
| BD12791 | L421QF427Y | | − |
| BD12794 | L258PF298IL332Q | + | − |
| BD12641 | L258PL332Q | + | + |
| BD12957 | Y372A | −* | − |
| BD12957 | Y372A | −* | − |
| BD12959 | L388A | −* | − |
| BD12961 | L421A | * | − |
| BD12962 | F427A | −* | − |
| BD12964 | Y459A | (−)* | − |
| BD12613 | F264Y | * | + |
| BD12634 | Y459H | − | − |
| BD12635 | L202I | | |
| BD12637 | Y319N | | − |
| BD12638 | L421QT178S | | − |
| BD12639 | L421QL258P | | − |
| BD12640 | L421QL332Q | | − |
| BD12792 | L421QL202I | | |
| BD12793 | F427YT178S | | |
| BD12796 | L388VL332Q | | − |
| BD12623 | Y372H | * | − |
| BD12632 | Y436T | * | − |
| BD12795 | F298IL332Q | | |
| BD12953 | L329A | * | |
| BD12954 | W336A | −* | − |
| BD12955 | L337A | −* | |
| BD12956 | Y342A | +* | |
| BD12958 | L374A | * | − |
| BD12960 | L391A | * | − |
| BD12963 | F428A | * | − |
| BD12965 | L429A | +* | − |
| BD12966 | L433A | +* | − |
| BD12967 | F446A | +* | − |
| BD12612 | L257I | * | * |
| BD12614 | L265I | * | |
| BD12615 | F266Y | * | |
| BD12616 | F298I | * | |
| BD12617 | Y301H | * | − |
| BD12618 | L329R | * | − |
| BD12621 | L337M | * | − |
| BD12620 | W336Y | * | − |
| BD12622 | Y342H | * | |
| BD12624 | L374I | * | − |
| BD12626 | L391M | * | |
| BD12627 | Y414H | * | − |
| BD12630 | F428I | * | − |
| BD12631 | L429V | * | |
| BD12633 | L433I | * | − |
| BD12943 | T178A | * | −* |
| BD12944 | L202A | * | * |
| BD12945 | L257A | −* | * |
| BD12946 | L258A | +* | /−* |
| BD12947 | F264A | +* | |
| BD12948 | L265A | * | − |
| BD12949 | F266A | * | − |
| BD12950 | F298A | * | /− |
| BD12951 | Y301A | * | |
| BD12952 | Y319A | * | |

Tables 3A and 3B shows the ELISA results after pepsin digestion at pH 3, pH 2 and pH 2.5 of the wildtype and the mutated antibody molecules. The parent antibody molecule as well as the mutants were expressed in mammalian cells, purified, and dialyzed. 1 μg was digested for the time indicated with pepsin (×0.005) at 37° C. at pH 2, pH 2.5, and pH 3. Two separate tests were performed: one to detect the remaining constant domain and a test to assess the remaining binding activity of the antibody molecule. In all cases, antibody degradation was determined by measuring by ELISA the amount of antibody remaining after digestion. Mutations are listed below.

Table 3A shows the ELISA results after pepsin digestion of the wildtype and mutants. The percentage of Fc remaining after digestion as well as the percentage of antibody binding to toxin A are reported after 0.5 hour (h), 1 h, and 4 h digestion with pepsin at pH 3, 2.5 and 2.

Table 3B shows the ELISA results after pepsin digestion at pH 2, 2.5 and 3 of the mutant combination. The percentage of toxin A binding activity remaining after digestion is reported after 0.5 h, 1 h, and 4 h digestion with pepsin.

TABLE 3A

| | | pH 3 | | | | pH 2.5 | | pH 2 | |
|---|---|---|---|---|---|---|---|---|---|
| BD # | Mutation | 0.5 h | 1 h | 4 h | 7 h | 2 min | 5 min | 2 min | 5 min |
| 12584 | Wildtype | 14% | 0% | 0% | 0% | 0% | 0% | 0% | 0% |
| 14079 | L258P, L332Q, F427Y, F264Y, L202I, L421Q | 91% | 85% | — | — | 0% | 0% | 0% | 0% |
| 13964 | L258P, L332Q, F427Y, F264Y, L202I, T178S | 100% | 96% | — | — | — | — | — | — |
| 13936 | L258P, L332Q, F427Y, L202I | 69% | 71% | 100% | 94% | 0% | 0% | 0% | 0% |
| 14487 | L257I, L258P, L332Q, F427Y, F264A, L202I, L421Q, T178S | 0% | 0% | 0% | — | — | — | — | — |
| 14357 | L258P, L332Q, F427Y, F264Y, L202I, L421Q, T178S | 100% | 100% | 100% | — | — | — | — | — |
| 12639 | L421Q, L258P | 98% | 72% | 100% | 100% | — | — | — | — |
| 14568 | L257I, L258P, L332Q, F427Y, F264A, L202I, L421Q, T178S, | — | — | — | — | — | — | 0% | 0% |
| | L265I, Y342H | | | | | | | | |
| 14563 | L257I, L258P, L332Q, F427Y, F264A, L202I, L421Q, T178S, | — | — | — | — | — | — | 0% | 0% |
| | L265I, L429V | | | | | | | | |

TABLE 3B

| | | pH 3 | | pH 2.5 | | | pH 2 | | |
|---|---|---|---|---|---|---|---|---|---|
| BD # | Mutation | 1 h | 4 h | 0.5 | 1 h | 4 h | 0.5 h | 1 h | 4 h |
| 12584 | Wildtype | 78-100% | 66-100% | 93% | 71% | 58% | 66% | 41% | 11% |
| 14079 | L258P, L332Q, F427Y, F264Y, L202I, L421Q | 93% | — | 72% | 52% | 17% | 53% | 26% | 8% |
| 13936 | L258P, L332Q, F427Y, L202I | 90% | — | 92% | 82% | 27% | — | — | — |
| 14487 | L257I, L258P, L332Q, F427Y, F264A, L202I, L421Q, T178S | 97% | 100% | — | — | — | — | — | — |
| 14357 | L258P, L332Q, F427Y, F264Y, L202I, L421Q, T178S | 100% | 100% | — | — | — | — | — | — |
| 12639 | L421Q, L258P | 55% | 69% | — | — | — | — | — | — |
| 13936 | L258P, L332Q, F427Y, L202I | 100% | 100% | — | — | — | — | — | — |
| 14568 | L257I, L258P, L332Q, F427Y, F264A, L202I, L421Q, | — | — | — | — | — | 37% | 31% | 6% |
| | T178S, L265I, Y342H | | | | | | | | |
| 14563 | L257I, L258P, L332Q, F427Y, F264A, L202I, L421Q, | — | — | — | — | — | 62% | 31% | 13% |
| | T178S, L265I, L429V | | | | | | | | |

Development of screening protocols: Conditions for screening the heavy chain antibody library were tested using the recombinant hPBA-3 antibody expressed and purified from mammalian cell cultures as shown in FIG. 5. In FIG. 5(A) illustrates ELISA detection of the remaining quantity of rPBA3 after incubation at pH 1, 2 and 3 using solutions containing hydrochloric and detected with AP-labeled anti-Fc. In FIG. 5(B) rPBA3 was digested by 0.005×SGF at 37° C. for various incubation periods. Two separate detection antibodies were used, AP-labeled anti-Fc and AP-labeled anti-Fab₂, in order to discriminate between Fc degradation and hinge clipping. Thermotolerance of rPBA3 incubated at various temperatures for both 10 and 30 minutes. In all cases, antibody degradation was determined by measuring by an ELISA the amount of rPBA3 remaining after digestion.

Screening: Expression and thermotolerance screening was performed for every member of the library to determine whether mutation at each pepsin-labile position was tolerated. The majority of the library members were also tested for tolerance at low pH. DNAs derived from the 72 variants were transfected into mammalian cells and the resulting supernatants were screened for thermotolerance, pH and pepsin-resistance. All antibodies demonstrated a similar pH tolerance compared to wildtype. However, many mutants demonstrated inferior thermotolerance and/or expression compared to the wildtype molecule. Approximately 46% of the database selected mutants were destabilizing while 64% of the Alanine mutations were destabilizing (Table 4). Interestingly, this mutational strategy (based on an IgG sequence database developed for this invention) provided a significantly greater proportion of tolerable residue replacements relative to alanine scanning. Destabilizing mutations were eliminated from the mutant combinations described below.

Table 4, below, shows the ELISA results after pepsin digestion at pH 3 the single mutants. The percentage of Fc remaining after digestion as well as the percentage of antibody binding to toxin A are reported after 0.5 h, 1 h, and 4 h digestion with pepsin at pH 3.

TABLE 4

| | | Fc | | | | |
|---|---|---|---|---|---|---|
| | | detection | | | Binding to Toxin A | |
| BD | | 0.5 h | 1 h | 4 h | 1 h | 4 h |
| 12611 | T178S | 62% | 39% | 13% | 98% | 94% |
| 12636 | L258P | 46% | 52% | 60% | 86% | 87% |
| 12635 | L202I | 69% | 70% | 0% | 82% | 87% |
| 12632 | Y436T | 17% | 0% | 0% | 77% | 76% |
| 12629 | F427Y | 63% | 30% | 52% | 100% | 96% |
| 12623 | Y372H | 67% | 15% | 0% | 88% | 86% |
| 12613 | F264Y | 43% | 23% | 0% | 65% | 48% |

Design of up-mutants: Up-mutants containing multiple pepsin resistance sites were designed based on the initial 66 member library screen. Positions that tolerate mutation were prioritized and combined. The highest priority was put on sites discovered via proteomic analysis as well as those that are highly exposed to solvent. Below is a list of exemplary antibody mutant combinations cloned and transfected into mammalian cells for IgG expression:

TABLE 5

| Identification | |
|---|---|
| designation | Mutant combination. |
| BD13936 | L258P, L332Q, F427Y, L202I |
| BD13964 | L258P, L332Q, F427Y, F264Y, L202I, T178S |
| BD14078 | L258P, L332Q, F427Y, F264Y, L202I |
| BD14079 | L258P, L332Q, F427Y, F264Y, L202I, L421Q |
| BD14358 | L258P, L332Q, F427Y, F264A, L202I, L421Q, T178S |
| BD14359 | L258P, L332Q, F427Y, F264A, L202I, L421Q |
| BD14487 | L257I, L258P, L332Q, F427Y, F264A, L202I, L421Q, |
| | T178S |

All up-mutants tested expressed comparably to the wildtype protein and demonstrated similar or better thermotolerance profiles. Examples of pepsin digestions are shown in FIGS. 6 and 7. FIG. 6 illustrates the pepsin digestion profile of wildtype and mutant antibodies at pH 1.2. The mutants carried either 4 mutations in the heavy chain constant domain (L258P, L332Q, F427Y, F264Y) or 6 mutations in the heavy chain constant domain (L258P, L332Q, F427Y, F264Y, L202I, L421Q). The antibodies were expressed in mammalian cells, purified, and dialyzed. Pepsin digestion time points were 0, 2, 5, 10 and 20 min (×0.005 SGF) at pH 1.2. FIG. 6(A) illustrates an SDS-Page Analysis of IgG digests with pepsin at pH 1.2. DTT was added to the samples prior to loading each 800 ng time point to wells in a 4-12% Bis-Tris gel. Gels were silver-stained. FIG. 6(B) illustrates an ELISA detection of each recombinant IgG after digestion.

FIG. 7 illustrates the pepsin digestion profile of wildtype and mutant antibodies at pH 3.0. FIG. 7(A) illustrates an SDS-Page/Silver stain analysis of pH 3, pepsin digestion. Wildtype rPBA3 along with BD13964 (6 mutants) and BD14079 (6 mutants) were subjected to digestion with pepsin (0.005×SGF). Time points included (Lanes 5-12, respectively) are 0, 2, 5, 10, 20, 30, 60 and 120 minutes. DTT was added to the samples prior to loading each 800 ng timepoint to wells in a 10% Bis-Tris gel. Gels were silver-stained. Lane 1 is the SEEBLUEPLUS2™ standard, Lane 2 is reduced Fab fragment standard, Lane 3 is reduced Fc fragment standard, Lane 4 is each recombinant protein loaded at 1 μg. FIG. 7(B) illustrates an ELISA analysis of various recombinant IgGs digested by pepsin at pH 3. Digestion of the wildtype protein begins to generate Fc at 5 minutes. A second, lower molecular weight band (˜1 0-12 kDa) also begins to form due to heavy chain degradation at the 10 mn time point. BD13964 and BD14079 were completely resistant to digestion for 2 hours at pH 3, 37° C.

In one example, a clear difference in the digestibility of BD14079 and BD13964 compared to wildtype rPBA3 antibody was observed. At pH 1.2, the wildtype rPBA3 antibody completely disappeared at initial time point (2 min); however, BD14079 partially survived through 2 minutes. The banding pattern between the wildtype and BD14079 was also different at pH 1.2. BD14079 exhibited larger molecular weight bands after digestion than the wildtype protein suggesting that one or more of the mutants hindered the formation of lower molecular weight fragments (FIG. 6). Corroborating results were obtained by ELISA analysis. The antibody degraded quickly at pH 1.2, and appeared to completely unfold between pH 2 and 3 (FIG. 4).

While most acid proteases are active below pH 4 where they maintain the protonated and deprotonated forms on two separate carboxyl groups (Asp32 and 215, respectively, for pepsin) at the active site of hydrolysis, the antibody molecule did not unfold until the pH was lowered below pH 3, however (Suguna et al., 1987). Therefore, the pepsin digestibility of the wildtype antibody and the mutant combinations at a pH value (pH 3) where the molecule remains folded was measured. Drastic differences in digestibility were observed between the wildtype protein and two six mutant combinations at pH 3 (FIG. 7). The wildtype protein was over 80% degraded within 30 minutes of exposure to 0.005×SGF at 37° C., pH 3. SDS-page analysis of the W.T. rPBA3 digestion indicated the appearance of Fc fragment even at the earliest time point, 2 minutes. Several other smaller molecular weight bands were apparent after 10 minutes. However, the two mutant combinations were completely undisturbed after two hours of exposure to pepsin under the same conditions. These results clearly demonstrated that the modification of the targeted pepsin cleavage sites within the IgG₁ framework allowing the molecule to survive for longer durations under both native (pH 3) and denaturing (pH 1.2) pH conditions.

Strategies to Engineer Antibody Molecule for pH Tolerance

The results described above demonstrated that using the methods of the invention an IgG molecule was successfully and selectively engineered to resist pepsin degradation while in its native conformation (i.e., pH 3 and above). Further engineering using methods of the invention can focus on making an IgG molecule resistant to pH dependent unfolding. As demonstrated by CD experiments, the antibody unfolds below pH 3. Thus, this unfolding event is brought on by the protonation of aspartic and glutamic acid residues involved in salt bridges and hydrogen bonds within the folded structure of the antibody. In general, residues involved in salt bridges and hydrogen bond networks demonstrate highly correlated covarying dependencies on other residues involved in the interaction (Clarke, 1995). This is untrue for most hydrophobic residues, such as leucine, which was one of the targeted residues for replacement to avoid pepsin recognition. To make modifications of the pH dependence of an antibody, however, mutagenesis of co-varying pairs or combinations of residues are performed.

In one aspect, an accurate antibody fold sequence alignment based on the available crystal structures of the immunoglobulin subclasses and cell surface receptors is built. Most antibody folds whose structures are known will be structurally aligned to the lowest root-mean-squared deviation to create optimal sequence alignment. Sequences without crystal structures are aligned to the next most homologous antibody fold sequence whose structure is solved. The resulting sequence alignment is analyzed for co-varying pairs as described by Davidson and coworkers (Larson et al., 2000). This co-variation analysis identifies adequate residue replacements for salt bridges and aspartic acid and glutamic acid residues within IgG₁ that lead to the denaturation of the molecule at pH values below 3.

Material and Methods

Digestibility of IgG subclasses in gastric fluid: All antibodies purchased from Calbiochem were isolated from human myelomas: IgG1 with kappa light chain (CalBiochem Cat #400120), IgG2 with kappa light chain (CalBiochem Cat#400122), IgG3 with lambda light chain (CalBiochem Cat#400124), and IgG4 with lambda light chain (CalBiochem Cat#400126). Simulated gastric fluid (SGF) was prepared fresh daily as described in the United States Pharmacopoeia. 1×SGF buffer comprised 3.2 mg/mL pepsin (Sigma Chemical Co., St. Louis, Mo.), NaCl (2 mg/mL) at pH 1.2. Dilutions were prepared in the same buffer. A master tube was prepared in a 1.5 mL microcentrifuge tube containing 60 μg of antibody and 120 μL 0.001×SGF in a final volume of 180 μL. The reaction was incubated at 37° C. At intervals of 0, 2, 5, 10, 20, and 30 min, aliquots of 30 μL containing 10 μg of antibody were removed from the master tube and added immediately to 7 μL 4× NUPAGE™ LDS sample buffer (Invitrogen) and heated for 5 min at 100° C. Samples were subjected to SDS-PAGE using precast 4-12% Bis-Tris NUPAGE™ gels (Invitrogen, Carlsbad, Calif.). Gels were run at a 160 V for ˜40 minutes using MES running buffer according to the manufacturer's instruction. Proteins were visualized using GELCODE™ Blue Stain Reagent (PIERCE, Rockford, Ill.). The protein MW Marker SEEBLUE PLUS2™ was purchased from Invitrogen.

Hybridoma culture: Hybridoma cell line PBA3 expressing a Clostridium difficile anti-toxin A recognizing antibody was obtained from ATCC. Cell lines were grown in DMEM (Dulbecco's Minimal Essential Medium with high glucose, Gibco, Invitrogen, Carlsbad, Calif.), 10% FBS (Sterile Fetal Bovine Serum, Sigma Chemical, St. Louis, Mo.), and 1× glutamine/Penicillin/Streptomycin (Gibco, Invitrogen, Carlsbad, Calif.) and cryopreserved.

Antibody gene cloning: Total RNA was isolated from 10⁷ hybridoma cells using a procedure based on the RNeasy Mini kit (Qiagen, Hilden Germany). The poly-A+ RNA fraction was purified using an Oligotex mRNA mini kit (Qiagen) and used to generate first strand cDNA (Clontech cDNA synthesis kit, Clontech Laboratories, Inc., Palo Alto, Calif.). Primers used for the amplification of the variable region from both the light chain and the heavy chains were designed as described previously (Coloma et al., 1992; Dattamajumdar et al., 1996). Primers MLALT5 and 33615 were used for amplification of the variable region from the light chain (MLALT5: 5′-CACCATGAAGTTGCCTGTTAGGCTGTTG-3′ (SEQ ID NO:10); 33615:5′-GAAGATCTAGACTTACTATGCAGCATCAGC-3′) (SEQ ID NO:11). Primers MVG1R and MH1 were used for the amplification of the heavy chain variable region (MH1: 5′-ATATCCACCATGGRATGSAGCTGKGTMATSCTCTT-3′ (SEQ ID NO: 12); MVG1R: 5′-GGCAGCACTAGTAGGGGCCAGTGGATA-3′) (SEQ ID NO:13). Sense primers (based on the FR1 region) and antisense primers (based on the 5′-end of the constant region) were then designed for both chains following sequencing of the PCR products. PCR products obtained using these primers were cloned into the modified mammalian expression vector pCEP4 (Invitrogen, Carlsbad, Calif.). The modified vector either contained the signal peptide and the constant domain region of the heavy chain or the signal peptide and the constant domain of the light chain. The constant domain of the human IgG1 was constructed by subcloning the appropriate heavy chain and light chain domains into pCEP4 from a human spleen cDNA library. The plasmid containing the light chain variable domain and its constant domain was designated BD12585. The plasmid containing the variable domain and the constant domain of heavy chain was designated BD12584. Both plasmids were sequenced. The chimeric antibody protein is referred as rPBA3 in the text.

IgG1 mutagenesis: Site-directed mutagenesis on IgG1 was used to generate IgG1 variants in which all solvent-exposed residues in the CH1, CH2, and CH3 domains were individually altered to Ala or another residue (as specified in the list). All mutants were confirmed by DNA sequencing.

Transfection of rPBA3 library into 293F mammalian cell expression host: All mutant plasmids were transformed into XL1-blue bacteria and stocked in glycerol. Plasmid DNA from every mutant was prepared as described by the manufacturer (Qiagen, endotoxin-free MaxiPrep kit Cat#12362). Plasmids were transfected into the adenovirus-transformed human embryonic kidney cell line 293F using 293fectin in 12-well microtiter plates and using 293F-FreeStyle Media for culture. Light and heavy chain plasmids were transfected at 0.5 μg/mL for each plasmid and using a 1:1 light chain plasmid versus heavy chain plasmid ratio. Supernatants were collected 7 days after transfection. Expression levels varied from ˜0.25-1.5 μg/mL. For larger transfections, the cells were spun down after 3 days and ½ the media was replenished with fresh media. Cell density upon transfection was generally 10⁶ cells/mL. Supernatants were then spun down at 1200 rpm for 8 minutes at room temperature.

Medium Scale Expression and Purification of monoclonal IgG1 from cell culture: Transfection and tissue-culture was performed as described above with the exception that 100 mL supernatants from mammalian cell cultures were collected and passed through a 0.22 μm filter. Final supernatant volumes were between 100-1000 mL serum-free medium. Supernatants containing antibody were applied directly to 5 mL HITRAP™ Protein G Columns (Amersham Biosciences, Piscataway, N.J., cat#17-0405-01) at 5 mL/min. Multiple passage of supernatants over the columns was unnecessary as >95% of all IgG1 material from each supernatant bound to the column on the first pass. Mobile phases comprised 1×PBS-Tween (Sigma Aldrich, Running Buffer, cat# P-3563) and 0.1 M glycine pH 2.7 (Fisher Chemicals, Elution Buffer, cat# G48-500). Antibody collections in 0.1 M glycine were diluted 20% (v/v) with 1 M TrisHCl, pH 8.0, for neutralization. IgG1 collections were pooled and dialyzed exhaustively against 1×PBS (Pierce SLIDE-A-LYZER™ Cassette, 3500 MWCO, cat#66110). The concentration of each IgG1 stock solution was determined by Bradford analysis (Bio-Rad Protein Assay, Hercules, Calif. cat#500-0006) using a commercial myeloma IgG1 stock solution (2 mg/ml—Calbiochem, cat#400120) as a standard and by UV-absorbance at 280 nm using the method of Pace and coworkers (1995).

SGF digestion stability assay for the mutants: Simulated gastric fluid (SGF) was prepared fresh daily as described (Privalle et al., 2000) using 0.1×SGF buffer at pH 1.2 or pH 3 (3.2 mg/ml pepsin, 2 mg/ml NaCl; Sigma Chemical Co., St. Louis, Mo.). All recombinant antibodies were dialyzed into PBS and stored at 4° C. For all digestions, a master tube was prepared containing 1 μg/mL recombinant antibody and 0.0025×SGF at pH 1.2 and 0.005×SGF at pH 3.0. The pH of each reaction was monitored by first making appropriate dilutions of PBS with SGF and measuring the pH before and after neutralization with TrisHCl, pH 9. Antibodies were incubated at 37° C. for intervals of 0, 2, 5, 10 and 20 min at pH 1.2 or at intervals of 0, 2, 5, 10, 20, 30, 60 and 120 min at pH 3.0. The reaction was neutralized before aliquots were taken either for ELISA analysis or for SDS-Page/Silver staining. SDS-Page gels were run as described above, except, under reducing conditions, 10% gels provided superior separation. The amount of protein added to the gel was limited to 0.8 μg/well; therefore, protein bands were visualized using the SILVERQUEST™ Silver Staining Kit (Invitrogen cat#LC6070). 1 μg of IgG Fc and Fab standards (Pierce cat#31205 and #31203, respectively) were reduced with 100 mM DTT and added to the gel to allow for the discrimination of intact recombinant heavy chain, recombinant light chain and hinge proteolyzed recombinant Fc fragment. ELISA assays were performed as described below. 200 ng of recombinant antibody was used per well in order for the protein to be at the top of the linear range of detection.

ELISA: Protein G (Sigma, cat# P-4689) was biotinylated using the EZ-LINK-BIOTIN-LC-ASA™ kit (PIERCE catalog #29982). Briefly, EZ-LINK-BIOTIN-LC-ASA™ was dissolved in DMSO and added individually to protein G at a 5:1 molar ratio. Protein G/biotin conjugation was induced for 20 minutes under a UV lamp in a PBS buffer. Conjugated protein G was removed from unreacted biotin by application of the reaction mixture to a desalting column (PIERCE D-Salt Dextran Plastic Desalting Columns, catalog #43230). 500 μL fractions from the desalting procedure were tested for protein absorption at 280 nm to detect the presence of biotinylated protein G.

Microtiter Streptavidin plates (Sigma Chemical, St. Louis, Mo., catalog #M5432) were coated with 200 ng per well of biotinylated protein G diluted into PBS buffer and incubated at 4° C. overnight. The plates were then washed 3 times with TBST buffer. All samples were diluted in Tris buffer, pH 8.0 TBST buffer (Sigma, cat#T9039). Aliquots of 100 μL of each diluted sample were transferred to the protein G-coated plates and incubated for 1-2 hours at room temperature. Following 3 washes with TBST, Alkaline phosphatase-conjugated IgG heavy chain-specific mouse anti-human IgG (Zymed, cat#05-4222) was added to each well at a 1:500 dilution. The reaction was carried out for 1 hr at room temperature, the plate(s) was washed 3 times with TBST and 100 μL of p-nitrophenylphosphate substrate was added (Sigma, Catalog # A3469). The absorption was determined at 405 nm using a Molecular Devices vₘₐₓ kinetic microplate reader. Protein concentrations were determined using the Bradford protein assay using quantified IgG1 as the standard and/or by UV-280 absorbance.

Expression and Thermotolerance Analysis of constant domain mutant library: Expression of the mutant library was performed in a 12-well plate format as described above. One well of each 12-well plate was dedicated to the wildtype antibody as an internal control. The expression of each mutant variant was tested by ELISA (Table 3). A score was given to each variant to describe its expression (see Table 3 legend).

The wildtype antibody began to unfold when heated to 75° C. for 10 minutes and is completely unfolded when subjected to 80° C. for the same time period (FIG. 5c). The unfolding was irreversible as cooling for any length of time did not result in the regeneration of signal in this ELISA format. The thermotolerance of each member of the constant domain mutant library was compared to the wildtype molecule by heating (side-by-side with the wildtype protein) to 70° C., 75° C. and 80° C. for 10 minutes. The amount of folded antibody remaining after heating was tested by ELISA (Table 3). Each antibody variant was given a thermotolerance score (see table 3).

Clostridium difficile toxin A is well described in the art, see, e.g., Wren, et al., (1990) “Nucleotide sequence of Clostridium difficile toxin A gene fragment and detection of toxigenic strains by polymerase chain reaction,” FEMS Microbiol. Lett. 70:1-6 (1990) (NCBI accession no. A37052):

(SEQ ID NO: 14)

| | |
|---|---|
| 1 | msliskeeli klaysirpre neyktiltnl deynklttnn |
| | nenkylqlkk lnesidvfmn |
| 61 | kyktssrnra lsnlkkdilk eviliknsnt spveknlhfv |
| | wiggevsdia leyikqwadi |
| 121 | naeyniklwy dseaflvntl kkaivesstt ealqlleeei |
| | qnpqfdnmkf ykkrmefiyd |
| 181 | rqkrfinyyk sqinkptvpt iddiikshlv seynrdetvl |
| | esyrtnslrk insnhgidir |
| 241 | anslfteqel lniysqelln rgnlaaasdi vrllalknfg |
| | gvyldvdmlp gihsdlfkti |
| 301 | srpssigldr wemikleaim kykkyinnyt senfdkldqq |
| | lkdnfkliie sksekseifs |
| 361 | klenlnvsdl eikiafalgs vinqaliskq gsyltnlvie |
| | qvknryqfln qhlnpaiesd |
| 421 | nnftdttkif hdslfnsata ensmfltkia pylqvgfmpe |
| | arstislsgp gayasayydf |
| 481 | inlqentiek tlkasdlief kfpennlsql teqeinslws |
| | fdqasakyqf ekyvrdytgg |
| 541 | slsedngvdf nkntaldkny llnnkipsnn veeagsknyv |
| | hyiiqlqgdd isyeatcnlf |
| 601 | sknpknsiii qrnmnesaks yflsddgesi lelnkyripe |
| | rlknkekvkv tfighgkdef |
| 661 | ntsefarlsv dslsneissf ldtikldisp knvevnllgc |
| | nmfsydfnve etypgkllls |
| 721 | imdkitstlp dvnknsitig anqyevrins egrkellahs |
| | gkwinkeeai msdlsskeyi |
| 781 | ffdsidnklk aksknipgla sisediktll ldasvspdtk |
| | filnnlklni essigdyiyy |
| 841 | eklepvknii hnsiddlide fnllenvsde lyelkklnnl |
| | dekylisfed isknnstysv |
| 901 | rfinksnges vyvetekeif skysehitke istiknsiit |
| | dvngnlldni qldhtsqvnt |
| 961 | lnaaffiqsl idyssnkdvl ndlstsvkvq lyaqlfstgl |
| | ntiydsiqlv nlisnavndt |
| 1021 | invlptiteg ipivstildg inlgaaikel ldehdpllkk |
| | eleakvgvla inmslsiaat |
| 1081 | vasivgigae vtifllpiag isagipslvn nelilhdkat |
| | svvnyfnhls eskkygplkt |
| 1141 | eddkilvpid dlviseidfn nnsiklgtcn ilameggsgh |
| | tvtgnidhff sspsisship |
| 1201 | slsiysaigi etenldfskk immlpnapsr vfwwetgavp |
| | glrslendgt rlldsirdly |
| 1261 | pgkfywrfya ffdyaittlk pvyedtniki kldkdtrnfi |
| | mptittneir nklsysfdga |
| 1321 | ggtyslllss ypistninls kddlwifnid nevreisien |
| | gtikkgklik dvlskidink |
| 1381 | nkliignqti dfsgdidnkd ryifltceld dkisliiein |
| | lvaksyslll sgdknylisn |
| 1441 | lsntiekint lgldskniay nytdesnnky fgaisktsqk |
| | siihykkdsk nilefyndst |
| 1501 | lefnskdfia edinvfmkdd intitgkyyv dnntdksidf |
| | sislvsknqv kvnglylnes |
| 1561 | vyssyldfvk nsdghhntsn fmnlfldnis fwklfgfeni |
| | nfvidkyftl vgktnlgyve |
| 1621 | flcdnnknid iyfgewktss skstifsgng rnvvvepiyn |
| | pdtgedists ldfsyeplyg |
| 1681 | idryinkvli apdlytslin intnyysney ypeiivlnpn |
| | tfhkkvninl dsssfeykws |
| 1741 | tegsdfilvr yleesnkkil qkirikgils ntqsfnkmsi |
| | dfkdikklsl gyimsnfksf |
| 1801 | nseneldrdh lgfkiidnkt yyydedsklv kglininnsl |
| | fyfdpiefnl vtgwqtingk |
| 1861 | kyyfdintga altsykiing khfyfnndgv mqlgvfkgpd |
| | gfeyfapant qnnniegqai |
| 1921 | vyqskfltln gkkyyfdnns kavtgwriin nekyyfnpnn |
| | aiaavglqvi dnnkyyfnpd |
| 1981 | taiiskgwqt vngsryyfdt dtaiafngyk tidgkhfyfd |
| | sdcvvkigvf stsngfeyfa |
| 2041 | pantynnnie gqaivyqskf ltlngkkyyf dnnskavtgw |
| | qtidskkyyf ntntaeaatg |
| 2101 | wqtidgkkyy fntntaeaat gwqtidgkky yfntntaias |
| | tgytiingkh fyfntdgimq |
| 2161 | igvfkgpngf eyfapantda nniegqaily qnefltlngk |
| | kyyfgsdska vtgwriinnk |
| 2221 | kyyfnpnnai aaihlctinn dkyyfsydgi lqngyitier |
| | nnfyfdanne skmvtgvfkg |
| 2281 | pngfeyfapa nthnnniegq aivyqnkflt lngkkyyfdn |
| | dskavtgwqt idgkkyyfnl |
| 2341 | ntaeaatgwq tidgkkyyfn lntaeaatgw qtidgkkyyf |
| | ntntfiastg ytsingkhfy |
| 2401 | fntdgimqig vfkgpngfey fapantdann iegqailyqn |
| | kfltlngkky yfgsdskavt |
| 2461 | glrtidgkky yfntntavav tgwqtingkk yyfntntsia |
| | stgytiisgk hfyfntdgim |
| 2521 | qigvfkgpdg feyfapantd anniegqair yqnrflylhd |
| | niyyfgnnsk aatgwvtidg |
| 2581 | nryyfepnta mgangyktid nknfyfrngl pqigvfkgsn |
| | gfeyfapant danniegqai |
| 2641 | ryqnrflhll gkiyyfgnns kavtgwqtin gkvyyfmpdt |
| | amaaagglfe idgviyffgv |
| 2701 | dgvkapgiyg |

See also von Eichel-Streiber, et al., (1992) “Comparative sequence analysis of the Clostridium difficile toxins A and B (1992) Mol. Gen. Genet. 233:260-268; NCBI accession no. CAA43302; NCBI accession no. BAA25318.

### REFERENCES:

Simulated Gastric Fluid and Simulated Intestinal Fluid, TS. In The United States Pharmacopeial Convention, Inc.: Rockville, Md., 1995; p. 2053
Coloma M J, Hastings A, Wims L A, Morrison S L. (1992) Novel vectors for the expression of antibody molecules using variable regions generated by polymerase chain reaction. J Immunol Methods. 152:89-104.
Delano, W. L., Ultsch, M. H., de Vos, A. M. & Wells, J. A. (2000) Convergent solutions to binding at a protein-protein interface. Science, 287: 1279-1283.
Demarest, S. J., Rogers, J. & Hansen, G. (2004) Optimization of the antibody CH3 domain by residue frequency analysis of IgG sequences. J. Mol. Biol. 335: 41-48.
Dattamajumdar A K, Jacobson D P, Hood L E, Osman G E. (1996) Rapid cloning of any rearranged mouse immunoglobulin variable genes. Immunogenetics 43:141-51.
Keil, B. (1992) Specificity of proteolysis. Springer-Verlag Berlin-Heidelberg-NewYork, pp. 335.
Pace, C. N., Vajdos, F., Fee, L., Grimsley, G. & Gray, T. (1995) How to measure the molar absorption coefficient of a protein. Protein Sci. 4: 2411-2423.
Privalle S. L., Wright, M., Reed, J., Hansen, G., Dawson, J., Dunder, E. M., Chang, Y.-F., Powell, L., & Meghji, M. (2000) Phophomannose isomerase-a novel system for plant selection. Proceedings of the 6^{th} International Symposium on The Biosafety of Genetically Modified Organisms. Ed. Fairbairn, C. Scoles, G. McHughen, A.
Suguna, K., Padlan, E. A., Smith, K. W., Carlson, W. D. and Davies, D. R. (1987) Binding of a reduced peptide inhibitor to the aspartic proteinase from Rhizopus chinensis: Implications for a mechanism of action. Proc. Natl. Acad. Sci. USA, 84: 7009-7013.

### Example 2

### Antibody Treatment of Clostridium difficile Induced Toxicity

This example demonstrates that antibodies made by methods of the invention can bind and neutralize Clostridium difficile toxin.

Antibodies raised against the C-terminal domains of toxins A and B were tested for their ability to bind and neutralize the affect of the toxins. Toxin neutralization in cell assays, antibody affinity measurements, epitope mapping and antibody competition experiments were all used to characterize and prioritize antibody candidates. One of the most neutralizing antibody against toxin A, denoted 3358 or 543, demonstrated the ability to bind the full-length CWB-domain at approximately fourteen high affinity sites. In contrast, the PCG-4 antibody (Lyerly (1986) Infect Immun. 54:70-76) was shown to contain between four and six high affinity binding sites. No single monoclonal antibody was fully neutralizing using the in vitro toxin neutralization assays. Calcium dependent flow cytometry analyses have enabled us to determine differential mechanisms of neutralization for monoclonal antibodies recognizing different epitopes of the CWB-domains. The most neutralizing monoclonal antibodies (including 3358 or 543, and rPCG-4) did not abrogate CWB-domain association with CHO cell surfaces, but retarded the internalization or reorganization of the toxin at the cell surface. Another neutralizing antibody, denoted 3350 or 227, did not recognize high affinity epitopes which overlap with those recognized by 543 and rPCG-4. The 3350 or 227 antibody inhibited cell surface association of the CWB-domains, demonstrating a separate mechanism of toxin A neutralization. Monoclonal antibodies representing both mechanisms of neutralization were shown to synergistically enhance each others binding to the CWB-domain of toxin A and in combination increased neutralization to levels similar to a commercial polyclonal standard.

Cloning and Expression of Toxin A and B Domains

Toxins A and B were cloned directly from genomic DNA preparations of a control strain of C. difficile (ATCC #51695). C. difficile was cultured at 37° C. in a thioglycollate anaerobic broth (BBL#273127) for 48-72 hours. Cultures were pelleted and genomic DNA was extracted using a RNA/DNA Maxi Prep Kit (Qiagen, Cat#14162). DNA inserts were generated for the toxin A and toxin B C-terminal repeat regions using primers designed to match the NCBI deposited sequences (Toxin B ATCC accession #BCAA80815.1; toxin A #AAA23283.1). Inserts were cloned into the pSE420 plasmid (Invitrogen, Cat#V4020) at the NcoI/BgIII using a BsaI cloning strategy (New England BioLabs, Cat#R0535S). The plasmid was modified to include a C-terminal, thrombin-cleavable hexa-histidine tag. The cloned toxin B sequences matched the NCBI reference sequence perfectly. The toxin A clones had 4 amino acid mutations (N1939D, L2080W, D2426H and A2427N) which were consistently amplified and can be strain specific. The plasmids containing the toxin inserts were given the following designations: ToxA:1800-2710, BD11822; ToxA:1800-1945, BD15049; ToxA:2078-2234, BD15050; ToxA:2459-2710, BD11711; ToxB:1808-2366, BD11713; ToxB:2207-2366, BD11712.

Each toxin containing plasmid was transformed into BL21(DE3) (Invitrogen, cat#C60000-03) or recA1 deficient XL1-blue (Stratagene, cat#200236) for improving the insert stability of the repeat domains. Protein expression was performed following standard protocols (1). Transformed cells were cultured in 2-6 L Luria Broth with 100 μg/mL carbenicillin at 37° C. until cell densities of 0.7-0.9 were reached. At this point, expression was induced with 1 mM IPTG and cultures were grown for 12 hours at 25° C. before harvesting. Cells were pelleted at 4000 g and stored at −20° C. Pellets were resuspended in PBS (Sigma, Cat#P-3813) and sonicated. The soluble fraction of all cell pellets was used for purification.

Production of Recombinant Anti-Toxin A rPBA-3, r543, r227 and rPCG-4 Antibodies

Anti-C. difficile toxin A hybridoma cell line PBA-3 (ATCC# HB-8713) was purchased from the ATCC. The cell line was grown in DMEM (Dulbecco's Minimal Essential Medium with high glucose (Gibco/Invitrogen, Carlsbad, Calif.), 10% FBS (Sterile Fetal Bovine Serum, Sigma Chemical, St. Louis, Mo.), and 1× glutamine/Penicillin/Streptomycin (Gibco/Invitrogen, Carlsbad, Calif.) and cryopreserved. Total RNA was isolated from 10⁷ hybridoma cells using a procedure based on the RNeasy Mini kit (Qiagen, Hilden Germany). The poly-A+ RNA fraction was purified using an Oligotex mRNA mini kit (Qiagen) and used to generate first strand cDNA (Clontech cDNA synthesis kit, Clontech Laboratories, Inc., Palo Alto, Calif.). Primers used for the amplification of the variable region from both the light chain and the heavy chains were designed as described previously (58,59). Primers MLALT5 and 33615 were used for amplification of the variable region from the light chain (MLALT5: 5′-CACCATGAAGTTGCCTGTTAGGCTGTTG-3′ (SEQ ID NO:10); 33615: 5′-GAAGATCTAGACTTACTATGCAGCATCAGC-3′) (SEQ ID NO:11). Primers MVG1R and MH1 were used for the amplification of the heavy chain variable region (MH1: 5′-ATATCCACCATGGRATGSAGCTGKGTMATSCTCTT-3′ (SEQ ID NO: 12); MVG1R: 5′-GGCAGCACTAGTAGGGGCCAGTGGATA-3′) (SEQ ID NO:13). Sense primers (based on the FR1 region) and antisense primers (based on the 5′-end of the constant region) were then designed for both chains following sequencing of the PCR products. PCR products obtained using these primers were cloned into the modified mammalian expression vector pCEP4 (Invitrogen, Carlsbad, Calif.). The modified vector either contained the signal peptide and the constant domain region of the heavy chain or the signal peptide and the constant domain of the light chain. The constant domain of the human IgG₁ was constructed by subcloning the appropriate heavy chain and light chain domains into pCEP4 from a human spleen cDNA library. The plasmid containing the light chain variable domain and its constant domain was designated BD12585. The plasmid containing the variable domain and the constant domain of heavy chain was designated BD12584.

The 3358 (or 543) and 227 (or 3359) antibody variable domains were subcloned into pCEP4 using the same protocol as outlined for rPBA-3. Below are the amino-acid sequences of the variable region antibodies. CDR regions are underlined. The “completed” or complete antibodies comprise human sequence constant regions.

Antibody 227 (or 3359)
variable heavy chain (IgG1)

(SEQ ID NO: 1)

EVQLVESGGGLMKPGGSLKLSCAASGFAFGSYDMSWVRQTPEKRLEWVAY
ISSGGGITFYPDSVRGRFTISRDNAKNSLYMEMSSLRSEDTAMYYCARWD
WDLFAYWGQGTLVTVSAAAS
variable light chain (kappa)

(SEQ ID NO: 2)

DIKMTQSPSSMYTSLGERVTITCKASQDINSCLSWFQQKPGKSPKALIFR
ANILVDGVPSRFSGSGSGQDYSLTISSLEYEDLGIYYCLQYDEFPWTFGG
GTRLEIK
Antibody 3358 (or 543)
variable heavy chain (IgG2a)

(SEQ ID NO: 3)

QVQLQQPGAELVKPGASVRLSCKAGGYTFTSYWLHWVKQRPGQGLEWIGM
IHPNSGSYDYSETFRTKATLTVDKSSDTAYMQLTSLTSEDSAVYYCARGG
SNYDIFAYWGQGTTLTVSS
variable light chain (kappa)

(SEQ ID NO: 4)

NIVMTQSPKSMSMSVGERVTFNCRASENVGTYVFWYQQKPEQSPRLLIYG
ASNRYTGVPDRFTGSGSATDFTLTISGVQAEDLADYHCGQSYRHLTFGGG
TKLEIK
Antibody F87
variable heavy chain

(SEQ ID NO: 5)

QVQLQQPGTELVKPGASVKLSCKASGYTFTNYWMHWVKQRPGQGLEWVGN
INPSNGGTNYNEKFKSKATLTVDKSSSTAYMQLSSLTSEDSAFYYCARGR
GPPYYSDYWGQGSTLTVSS
variable light chain

(SEQ ID NO: 6)

NIQMTQSPASLSASVGETVTITCRASGNIHNYLAWYQQKQGKSPQLLVYN
AKTLADGVPSRFSGSGSGTQYSLKINSLQPEDFGSYYCQHFWSTPFTFGS
GTKLEIK
rPBA-3
variable heavy chain (IgG₁)

(SEQ ID NO: 7)

QVQLQQSGPELVKPGASVRISCKASGFTFTSYHVNWVKQRPGQGLEWIGW
IYPGNVNTEYNEKFKGKATLTADKSSSTAYMQLSSLTSEDSAVYFCASHE
YYGSDWYFDVWGAGTTVTVSS
variable light chain (kappa)

(SEQ ID NO: 8)

DAVMTQTPLSLPVSLGDQASISCRSSQSLENRNGNTYLNWYLQKPGQSPQ
LLIYRVSNRFSGVLDRFSGSGSGTDFTLKISRVEAEDLGVYFCLQVTHVP
YTFGGGTKLEIK
Recombinant Antibody 227 (or 3359)
heavy chain full length sequence

(SEQ ID NO: 26)

EVQLVESGGGLMKPGGSLKLSCAASGFAFGSYDMSWVRQTPEKRLEWVAY
ISSGGGITFYPDSVRGRFTISRDNAKNSLYMEMSSLRSEDTAMYYCARWD
WDLFAYWGQGTLVTVSATKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKRVEPKSCDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK
light chain (kappa) full length sequence

(SEQ ID NO: 27)

DIKMTQSPSSMYTSLGERVTITCKASQDINSCLSWFQQKPGKSPKALIFR
ANILVDGVPSRFSGSGSGQDYSLTISSLEYEDLGIYYCLQYDEFPWTFGG
GTRLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Recombinant Antibody 3358 (or 543)
heavy chain (IgG2a) full length sequence

(SEQ ID NO: 28)

QVQLQQPGAELVKPGASVRLSCKAGGYTFTSYWLHWVKQRPGQGLEWIGM
IHPNSGSYDYSETFRTKATLTVDKSSDTAYMQLTSLTSEDSAVYYCARGG
SNYDIFAYWGQGTTLTVSSTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
light chain (kappa) full length sequence

(SEQ ID NO: 29)

NIVMTQSPKSMSMSVGERVTFNCRASENVGTYVFWYQQKPEQSPRLLIYG
ASNRYTGVPDRFTGSGSATDFTLTISGVQAEDLADYHCGQSYRHLTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
Recombinant Antibody F87
heavy chain full length sequence

(SEQ ID NO: 30)

QVQLQQPGTELVKPGASVKLSCKASGYTFTNYWMHWVKQRPGQGLEWVGN
INPSNGGTNYNEKFKSKATLTVDKSSSTAYMQLSSLTSEDSAFYYCARGR
GPPYYSDYWGQGSTLTVSSTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLPPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALNNHYTQKSLSLSPGK
light chain full length sequence

(SEQ ID NO: 31)

NIQMTQSPASLSASVGETVTITCRASGNIHNYLAWYQQKQGKSPQLLVYN
AKTLADGVPSRFSGSGSGTQYSLKINSLQPEDFGSYYCQHFWSTPFTFGS
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The variable heavy and variable light chain domains of PCG-4 were created synthetically based on the sequences for the light and heavy chains obtained from Genbank (accession numbers X82691 and X82692). The variable domain of both the heavy and the light chains were individually synthesized from 12 synthetic oligos by overlap extension PCR (57). The full-length product was cut with SacI and BbsI (sites included in terminal PCR primers; BbsI site designed to generate CCTC overhangs compatible with vector) and cloned between the AppA leader sequence (for periplasmic export) and domain I of the human IgG heavy chain in vector pKW-1 (PBK-CMV derivative).

An appA leader sequence was added to the light chain before the insert was cut with BamHI and XhoI and cloned into pBAD33 for Fab expression. The variable domains were subcloned from the Fab construct into the same pCEP4 vector system described for rPBA-3 for production of a chimeric full-length IgG construct.

The constant region sequences are human sequences known in the art. Any constant region sequence can be used, e.g., any human constant region sequence can be used to design and make an antibody of the invention.

### Generation and Screening of Anti-Toxin A and B Monoclonal Antibodies

In one aspect, the invention provides antibodies comprising the heavy chain variable region sequence encoded in SEQ ID NO:1 and the light chain variable region sequence encoded in SEQ ID NO:2 and the remainder of the antibody (e.g., constant region) comprising human sequence, thus making a “humanized” chimeric antibody (similarly; and in alternative aspects the “humanized” chimeric antibody comprises the variable region sequence combinations SEQ ID NO:3 and SEQ ID NO:4, or, SEQ ID NO:5 and SEQ ID NO:6, or, SEQ ID NO:7 and SEQ ID NO:8). Exemplary methods to make antibodies of the invention are described herein. Any constant region sequence can be used, e.g., any human constant region sequence can be used to design and make an antibody of the invention, e.g., and antibody having a variable region comprising SEQ ID NO:1, SEQ ID NO:2, SEQ ID NO:3, SEQ ID NO:4, SEQ ID NO:5, SEQ ID NO:6, SEQ ID NO:7 and/or SEQ ID NO8, or substantially similar sequences, as set forth herein. Human constant region sequences are well known in the art, e.g., see discussion on Kabat sequences and Ab databases, above.

A total of 30 mice (5 BALB/c and 10 Swiss-Webster) were immunized by 4 25 μg injections every 21 days with either ToxA:1800-2710 or ToxB:1807-2366 at Strategic BioSolutions (Newark, Del.). Removal of the hexhistidine tags was performed by thrombin cleavage and dialysis overnight in MWCO 10000 dialysis tubing prior to injection. After 12 weeks, all mice had developed anti-toxin A and/or anti-toxin B antibody titers. Sera of the third bleed were tested in toxin neutralization cell assays and by surface plasmon resonance to rank the mice. Fusions were initiated with spleen cells of mice demonstrating high anti-toxin titer, toxin neutralization in cell assays and cross-reactivity for toxin A and toxin B in toxin neutralization assays and in ELISA.

Transfection of rPBA-3 and rPCG-4 into 293F Mammalian Cell Expression Host

The heavy and light chain plasmids of both rPBA-3 and rPCG-4 were transformed into XL1-blue bacteria and stocked in glycerol. Large scale plasmid DNA was prepared as described by the manufacturer (Qiagen, endotoxin-free MAXIPREP™ kit Cat#12362). Plasmids were transfected into the adenovirus-transformed human embryonic kidney cell line 293F using 293fectin and using 293F-FreeStyle Media for culture. Light and heavy chain plasmids were both transfected at 0.5 μg/mL. Generally, the cells were spun down after 3 days and ½ the media was replenished with fresh media. Transfections were performed at a cell density of 10⁶ cells/mL. Supernatants were collected by centrifugation at 1200 rpm for 8 minutes at 25° C. 7 days after transfection. Expression levels varied from ˜0.25-1.5 μg/mL. Supernatants were stored as described above for hybridoma cultures.

### Protein Purification/Quantification

Native toxin domains were purified on an AKTA FPLC (Amersham Biosciences) using a two-step procedure. The supernatants from sonicated cell pellets were first applied at 3 mL/min onto a Ni²⁺-bound HITRAP™ chelating column (Amersham, Cat#17-0409-01). Toxin domains were eluted by applying a 50-300 mM imidazole gradient. HisTagged toxins were eluted between 200 and 250 mM imidazole and collected in a 96-well plate. Toxins A and B have very different pIs, therefore the CWB-constructs of each toxin were applied to different ion exchange resins. Ni²⁺-purified toxin A domains were dialyzed overnight against a 50 mM MES, 100 mM NaCl, pH 7.0 buffer and applied a HITRAP-CM™ prepacked ion exchange resin (Amersham, Cat#17-5056-01). The toxin A constructs were eluted with a gradient of 0.1-1.0 M NaCl. Ni²⁺-purified toxin B domains were dialyzed overnight against a 50 mM Tris, 100 mM NaCl, pH 7.9 buffer and applied to a HITRAP-SP™ prepacked ion exchange resin (Amersham, Cat#17-1151-01). The toxin B constructs were eluted with a gradient of 0.1-1.0 M NaCl. All purified toxin domains were dialyzed extensively against a 5 mM phosphate buffer, pH 7.4 for storage at 4° C. and future analysis (Pierce SLIDE-A-LYZER™ Cassette, 3500 MWCO, cat#66110). Stock concentrations were determined by UV absorbance using the method of Pace and coworkers (2).

ToxinA:1800-1945, ToxinA:2078-2234 and ToxinB:2207-2366 were found primarily (˜90%) in the insoluble fraction of the cell pellet. To test whether these domains can be refolded, insoluble ToxA:1800-1945 and ToxA:2078-2234 were solubilized with urea at pH 7.5 and captured with a Ni²⁺-NTA resin (Qiagen). The protein material was eluted from the resin with EDTA and dialyzed overnight against PBS. Alternately, the EDTA extractions were injected directly onto a reverse phase C5 JUPITER™ HPLC column (Phenomenex Inc.). H₂O/acetonitrile gradients with 0.1% trifluoroacetic acid were used to elute the toxin domains from the hydrophobic matrix. Denatured toxins eluted between 80% and 90% acetonitrile in a broad peak typical of aggregated/unfolded protein material. No purified toxin domains from the insoluble fractions of the cell pellets ever refolded.

PBA-3, 3358 (or 543), 227 and 251 mouse monoclonal antibodies and rPBA-3 and rPCG-4 chimeric antibodies were purified by passing culture supernatants over protein G columns (Amersham, cat#17-0405-01) at 4 mL/min. Multiple passage of supernatants over the columns was unnecessary as >95% of all IgG material from each supernatant bound to the column on the first pass. Mobile phases consisted of 1×PBS-Tween (Sigma Aldrich, Running Buffer, cat# P-3563) and 0.1 M glycine pH 2.7 (Fisher Chemicals, Elution Buffer, cat# G48-500). Antibody collections in 0.1 M glycine were diluted 20% (v/v) with 1 M TrisHCl, pH 8.0, to neutralization the pH. IgG1 collections were pooled and dialyzed exhaustively against 1×PBS (Pierce SLIDE-A-LYZER™ Cassette, 3500 MWCO, cat#66110). The concentration of each IgG1 stock solution was determined by Bradford analysis (Bio-Rad Protein Assay, Hercules, Calif. cat#500-0006) using a commercial myeloma IgG1 stock solution as a standard and by UV absorbance.

### ELISA Testing for Anti-Toxin A and Anti-Toxin B Antibodies

ToxA:1800-2710 and ToxB:1808-2366 were biotinylated using the EZ-LINK-Biotin-LC-ASA™ kit (PIERCE catalog #29982). Briefly, EZ-LINK-BIOTIN-LC-ASA™ was dissolved in DMSO and added to toxin A or toxin B at a 4:1 molar ratio. Protein/biotin conjugation was induced for 20 minutes under a UV lamp in a PBS buffer. Conjugated toxins were removed from unreacted biotin by application of the reaction mixture to a desalting column (PIERCE D-Salt Dextran Plastic Desalting Columns, catalog #43230). 500 μL fractions from the desalting procedure were tested for protein absorption at 280 nm to detect the presence of biotinylated toxins.

Microtiter Streptavidin plates (Sigma Chemical, St. Louis, Mo., catalog #M5432) were coated with 200 ng per well of biotinylated ToxA:1800-2710 or ToxB:1808-2366 diluted into PBS buffer and incubated at 4° C. overnight. The plates were then washed 3 times with TBST buffer. All samples were diluted in Tris buffer, pH 8.0 TBST buffer (Sigma, cat#T9039). Aliquots of 100 μL of each diluted serum sample or fusion supernatant were transferred to the toxin-coated plates and incubated for 1-2 hours at room temperature. Following 3 washes with TBST, alkaline phosphatase-conjugated rabbit anti-mouse IgG(H+L) (Zymed, cat#61-6522) was added to each well at a 1:1000 dilution. The reaction was carried out for 1 hr at room temperature, plates were washed 3 times with TBST and 100 μL of p-nitrophenylphosphate substrate was added (Sigma, Catalog # A3469). The absorption was determined at 405 nm using a Molecular Devices vₘₐₓ kinetic microplate reader.

### Gel Filtration Analysis of Toxin A Domains

Analyses were run using a Waters BREEZE™ HPLC system equipped with a dual channel absorbance detector and autosampler. A BIOSEP-SEC-S-2000™ Gel filtration column (Phenomenex) was used for separation with a flow rate of 1 mL/min. The mobile phase buffer was PBS alone or supplemented with either 10 mM CaCl₂ or 5 mM choline. The column was standardized using IgG1 (150 kDa), BSA (66 kDa), bovine CH3 (31 kDa; 10), carbonic anhydrase (28 kDa), lysozyme (14 kDa), and ubiquitin (8.5 kDa).

### Circular Dichroism (CD) Spectroscopy

CD spectra were taken on an Aviv model 215™ spectrophotometer equipped with a thermoelectric cuvette holder. All final spectra were the average of at least three scans utilizing a signal averaging time of 3 s/λ and a 1 nm bandwidth. Temperatures were held constant using a Peltier heating/cooling device coupled to a circulating water bath maintained at 20° C. All scans were performed in a 1 mm cuvette at 100 μg/mL toxin concentrations and using a 5 mM phosphate, 10 mM NaCl buffer, pH 7.4. Near-UV spectra were performed in a 1 cm cuvette and were the average of 5 scans with a 3 s/λ signal averaging time and a 2 nm bandwidth.

### Protein Stability and Small Molecule Binding

Temperature dependent far-UV CD spectra were collected at 5 C° intervals from 25 to 75° C. on all three samples using a 1 mm cuvette. Near-UV CD scans of ToxA:2459-2710 were performed as described above using the same temperature range. Precise thermal denaturations of ToxA:2459-2710 were performed by monitoring the far-UV CD signal at 230 nm between 10 and 90° C. using a 1 cm cuvette with constant stirring and 1° C. temperature intervals. The temperature equilibration period was 3 minutes/deg and the UV-averaging time was set to 30 s/° C. Far-UV CD curves of ToxA:2459-2710 were fit to a two-state unfolding model (Privalov, 1979). A theoretical ΔCₚ°, 4064 cal/mol K (1 cal=4.184 J), was calculated based on an estimate of the change in accessible surface area (ΔASA) between the folded and unfolded state of the toxin domain (Myers et al., 1995). Equilibrium dissociation constants at 25° C. for small molecules bound to ToxA:2459-2710 were determined by measuring the apparent unfolding equilibrium constants of the domain in the absence and presence of ligand (Brandts and Lin, 1990):

$${K}_{app} ( 25 ° C . ) = \frac{{K}_{U} ( 25 ° C . )}{1 + {K}_{A} ( 25 ° C . ) [ L ]} ; {K}_{d} ( 25 ° C . ) = \frac{{K}_{U} - {K}_{app}}{{K}_{app} [ L ]} ,$$

where K_{U} is the equilibrium unfolding constant of ToxA:2459-2710 in the absence of ligand, Kₐₚₚ is the apparent equilibrium unfolding constant in the presence of ligand and [L] is the ligand concentration. Choline dihydrogencitrate was purchased from Sigma. Unconjugated linear B2-trisaccharide (Galα1-3Galβ1-4GlcNac) was purchased from V-LABS, Inc and B2-trisaccharide conjugated to BSA was purchased from Calbiochem (cat#436200).

Determination of Antibody Binding Kinetics by Surface Plasmon Resonance (SPR)

All SPR experiments were performed on a Biacore3000 instrument set to 31° C. Toxins were immobilized to research grade CM5 Chip surfaces using the immobilization programs within the Biacore3000 Software. Carboxymethyl-moieties on the surface of each flow cell were activated using standard EDC/NHS chemistry. Toxins were immobilized to Chip surfaces by primary amine coupling to the activated surfaces with a 10 mM Acetate buffer, pH 4. Toxin B repeat domains degrade rapidly at low pH and were coupled immediately (within seconds after lowering the pH). Kinetic analysis of each anti-Toxin Fab or monoclonal antibody was performed by injecting a series of concentrations over each toxin-bound flow cell. Typical antibody concentration series were 0.5, 2, 6, 20 and 100 nM injections. Chip surfaces regenerated reliably with a 10 μL injection of 0.1 M glycine, pH 1.5 followed by a 10 μL injection of 50 mM NaOH. The flow rate was 30 μL/min. For all runs, there was a flow cell dependent baseline drift between 0.0002 and 0.001 RU/sec, which could be accounted for in the 1:1 fitting model used to analyze the kinetics.

### Epitope Mapping and Antibody Competition Studies

Monoclonal antibodies 3358 (or 543), 3350 (or 227) and rPCG4 were immobilized to 3 separate surfaces on a research grade CM5 Chip. 30 nM ToxinA:1800-1945 and 15 nM ToxinA:1800-2710 were separately injected over the flow cells at 10 μL/min. Prior to injection, the toxins were incubated with 0, 1, 3, 5, 8, 12, 15, 20, 25, 30, 35, 40, 50, 60, 70, 80, 90, 105 and 120 nM concentrations of 3358 (or 543), 227, rPCG4 (full-length antibody) and rPCG4 (Fab format). After each injection, the flow rate was increased to 30 mL/min and the antibody surfaces were regenerated with 10 μL of 0.1 M glycine, pH 2.0. Regeneration did not affect the 3358 (or 543) and 227 surfaces, but resulted in approximately 0.5% signal loss per injection for the rPCG-4 antibody. Every sixth injection was performed with 100% free toxin, to continually monitor the free toxin signal.

### Toxin Neutralization Assay

CHO-K1 cells (ATCC CCL-61) were maintained in Dulbecco's modified Eagle's medium (DMEM—Gibco, cat#12430-054) supplemented with 10% fetal bovine serum at 37° C. in a CO₂ incubator. Prior to the experiment, cells were split into T75 flasks at 1.5×10⁶ cells/flask. Cells reached confluency within 2 days. CHO cells were seeded from these stocks into 96-well cell culture plates (Costar, cat#3598) and incubated for 4-6 hours. Toxin A and toxin B were purchased from List Biological Laboratories. Toxin and toxin/antibody mixtures were incubated at 37° C. in DMEM with 10% FBS (Gibco, cat#10082-147) for 1 hour and added to the 96-well plates. Cell rounding was monitored over a 48 hour period. At 48 hours, 10 μL WST-1 (Roche, cat#1644807) was added to each well and the plates were incubated for 1 hour at 37° C. Absorbance at 450 nm was determined using a Molecular Devices SPECTRAMAX PLUS 384™ 96-well plate reader.

Kinetic measurement of toxin A mediated cell rounding at various calcium concentrations was performed by culturing the CHO cells in calcium depleted DMEM (Sigma, cat#M8167) doped with L-glutamine and 5% FBS. 96-well plates were seeded with CHO cells as described above. CaCl₂ was doped into the solution to create a calcium gradient consisting of 100 μM, 300 μM, 700 μM, 1 mM, 2 mM, 5 mM and 10 mM Ca²⁺. Data from the 10 mM cell killing assay was discarded due to significant levels of precipitated CaCl₂. Time points were taken every hour after the addition of toxin A (up to 6 hrs) and after overnight incubation. The percentage of cell killing was determined by counting the number of flat versus round cells. At least 150 cells were counted and at least three separate spots on each well were used to account for any variability in killing throughout each well.

### Flow Cytometry

CHO cells were cultivated at a ratio of 1:10 and grown in 10 cm dishes, washed two times with PBS, scraped, and pelleted at 1100 rpm at 4° C. for 5 minutes. Cell staining was performed with 5×10⁵ cells/tube in wash buffer (PBS supplemented with 2.5 mM Hepes, 0.1% sodium azide, and 2% FBS). ToxA:1800-1945, ToxA:2078-2234, ToxA:2459-2710 and ToxA:1800-2710 were all tested for CHO-cell binding at various protein and Ca²⁺ concentrations. ToxA:2459-2170 (50 μg/mL) was chosen for the flow cytometry assay based on its native folding properties and cell binding characteristics. To study antibody neutralization mechanisms, 250 or 500 μg/mL antibody was combined with ToxA:2459-2710 to a total volume of 100 μL and incubated with the cells for 20 minutes. Cells were washed two times with 2 mL wash buffer. Adhered toxins were detected via their histidine tags by incubation with the Alexafluor 488 conjugated PENTA-HIS monoclonal antibody (Qiagen, cat#35310) for 20 minutes. Cells were washed twice with 2 mL wash buffer and resuspended in 500 μL wash buffer. The CaCl₂ concentration was held strictly to 1 mM where the addition of ToxA:2459-2710 consistently led to a 35-50% population of fluorescently labeled cells. Ca²⁺ contributed by the 2% FBS was less than 100 μM, Invitrogen/Gibco.

Flow cytometry analysis was performed on a DakoCytomation MoFlo flow cytometer (Fort Collins, Colo.) equipped with a Coherent ENTERPRISE II™ (Santa Clara, Calif.) water-cooled argon ion multi-line laser. The 488 nm line was used as the excitation source. Forward scatter (FSC), side scatter (SSC) and fluorescent properties were detected by R928™ photomultiplier tubes (Hammamatsu, Shizuoka-ken, Japan). Fluorescence was detected between 510 and 550 nm. Data was collected for 10,000 events and was analyzed using DakoCytomation SUMMIT v3.1™ software.

### Rat Ileal Loop Study

Experiments were conducted as described in the protocol approved by IAACAC. Briefly, 5 μg of native toxin A was mixed with various amounts of test antibody in a final volume of 400 μl and injected in the ligatured ileal loop. Animals were fasted overnight and were given water ad libitum. Ligated rat small intestinal segments (5 cm) were injected with 400 μl of toxin A with or without test article, with cholestyramine, or control material (saline or test article alone). 4 h after injection, the weight and length of ileal loops were measured. A polyclonal antibody against toxin A shown to work 100% in toxin neutralization cell assays was also tested. Male rats were used in all studies. 2 loops were injected per rat. Groups of 2 or 3 rats were used per test article.

### Results

Toxin A and B CWB-Motif Structural Analysis

No structure of the toxin A or toxin B CWB-domains has previously been determined. The primary sequences of the toxin A and toxin B repeats are similar to the 20 residue repeat sequences of LytA from streptococcus pneumoniae (FIG. 8). FIG. 8A illustrates the CLUSTALW alignment of repeat domains of several CWBs, including Streptococcal mutans, downei, LytA, ToxA, ToxB, ToxL: (“x” in the CDiffToxB sequence below, and FIG. 8A, is only a space-holding indicator to facilitate sequence alignment of the several CWBs):

| | |
|---|---|
| StreptmutansGtfC | |
| GTVTFNGQRLYFKPNGVQAK | (SEQ ID NO: 15) |
| StreptmutansGtfB | |
| GARTINGQLLYFRANGVQVK | (SEQ ID NO: 16) |
| StreptsobrinasGtfI | |
| GAQTIKGQKLYFKANGQQVK | (SEQ ID NO: 17) |
| PhageCP-1 | |
| GWVKIGDGWYYFDNSGAMAT | (SEQ ID NO: 18) |
| StreptpneumoniaLytA | |
| GWIKIADGWYYFDSDGAMAT | (SEQ ID NO: 19) |
| CDiffToxA | |
| GWQTINGKKYYFNTNTAAAA | (SEQ ID NO: 20) |
| CDiffToxB | |
| GLVXIDDKKYYFDDDGIMQX | (SEQ ID NO: 21) |
| CSordeliiToxL | |
| GLITIDDKKYYFDDNGIMQV | (SEQ ID NO: 22) |
| CDiffToxA | |
| GVFKGPNGFEYFAPANTDNNNIEGQAIVYI | (SEQ ID NO: 23) |
| CDiffToxB | |
| GVFNTEDGFKYFAPANTLDENLEGEAINYI | (SEQ ID NO: 24) |
| CSordeliiToxL | |
| GVFNTPDGFKYFAPXNTLDENXEGESVNYT | (SEQ ID NO: 25) |

FIG. 8B illustrates Far-UV CD spectra of Toxin Domains. A positive peak at 230 nm is only present for the full-length CWB domains of toxins A and B and the truncated domain, ToxA:2459-2710. C. Far-UV CD of ToxA:2459-2710, ToxA:1800-1945 and ToxA:2078-2234 at 25 and 75° C. The spectra of ToxA:1800-1945 and ToxA:2078-2234 does not demonstrate a thermal unfolding transition as is observed for ToxA:2459-2710. The denatured spectrum of ToxA:2459-2710 at 75° C. is similar to the spectra of ToxA:1800-1945 and ToxA:2078-2234 at both 25 and 75° C.

LytA forms a unique β-solenoid structure using a minimum of six repeats (5). The CWB-domains of toxin A and B had the added complexity of a second-type of repeat sequence occurring after approximately every six classical repeats.

Three constructs of Toxin A and B (ToxA:1800-1945, ToxA:2078-2234 and ToxB:2208-2366) were composed of six or seven repeats (similar to the folded LytA protein) that formed a β-solenoid-type secondary structure based on CD measurements. The spectra of all three protein constructs contain a characteristic minimum at 212 nm (FIG. 8B). However, the lack of a near-UV CD signal and thermal unfolding transition indicated these domains were not cooperatively folded (i.e., are not miniature representatives of the overall folded structure of the molecule). The expressed domains were also found primarily in inclusion bodies, not the soluble fraction, another indication that they were not properly folded. ToxA:1800-1935 and ToxA:2078-2234 isolated from inclusion bodies using Ni²⁺ capture in urea and/or reverse phase HPLC did not refold to the native secondary structure of the limited soluble protein fraction and had far-UV CD spectra indicative of random coil.

An eleven repeat construct, ToxA:2459-2710, was cooperatively folded with a far-UV CD spectrum identical to that of the full-length CWB-domains of both toxin A and B. The far-UV CD spectra of the folded domains were distinguishable from the 6 and 7 repeat toxin domains with an added characteristic maximum at 230 nm (FIG. 8b). ToxA:2459-2710 had a negative near-UV CD peak at 284 nm indicating the burial of aromatic residues within a tertiary fold. The folded toxin domains ToxA:1800-2710, ToxB:1807-2366 and ToxA:2459-2710 were all monomeric as judged by gel filtration analysis. The domains with 6 and 7 repeats tended to associate with the gel filtration matrix, typical of partially folded proteins with exposed hydrophobics. ToxA:2459-2710 unfolded in a two-state fashion. The melting temperature (T_{M}) measured by near (284 nm) and far (230 nm) UV CD was identical, 49° C. (FIG. 11A). The spectrum of thermally unfolded ToxA:2459-2710 resembled the spectra of the smaller, 6-7 repeat indicating the presence of β-solenoid-like secondary structure even in the denatured state of ToxA:2459-2710, see FIG. 8C. This unfolding temperature agreed with early literature demonstrating that toxins A and B are no longer active at 56° C. (7).

FIG. 9 illustrates photographs of adherent CHO cells cultured in the absence (media only) and presence of 20 ng (100 μL total volume) toxin A with and without anti-toxin A antibodies present. Antibody concentrations are provided on the images. Antibodies provided significant protection from toxin A at concentrations of 2 μg. Interestingly, antibody combinations, i.e., 543 and 227, 227 and rPCG-4 at 1 μg are each more neutralizing that the single antibodies alone.

FIGS. 10A to E illustrate the antibody competition for toxin binding sites using static concentrations of toxin and titrating the amount of antibody in solution. The amount of toxin with available binding sites was determined by capture of toxin with available binding sites to antibodies immobilized to CM5 chip surfaces. Titration of ToxA:1800-1945 with rPCG-4 Fab (A). Titration of ToxA:1800-2710 with rPCG-4 Fab (B), full-length rPCG-4 antibody (C), antibody 227 (D) and antibody 543 (F).

FIGS. 11A to F illustrate: FIG. 11(A) the thermal denaturation of ToxA:2459-2710 in the absence and presence of CWB-binding ligands monitored by the CD signal of the protein at 230 nm. (FIG. 11B-E) Calcium dependent binding of ToxA:2459-2710 to CHO cell surfaces determined by flow cytometry. 100 μL of 50 μg/mL ToxA:2459-2710 was incubated with 0.5×10⁶ CHO cells for 20 minutes on ice. The cells were washed twice with 2 mL buffer and added to 100 mL of an anti-His tag ALEXAFLUOR™-conjugated antibody (1:500 dilution) for 20 minutes. Cells were washed again and applied to the flow cytometer. A gate was used to visualize live cells based on photomultiplier counts between 10³ and 10⁴ for both side-scatter and forward scatter. FIG. 11(F) The ratio of rounded CHO cells after incubation for 5 hours with 80 ng toxin A was determined in the presence of 100 μM, 300 μM, 700 μM, 1 mM, 2 mM and 5 mM CaCl₂ concentrations.

ToxA:2459-2710 bound choline (similar to pneumococcal LytA), but did not bind Galα1-3Galβ1-4GlcNac as has been reported for full-length toxin A. Binding was assessed by addition of various concentrations of choline or Galα1-3Galβ1-4GlcNac and testing for an increase in the T_{M} of the protein upon binding (FIG. 11A). The CWB-domain bound choline with an apparent dissociation constant of 13±5 mM assuming a 1:1 interaction. The domains almost certainly bound numerous choline moieties considering what was known for LytA (5). Binding of Galα1-3Galβ1-4GlcNac was very weak with an estimated K_{d}>100 mM in the absence or presence of additional Ca²⁺. Additionally, surface plasmon resonance experiments did not detect any interaction between all four toxin A CWB-domains and a CM5 surface coated with Galα1-3Galβ1-4GlcNac conjugated BSA. The toxin A CWB-domains were tested at concentrations as high as 1 μM against the trisaccharide-conjugated surface.

### Antibody Mediated Toxin Neutralization

Recombinant forms are produced from the publicly available antibodies PCG-4 (see, e.g., Lyerly (1986) Infect Immun. 54:70-76; Frey (1992) Infect. Immun. 60: 2488-2492) and PBA-3, were produced. Antibodies were raised against ToxA:1800-2710 and ToxB:1807-2366 in Swiss-Webster mice. Two hybridoma cell lines, 3358 (or 543) and 227, produced the most neutralizing anti-toxin A antibodies. One anti-toxin B monoclonal was found to effectively neutralize toxin B.

All anti-toxin A antibodies recognized multiple CWB-mini-domains as determined by surface plasmon resonance (Table 6). Each monoclonal antibody (and rPCG-4 Fab) was tested for binding to all four recombinant toxin A domains. Although the monoclonal antibodies were functionally bivalent and ToxA:1800-2710 certainly had multiple antibody binding sites, the data sets fit well to a 1:1 model. The apparent kinetic/equilibrium constants were used to evaluate relative strength of antibody binding. 3358 (or 543) and rPCG-4, recognized all toxin A repeat domains with high affinity (K_{Dapp}<30 nM) demonstrating that the tertiary structure observed for ToxA:2459-2710 and ToxA:1800-2710 was not crucial for high affinity binding. The β-solenoid secondary structure, however, was required for high affinity recognition. rPCG-4 binding to denatured toxin A CWB-domains isolated from inclusion bodies was at least five-fold weaker than its binding to the same toxin A domains isolated under entirely native conditions.

Table 6 shows the apparent antibody affinities for toxin A CWB as determined by surface plasmon resonance. The potential number of toxin A-binding sites was estimated based on concentration dependent antibody competition studies. Toxin neutralization was determined using CHO cells with a concentration of toxin A killing 100% of the cells. The anti-toxin polyclonal antibody was used as a positive control with approximately 100% staying alive in presence of the mixture toxin A and antibody.

TABLE 6

| | Binding Affinity K_{d} (nM) | | | | | |
|---|---|---|---|---|---|---|
| | ToxA | ToxA | ToxA | ToxA | #Potential | |
| Antibody Lines | 1800-1945 | 2078-2234 | 2459-2710 | 1800-2710 | binding sites | Neutralization |
| rPCG-4 (2) | 1.2 | 0.3 | 29 | 0.5 | 4-6 | +++ |
| rPBA-3 (0) | — | 55 | 60 | 16 | N.D. | + |
| 3358 (or 543)(3) | 0.6 | 3.4 | 0.7 | 0.3 | Up to 14 | +++ |
| 3359 (or 227)(1) | 100 | 13 | 12 | 1.2 | >2 | ++ |
| 251 (1) | 3.2 | 106 | 20 | 0.3 | N.D. | + |
| rPCG-4/3358 | — | — | — | — | — | +++ |
| (or 543) | | | | | | |
| rPCG-4/3359 | — | — | — | — | — | +++++ |
| (or 227) | | | | | | |
| 3358 (or 543)/ | — | — | — | — | — | ++++ |
| 3359 (or 227) | | | | | | |
| Polyclonal | — | — | — | — | — | +++++ |
| IgG | | | | | | |
| Toxin alone | — | — | — | — | — | − |

Further epitope mapping was performed by competition analysis using surface plasmon resonance. The percentage of free toxin in antibody/toxin mixtures was determined by injection of these solutions over CM5 chips immobilized with anti-toxin A antibodies. As expected, the rPCG-4 Fab competed with immobilized rPCG-4 antibody. At 30 nM ToxA:1800-1945 concentrations, the Fab saturates the toxin at a 1:1 toxin:antibody ratio (FIG. 10a). The full-length rPCG-4 antibody saturated ToxA:1800-1935 at a toxin:antibody ratio of 2:1 demonstrating its bivalency. rPCG-4 Fab saturated the full-length CWB-domain, ToxA:1800-2710, at a 1:6 toxin:antibody ratio while the full-length antibody saturates at a 1:3 ratio (FIG. 10B,C). This data suggested that PCG-4 recognized a maximum of six separate sites within the CWB-domain of toxin A with high affinity. The existence of weak binding sites (i.e., K_{D}>10⁻⁸) as observed for ToxA:2457-2710 predicated there may be as few as four sites; however, the fact that bivalent rPCG-4 saturated at exactly half the concentration of its Fab counterpart suggests that six high affinity binding sites was more likely.

The two neutralizing monoclonal antibodies 3359 (or 227) and 3358 (or 543) performed differently than rPCG-4 in the competition assay. The 3359 (or 227) antibody did not saturate the toxin even at an 8 molar excess of antibody (FIG. 10D). The 3359 (or 227) antibody also did not compete with 3358 (or 543), but instead synergistically enhanced 3358 (or 543)'s binding to ToxA:1800-2710. The 3359 (or 227) antibody only weakly competed with rPCG-4. The 3358 (or 543) antibody saturated its own binding sites at a 1:7 toxin:antibody ratio suggesting that 3358 (or 543) had a maximum of approximately fourteen high affinity binding sites (FIG. 10E). 3358 (or 543) competed for rPCG-4 binding sites exactly as it competed against itself; an indication that the two antibodies had strongly overlapping binding sites. 3358 (or 543) did not displace 3359 (or 227) at concentrations below 40 nM and only partially displaced 3359 (or 227) at higher concentrations suggesting the two antibodies had at least one or two fully independent binding sites.

The anti-toxin A and anti-toxin B antibodies were tested for their ability to neutralize full-length, active toxin A and B in a cell-killing assay. In general, 4, 0.8 and 0.2 μg/mL toxin A and 60 and 20 ng/mL toxin B were used in the assays. All three toxin A concentrations and 60 ng/mL toxin B were 100% killing in the cell assay while 20 ng/mL toxin B was generally partially killing when incubated with CHO cells. To test for neutralizability, the antibodies were introduced with toxin at 20, 10 and 5 μg/mL concentrations. At 2 μg/mL, the rPCG-4, 3358 (also designated 543) and 3359 (also designated 227) antibodies are all capable of partially neutralizing 0.2 μg/mL concentrations of toxin A (Table 6). rPBA-3 only weakly neutralized at a concentration of 8 μg/mL. While some variability was observed in the cell neutralization assay depending upon the specific batch of toxin A and the age and/or healthiness of the CHO cells used in the assay, the indicated trends were observed over multiple (3 or more) separate cell assays.

The 3358 (or 543) and 3359 (or 227) B-cell lines were selected originally from murine B-cell supernatants based on a synergistic neutralizing affect discovered when testing the two supernatants in combination. The clonally selected, purified antibodies demonstrated a similar synergistic ability to neutralize toxin at antibody concentrations lower than the concentrations necessary to observe partial neutralization by each antibody alone (Table 6, FIG. 9). The 3359 (or 227) antibody also combined favorably with rPCG-4 towards toxin neutralization. rPCG-4 and 3358 (or 543) were weakly synergistic in their ability to neutralize toxin A, however the neutralization was generally much weaker than what is observed for the 3359 (or 227)/3358 (or 543) and 3359 (or 227)/rPCG-4 pairs. Photographs of cell cultures grown in the presence and absence of toxin and antibodies are shown in FIG. 9. The presence of unique epitopes recognized by the 3359 (or 227) antibody can explain how it preferentially coupled with 3358 (or 543) and rPCG-4 for enhanced toxin neutralization. The fact that 3358 (or 543) and rPCG-4 competed for overlapping binding sites also provides an explanation as to why these antibodies did not display as strong a synergistic effect.

Similar to what was observed for the anti-toxin A monoclonals, the epitope, but not the affinity, of a monoclonal antibody was most important for toxin B neutralization (Table 7). Monoclonal F85 was as neutralizing as the polyclonal TechLab standard. This antibody actually had the second lowest apparent affinity for toxin B of the 6 final anti-toxin B candidates. Another antibody raised against ToxB:1807-2366, denoted F2, demonstrated fairly high affinity binding to both toxins A and B. This antibody is weakly neutralizing for toxin B in the cell-killing assay. Antibodies which recognized both toxins have been reported as ineffective for toxin neutralization (7).

Table 7 shows the apparent affinities and toxin neutralization ability of anti-toxin B antibodies. Toxin neutralization was determined using CHO cells with a concentration of toxin B killing 100% of the cells. The Techlab polyclonal antibody was used as a positive control with approximately 100% staying alive in presence of the mixture toxin A and antibody.

TABLE 7

Antibody binding
Kd (nM)

| Antibody | ToxB | ToxA | Partial |
|---|---|---|---|
| Lines | 1807-2366 | 1800-2710 | Neutralization |
| F2 | 1.4 | 100 | ++ |
| F61 | >100 | None | − |
| F67 | 0.95 | None | − |
| F73 | 0.16 | >1000 | + |
| F85 | 3.4 | None | +++++ |
| F87 | 0.05 | None | ++ |
| F2/F73 | — | — | + |
| F2/F85 | — | — | +++++ |
| F2/F87 | — | — | ++ |
| F73/F85 | — | — | +++++ |
| F73/F87 | — | — | +++ |
| F87/F85 | — | — | +++++ |
| Toxin Alone | — | — | − |
| Polyclon.IgG | — | — | +++++ |

The ability of both monoclonal antibodies 3359 (or 227) and 3358 (or 543) to neutralize toxin A in vivo was assessed using a rat ileal loop model (see FIG. 13). The loops were treated with saline, 5 μg toxin A independently and in the presence of various antibodies and antibody mixtures. Selected cross-sections of the rat ileal loops are shown in FIG. 14A. FIGS. 14A to 14D show the activity of anti-Clostridium difficile toxin A antibodies in the ileal loop model. Panels A-D show individual experiments. Neutralizing antibodies identified in the toxin neutralization cell assays were tested in the ileal loop model. An ileal loop model was developed to assess the ability of the anti-toxin A antibody to neutralize the activity of native C. difficile toxin A.

Following 4 hours (h) of incubation with 5 μg toxin A, there is clear disruption of the intestinal mucosal layer. Ileal loop incubation with 5 μg toxin A in the presence of the 3359 (or 227) or 3358 (or 543) antibodies leads to no visible disruption of the mucosal layer as compared to treatment with saline alone (FIG. 14A). Next, the antibody dosage necessary to neutralize 5 μg toxin A was investigated (FIG. 14B.). A phenotypic trait of the intestinal loops when treated with 5 μg toxin A is swelling and the accumulation of liquid. The weight/length ratio gives an accurate depiction of the activity of toxin A within the loop. Addition of monoclonal antibody levels as low as 2 μg led to complete toxin A neutralization for both the 3359 (or 227) and 3358 (or 543) antibodies. Combination of the 3359 (or 227) and 3358 (or 543) antibodies at levels as low as 0.5 μg led to complete neutralization of 5 μg toxin A. Thus, use of the combination of the 3359 (or 227) and 3358 (or 543) antibodies led to synergistic effect, e.g., their combined effect was greater than if only each were used individually.

### Ca²⁺ Regulates the Ability of the CWB-Domains to Bind Cell Surfaces and Accelerates Toxin Mediated Cell Rounding.

Calcium, but not magnesium, bound ToxA:2459-2710 as judged by the stability of the protein domain in the presence and absence of the metal ions. Addition of 10 mM Mg²⁺ had no affect on the stability of the domain; whereas a strong Ca²⁺-dependent increase in the protein's T_{M} was observed between 1-20 mM concentrations of Ca²⁺. An apparent dissociation constant, K_{D}=8±2 mM at 25° C., was calculated based on the assumption of a 1:1 binding model (FIG. 11A). These results demonstrate that the toxin A CWB-domain is probably sensitive to normal environmental fluctuations in Ca²⁺ levels with intestinal fluid and sera (18).

The CWB-domains of toxin A bound CHO cell surfaces in a calcium-dependent fashion as determined using flow cytometry. In the absence of Ca²⁺, the full length CWB-domain of toxin A, ToxA:1800-2710, and the folded 11 repeat domain of toxin A, ToxA:2459-2710, did not bind CHO cells (FIG. 11B,C). Addition of up to 10 mM Ca²⁺ induced a strong interaction between ToxA:2459-2710 and CHO-cell surfaces (FIG. 11B-E). Calcium-dependent binding of ToxA:1800-2710 to cell surfaces was also detectable, but the binding was limited by the comparatively low molar amount of protein in the assay. ToxA:1800-1945 and ToxA:2078-2234 bind weakly to cell surfaces in the absence of Ca²⁺. This may be a result of their partially folded nature, however, and not necessary an intrinsic function of these domains (12,13). The addition of calcium moderately increased the binding of ToxA:1800-1945 and ToxA:2078-2234 to cell surfaces, but not to the same extent observed for the folded ToxA:2459-2710 domain. The control His-tagged protein, bovine IgG1 CH3 (10) was not found associated with CHO cell surfaces at protein concentrations as high as 200 μg/mL and calcium concentrations as high as 10 mM.

Considering that calcium binds directly to the CWB-domain of toxin A and induces toxin association with CHO cell surfaces, calcium was introduced as a variable in toxin A mediated CHO cell killing assays. CHO cells were treated with 0.4 and 0.08 μg of toxin A and incubated anaerobically at 37° C. Both toxin concentrations were 100% lethal after overnight incubation. At 0.08 μg toxin A concentrations, Ca²⁺ had a repeatable and definitive affect on the kinetics of cell rounding. The largest disparity in cell rounding between cells incubated in Ca²⁺-depleted versus calcium rich media appeared at the 5 hour time point (FIG. 11F). Toxin A mediated cell rounding was more rapid at calcium concentrations above 1 mM. The level of calcium necessary for accelerating cytotoxicity correlates well with the apparent affinity of calcium for the CWB-domain of toxin A. Low levels of calcium (100 μM and below) also led to toxin susceptibility, potentially due to cellular responses to low calcium rather than direct calcium induced structural or physical changes within the CWB-domain itself. At 0.4 μg toxin A, cells rounded rapidly independent of the calcium concentration probably due to the saturating level of toxin.

### Mechanistic Variability of Toxin Neutralizing Antibodies

The neutralizing anti-toxin A antibodies demonstrated lead to various changes in the ability of ToxA:2459-2710 to bind CHO cell surfaces (FIG. 12). Both the 3358 (or 543) and rPCG-4 antibodies significantly increased the amount of CWB-domain detected at the cell surface. These two antibodies also demonstrated overlapping binding epitopes, suggesting a similar mechanism for neutralization. Alternatively, the 3359 (or 227) antibody inhibited cell surface association of ToxA:2459-2710. Its epitope was different according to the competition and mini-domain binding studies. At both 200 μg/mL of 3358 (or 543) and 3359 (or 227) antibody, the combination inhibited ToxA:2459-2710 binding the CHO cell surface similar to the behavior of 3359 (or 227) alone. rPCG-4 combined with 3359 (or 227) increases ToxA:2459-2710 binding to cell surfaces indicating that rPCG-4 dictated the behavior of the two antibodies when introduced at identical concentrations. As mentioned above, rPCG-4 and 3359 (or 227) weakly competed with one another while 3358 (or 543) and 3359 (or 227) appeared to have non-overlapping binding sites providing an explanation for the differential behavior of the two combinations (FIG. 10).

### Discussion

The structural features of toxins A and B are unknown at this time. Contrary to what is observed for LytA (5), 6-7 contiguous toxin A or B repeats did not contain all the necessary elements for forming the native tertiary structure of the full-length toxin CWB-domains, even though they form non-random, probably β-solenoid-like secondary structure. Toxins A and B had a unique 30 residue peptide stretch after approximately every sixth 20 residue P-motif. This unique motif can add complexity to the tertiary fold of the CWB-domains of toxins A and B differentiating these domains from LytA which is capable of forming a tertiary fold using only 6 repeats. Interestingly, the nuclear magnetic resonance assignments of a 5-repeat stretch of toxin A were recently deposited into the BMRB (Biological Magnetic Resonance Data Bank, Madison, Wis.) (30). The spectrum of the 5-repeat domain of toxin A is well dispersed and certainly suggests that the CROP polypeptide is folded. However, the CD data reported here indicates that there may be a super-secondary or tertiary structure consequence to having additional CROPs. The fact that all 6 and 7 repeat CWB-domains lack a near-UV CD signal, lack the positive far-UV signal at 230 nm present in the full-length CWB-domains and do not thermally denature demonstrates that these domains do not have all the structural aspects of the full-length CWB-domains.

The toxin A CWB-domains alone were capable of binding CHO cell surfaces. While cell surface association of toxin A to the CWB-domains has not been definitively linked, a recent study by Pfeifer et al. (32) demonstrated the localization of residues 547-2366 of toxin B in membrane fractions of Vero cells using radioactively labeled-protein. A recent study by Pfeifer et al. (32) demonstrated the localization of residues 547-2366 of toxin B in membrane fractions of Vero cells using radioactively labeled-protein. This construct of toxin B lacks the cytotoxic domain of the molecule but includes the putative transmembrane region, the 700 residues domain of unknown function as well as the CWB-domain. This large fragment of toxin B has been reported to form pores within membranes by a pH inducible mechanism (55). Another study by Aktories and coworkers demonstrated that a construct very similar to ToxA:2459-2710, designated REP231, could bind F9-cell surfaces, but only at relatively high concentrations, 200 μg/mL, and with pretreatment with 4% paraformaldehyde (34). No binding was ever detected in their assay with CHO cells. REP231 weakly inhibited the association of toxin A with F9-cells potentially demonstrating CWB-domain importance for cell surface binding. Other studies report the effect of deleting the CWB-domains from the toxins in an attempt to more fully characterize their function. Deletion of the entire CWB-domain of toxin B only attenuates its toxicity 10-fold (45), while removal of even half the CWB-domain of toxin A appears to completely neutralize the enterotoxin (25). Similar to what has been observed for toxin B, removal of one or two 20-residue repeats from the LytC choline binding domain only attenuates its function, but does not delete it. Thus, the in vivo function of the CWB-domain and the necessity for various numbers of repeats for the potency of toxins A and B is not entirely clear.

Cell surface binding was highly dependent upon millimolar concentrations of Ca²⁺. The importance of Ca²⁺ for the function of the CWB-domain of toxin A is a novel property associated with toxin A. In some aspects, calcium binding can be important for CWB-domain binding to cell surfaces. In some aspects, calcium binding also can be an important factor influencing the kinetics of full-length toxin A cytotoxicity. This affect appears to be a direct consequence of calcium binding to the CWB-domains themselves. Calcium binding did not induce a noticeable structural change as judged by CD or oligomerization as measured by gel filtration; therefore, the functional role of calcium binding remains undetermined.

The CWB-domain of toxin A does bind choline as has been described for LytA (51). The fact that 2% choline (˜300 mM) is necessary to inhibit the cell wall binding of LytA to pneumococci (52) indicates that its relative affinity for choline may be low, similar to the millimolar affinity determined for the CWB-domain of C. difficile toxin A. Dimerization/oligomerization of the choline binding domain of LytA is functionally important for positioning its amidase domain into the peptidoglycan layer (52, 54). ToxA:1800-2710, ToxB:1807-2366 and ToxA:2459-2710 are all monomeric both in the presence of 10 mM Ca²⁺ and in the presence of 10 mM choline suggesting that calcium or choline induced oligomerization may not be a function of the CWB-domains. Choline binding does suggest that toxin A (and potentially toxin B) is linked to the lipoteichoic acid layer on the surface of C. difficile before secretion or at an early stage of delivery to targeted mammalian cells.

Monoclonal antibodies directed at the CWB-domains of toxin A and B do not appear to be as neutralizing as polyclonal antibody mixtures in toxin neutralization cell assays. Poor kinetic association/dissociation profiles, as observed for rPBA-3, were not necessarily predictive of an antibody's ability to neutralize. Instead, neutralization appeared to depend upon the number and exact location of individual epitopes recognized by an antibody. One toxin A CWB-binding antibody, 251, demonstrated promising kinetic properties (i.e., rapid association and very slow dissociation); however, this antibody behaved similarly to rPBA-3 in the cell assays. Binding of 251 was limited to only one of the three purified mini-domains. The two most effective antibodies, 3358 (or 543) and rPCG-4, recognized numerous epitopes of the toxin A CWB-domain with high affinity. The exact surface recognized by these two antibodies was shown to be overlapping. rPCG-4 is known to bind two epitopes in particular (6). These experiments demonstrate that rPCG-4 binds between 2 and 4 additional epitopes with high affinity, one of which is located within residues 1800-1945. rPCG-4 also has a relatively weak (˜30 nM) affinity for ToxA:2459-2710.

As demonstrated by the data presented and studies discussed herein the combination of monoclonal antibodies which recognize different epitopes proved to be a more effective means of neutralizing both toxin A and toxin B than treatment with monoclonals alone. The 3358 (or 543) and 3359 (or 227) antibodies were demonstrated to have a synergistic effect when used together.

The 3358 (or 543) and 3359 (or 227) antibodies were shown to bind separate epitopes in competition experiments. Interestingly, combination of these two antibodies results in neutralization similar to what can be achieved with the standard TechLab polyclonal antibody. Combination of 3359 (or 227) with rPCG-4 was also highly effective. Combination of 3358 (or 543) with rPCG-4 was effective as well, but to a lesser extent than the 3358 (or 543)/3359 (or 227) or rPCG-4/3359 (or 227) mixtures, perhaps because they compete with one another for binding to toxin A. Similar synergies were uncovered for toxin B binding antibodies (Table 7).

The fact that multiple monoclonal antibodies are more effective at neutralizing toxins A and B suggests that the CWB-domains do not contain one or two specific receptor binding sites for human cells, if indeed the CWB-domains are the primary toxin component responsible for binding. The multiplicity of the repeat domains themselves suggests their effects can be additive and not entirely receptor/protein specific.

The flow cytometry experiments demonstrate different mechanisms of antibody mediated toxin neutralization. Surprisingly, the most neutralizing antibodies, 3358 (or 543) and rPCG-4 do not abrogate the binding of toxin to cell surfaces. Instead, they induce an accumulation of toxin on the cell surface. One plausible mechanism is that the antibody inhibits the internalization of the toxin into endosomal compartments for processing and potential release of the cytotoxic portion of the molecule within the cell. This would imply that the level of toxin binding observed at 1 mM Ca²⁺ concentrations is a steady state amount in equilibrium between cell surface binding, cell surface release and internalization into endosomal compartments. Shedding the antibodies may be necessary before toxin can be internalized. Both 3358 (or 543) and rPCG-4 have very slow (<10⁻⁵/s) kinetic dissociation rates when bound to ToxA:1800-2710. 3358 (or 543) and rPCG-4 have highly overlapping binding sites on the toxin surface according to these competition studies. Therefore, it is not surprising that the two antibodies display a similar apparent mechanism for toxin neutralization. The 3359 (or 227) antibody recognizes a different epitope(s) than 3358 (or 543) and has a partially overlapping epitope with rPCG-4. It neutralizes toxin by abrogating the binding of toxin to cell surfaces. 3358 (or 543)/3359 (or 227) combinations and rPCG-4/3359 (or 227) combinations can be the most effective combinations because they incorporate both neutralization mechanisms and recognize non-overlapping toxin epitopes. With multiple antibodies, these studies demonstrate the ability to provide more superior toxin/epitope coverage and incorporate multiple mechanisms of toxin neutralization for additional synergy.

### REFERENCES FOR EXAMPLE 2

1. Sambrook, J., Fritsch, E. F. and Maniatis, T. In: C. Nolan, Editor, Molecular Cloning: Preparation and Transformation of Competent E. coli Molecular Cloning: Preparation and Transformation of Competent E. coli vol. 1.74, Cold Spring Harbor Laboratory Press, Cold Spring Harbor, N.Y. (1989).
2. Pace, C. N., Vajdos, F., Fee, L., Grimsley, G. and Gray, T. (1995) How to measure the molar extinction coefficient of a protein. Protein Sci. 4, 2411-2423.
3. Myers, J. K., Pace, C. N. and Scholtz, J. M. (1995) Denaturant m values and heat capacity changes: relation to changes in accessible surface area of protein unfolding. Protein Sci. 4, 2138-2148.
4. Brandts, J. F. and Lin, L.-N. (1990) Study of strong to ultratight protein interactions using differential scanning calorimetry. Biochemistry, 29, 6927-6940.]
5. Fernandez-Tornero, C., Lopez, R., Garcia, E., Gimenez-Gallego, G. and Romero, A. (2001) A novel solenoid fold in the cell wall anchoring domain of the pneumonococcal virulence factor LytA. Nature Struct. Biol. 8, 1020-1024.
6. Frey, S. M. and Wilkins, T. D. (1992) Localization of two epitopes recognized by monoclonal antibody PCG-4 on Clostridium difficile toxin A. Infect. Immun. 60, 2488-92.
7. Lylerly, D. M., Krivan, H. C. and Wilkins, T. D. (1988) Clostridium Difficile: Its Disease and Toxins. Clin. Microbiol. Rev. 1, 1-18.
9. Hofmann, F., Busch, C., Prepens, U., Just, I. And Aktories, K. (1997) Localization of the glucosyltransferase activity of Clostridium difficile toxin B to the N-terminal part of the holotoxin. J. Biol. Chem. 272, 11074-11078.
10. Demarest, S. J., Rogers, J. and Hansen, G. (2004) Optimization of the antibody CH3 domain by residue frequency analysis of IgG sequences. J. Mol. Biol. 335, 41-48.
11. Ciesla, W. P. and Bobak, D. A. (1998) Clostridium difficile toxins A and B are cation-dependent UDP-glucose hydrolases with differing catalytic activities. J. Biol. Chem. 273, 16021-16026.
12. Bychkova, V. E., Pain, R. H. and Ptitsyn, O. B. (1988) The ‘molten globule’ state is involved in the translocation of proteins across membranes? FEBS Lett. 238, 231-234.
13. Ren, J., Kachel, K., Kim, H., Malenbaum, S. E., Collier, R. J. and London, E. (1999) Interaction of diphtheria toxin T domain with molten globule-like proteins and its implications for translocation. Science, 284, 955-957.
14. Cho, W. (2001) Membrane targeting by C1 and C2 domains. J. Biol. Chem. 276, 32407-32410.
15. Dodd, R. B. and Drickamer, K. (2001) Lectin-like proteins in model organisms: implications for evolution of carbohydrate-binding activity. Glycobiol. 11, 71R-79R.
16. Caspar, M., Florin, I. and Thelestam, M. (1987) Calcium and calmodulin in cellular intoxication with Clostridium difficile toxin B. J. Cell Physiol. 132, 168-172.
17. Fiorentini, C., Donelli, G., Nicotera, P. and Thelestam, M. (1993) Clostridium difficile toxin A elicits Ca²⁺-independent cytotoxic effects in cultured normal rat intestinal crypt cells. Infect. Immun. 61, 3988-3993.
18. Kretsinger, R. H. (1976) Calcium-binding proteins. Anl. Rev. Biochem. 45, 239-266.
19. Wren, B. W. (1991) A family of clostridial and streptococcal ligand-binding proteins with conserved C-terminal repeat sequences. Mol. Microbiol. 5, 797-803.
20. Sánchez-Beato, A. R., Ronda, C. and García, J. L. (1995) Tracking the evolution of the bacterial choline-binding domain: molecular characterization of the Clostridium acetobutylicum NCIB 8052 cspA gene. J. Bacteriol. 177, 1098-1103.
21. Stubbe, H., Berdoz, J., Kraehenbuhl, J.-P., Corthésy, B. (2000) Polymeric IgA is superior to monomeric IgA and IgG carrying the same variable domain in preventing Clostridium difficile toxin A damaging of T84 monolayers. J. Immunol. 164, 1952-1960.
22. Krivan, H. C., Clark, G. F., Smith, D. F. and Wilkins, T. D. (1986) Cell surface binding site for Clostridium difficile enterotoxin: evidence for a glycoconjugate containing the sequence Galα1-3Galβ1-4GlcNAc. Infect. Immun. 53, 573-581.
24. Kink, J. A. and Williams, J. A. (1998) Antibodies to recombinant Clostridium difficile toxins A and B are an effective treatment and prevent relapse of C. difficile-associated disease in a hamster model of infection. Infect. Immun. 66, 2018-2025.
25. Frisch, C., Gerhard, R., Aktories, K., Hofmann, F. and Just, I. (2003) The complete receptor-binding domain of Clostridium difficile toxin A is required for endocytosis. Biochem. Biophys. Res. Comm. 300, 706-711.
26. Garcia, J. L., Diaz, E., Romero, A. and Garcia, P. (1994) Carboxy-terminal deletion analysis of the major pneumonococcal autolysin. J. Bacteriol. 176, 4066-4072.
27. Lyerly, D. M., Phelps, C. J., Toth, J. and Wilkins, T. D. (1986) Characterization of toxins A and B of Clostridium difficile with monoclonal antibodies. Infect. Immun. 54, 70-76.
28. Lyerly, D. M., Phelps, C. J. and Wilkins, T. D. (1985) Monoclonal and specific polyclonal antibodies for immunoassay of Clostridium difficile toxin A. J. Clin. Microbiol. 21, 12-14.
29. Lima, A. A. M., Lyerly, D. M., Wilkins, T. D., Innes, D. J. and Guerrant, R. L. (1988) Effects of Clostridium difficile toxins A and B in rabbit small and large intestine in vivo and on cultured cells. Infect. Immun. 56, 582-588.
30. Mathieu, R., Lim, J., Simpson, P., Prasannan, S., Fairweather, N. and Matthews, S. (2003) Letter to the editor: resonance assignment and topology of a clostridial repetitive oligopeptide (CROP) region of toxin A from Clostridium difficile. J. Biomol. NMR, 25, 83-84.
32. Pfiefer, G., Schirmer, J., Leemhuis, J., Busch, C., Meyer, D. K., Aktories, K. and Barth, H. (2003) Cellular uptake of Clostridium difficile toxin B. J. Biol. Chem. 278, 44535-44541.
33. von Eichel-Streiber, C. and Sauerborn, M. (1990) Clostridium difficile toxin A carries a C-terminal repetitive structure homologous to the carbohydrate binding region of streptococcal glycosyltransferases. Gene, 96, 107-113.
34. Sauerborn, M., Leukel, P. and von Eichel-Streiber, C. (1997) The C-terminal ligand-binding domain of Clostridium difficile toxin A (TcdA) abrogates TcdA-specific binding to cells and prevents mouse lethality. FEMS Microbiol. Lett. 155, 45-54.
35. Castagliuolo, I., LaMont, J. T., Qiu, B., Nikulasson, S. T. and Pothoulakis, C. (1996) A receptor decoy inhibits the enterotoxic effects of Clostridium difficile toxin A in rat ileum. Gastroenterol. 111, 433-438.
36. Just, I., Hofmann, F., Genth, H. and Gerhard, R. (1995) Bacterial protein toxins inhibiting low-molecular-mass GTP-binding proteins. Int. J. Med. Microbiol. 291, 243-250.
37. Donelli, G. and Fiorentini, C. (1994) Bacterial protein toxins acting on the cell cytoskeleton. New Microbiol. 17, 345-362.
38. Aktories, K. and Just, I. (1995) Monoglucosylation of low-molecular-mass GTP-binding Rho proteins by clostridial cytotoxin. Trends Cell. Biol. 5, 441-443.
39. Pfam Web Page (2001), Washington University, St. Louis, Mo.
41. García, E., García, J. L., García, P., Arrarás, A., Sánchez-Puelles, López, R. (1988) Molecular evolution of lytic enzymes of Streptococcus pneumoniae and its bacteriophages. Proc. Natl. Acad. Sci. USA, 85, 914-918.
42. Ferretti, J. J., Gilpin, M. and Russell, R. R. B. (1987) Nucleotide sequence of a glucosyltransferase gene from Streptococcus sobrinus Mfe28. J. Bacteriol. 169, 4271-4278.
43. Fischer, W. In Streptococcus pneumoniae (ed. Tomasz, A.) 155-177 (Mary Ann Liebert, Inc., Larchmont; 2000).
44. Kyne, L., Farrell, R. J. and Kelly, C. P. (2001) Clostridium difficile. In Infectious Diarrhea. Gastroenterol. Clin. North Am. 30, 753-777.
45. Pothoulakis, C. and LaMont, J. T. (2001) Microbes and microbial toxins: paradigms for microbial mucosal interactions. II. The integrated response of the intestine to Clostridium difficile toxins. Am. J. Physiol. Gastrointest. Liver Physiol. 280, G178-G183.
46. (1995) Recommendations for preventing the spread of vancomycin resistance: Recommendations of the Hospital Infection Control Practices Advisory Committee (HICPAC). Am. J. Infect. Control, 23, 87-94.
47. Kreutzer, E. W. and Milligan, F. D. (1978) Treatment of antibiotic-associated pseudomembranous colitis with cholestyramine resins. John Hopkins Med. J. 143, 67-72.
48. Surawicz, C. M., Elmer, G. W., Speelman, P., McFarland, L. V., Chinn, J. and van Belle, G. (1989) Prevention of antibiotic-associated diarrhea by Saccharomyces boulardii: a prospective study. Gastroenterology, 96, 981-988.
49. Chia, J. K., Chan, S. M. and Goldstein, H. (1995) Baker's yeast as adjunctive therapy for relapses of Clostridium difficile diarrhea. Clin. Infect. Dis. 20, 1581.
50. Gorbach, S. L., Chang, T. W. and Goldin, B. (1987) Successful treatment of relapsing Clostridium difficile colitis with Lactobacillus GG. Lancet. 2, 1519.
51. Briese, T. and Hakenbeck, R. (1985) Interaction of the pneumonococcal amidase with lipoteichoic acid and choline. Eur. J. Biochem. 146, 417-427.
52. Höltje, J. V. and Tomasz, A. (1975) Specific recognition of choline residues in the cell wall teichoic acid by the N-acetylmuramyl-L-alanine amidase of pneumonococcus. J. Biol. Chem. 250, 6072-6076.
54. Fernández-Tornero, C., García, E., Lopez, R., Giménez-Gallego, G., Romero, A. (2002) Two new crystal forms of the choline-binding domain of the major pneumonococcal autolysin: insights into the dynamics of the active homodimer. J. Mol. Biol. 321, 163-173.
55. Barth, H., Pfeifer, G., Hofmann, F., Maier, E., Benz, R. and Aktories, K. (2001) Low pH-induced formation of ion channels by Clostridium difficile toxin B in target cells. J. Biol. Chem. 276, 10670-10676.
56. Brazier, J. S., Fawley, W., Freeman, J. and Wilcox, M. H. (2001) Reduced susceptibility of Clostridium difficile to metronidazole. Antimicrob. Chemother. 48, 741-742.
57. Casimiro, D. R., Toy-Palmer, A., R. C. Blake and Dyson, H. J. (1995) Gene synthesis, high-level expression, and mutagenesis of Thiobacillus ferrooxidans rusticyanin: His 85 is a ligand to the blue copper center. Biochemistry, 34, 6640-6648.
58. Coloma, M. J., Hastings, A., Wims, L. A. and Morrison, S. L. (1992) Novel vectors for the expression of antibody molecules using variable regions generated by polymerase chain reaction. J. Immunol. Methods, 152, 89-104.
59. Dattamajumdar, A. K., Jacobson, D. P., Hood, L. E. and Osman, G. E. (1996) Rapid cloning of any rearranged mouse immunoglobulin variable genes. Immunogenetics, 43, 141-151.

### Example 3

### Engineering Antibodies for Resistance in Simulated Intestinal Fluid

This example describes exemplary methods of the invention for engineering antibodies that are effective when administered orally.

Materials and Methods: Simulated intestinal fluid (SIF) was prepared fresh daily as described in the United States Pharmacopoeia. 1×SIF buffer consisted of 10 mg/mL pancreatin, (Sigma Chemical Co., St. Louis, Mo.), and 6.8 mg/ml KH₂PO₄. A master tube was prepared in a 1.5 mL microcentrifuge tube containing 18.6 uL of sample, 70 uL of 10×SIF (10×SIF was centrifuged before use) in a final volume of 770 uL. The reaction was incubated at 37° C. At intervals of 0, 2, 10, 30, 60, 120, and 240 min, aliquots of 110 μL were removed from the master tube and 5.5 uL of PEFABLOC™ (Roche) was added immediately to halt further digestion. Antibodies were expressed in 12-well plates. The overall expression level ranged between 0.5-4 μg/mL. Expression varied from plate to plate, but an internal wildtype control was transfected within each plate to insure that expression level did not affect the digestion results. In general, expression was quite uniform within each plate with a standard deviation of ±26.1% of the average expression within each plate. The amount of antibody remaining after digestion was determined by quantitative ELISA. Some samples from the first round of digestion were also subjected to SDS-PAGE analysis using precast 4-12% Bis-Tris NUPAGE™ gels (Invitrogen, Carlsbad, Calif.) and Silver staining (SILVERQUEST™ Kit, Invitrogen). Results of the SDS-PAGE analysis correlated well with ELISA results; therefore, ELISA was used for the remaining antibody samples as it provided a more accurate quantitation of the digestion results.

ELISA detection of remaining IgG after pancreatin digestion: Microtiter Streptavidin plates (Sigma Chemical, St. Louis, Mo., catalog #M5432) were coated with 200 ng per well biotinylated protein G in PBS buffer and incubated at 4° C. overnight. The plates were then washed 3 times with Tris buffered saline, pH 8.0 with Tween-20 (TBST—Sigma, cat#T9039). Aliquots of 100 μL of each antibody sample (diluted into TBST) were transferred to the protein G-coated plates and incubated for 1-2 hours at room temperature. Following 3 washes with TBST, alkaline phosphatase-conjugated goat anti-human Fab (Pierce, 31312) was added to each well at a 1:1000 dilution. The reaction was carried out for 1 hr at room temperature, the plate(s) was washed 3 times with TBST and 100 μL of p-nitrophenylphosphate substrate was added (Sigma, Catalog # A3469). The absorption was determined at 405 nm using a Molecular Devices vₘₐₓ kinetic microplate reader.

Various antibody classes were tested in simulated intestinal fluids. All antibody classes, IgG1, IgG2, IgG3 and IgG4 were proteolyzed by pancreatin (see also, FIG. 1). Interestingly, the pattern of degradation appeared similar at 0 and 30 min.

Determination of pancreatin cleavage sites to mutate: Trypsin and chymotrypsin are the most abundant enzymes present in pancreatin. Potential pancreatin cleavage motifs in the sequence of human IgG were determined based on known trypsin and chymotrypsin cleavage rules (Table 8). Trypsin specifically recognizes Arg and Lys residues at the site where it cleaves peptide bonds. Arg and Lys residues with greater than 40% solvent exposure were identified as potential candidates for directed mutagenesis in the heavy chain and light chain constant domains. Chymotrypsin specifically recognizes Phe, Tyr or Trp. Therefore, Phe, Tyr and Trp residues with greater than 25% solvent exposure were identified as potentially candidates for directed mutagenesis in the heavy chain and light chain constant domains. The selection of residues to replace the potential cleavage sites was based on information from an “unbiased” database of IgG Fc sequences. Mutations were made to the next most frequently observed residue within the dataset of IgG sequences.

Table 8 shows a list of chymotrypsin and trypsin putative cleavage sites in the constant domain region of the light and heavy chains. The position of the cleavage sites for chymotrypsin (CT) and trypsin (T) and the residue surface exposure estimated on crystal structure are listed below. MFR: most frequent residue; SMFR: second most frequent residue; Antibody region: CL: light chain constant domain; CH: heavy chain constant domain; X ray #; residue position based on crystal structure; Replacement: residue selected to replace the cleavage site.

TABLE 8

| | | | Residue | | | | | | |
|---|---|---|---|---|---|---|---|---|---|
| | | | number | | Kabat/ | | | | |
| Antibody | | Residue | for Clone | | EU | % Residue | | | |
| Region | Protease | Type | 2934 | X ray# | Numbering | Exposed | MFR | SMFR | Replacement |
| | T | K | | 107 | | 31 | | | |
| | T | R | | 108 | | 27 | | | |
| | CT | F | 143 | 116 | F116S | 27 | F54% | S40% | S |
| | T | K | 153 | 126 | K126A | 47 | A35% | K30% | A |
| | CT | F | | 139 | | 0 | | | |
| | T | R | 169 | 142 | R143S | 40 | R46% | K42%, | S |
| | | | | | | | | S5% | |
| | T | K | | 145 | | 36 | | | |
| C_{L} | CT | W | | 148 | | 1 | | | |
| | T | K | | 149 | | 20 | | | |
| | T | K | 196 | 169 | K169G | 54 | K93% | G3% | G |
| | CT | Y | | 173 | | 6 | | | |
| | T | K | | 183 | | 24 | | | |
| | CT | Y | | 186 | | 2 | | | |
| | T | K | 210 | 188 | K183S | 43 | S50% | R20% | S |
| | T | K | | 190 | | 23 | | | |
| | CT | Y | | 192 | | 0 | | | |
| | T | K | | 207 | | 25 | | | |
| | CT | F | | 209 | | 7 | | | |
| | T | R | | 211 | | 26 | | | |
| | T | K | | 124 | | 33 | | | |
| | T | K | 155 | 136 | K133G | 62 | G49% | R26% | G |
| | T | K | | 150 | | 21 | | | |
| | CT | Y | | 152 | | 0 | | | |
| | CT | W | | 161 | | 1 | | | |
| | CT | F | | 183 | | 16 | | | |
| | CT | Y | | 201 | | 4 | | | |
| | T | K | 227 | 208 | K205P | 42 | P69% | K15% | P |
| | T | K | 232 | 213 | K210T | 48 | K90% | R5%, T2% | T |
| | T | K | | 216 | | 26 | | | |
| C_{H} | T | R | | 217 | | 24 | | | |
| | CT | F | 263 | 241 | | 27 | | L2% | |
| | T | K | | 248 | | 18 | | | |
| | T | R | | 255 | | 29 | | | |
| | T | K | 296 | 274 | K274Q | 45 | Q62% | K19% | Q |
| | CT | F | | 275 | | 13 | | | |
| | CT | W | | 277 | | 0 | | | |
| | CT | Y | | 278 | | 9 | | | |
| | T | K | | 288 | | 31 | | | |
| | T | K | 314 | 292 | K326N | 53 | R70% | K20% | N |
| | | | | | | | | N1% | |
| | CT | Y | 318 | 296 | | 27 | S1% | F0.67%, | |
| | | | | | | | | Y0.17% | |
| | CT | Y | | 300 | | 9 | | | |
| | T | R | | 301 | | 21 | | | |
| | CT | W | | 313 | | 7 | | | |
| | T | K | | 317 | | 20 | | | |
| | CT | Y | | 319 | | 1 | | | |
| | T | K | | 320 | | 19 | | | |
| | T | K | | 322 | | 17 | | | |
| C_{H} | T | K | 348 | 326 | K340A | 56 | E10%, | A3%, | A |
| | | | | | | | | V1% | |
| | T | K | | 334 | | 19 | | | |
| | T | K | | 338 | | 9 | | | |
| | T | K | 362 | 340 | | 54 | I1% | K0.69%, | |
| | | | | | | | | R0.12% | |
| | T | R | | 344 | | 31 | | | |
| | CT | Y | 371 | 349 | | 25 | Y100% | | |
| | T | R | 377 | 355 | R355P | 66 | R60% | P10% | P |
| | | | | | | | | Q9% | |
| | T | K | 382 | 360 | K360Q | 42 | K 67% | Q6% | Q |
| | T | K | | 370 | | 27 | | | |
| | CT | F | | 372 | | 0 | | | |
| | CT | W | | 381 | | 1 | | | |
| | CT | Y | | 391 | | 18 | | | |
| | T | K | 414 | 392 | K392A | 47 | K30% | R24% | A |
| | | | | | | | | A7% | |
| | CT | F | | 404 | | 10 | | | |
| | CT | F | | 405 | | 19 | | | |
| | CT | Y | 429 | 407 | | 26 | Y100% | | |
| | T | K | | 409 | | 32 | | | |
| | T | K | | 414 | | 17 | | | |
| | T | R | | 416 | | 20 | | | |
| | CT | W | | 417 | | 2 | | | |
| | CT | F | | 423 | | 0 | | | |
| | CT | Y | | 436 | | 23 | | | |
| | T | K | | 439 | | 28 | | | |

Screening of single mutants for resistance to pepsin: Expression and thermotolerance screening was performed for every member of the library to determine whether mutation at each chymotrypsin and trypsin-labile position was tolerated. For thermotolerance, supernatants with recombinant antibody were heat-challenged for 10 minutes at 70, 75 and 80° C. (Antibodies can denature irreversibly with heat.) The amount of antibody remaining in the supernatant subsequent to thermal challenge was detected by ELISA and compared to ELISA data obtained with the wildtype protein. Most mutants demonstrate comparable thermotolerance and/or expression compared to the wildtype antibody. Interestingly, even single mutations can confer some degree of resistance to pancreatin digestion.

Up-mutants containing multiple trypsin and chymotrypsin resistance sites were also tested for resistance to pancreatin. All up-mutants expressed comparably to the wildtype gene in mammalian cells and demonstrated similar thermotolerance profiles. Results are reported in Table 9. Several combinations of mutations were identified to confer resistance to pancreatin digestion.

Table 9 shows ELISA results after pancreatin digestion of the wildtype and the mutated antibody molecules. The parent antibody molecule (2934) as well as the mutants were expressed in mammalian cells, purified, and dialyzed. Antibody mutants were digested with pancreatin at 37° C. for the time indicated. ELISA assays were performed to measure the amount of remaining antibody. Mutations are listed below. A score was given to each variant to describe its expression (Ex): +: Expression was greater than wildtype; : Equivalent expression compared to wildtype; −: Less material was expressed than the wildtype; −: No expression. Each antibody variant was given a thermotolerance score (T) according to the following criteria: +: A greater percentage of folded protein remaining at 75° C. and/or 80° C. compared to wildtype; : Equivalent percentage of folded protein remaining at each temperature point compared to wildtype; −: A lesser percentage of folded protein remaining at 75° C. than wildtype; −: Thermal unfolding observed at 70° C.

TABLE 9

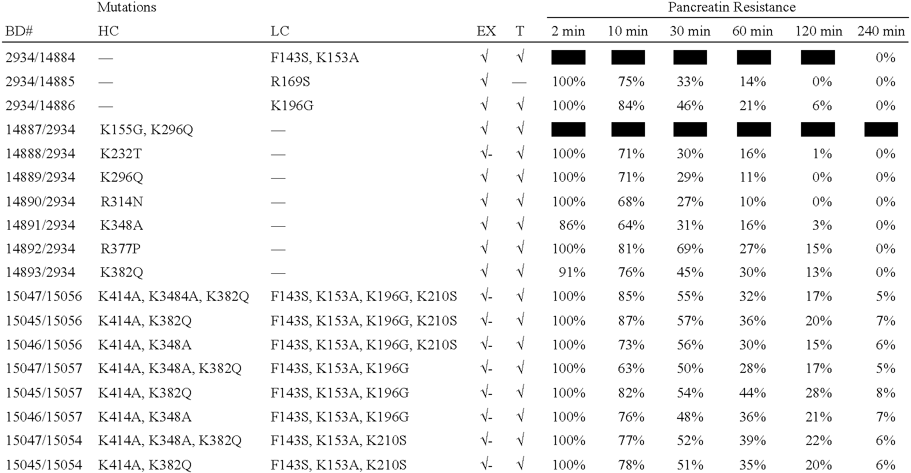

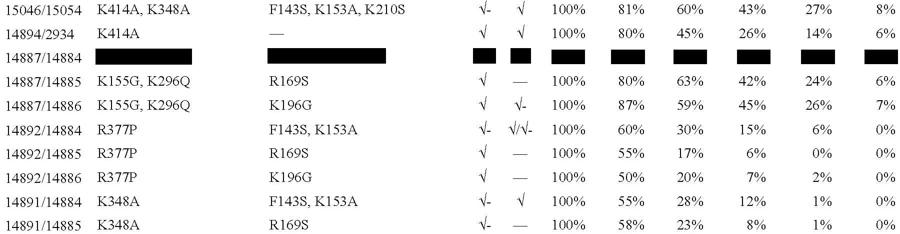

These results demonstrated the successful targeting of chymotrypsin and trypsin cleavage sites within the IgG1 Ab framework allowed the molecule to survive for longer durations in simulated intestinal fluids. Accordingly, these results demonstrate that the methods of the invention are effective in engineering antibodies that are more effective for use when orally administered because of their ability to survive longer in intestinal fluids.

### Example 4

### Anti-Clostridium difficile Antibodies in the Ileal Loop Model

This example provides studies that demonstrate that anti-toxin A antibodies of the invention are effective in neutralizing the activity of native C. difficile toxin A.

Neutralizing antibodies identified in the toxin neutralization cell assays were tested in the ileal loop model. An ileal loop model was developed to assess the ability of the anti-toxin A antibody to neutralize the activity of native C. difficile toxin A. See FIG. 13.

Experiments were conducted as described in the protocol approved by IAACAC. Briefly, 5 μg of native toxin A was mixed with various amount of test antibody in a final volume of 400 μl and then injected in the ligatured ileal loop. Animals were fasted overnight and were given water ad libitum. Ligated rat small intestinal segments (5 cm) were injected with 400 μl of toxin A with or without test article, with cholestyramine, or control material (saline or test article alone). 4 h after injection, weight and length of ileal loops was measured. A polyclonal antibody against toxin A shown to work 100% in toxin neutralization cell assays was also tested. Male rats were used in all studies. 2 loops were injected per rat. Groups of 2 or 3 rats were used per test article. The ability of the monoclonal antibodies to synergistically neutralize the effects of C. difficile toxin A are shown in FIGS. 14-16.

FIGS. 15A to F illustrate the histology of rat intestinal mucosa. Cross-section of rat ileal loop after the addition of (A) saline; (B) 5 μg toxin A; (C) 5 μg toxin A and 1 mg mouse isotype control antibody; (D) 5 μg toxin A and 1 mg 3359 (or 227) antibody; (E) 5 μg toxin A and 1 mg 543 antibody; and (F) 5 μg toxin A and 1 mg TechLab antibody.

FIG. 16 shows weight versus length measurement for rat ileal loops incubated with saline, 5 μg toxin A independently and in the presence of various concentrations of 3359 (or 227) and 543 antibodies (alone and in combination).

### Example 5

### Neutralization of Clostridium difficile Toxin A Using a Combination of Monoclonal Antibodies with Unique Modes of Action

This example provides studies that demonstrate neutralization of Clostridium difficile Toxin A using a combination of monoclonal antibodies with different binding specificities.

The pathogenicity of Clostridium difficile (C. difficile) is mediated by the release of two related toxins, A and B. It is believed that toxin binding to mammalian epithelial cell surfaces is mediated by the C-terminal region of both toxins. Their C-termini comprise a large cluster of repeats known as cell wall binding (CWB) domains. In these studies, monoclonal murine antibodies were raised against the CWB-domain of toxin A and screened for their ability to neutralize the full-length toxin both individually and in combination. All monoclonal antibodies capable of neutralizing toxin A recognized multiple sites on the CWB-repeats as judged by surface plasmon resonance experiments.

Flow cytometry results revealed differing neutralization mechanisms for two antibodies of the invention: designated 3358 (or 543, see above) and 3359 (or 227, see above), which recognize unique, non-overlapping epitopes of the toxin A CWB-domain. While in some aspects the invention is not limited by any particular mechanism of action, the 3358 antibody appeared to impede the internalization and/or promote the reorganization of the toxin at the cell surface of mammalian cells. The second neutralizing antibody, 3359, was found to inhibit toxin cell surface association. Interestingly, a mixture of these two antibodies with distinct mechanisms of toxin A neutralization led to highly increased (synergistic) toxin neutralization in an in vitro toxin A neutralization assay. The level of protection against toxin A provided by the antibody combination was similar to the level observed with a positive polyclonal antibody control and significantly greater than any single monoclonal antibody tested in isolation.

Increased efficacy using this antibody combination of the invention (3358 and 3359) was also observed in a rat ileal loop model. Full protection in a hamster infection model required an additional antibody raised against the toxin B CWB-domains. Overall, these results demonstrate that antibody combinations which utilize multiple mechanisms of neutralization and provide a broader epitope coverage than is possible with single monoclonals may lead to enhanced protection against C. difficile associated diarrhea.

Thus, the invention provides and these studies demonstrate the efficacy of a “synthetic polyclonal” cocktail of two or more neutralizing monoclonal antibodies which target non-overlapping epitopes of a bacterial toxin, e.g., C. difficile toxin A. C. difficile toxin A is more enterotoxigenic than C. difficile toxin B and plays a more dominant role in triggering antibiotic associated diarrhea. Mouse monoclonal antibodies against the CWB-domains of toxin A were tested for their ability to neutralize toxin A individually and in combination with one another. In parallel, they were characterized thoroughly in competitive binding studies. Two antibodies denoted 3358 and 3359 (i) bound to non-overlapping epitopes of the CWB-domain of toxin A, (ii) demonstrated alternate mechanisms of toxin A neutralization and (iii) combined favorably to neutralize toxin A in a neutralization assay. These findings demonstrate that treating C. difficile associated disease using combinations of monoclonal antibodies as provided for in the compositions and methods of this invention provide superior efficacy and dosing compared to single monoclonal antibody therapy.

### Methods:

Production of Recombinant Anti-Toxin A rPBA-3, 3358, 3359 and rPCG-4 antibodies: Anti-C. difficile toxin A hybridoma cell line PBA-3 (ATCC# HB-8713) was purchased from the ATCC. The cell line was grown in DMEM (Dulbecco's Minimal Essential Medium with high glucose (Gibco/Invitrogen, Carlsbad, Calif.), 10% FBS (Sterile Fetal Bovine Serum, Sigma Chemical, St. Louis, Mo.), and 1× glutamine, penicillin and streptomycin (Gibco/Invitrogen, Carlsbad, Calif.) and cryopreserved. Total RNA was isolated from 10⁷ hybridoma cells using a procedure based on the RNeasy Mini kit (Qiagen, Hilden Germany). Primers used for the amplification of the variable region from both the light chain and the heavy chains were designed as described previously (7,9). Primers MLALT5 and 33615 were used for amplification of the variable region from the light chain (MLALT5: 5′-CACCATGAAGTTGCCTGTTAGGCTGTG-3′ (SEQ ID NO:10); 33615: 5′-GAAGATCTAGACTTACTATGCAGCATCAGC-3′ (SEQ ID NO:11)). Primers MVG1R and MH1 were used for the amplification of the heavy chain variable region (MH1: 5′-ATATCCACCATGGRATGSAGCTGKGTMATSCTCTT-3′ (SEQ ID NO:12); MVG1R: 5′-GGCAGCACTAGTAGGGGCCAGTGGATA-3′ (SEQ ID NO:13)). The PCR products were cloned into the modified mammalian expression vector pCEP4 (Invitrogen, Carlsbad, Calif.). The modified vector either contained the signal peptide and the constant domain region of the heavy chain or the signal peptide and the constant domain of the light chain. The constant domain of the human IgG1 was constructed by subcloning the appropriate heavy chain and light chain domains into pCEP4 from a human spleen cDNA library.

The variable heavy and variable light chain domains of PCG-4 (Lyerly (1986) Infect. Immun. 54:70-76) were created synthetically based on the sequences for the light and heavy chains obtained from Genbank (accession numbers X82691 and X82692). The variable domain of both the heavy and the light chains were individually derived from synthetic oligonucleotides by overlap extension PCR (4) and cloned for periplasmic export in vector pBK-CMV. The variable domains were also subcloned from the Fab plasmid constructs into the same pCEP4 vector system described for rPBA-3 in order to produce a chimeric full-length IgG construct.

Generation and screening of anti-Toxin A and B monoclonal antibodies: A total of 15 mice (5 BALB/c and 10 Swiss-Webster) were immunized by 4 injections of 25 μg every 21 days with ToxA:40R at Strategic BioSolutions (Newark, Del.). C. difficile Toxin CWB-domains, ToxA:40R, ToxA:6R, ToxA:7R, and ToxA:11R were cloned, expressed in E. coli and purified as described previously (10). Removal of the hexa-histidine tags was performed by thrombin cleavage and dialysis overnight in MWCO 10000™ dialysis tubing prior to injection. After 12 weeks, all mice had developed anti-toxin A antibody titers. Sera of the third bleed were tested in toxin neutralization cell assays and by surface plasmon resonance to rank the mice. Fusions were initiated with spleen cells of mice demonstrating a high anti-toxin A titer and toxin A neutralization in cell assays. A total of 1920 cell lines were plated from the fusion (20 plates×96 wells). Out of 13 lines that were ELISA positive, 10 lines showed a strong ELISA titer. The supernatants of these 10 lines were further tested in toxin neutralization assays. Similarly, anti-toxin B antibodies were raised with ToxB:25R (residues 1807-2366) (8).

Hybridoma antibody production: Hybridoma cell lines were cultured as described above. For maximal antibody production, cells were allowed to reach the plateau phase and were incubated for an additional 3 or 4 days. Cell-suspensions were spun down and the supernatants were collected, filtered, given protease inhibitors and stored at 4° C. until purified.

Transfection of rPBA-3 and rPCG-4 into 293F mammalian cell expression host: The heavy and light chain plasmids of both rPBA-3 and rPCG-4 were transformed into XL1-blue bacteria. Large scale plasmid DNA was prepared as described by the manufacturer (Qiagen, endotoxin-free MaxiPrep kit Cat#12362). Plasmids were transfected into the adenovirus-transformed human embryonic kidney cell line 293F using 293fectin and the 293F-FREESTYLE™ Media for culture. Light and heavy chain plasmids were both transfected at 0.5 μg/mL with cell density of 10⁶ cells/mL. Supernatants were collected by centrifugation at 1200 rpm for 8 minutes at 25° C. 7 days after transfection. Expression levels varied from ˜0.25-1.5 μg/mL.

Antibody Purification/Quantification: PBA-3, 3358, 3359 and 3356 mouse monoclonal antibodies and rPBA-3 and rPCG-4 chimeric antibodies were purified by passing culture supernatants over protein G columns (Amersham, cat#17-0405-01) at 4 mL/min. Mobile phases consisted of 1×PBS-Tween (Sigma Aldrich, Running Buffer, cat# P-3563) and 0.1 M glycine pH 2.7 (Fisher Chemicals, Elution Buffer, cat# G48-500). Antibody collections in 0.1 M glycine were diluted 20% (v/v) with 1 M TrisHCl, pH 8.0, to neutralization the pH. IgG1 collections were pooled and dialyzed exhaustively against 1×PBS (Pierce Slide-A-Lyzer Cassette, 3500 MWCO, cat#66110). The concentration of each IgG1 stock solution was determined by Bradford analysis (Bio-Rad Protein Assay, Hercules, Calif. cat#500-0006) using a commercial myeloma IgG1 stock solution as a standard and by UV (280 nm) absorbance.

ELISA testing for anti-toxin A antibodies: ToxA:40R was biotinylated using the EZ-LINK-Biotin-LC-ASA™ kit (PIERCE catalog #29982). Briefly, EZ-LINK-Biotin-LC-ASA™ was dissolved in DMSO and added to toxin A at a 4:1 molar ratio. Protein/biotin conjugation was induced for 20 minutes under a UV lamp in a PBS buffer. Conjugated toxins were removed from unreacted biotin by application of the reaction mixture to a desalting column (Pierce D-Salt Dextran Plastic Desalting Columns, catalog #43230). 500 μL fractions from the desalting procedure were tested for protein absorption at 280 nm to detect the presence of biotinylated toxins.

Microtiter Streptavidin plates (Sigma Chemical, St. Louis, Mo., catalog #M33582) were coated with 200 ng per well of biotinylated ToxA:40R diluted into PBS buffer and incubated at 4° C. overnight. The plates were then washed 3 times with TBST buffer. All samples were diluted in Tris buffer, pH 8.0 TBST buffer (Sigma, cat#T9039). Aliquots of 100 μL of each diluted serum sample or fusion supernatant were transferred to the toxin-coated plates and incubated for 1-2 hours at room temperature. Following 3 washes with TBST, Alkaline phosphatase-conjugated rabbit anti-mouse IgG(H+L) (Zymed, cat#61-6522) was added to each well at a 1:1000 dilution. The reaction was carried out for 1 hr at room temperature, plates were washed 3 times with TBST and 100 μL of p-nitrophenylphosphate substrate was added (Sigma, Catalog # A3469). The absorption was determined at 405 nm using a Molecular Devices vₘₐₓ kinetic microplate reader.

Determination of the Epitopes and Relative Affinities of the anti-Toxin Antibodies: All Surface Plasmon Resonance (SPR) experiments were preformed on a BIACORE3000™ instrument set to 31° C. Toxin CWB-domains were immobilized to research grade CM5 Chip surfaces using the immobilization programs within the BIACORE3000™ Software. Carboxymethyl-moieties on the surface of each flow cell were activated using standard EDC/NHS chemistry. Toxin A CWB-domains were covalently attached to the chip surfaces by primary amine coupling to the activated surfaces with a 100 mM Acetate buffer, pH 4. Toxin B CWB-domains were sensitive to low pH and immobilized at pH 5.5 immediately following dilution into the glycine buffer. Kinetic analysis of each anti-toxin A Fab or monoclonal antibody was performed by injecting a series of concentrations over each toxin A-coated flow cell. A typical antibody concentration series consisted of 0.5, 2, 6, 20 and 100 nM injections. Chip surfaces regenerated reliably with a 10 μL injection of 0.1 M glycine, pH 1.5 followed by a 10 μL injection of 50 mM NaOH. The flow rate was 30 μL/min. For all runs, there was a flow cell dependent baseline drift between 0.0002 and 0.001 RU/sec which could be accounted for in the 1:1 fitting model used to analyze the kinetics.

All constructs were described previously (10). The first construct, ToxA:6R encompassed the N-terminal portion of the toxin A CWB-domains (residues 1800-1945); the second construct ToxA:7R was derived from a central region of the repeats (residues 2078-2234) and includes a known rPCG-4 binding site (15); the third construct ToxA:11R contained the C-terminal 11 repeats (residues 2459-2710) and the last construct ToxA:40R contained the entire toxin A CWB-domain (residues 1800-2710).

Antibody Competition Studies: The protocol utilized immobilized antibodies to detect soluble ToxA:40R (comprising the entire toxin A CWB-domain). Monoclonal antibodies 3358, 3359 and rPCG4 were each immobilized to separate CM5 surfaces to a response level at least 6000 RU above the baseline. The concentration of soluble ToxA:40R was 15 nM, at least 10-fold greater than the apparent K_{D} of each monoclonal antibody. In an attempt to saturate ToxA:40R prior to injection over the chip surfaces containing immobilized antibody, ToxA:40R was incubated with 0, 1, 3, 5, 8, 12, 15, 20, 25, 30, 35, 40, 50, 60, 70, 80, 90, 105 and 120 nM concentrations of 3358, 3359, rPCG4 (full-length antibody) and rPCG4 (Fab format). 200 μL injections were preformed at flow rates of 10 μL/min. The linear portion of the binding curves (under mass transfer conditions) was used to measure the percentage of toxin saturated by each antibody. After each injection, the flow rate was increased to 30 μL/min and the antibody surfaces were regenerated with 2×10 μL of 0.1 M glycine, pH 2.0. Regeneration did not degrade the immobilized 3358 and 3359, but resulted in approximately 0.5% signal loss per injection for the rPCG-4 antibody surface. Every sixth injection was performed with 100% free toxin to monitor the maximum signal in response to free ToxA:40R.

Toxin Neutralization Assay: CHO-K1 cells (ATCC CCL-61) were maintained in Dulbecco's modified Eagle's medium (DMEM—Gibco, cat#12430-054) supplemented with 10% fetal bovine serum at 37° C. in a CO₂ incubator. Prior to the experiment, cells were split into T75 flasks at 1.5×10⁶ cells/flask. Cells reached confluency within 2 days. CHO cells were seeded from these stocks into 96-well cell culture plates (Costar, cat#3598) and incubated for 4-6 hours. Toxin A and toxin B were purchased from List Biological Laboratories. Toxin and toxin/antibody mixtures were incubated at 37° C. in DMEM with 10% FBS (Gibco, cat#10082-147) for 1 hour and added to the 96-well plates. Cell rounding was monitored over a 48 hour period. At 48 hours, 10 μL WST-1 (Roche, cat#1644807) was added to each well and the plates were incubated for 1 hour at 37° C. Absorbance at 450 nm was determined using a Molecular Devices Spectramax Plus 96-well plate reader.

Flow Cytometry: CHO cells were cultivated at a ratio of 1:10 and grown in 10 cm dishes. Prior to cell staining, the cells were washed two times with PBS, scraped, and pelleted at 1100 rpm at 4° C. for 5 minutes. Cell staining was performed with 5×10⁵ cells/tube in wash buffer (PBS supplemented with 2.5 mM Hepes, 1 mM CaCl₂, 0.1% sodium azide, and 2% FBS). The cells were resuspended with 100 μL of ToxA:11R in wash buffer. The CaCl₂ and ToxA:11R concentrations were held to 1 mM and 50 μg/mL, respectively, leading to a reproducible shift in the fluorescently labeled cell population between 35-60% (8). Cells were subsequently washed two times with 2 mL wash buffer. ToxA:11R was detected on the surface of cells via its C-terminal hexahistidine tag by subsequent incubation with the Alexafluor 488 conjugated PENTA-HIS monoclonal antibody (Qiagen, cat#35310) for 20 minutes. Cells were washed twice with 2 mL wash buffer and resuspended in 500 μL wash buffer before sorting by flow cytometry. To study antibody neutralization mechanisms, anti-toxin A antibodies were combined with ToxA:11R during the staining step at concentrations of 250 or 500 μg/mL. These concentrations yielded molar ToxA:11R:antibody ratios of 1:1 and 1:2. Flow cytometry analysis was performed on a DAKOCYTOMATION MOFLO™ flow cytometer (Fort Collins, Colo.) equipped with a Coherent Enterprise II (Santa Clara, Calif.) water-cooled argon ion multi-line laser. The 488 nm line was used as the excitation source. Forward scatter (FSC), side scatter (SSC) and fluorescent properties were detected by R928 photomultiplier tubes (Hamamatsu, Shizuoka-ken, Japan). Fluorescence was detected between 510 and 550 nm. Data was collected for 10,000 events and was analyzed using DAKOCYTOMATION SUMMIT v3.1™ software.

Rat ileal loop studies: A rat ileal loop model for C. difficile infection was used to assess antibody efficacy. In this model, the enterotoxic effect of toxin A leads to an increase of the ileal loop weight-to-length ratio. Of note, this model does not allow for the evaluation of anti-toxin B antibodies because rat ileal do not harbor the pertinent receptors for toxin B (29). A midline abdominal incision was performed on Sprague-Dawley rats maintained under anesthesia by inhalation of isoflurane. Two 5-cm-long closed ileal loops were formed in each animal using sutures. Ileal loops were then injected (400 μl) with saline, 5 μg toxin A or 5 μg toxin A preincubated for 5 min with TechLab anti-toxin A polyclonal antibody (Techlab, Blacksburg), monoclonal antibody 3358, 3359 or 3358/3359 antibody mixtures. The abdominal incision was then closed and animals were allowed to recover. After 4 h, animals were sacrificed by an overdose of anesthetic and the loops were removed. Intestinal fluid accumulation was measured as the ratio of loop weight (milligrams) to length (centimeter). Cholestyramine (80 mg) was used as a comparator (SIGMA, C4650) as previously reported (40). Each antibody and antibody mixture was evaluated in a separate study with the appropriate controls. The 3358 and 3359 antibodies were also injected without toxin A at 1 mg/mL to determine if these antibodies had direct effects on fluid accumulation in the rat ileal loop. Treatments were done in duplicate or triplicates within an experiment, and means and standard errors were determined. The study protocol was approved by the Institutional Animal Care and Use Committee.

Hamster efficacy studies: A hamster model for C. difficile infection was used to assess antibody efficacy (23,44). Golden Syrian hamsters, 90 to 110-gram in weight, were obtained from Charles River Laboratories and were housed individually in cages with free access to diet and water. Groups of 5 hamsters were injected intraperitoneally (IP) with 10 mg/kg clindamycin phosphate, 48 h prior to C. difficile challenge. On day 0, the animals received an oral dose with 100 spores of C. difficile (ATCC 43596) from previously prepared and tested frozen spore stock. Antibody solutions were resuspended in PBS buffer (pH 7.4) and were administered once daily IP for 4 consecutive days. The first dose was administered 4 h prior to the clindamycin treatment. Oral vancomycin (50 mg/kg, SIGMA, V8138) once daily for 3 days was used as a comparator. The animals were then observed for morbidity and the presence of diarrhea. In this model, untreated animals develop symptoms within 48-72 h post challenge. The animals judged to be in extremis were euthanized, and their cecal contents and cecal tissues were harvested for further analysis. All animal procedures were approved by the Institutional Animal Care and Use Committee.

### Results

Production and Characterization of Neutralizing Antibodies Against Toxin A: High-titer monoclonal antibodies were generated by immunizing mice against a fragment of toxin A encompassing the full CWB domain. Supernatants from selected hybridoma-cell cultures were individually tested for their ability to bind ToxA:40R in the ELISA format and for their neutralization properties in the toxin A neutralization assay. From this pool, 13 total hybridoma fusions were created whose supernatants exhibited some level of anti-toxin A activity. 10 monoclonals with the highest titers against ToxA:40R were tested alone or in combination with other supernatants in the toxin A neutralization assay. Complete abrogation of the cytotoxic properties of toxin A in this assay using a single antibody was generally not observed. Similar results have been reported for rPCG-4 and other monoclonal antibodies (34).

Of the 10 high titer monoclonals studied in detail, one monoclonal, denoted 3358, was the most neutralizing in the toxin A neutralization assay. A second monoclonal antibody, denoted 3359, was moderately neutralizing on its own, but appeared to pair well in combination with several other monoclonals including 3358 for increased toxin A neutralization. This result was more noticeable when observing CHO cell morphology after treatment with toxin A/antibody mixtures as opposed to the quantitative numbers generated using the WST-1 assay format. Addition of certain monoclonal combinations, including the 3358/3359 combination, led to an overall cell morphology which more closely resembled untreated CHO cells or CHO cells treated with toxin A/polyclonal anti-toxin A. However, no comparative dose of monoclonal antibody or antibody combination was as effective as the polyclonal anti-toxin A control for neutralizing toxin A.

As the 3358 and 3359 monoclonals provided the most interesting toxin A neutralization properties, the two antibodies were further titrated against two toxin A concentrations in the neutralization assay, see Table 1, below, summarizing toxin neutralization assay data. Toxin A neutralization by anti-toxin A antibodies was determined using an in vitro neutralization assay. CHO cells were incubated with two concentrations of toxin A (4.0 and 0.8 μg/mL) which both kill 100% of the cells over a 48 h time period. Monoclonal antibodies from this study as well as the rPCG-4 and rPBA-3 antibodies were co-incubated with toxin A to investigate their ability to neutralize. The positive control was a goat anti-toxin A polyclonal antibody. The negative control was a non-specific mouse IgG monoclonal antibody. The data represent are reported as a percentage of cells surviving in the toxin neutralization assay. The experiments were performed in triplicate.

In parallel, recombinant forms of the publicly available anti-toxin A monoclonal antibodies PCG-4 and PBA-3 were included in the neutralization assay. The polyclonal antibody against native toxin A completely protected CHO cells at both toxin A concentrations. The 3358, 3359, and rPCG-4 antibodies exhibited some level of toxin A neutralization at 4 μg/mL toxin A (Table 1).

TABLE 1

| | 4.0 μg/mL | | 0.8 μg/mL | |
|---|---|---|---|---|
| | Average | | Average | |
| | (%) | +/−SD | (%) | +/−SD |
| Media only | 98.0 | 2.0 | 97 | 3.61 |
| Toxin A (0.4 μg) | 2.7 | 2.1 | 6.7 | 1.53 |
| Control Polyclonal (5 μg) | 77.7 | 2.5 | 92.0 | 2.65 |
| Control Polyclonal (2.5 μg) | 76.0 | 3.6 | 92.3 | 2.08 |
| Control mouse IgG (2.5 μg) | 4.7 | 0.6 | 6.0 | 1.00 |
| 3358 (2 μg) | 37.3 | 2.5 | 63.3 | 7.23 |
| 3358 (1 μg) | 20.0 | 2.0 | 43.0 | 10.44 |
| 3358 (0.5 μg) | 14.7 | 1.5 | 20.0 | 2.00 |
| 3359 (2 μg) | 26.3 | 1.5 | 38.7 | 3.21 |
| 3359 (1 μg) | 16.3 | 1.5 | 26.3 | 1.53 |
| 3359 (0.5 μg) | 14.0 | 1.7 | 17.0 | 2.65 |
| rPCG-4 (2 μg) | 22.3 | 2.5 | 37.0 | 3.00 |
| rPCG-4 (1 μg) | 17.7 | 2.5 | 26.3 | 1.53 |
| rPCG-4 (0.5 μg) | 13.3 | 1.5 | 17.7 | 2.52 |
| PBA3 (2 μg) | 2.0 | 1.0 | 15.3 | 2.52 |
| PBA3 (1 μg) | 1.3 | 1.5 | 13.0 | 4.58 |
| PBA3 (0.5 μg) | 2.7 | 1.5 | 11.3 | 3.21 |
| 3358 & 3359(2 μg + 2 μg) | 49.0 | 5.6 | 73.0 | 6.24 |
| 3358 & 3359(1 μg + 1 μg) | 31.7 | 1.5 | 52.7 | 4.04 |
| 3358 & 3359(0.5 μg + 0.5 μg) | 22.0 | 2.6 | 46.7 | 4.04 |
| 3358 & rPCG-4 (1 μg + 1 μg) | 41.7 | 2.1 | 73.3 | 5.69 |
| 3359 & rPCG-4 (1 μg + 1 μg) | 61.0 | 6.6 | 64.3 | 8.14 |

Antibody pairs were found to be between 1.3 and 3-fold more efficient in their ability to neutralize than equivalent concentrations of individual monoclonal antibodies, see FIG. 17. FIG. 17(A) illustrates the titration of antibody 3358 in the presence of fixed amounts of the 3359 antibody. FIG. 17(B) illustrates the titration of antibody 3359 in the presence of fixed amounts of the 3358 antibody. To demonstrate the plateau level of neutralization achieved for using the 3358 and 3359 antibodies in isolation, the 4-parameter fit for each antibody in the absence of the other is extended out to higher antibody dose regimens than were performed for the combination study.

The most effective combinations were 3358/3359 and rPCG-4/3359. In this assay, each monoclonal antibody was found to reach a plateau level of neutralization, usually well below 100%, that could not be overcome by increasing the antibody dose. Combining antibodies appeared to raise the plateau neutralization level above what was observed for any of the monoclonal on their own suggesting that adding multiple antibodies may be important for more than simply obtaining dose enhancements (FIG. 17). A mixture of all three monoclonal antibodies further increased neutralization. These results were also confirmed by simple observation of the CHO cell morphologies. Full neutralization and partial neutralization can be easily visualized by the presence of differentiated (non-rounded) adherent cells. The various combinations of 3358, 3359 and rPCG-4 all came close to mimicking the differentiated cell morphology observed when toxin A was mixed with the anti-toxin A polyclonal control.

Epitope Mapping: The exact epitopes of the toxin A CWB-domain that must be targeted for neutralization have not been fully characterized. Therefore, a series of experiments was performed to map the epitopes and investigate the mechanism of action of the murine monoclonal antibodies 3358 and 3359. The study also included rPCG-4 and rPBA-3 (8,15,34).

The relative affinity of the antibodies 3359, 3358, rPCG-4 and rPBA-3 for four toxin A CWB-domain constructs was assessed by surface plasmon resonance. The four toxin constructs have been described previously (10). As shown in table 2, below, all anti-toxin A antibodies included in this experiment recognized multiple CWB-domain constructs with a range of binding affinities. Table 2 summarizes the apparent antibody affinities for the toxin A full and truncated CWB-domain. The K_{d} (nM) was determined by surface plasmon resonance. The potential number of toxin A-binding sites was estimated based on concentration dependent antibody competition studies.

TABLE 2

| | ToxA: | ToxA: | ToxA: | | # Potential |
|---|---|---|---|---|---|
| Antibody Lines | 6R | 7R | 11R | ToxA: 40R | binding sites |
| rPCG-4 Fab | 1.3 | 1.2 | 19 | 0.8 | N.D. |
| rPCG-4 | 1.2 | 0.3 | 29 | 0.5 | 4-6 |
| rPBA-3 | — | 55 | 60 | 16 | N.D. |
| 3358 (or 543) | 0.6 | 3.4 | 0.7 | 0.3 | Up to 14 |
| 3359 (or 227) | 100 | 13 | 12 | 1.2 | >2 |
| 3356 | 3.2 | 106 | 20 | 0.3 | N.D. |

Although the antibodies are functionally bivalent (excluding the rPCG-4 Fab construct) and the repeat domains potentially have many analogous antibody binding sites, the kinetic data for each antibody/toxin binding experiment invariably fit well to a 1:1 model. These apparent equilibrium K_{D} values were used to rank order the relative affinity of each antibody for each toxin domain. The two most singly neutralizing antibodies, 3358 and rPCG-4, recognized all toxin A repeat domains with high affinity (K_{Dapp}<30 nM).

Determination of Competing Versus Non-competing Antibody Epitopes: To determine approximate antibody/toxin stoichiometries for the 3358, 3359 and the rPCG-4 antibodies, a competition experiment was designed using surface plasmon resonance. The full-length toxin CWB domain, ToxA:40R, was titrated with both the rPCG-4 Fab expressed in E. coli and the rPCG-4 monoclonal antibody expressed in human 293F cells, see FIG. 182A, 18B. FIG. 18 illustrates data for antibody competition for toxin binding site experiments, studied by surface plasmon resonance. Competition studies were performed as described in the Methods, above. Soluble antibodies were titrated into a solution containing 15 nM ToxA:40R before injecting these solutions over surfaces with immobilized rPCG-4, 3359 and 3358. Titration of ToxA:40R with rPCG-4 Fab with detection on rPCG4, 3359 and 3358 surfaces 18(A). Titration of ToxA:40R with rPCG-4 antibody, FIG. 18(B); antibody 3359 FIG. 18(C) and antibody 3358 FIG. 18(D).

As expected, addition of rPCG-4 Fab to a solution with 15 nM ToxA:40R blocked toxin binding to the immobilized rPCG-4 antibody. The rPCG-4 Fab saturated the full-length CWB-domain, ToxA:40R, at a 1:6 toxin:antibody ratio while the full-length antibody saturated at a 1:3 ratio (FIG. 18B). This data suggests that PCG-4 recognizes a maximum of 6 separate sites within the CWB-domain of toxin A with high affinity. The existence of weak rPCG-4 binding sites (i.e., K_{D}>10⁻⁸) as observed for ToxA:11R indicates that there are a minimum of 4 high affinity binding sites.

The rPCG-4 antibody also competed with both the 3358 and 3359 antibodies for binding to ToxA:40R (FIG. 182B). To completely abrogate toxin binding to the 3359 derivatized surface, it was necessary to increase the rPCG4 antibody concentration above the concentration necessary for blocking its own binding. This result suggests that a low affinity binding site for rPCG-4 must be occupied to entirely occlude the 3359's ability to bind ToxA:40R (FIG. 2B). The rPCG-4 antibody blocked ToxA:40R binding to the derivatized 3358 surface at a concentration similar to its own saturation profile (FIG. 18B). However, in all competition experiments with soluble rPCG-4 antibody, addition of excess rPCG-4 beyond the level necessary to saturate all the 3358 binding sites led to an increase in 3358 signal. One explanation is that excess rPCG-4 may lead to an allosteric change within the toxin molecule allowing 3358 access to another binding site. Another explanation may be that occupation of multiple low affinity binding sites for rPCG-4 occludes its own ability to access the high affinity sites while leaving overlapping 3358 binding surface open.

The two neutralizing monoclonal antibodies, 3359 and 3358, behaved uniquely in the competition assay. Adding excess soluble 3359 antibody did not entirely block ToxA:40R from binding immobilized 3359 antibody even at an 8 molar excess of 3359 in solution (FIG. 18C). This may be explained by the existence of low affinity 3359 binding sites on the surface of ToxA:40R (see Table 2, above). Addition of excess soluble 3359 to ToxA:40R also did not abrogate the toxin's ability to bind the 3358 derivatized surface. In fact, an increase in signal above that of the toxin alone on the 3358 surface was detected. This signal correlated well with the additional mass of the 3359 antibody directly bound to the toxin as it interacted with the 3358 derivatized surface. The 3359 antibody competed only weakly with rPCG-4.

The 3358 antibody saturated its own ToxA:40R binding sites at a 1:7 toxin:antibody ratio suggesting that 3358 has a maximum of approximately fourteen high affinity binding sites (FIG. 18D). This result was not completely unexpected considering that ToxA:40R contains 40 individual repeats of 20 to 30 amino acids. 3358 competed for rPCG-4 binding sites exactly as it competed against itself in agreement with the inverse experiment with soluble rPCG-4 confirming that the two antibodies share overlapping binding sites. 3358 did not displace 3359 at concentrations below 40 nM and only partially displaced 3359 at higher concentrations suggesting the two antibodies have at least one or two fully independent binding sites. The presence of unique epitopes recognized by the 3359 antibody also can explain why it coupled favorably with 3358 or rPCG-4 for enhanced neutralization in the toxin A neutralization assay.

Mechanistic Variability of Toxin A Neutralizing Antibodies: A flow cytometry assay was used to study CWB-domain association with mammalian cells (10). ToxA:11R was chosen for the flow cytometry assay based on its native folding properties and cell binding characteristics. The neutralizing anti-toxin A antibodies led to various changes in ToxA:11R binding to CHO cell surfaces, as illustrated in FIG. 19, showing CHO cell surface binding of ToxA:11R as determined by flow cytometry. Forward scatter (FSC) and Side scatter (SSC) profiles of CHO cells A gate was used to visualize live cells based on photomultiplier counts between 10³ and 10⁴ for both side-scatter and forward scatter, see FIG. 19(A). The population of live cells was between 10³ and 10⁴ counts. SSC and Fluorescence of the CHO cell population with no PENTA-HIS Alexafluor 488 antibody (Qiagen), see FIG. 19(B); in the presence of a 1:500 dilution of the PENTA-HIS Alexafluor 488 antibody, see FIG. 19(C); in the presence of 50 μg/mL ToxA:11R and the PENTA-HIS Alexafluor 488 antibody, see FIG. 19(D).

In FIG. 20, the effect of anti-toxin A antibodies on ToxA:11R cell surface association was determined. Anti-toxin A antibodies were mixed with ToxA:11R prior to the addition to CHO cells. Each panel is the SSC and fluorescence profile of ToxA:11R in the presence of 3358, 250 μg/mL FIG. 20(A); 3359, 250 μg/mL FIG. 20(B); rPCG-4, 250 μg/mL FIG. 20(C); 3358 (250 μg/mL) and 3359 (250 μg/mL) FIG. 20(D); rPCG-4 (250 μg/mL) and 3359 (250 μg/mL) FIG. 20(E); and rPCG-4 (250 μg/mL) and 3358 (250 μg/mL) FIG. 20(F). Both the 3358 and rPCG-4 antibodies significantly increased the amount of CWB-domain detected at the cell surface, see FIGS. 20A, 20C. These antibodies may act by slowing the endocytosis of toxin A into the cell or by allowing the toxin to orient so that it increases the availability of the Histag for detection. 3358 and rPCG-4 also demonstrated overlapping binding epitopes, suggesting a similar mechanism for neutralization. The 3359 antibody inhibited cell surface association of ToxA:11R (FIG. 20B). Its toxin A CWB-domain epitope is different according to the surface plasmon resonance competition and mini-domain binding studies. The combination of the 3359 and 3358 antibody inhibits ToxA:11R binding the CHO cell surface similar to the behavior of 3359 alone (FIG. 20D).

Considering the two antibodies recognized different epitopes, it was not surprising that the binding inhibition behavior of 3359 dominates. rPCG-4 combined with 3359 increased ToxA:11R binding to cell surfaces indicating that rPCG-4 dictates the behavior of the two antibodies when introduced at identical concentrations in the assay (FIG. 20E). The increased fluorescence was not as high as with rPCG-4 alone, however, indicating that 3359 is still capable of exerting some influence on ToxA:11R binding. rPCG-4 and 3359 weakly competed with one another for binding to toxin A, perhaps sharing only a select number of epitopes.

Production of Murine Anti-toxin B Antibodies: While toxin A is certainly a primary factor in the development of the disease, neutralization of both toxins may be necessary to confer complete protection after the onset of the disease. Therefore, while one focus of this study was directed at investigating the properties of toxin A binding antibodies, murine monoclonal antibodies were generated against the toxin B CWB-domains using a similar approach to what was used in the anti-toxin A studies. Toxin B contains 25 C-terminal repeats compared to toxin A (which contains 40). The structure of expressed ToxB:25R was highly similar to what was observed for ToxA:40R (8). Six monoclonal antibodies with significant toxin B neutralization capabilities were isolated and subcloned. Similar to what was observed for the anti-toxin A monoclonal antibodies, the epitope but not the binding affinity of these monoclonal antibodies was most important for toxin B neutralization. Monoclonal 3592 (ATCC accession no. ______) neutralized toxin B in a neutralization assay in a similar manner to the polyclonal anti-toxin control. This antibody had the second lowest apparent affinity for ToxB:25R of the 6 final anti-toxin B antibodies. Another antibody raised against ToxB:25R, denoted F2, demonstrated relatively high affinity binding to both toxins A and B. This antibody was weakly neutralizing for toxin B in the neutralization assay but had no discernible affect on the activity of toxin A.

Testing of the anti-toxin antibodies in the Rat Ileal Loop and Hamster Infection Models: In vivo neutralization of toxin A's enterotoxic activity by monoclonal antibodies 3359 and 3358 was assessed using a rat ileal loop model. Ileal loops injected with 5 μg toxin A and 3359 or 3358 antibodies exhibited no visible disruption of the mucosal layer, as shown in FIG. 21. FIG. 21 illustrates the histology of rat intestinal mucosa after treatment with toxin A with or without anti-toxin A antibodies. Cross-section of rat ileal loop after the addition of A. Saline (FIG. 21A); B. 5 μg toxin A (FIG. 21B); C. 5 μg toxin A (FIG. 21C) and 10 μg mouse isotype control antibody; D, 5 μg toxin A and 12.5 μg 3359 antibody (FIG. 21D); E. 5 μg toxin A and 17 μg 3358 antibody (FIG. 21E); and 5 μg toxin A; and, F. 10 μg anti-toxin A polyclonal antibody (FIG. 21F).

In contrast, following a 4 h incubation with 5 μg toxin A, there was clear disruption of the intestinal mucosa and an increase in ileal loop weight-to-length ratio compared to loops injected with saline solution, see FIGS. 21 and 22. FIG. 22 illustrates data from a rat ileal loop assay showing that antibodies 3359 and 3358 prevent toxin A-induced intestinal fluid secretion in rat ileal loops. Rat ileal loops were exposed to saline solution, toxin A, or mixture of antibody with toxin A incubated for 5 min ex vivo. The concentrations of antibody 3359 and 3358 are indicated as well as the number of animals (n).

Antibody treatments were more efficacious than cholestyramine. Titration of the antibodies was performed to determine threshold doses of antibodies required for neutralization of 5 μg of toxin A was in the ileal loops. Addition of monoclonal antibody levels as low as 2 μg led to complete neutralization of toxin A for both the 3359 or 3358 antibodies tested individually. Antibodies 3359 and 3358 were also tested as a mixture. As shown in FIG. 22, a mixture of antibody 3359 and 3358 (0.5 μg each) was 100% effective in preventing fluid accumulation in the loops, suggesting the potential for synergy between the two antibodies.

The anti-toxin A antibodies 3358 and 3359 were also tested individually or in combination in the hamster infection model. In addition, the anti-toxin B antibody 3595 was evaluated together with the anti-toxin A antibodies. A single oral challenge with approximately 100 C. difficile spores resulted in 100% lethality within 4 days with or without diarrhea, accompanied by weight loss. In contrast, single daily oral doses of vancomycin, given for three days starting 4 h post C. difficile challenge, conferred 85% protection for the duration of the study. FIG. 23 shows the protective effects of antibodies 3359 and 3358 alone or in combination in the hamster challenge model. FIG. 23 illustrates data showing the efficacy of systemic dosing with anti-toxin A and anti-toxin B antibodies in C. difficile in hamsters. Groups of 5 hamsters were infected with C. difficile 48 h after clindamycin. Antibodies were administered IP for 4 consecutive days. The following regimens were used: no intervention (negative controls); vancomycin treatment (50 mg/kg); anti-toxin A antibody 3358 (2.5 mg/dose); anti-toxin A antibody 3359 (2.5 mg/dose); combination of anti-toxin A antibodies 3358 and 3359 (2.5 mg each/dose); combination of anti-toxin A antibodies 3358 and 3359 and anti-toxin B antibody 3595 (2.5 mg each/dose); polyclonal antibody against toxin A and toxin B. The hamsters were monitored for symptoms after bacterial challenge.

Four IP doses of 2.5 mg of the polyclonal antibody mixture or the monoclonal antibody 3358 (alone or combined with 3359) resulted in over 50% survival (p<0.05 for each regimen vs. untreated controls); see FIG. 23. Antibody 3358 at higher doses (5-10 mg) moderately delayed the onset of disease symptoms, but did not improve survival. When the two anti-toxin A monoclonal antibodies were combined with an antibody directed against toxin B (3595), 100% of hamsters survived the challenge (p<0.05 vs. untreated controls). Interestingly, hamsters treated with polyclonal goat anti-toxin A and B antibody did not appear to be protected from the C. difficile challenge as well as those treated with the triple combination of monoclonal antibodies.

Cross-sections from cecal tissues were examined to determine the effects of toxins and antibody treatment on mucosal surfaces, see FIG. 24, which illustrates the histology of hamster intestinal mucosa after C. difficile challenge as described in the legend of FIG. 23. FIGS. 24A to F illustrate photomicrographs of the histology of hamster intestinal mucosa after this C. difficile challenge. FIG. 24A illustrates infected but untreated; FIG. 24B illustrates treated with vancomycin; FIG. 24C illustrates treated with 3359; FIG. 24D illustrates treated with 3358; FIG. 24E illustrates treated with a combination of 3358 and 3359; FIG. 24F illustrates treated with a combination of 3358, 3359 and 3595.

Tissues from the untreated group exhibited complete destruction of normal villous architecture and submucosal congestion. The cecal tissues from the group of hamsters treated with vancomycin after C. difficile challenge did not show histological damage. Cecal tissues from hamsters treated with the triple combination exhibited an intact architecture very similar to the vancomycin-treated group. Of note, the tissues from animals treated with 3358 or 3359 showed only mild disruption of the mucosa. Mucosal damage varied within each anti-toxin A treatment regimen but correlated well with the severity of the disease. For instance, surviving hamsters from the study treated with one or two antibodies typically demonstrated mild to moderate inflammation of the mucosa. Most of these animals exhibited mild to moderate diarrhea. There was no substantial mucosal damage in hamsters treated with the combination of two anti-toxin A and one anti-toxin B antibodies. Only one animal in this group had mild diarrhea and none exhibited weight loss.

Analysis of murine anti-toxin antibodies in sera and cecal contents of hamsters revealed significant anti-toxin A and anti-toxin B levels as measured by quantitative ELISA. The level of toxin A detected in the cecum varied from an average of 0.1 μg/g cecal content in untreated hamsters to 0.001 to 0.0001 μg/g cecal content for hamsters treated with the triple antibody combination. Unlike the hamsters treated with vancomycin, hamsters treated with antibodies were still colonized with C. difficile but exhibited low toxin levels in the cecum.

### Discussion

While the antibody therapy approach to treat C. difficile infection has been described frequently in the literature, these studies demonstrate a novel combination strategy to generate an effective recombinant antibody cocktail for treating the disease based on the ideal in vitro properties of anti-toxin monoclonal antibodies. Surface plasmon resonance and flow cytometry was used to investigate previously undefined binding characteristics between antibodies and toxin A as well as the mechanism by which toxin neutralization occurs. Quantitative numbers were attributed to the stoichiometry of toxin A:antibody binding for two monoclonal antibodies developed in this study, 3358 and 3359, and for the previously described PCG-4 antibody. These results with PCG-4 are consistent with the previous work of Frey and Wilkins (15), and highlight the existence of additional high affinity epitopes for the PCG-4 antibody as well as the existence of one or more low affinity binding sites.

As the CWB-domains of toxins A and B are considered the main receptor binding domains of the toxins (see 42, 45, 47, below), it has been suggested that antibodies against the CWB-domains can inhibit interaction of the toxins with the cell surface receptors (11, 33). The flow cytometry experiments described here demonstrate multiple and complex mechanisms of neutralization by the monoclonal antibodies. Surprisingly, the most neutralizing antibodies, 3358 and rPCG-4 did not abrogate the binding of the CWB-domain to cell surfaces. Abrogated cell surface binding has been the most common mechanism of neutralization reported for antibodies and is believed to be the mechanism for PCG-4 in particular. Instead, 3358 and rPCG-4 increased cell surface binding of CWB-domain, resulting in the accumulation of CWB-domain at the cell surface. While the invention is not limited by any particular mechanism of action, one plausible mechanism is that these antibodies inhibit the internalization of the toxin into endosomal compartments for processing and potential release of the cytotoxic portion of the molecule within the cell. This would imply that the level of toxin binding observed at 1 mM Ca²⁺ concentrations was in equilibrium between cell surface binding, cell surface release, and internalization into endosomal compartments.

In contrast, antibody 3359 exhibited a different mechanism of action, namely inhibition of receptor binding. Addition of 3359 resulted in a dose dependent loss of detectable CWB-domain on the cell surface. Interestingly, Antibody 3359 was found to bind different epitopes of the toxin A CWB-domain than 3358 or rPCG-4.

Although the primary goal of this study (of Example 5) was to describe the antibody discovery and characterization process, compelling evidence is provided herein demonstrating that antibody 3359 of the invention works cooperatively with other neutralizing monoclonals against toxin A. The overall antibody concentration required to neutralize toxin A was reduced up to 4-fold in experiments where antibodies were used in combination. Each monoclonal antibody was found to reach a plateau level of neutralization, usually well below 100%, that could not be overcome by increasing the antibody dose. Combining antibodies appeared to raise the plateau neutralization level above what was observed for any of the monoclonal individually suggesting that adding multiple antibodies can improve efficacy over what can be achieved with a single antibody. Near equivalent enhancements (3-4 fold) in the dose dependence of toxin A neutralization were observed for the 3358/3359 combination in both the rat ileal loop experiments and in the toxin neutralization cell assays.

Enhanced efficacy for the 3358/3359 combination was not observed in the hamster C. difficile infection model, but that may be due to additional complications related to the presence of toxin B and/or other virulence factors. Previous studies, especially in the realm of anti-infectives, have demonstrated that the addition of two or more antibodies can have synergistic neutralizing effects (see 6, 37, 39, 41, 47, 50, below). There have been many studies reporting the discovery of weakly or moderately neutralizing antibodies against primary HIV isolates and botulism neurotoxins. Interestingly, many of the most effective HIV neutralizing antibody combinations such as the anti-envelope tailspike mAbs 2G12 and b12 do not share overlapping epitopes (41, 50).

In addition to working favorably for neutralization with 3358, the unique epitopes recognized by 3359 may also allow it to work favorably for neutralization in combination with 3358 and rPCG-4. Combinations of antibodies directed to different epitopes may provide a superior surface coverage over what can be achieved with individual monoclonal antibodies. The fact that multiple monoclonal antibodies are more effective at neutralizing toxin A also suggests that the CWB-domains may contain more than one or two specific receptor binding sites for binding to human cells. In support of this notion, published studies have shown that truncation of the CWB-domains of toxin B and Lyt-A merely attenuate their functions, but do not abrogate them (12, 24). The multiplicity of the repeat domains themselves suggests their effects can be additive and not entirely receptor/protein specific.

The neutralization of C. difficile toxin by the monoclonal antibodies was investigated in two art-recognized animal models. Both 3359 and 3358 antibodies inhibited the enterotoxic activity of toxin A inhibiting fluid accumulation in a rat ileal loop model. These observations were developed further in a hamster model of lethal C. difficile infection. In this model, a combination of 3 monoclonal antibodies directed against both toxins A and B was most effective when administered systemically. This provides a rationale for treatment of severe disease when transport of orally administered agents to the colon may be poor. The most effective combination resulted in a 3 to 4-fold increase in potency for the two mAbs in both the in vitro and in vivo assays. Interestingly, while the invention is not limited by any particular mechanism of action, it is possible that overcoming toxicity in C. difficile may mean simply attenuating the function of both toxins as opposed to completely neutralizing them.

While many studies have demonstrated that neutralization of toxin A is the most crucial factor for staving off the pathological effects of C. difficile, complete protection is often not achieved without concomitant neutralization of toxin B (and this invention also encompasses neutralization of the effects of toxin B). The dominant role of toxin A in the disease has been demonstrated by the fact that certain avirulent isolates of C. difficile do not carry an entirely functional toxin A gene (30). Other studies have shown complete or partial protection by vaccination with toxoid A (1, 20) or by the administration of polyclonal or monoclonal antibodies raised solely against toxoid A or nontoxic fragments of toxin A (8,35). However, there is significant data which suggests that complete protection against C. difficile requires the neutralization of both toxins (see 13, 17, 24, 28, below). Here, these studies demonstrate that neutralization of toxin B at the onset of infection is important for protection. Other studies with anti-toxin A and anti-toxin B vaccines have also shown that toxin B is involved in more than late stage pathogenesis (i.e. inducing cytotoxicity in cells exposed by toxin A), and must be neutralized early.

The precise mechanism by which systemic antibody administration mediates protection from C. difficile associated diarrhea in the hamster model is not very well understood. Toxin A may induce diarrhea following C. difficile challenge. In this study, parenteral delivery of anti-toxin antibody was shown to minimize or prevent the enterotoxic activity associated with C. difficile infection. The protection would probably require that antibodies react with the toxins at the level of the intestinal epithelium and/or in the intestinal lumen. It is possible that circulating toxin-neutralizing antibodies gain access to the gut lumen as a consequence of the mild inflammation of the mucosa observed in protected animals. However, cecal tissue from hamsters surviving the C. difficile challenge revealed only modest epithelial changes and mild inflammation of the mucosa. In addition, this would have to occur in the absence of observable fluid loss since most of the protected animals treated with the triple antibody combination did not develop diarrhea. Interestingly, the murine antibodies were detected in serum and in the cecal lumen following parenteral administration. This data may suggest that the antibodies were transported by intestinal secretions and/or by passive diffusion. In any case, this observation confirms the role of circulating antibodies providing mucosal and systemic protection from C. difficile disease (1).

In conclusion, these studies demonstrated that toxin A can be effectively neutralized by a combination of mAbs of the invention which bind to non-overlapping epitopes. A combination of selected high affinity monoclonal antibodies can offer advantages over either polyclonal or single monoclonal alternatives. Combinations of monoclonal antibodies demonstrated superior toxin/epitope coverage over single monoclonals and allowed for the incorporation of multiple mechanisms of toxin neutralization. An advantage over polyclonal antibodies is that monoclonal combinations can be produced recombinantly a necessary component of commercialization. Additionally, the properties of each recombinant antibody and/or antibody combination can be definitively controlled and studied providing a more solid efficacy/safety profile. For these reasons, the monoclonal antibody combinations of the invention offer a general route to more potent antigen neutralization, especially in the realm of anti-infectives.

### REFERENCES FOR EXAMPLE 5

1. Aboudola, S., Kotloff, K. L., Kyne, L., Warny, M., Kelly, E. C., Sougioultzis, S., Giannasca, P. J., Monath, T. P. and Kelly C P. 2003. Clostridium difficile vaccine and serum immunoglobulin G antibody response to toxin A. Infect Immun. 71:1608-10.
2. Aktories, K. and Just, I. 1995. Monoglucosylation of low-molecular-mass GTP-binding Rho proteins by clostridial cytotoxin. Trends Cell. Biol. 5, 441-443.
3. Brazier, J. S., Fawley, W., Freeman, J. and Wilcox, M. H. 2001. Reduced susceptibility of Clostridium difficile to metronidazole. J. Antimicrob. Chemother. 48:741-742.
4. Casimiro, D. R., Toy-Palmer, A., R. C. Blake and Dyson, H. J. 1995. Gene synthesis, high-level expression, and mutagenesis of Thiobacillus ferrooxidans rusticyanin: His 85 is a ligand to the blue copper center. Biochemistry, 34, 6640-6648.
5. Chia, J. K., Chan, S. M. and Goldstein, H. 1995. Baker's yeast as adjunctive therapy for relapses of Clostridium difficile diarrhea. Clin. Infect. Dis. 20, 1581.
6. Chou, T.-C. and Talalay, P. 1984. Quantitative analysis of dose-effect relationships: the combined effects of multiple drugs or enzyme inhibitors. Adv. Enzyme Regul. 22:27-55.
7. Coloma, M. J., Hastings, A., Wims, L. A. and Morrison, S. L. 1992. Novel vectors for the expression of antibody molecules using variable regions generated by polymerase chain reaction. J. Immunol. Methods, 152, 89-104.
8. Corthier, G., M. C. Muller, T. D. Wilkins, D. M. Lyerly and R. L'Haridon. 1991. Protection against experimental pseudomembranous colitis in gnotobiotic mice by use of monoclonal antibodies against Clostridium difficile toxin A. Infect. Immun. 59:1192-1195.
9. Dattamajumdar, A. K., Jacobson, D. P., Hood, L. E. and Osman, G. E. 1996. Rapid cloning of any rearranged mouse immunoglobulin variable genes. Immunogenetics, 43, 141-151.
10. Demarest, S. J., Salbato, J., Elia, M., Zhong, J., Morrow, T., Holland, T., Kline, K., Woodnutt, G., Kimmel, E., B. and Hansen, G. 2005. Structural characterization of the cell wall binding domains of Clostridium difficile toxins A and B; Evidence that Ca²⁺ plays a role in toxin A cell surface association. J. Mol. Biol. 346:1197-1206.
11. Dove, C. H., Wang, S. Z., Price, S. B., Phelps, C. J., Lyerly, D. M., Wilkins, T. D. and Johnson, J. L. 1990. Molecular characterization of Clostridium difficile toxin A gene. Infect. Immun. 58:480-488.
12. Fernandez-Tornero, C., R. Lopez, E. Garcia, G. Gimenez-Gallego and A. Romero. 2001. A novel solenoid fold in the cell wall anchoring domain of the pneumonococcal virulence factor LytA. Nature Struct. Biol. 8:1020-1024.
13. Fernie, D. S., R. O. Thomson, I. Batty and P. D. Walker. 1982. Active and passive immunization to protect against antibiotic-associated caecitis in hamsters. Dev. Biol. Stand. 53:325-332.
14. Fischer, W. 2000. In Streptococcus pneumoniae (Tomasz, A. ed.) pp. 155-177, Mary Ann Liebert Inc., Larchmont.
15. Frey, S. M. and Wilkins, T. D. 1992. Localization of two epitopes recognized by monoclonal antibody PCG-4 on Clostridium difficile toxin A. Infect. Immun. 60, 2488-92.
16. Garcia, J. L., Diaz, E., Romero, A. and Garcia, P. 1994. Carboxy-terminal deletion analysis of the major pneumonococcal autolysin. J. Bacteriol. 176, 4066-4072.
17. Giannasca P J, Zhang Z X, Lei W D, Boden J A, Giel M A, Monath T P and Thomas W D Jr. 1999. Serum antitoxin antibodies mediate systemic and mucosal protection from Clostridium difficile disease in hamsters. Infect Immun. 67, 527-38.
18. Gorbach, S. L., Chang, T. W. and Goldin, B. 1987. Successful treatment of relapsing Clostridium difficile colitis with Lactobacillus GG. Lancet. 2, 1519.
19. Hofmann, F., Busch, C., Prepens, U., Just, I. And Aktories, K. 1997. Localization of the glucosyltransferase activity of Clostridium difficile toxin B to the N-terminal part of the holotoxin. J. Biol. Chem. 272, 11074-11078.
20. Kim, P. H., J. P. Iaconis and R. D. Rolfe. 1987. Immunization of adult hamsters against Clostridium difficile-associated ileocecitis and transfer of protection to infant hamsters. Infect. Immun. 55:2984-2992.
21. Kink, J. A. and Williams, J. A. 1998. Antibodies to recombinant Clostridium difficile toxins A and B are an effective treatment and prevent relapse of C. difficile-associated disease in a hamster model of infection. Infect. Immun. 66:2018-2025.
22. Krivan, H. C., Clark, G. F., Smith, D. F. and Wilkins, T. D. 1986. Cell surface binding site for Clostridium difficile enterotoxin: evidence for a glycoconjugate containing the sequence Galα1-3Galβ1-4GlcNAc. Infect. Immun. 53, 573-581.
23. Kurtz C. B., Cannon, E. P., Brezzani, A., Pitruzzello, M, Dinardo, C., Rinard, E., Acheson, D. W., Fitzpatrick, R., Kelly, P., Shackett, K., Papoulis, A. T., Goddard, P. J., Barker, R. H. Jr., Palace, G. P., Klinger, J. D. 2001. GT160-246, a toxin binding polymer for treatment of Clostridium difficile colitis. Antimicrob Agents Chemother. 45:2340-7.
24. Kyne, L., Farrell, R. J. and Kelly, C. P. 2001. Clostridium difficile. In Infectious Diarrhea. Gastroenterol. Clin. North Am. 30, 753-777
25. Kyne, L., M. Warny, A. Qamar, and C. P. Kelly. 2000. Asymptomatic carriage of Clostridium difficile and serum levels of IgG antibody against toxin A. N. Engl. J. Med. 342:390-397.
26. Kyne, L., M. Warny, A. Qamar, and C. P. Kelly. 2001. Association between antibody response to toxin A and protection against recurrent Clostridium difficile diarrhoea. Lancet 357:189-193.
27. Libby, J. M., B. S. Jortner and T. D. Wilkins. 1982. Effects of the two toxins of Clostridium difficile in antibiotic-associated cecitis in hamsters. Infect. Immun. 36:822-829.
28. Libby, J. M. and T. D. Wilkins. 1982. Production of antitoxins to two toxins of Clostridium difficile and immunological comparison of the toxins by cross-neutralization studies. Infect. Immun. 35:374-376.
29. Lima, A. A. M., Lyerly, D. M., Wilkins, T. D., Innes, D. J. and Guerrant, R. L. 1988. Effects of Clostridium difficile toxins A and B in rabbit small and large intestine in vivo and on cultured cells. Infect. Immun. 56, 582-588.
30. Lyerly, D. M., L. A. Barroso, T. D. Wilkins, C. Depitre and G. Corthier. 1992. Characterization of a toxin A-negative, toxin B-positive strain of Clostridium difficile. Infect. Immun. 60:4633-4699.
31. Lyerly, D. M., E. F. Bostwick, S. B. Binioin and T. D. Wilkins. 1991. Passive immunization of hamsters against disease caused by Clostridium difficile by use of bovine immunoglobulin G concentrate. Infect. Immun. 59:2215-2218.
31. Lylerly, D. M., Krivan, H. C. and Wilkins, T. D. 1988. Clostridium Difficile: Its Disease and Toxins. Clin. Microbiol. Rev. 1, 1-18.
33. Lyerly, D. M., J. L. Johnson, S. M. Frey and T. D. Wilkins. 1990. Vaccination against lethal enterocolitis with a nontoxic recombinant peptide of toxin A. Curr. Microbiol. 21:29-32.
34. Lyerly, D. M., Phelps, C. J., Toth, J. and Wilkins, T. D. 1986. Characterization of toxins A and B of Clostridium difficile with monoclonal antibodies. Infect. Immun. 54, 70-76.
35. Lyerly, D. M., Phelps, C. J. and Wilkins, T. D. 1985. Monoclonal and specific polyclonal antibodies for immunoassay of Clostridium difficile toxin A. J. Clin. Microbiol. 21, 12-14.
36. McFarland, L. V. 2005. Alternative treatments for Clostridium difficile disease: what really works? J. Med. Microbiol. 54(Pt2), 101-111
37. Marks, J. D. 2004. Deciphering antibody proteprites that lead to potent botulinum neutrotoxin neutralization. Movement Disorders, 19, S101-S108.
38. Mylonakis, E., Ryan, E. T. and Calderwood, S. B. 2001. Clostridium difficile-Associated Diarrhea. Arch. Int. Med. 161, 525-533.
39. Nowakowski, A., C. Wang, D. B. Powers, P. Amersdorfer, T. J. Smith, V. A. Montgomery, R. Sheridan, R. Blake, L. A. Smith and J. D. Marks. 2002. Potent neutralization of botulinum neurotoxin by recombinant oligoclonal antibody. Proc. Natl. Acad. Sci. USA. 99:11346-11350.
40. Palace, G. P., Lazari, P. and Norton, K. 2001. Analysis of the physicochemical interactions between Clostridium difficile toxins and cholestyramine using liquid chromatography with post-column derivatization. Biochem. Biophys. Acta, 1546, 171-184.
41. Parren, P. W. H. I., P. A. Marx, A. J. Hessell, A. Luckay, J. Harouse, C. Cheng-Mayer, J. P. Moore and D. R. Burton. 2001. Antibody protects macaques against vaginal challenge with pathogenic R5 simian/human immunodeficiency virus at serum levels giving complete neutralization in vitro. J. Virol. 75:8340-8347.
42. Pothoulakis, C. and LaMont, J. T. 2001 Microbes and microbial toxins: paradigms for microbial mucosal interactions. II. The integrated response of the intestine to Clostridium difficile toxins. Am. J. Physiol. Gastrointest. Liver Physiol. 280, G178-G183.
43. Sauerborn, M., Leukel, P. and von Eichel-Streiber, C. (1997) The C-terminal ligand-binding domain of Clostridium difficile toxin A (TcdA) abrogates TcdA-specific binding to cells and prevents mouse lethality. FEMS Microbiol. Lett. 155, 45-54.
44. Small, J. D. 1968. Fatal enterocolitis in hamsters given lincomycin hydrochloride. Lab. Animal Care 18:411-420.
45. Surawicz, C. M., Elmer, G. W., Speelman, P., McFarland, L. V., Chinn, J. and van Belle, G. (1989) Prevention of antibiotic-associated diarrhea by Saccharomyces boulardii: a prospective study. Gastroenterology, 96, 981-988.
46. Tucker, K. D. and T. D. Wilkins. 1990. Toxin A of Clostridium difficile binds to the human carbohydrate antigens I, X, and Y. Infect. Immun. 59:73-78.
47. Volk, W. A., B. Bizzini, R. M. Snyder, E. Bernhard and R. R. Wagner. 1984. Neutralization of tetanus toxin by distinct monoclonal antibodies binding to multiple epitopes on the toxin molecule. Infect. Immun. 45:604-609.
48. Von Eichel-Streiber, C. and M. Sauerborn. 1990. Clostridium difficile toxin A carries a C-terminal repetitive structure homologous to the carbohydrate binding region of streptococcal glycosyltransferases. Gene. 96:107-113.
49. Wren, B. W. 1991 A family of clostridial and streptococcal ligand-binding proteins with conserved C-terminal repeat sequences. Mol. Microbiol. 5, 797-803.
50. Zwick, M. B., M. Wang, P. Poignard, G. Stiegler, H. Katinger, D. R. Burton, P. W. H. I, Parren. 2001. Neutralization synergy of human immunodeficiency virus type 1 primary isolates by cocktails of broadly neutralizing antibodies. J. Virol. 75:12198-12208.

### Example 6

### Anti-Toxin A Gastric-Stable Antibodies

This example describes the development of anti-toxin A gastric-stable antibodies of the invention, and provides studies that demonstrate their efficacy. The invention provides methods for modifying antibodies to generate new Ab sequences for oral delivery, wherein the modified antibodies are stable in the digestive-tract environment.

In an initial attempt to create an antibody molecule stable in gastric and intestinal fluids to target the Clostridium difficile toxins, mutations were built within IgG1 to replace potential pepsin and carboxypeptidase and trypsin cleavage sites using the next most frequently observed residue at each position. The antibodys ability to tolerate each individual mutation was evaluated by expression, thermotolerance and pH profiling. Several multi-site mutant combinations were created based on tolerance data, expression in mammalian cells and pepsin and pancreatin digestion profile. Various combinations were created with improved stability in vitro and in vivo. Thus, the invention provides methods for the development of anti-toxin A antibodies resistant to gastric fluids and intestinal fluids, and antibodies made by those methods.

1—Pepsin Mutations:

1a—Relative digestibility of various classes of antibodies to pepsin: In an initial attempt to create an antibody molecule stable in gastric fluids, various antibody classes were tested in simulated gastric fluids. Antibody molecules were incubated with pepsin at pH 1.2 in order to simulate the gastric phase of digestion. Initial time course digestibility profiles were performed with pepsin on human IgG1, IgG2, IgG3, and IgG4. All antibody classes, IgG1, IgG2, IgG3 and IgG4 were rapidly proteolyzed into small fragments, see FIG. 25.

FIG. 25 illustrates the results of the pepsin digestion profiles of IgG1, IgG2, IgG3 and IgG. Antibodies were digested for 0, 2, 5, 10, 20 and 30 min with pepsin at pH 1.2. The digestion were run on a non-reducing SDS-PAGE gel (8%). The letter C indicates the antibody without pepsin. Molecular weight markers (MW-kDa) are indicated between gels. IgG2 and IgG4 appeared to undergo extensive proteolysis at very early time points. However, IgG1 and IgG3 seemed to display superior “resistance” to pepsin digestion. On a reducing gel, IgG1 exhibited a higher proportion of light chain preserved throughout time when compared to IgG3.

Interestingly, the acidic conditions alone in the absence of pepsin led to decreases in functional antibody, as illustrated in FIG. 26. This decrease was not detectable by electrophoretic analysis of the digestion. The chimeric antibody hPBA3 was expressed in mammalian cells, purified, and dialyzed. 1 μg was digested for 0, 2, 5, 10 20, and 30 min with pepsin (×0.005) at pH 1.5. The molecular weight marker (MW-kDa) is indicated. Samples were either loaded on a 4% to 12% Bis-Tris gel and Coomassie stained or tested in ELISA. An AP-labeled mouse anti-human Fc antibody was used for detection in the ELISA. FIG. 26A1 and FIG. 26A2: pH 1.5 and pepsin; FIG. 26B1 and FIG. 26B2: pH 1.5.

1b—Determination of pepsin cleavage sites to mutate: Potential pepsin cleavage motifs were determined based on known pepsin cleavage rules. The human IgG Fc structure was analyzed for pepsin cleavage motifs (Phe, Leu, Tyr and Trp residues) with greater than 25% solvent exposure. Cleavage sites were also prioritized based on the known cleavage attenuation effects of flanking Pro, Lys, Arg and His residues. Following this approach, 10 sites were identified as potential candidates for directed mutagenesis in the heavy chain constant domain. The putative cleavage sites were present within the hinge region, the CH1 and CH2 domains. One potential cleavage site within the hinge region was also given high priority due to its flanking sequences and the known dynamic nature of this region of the protein. Protection of the hinge is essential for maintaining the bivalent nature of antibodies. Moreover, bivalency has been shown to be important for neutralization of C. difficile toxin A. In parallel, the antibody molecule was subjected to proteomic analysis after pepsin digestion. Among 4 sites found by this experimental approach, 2 sites matched the sites identified by sequence analysis. The two other sites determined by proteomic analysis were in the C_{H}1 domain. All together, 10 potential cleavage sites plus two proteomically derived cleavage sites (12 total) constituted the list of residues deemed significant to mutate for pepsin resistance.

In addition, a second set of mutants was engineered considering that denaturation of the entire antibody molecule was anticipated at low pH making the whole molecule vulnerable to pepsin attack. For these mutants, every amino acid residue in the constant domain that was a putative pepsin recognition site was mutated without consideration of surface exposure. Addition of this second set of residues provided a total of 31 potential sites for mutation within the constant domain of the heavy chain.

1c—Folding status of antibody molecule at various pH: It was anticipated that the antibody would be proteolyzed rapidly at pH 2, as the molecule appeared to completely unfold between pH 2 and 3, see FIG. 27A. FIG. 27 illustrates a graphic summary of data showing the pH dependence of IgG1 structure demonstrated by Circular Dichroism (CD) experiments. There is time dependence to the unfolding of the antibody molecule that is hastened as the pH is lowered. At pH 2.8, IgG1 unfolded slowly and the onset of unfolding was observed after 20 minutes (FIG. 27B). At pH 2.5, approximately 50% of the molecule was unfolded after 20 minutes (FIG. 27C). At pH 2.2, unfolding was rapid and almost complete for the entire antibody molecule within 20 minutes (FIG. 27D). In FIG. 27: all spectra were taken at 25° C. in a 1 mm cuvette at a protein concentration of 2 μM. FIG. 27A: The spectra of IgG1 at pH values of 3 and above are highly indicative of beta-sheet-like structure with a single minimum at 217 nm. At pH 2 and below, the spectra change radically to spectra highly indicative of random coil (unfolded) with the characteristic minimum at 197 nm. FIG. 27B: CD spectra of IgG1 at pH 2.8. Each scan began exactly 4, 9.3, 14.7, 20 and 25.2 minutes after titration to pH 2.8. FIG. 27C: CD spectra of IgG1 at pH 2.5. FIG. 27D: CD spectra of IgG1 at pH 2.2.

1d—Selection of replacement residues for pepsin vulnerable sites: The selection of residues to replace the potential cleavage sites was based on information from a database of IgG Fc sequences. Mutations were made to the next most frequently observed residue within the dataset of IgG sequences. No IgG constant domain sequence has more than 95% identity to any other member in the database. The database was limited to IgG to limit the co-variation of residues between sequences which could lead to the necessity of linked mutations along with the individual site mutations of the invention. In order to understand the mutational tolerance of each position, the residues were also mutated to alanine and tested for expression and tolerance to low pH.

1e—Screening of single mutants for resistance to pepsin: Thermotolerance and assessment of the expression level was used as a first screen to insure that mutations directed towards pepsin resistance was tolerated and did not impair the stability of the molecules. DNAs derived from the 66 variants were transfected into mammalian cells and the resulting supernatants were screened for expression, thermotolerance, and pH tolerance. All members demonstrated a similar pH tolerance compared to wildtype. This was expected as no charged residues that may affect the pH stability of the antibody molecule were chosen for mutation. Many mutants demonstrated inferior thermotolerance and/or expression compared to the wildtype molecule. Approximately 46% of the database selected mutants were destabilizing while 64% of the alanine mutations were destabilizing. Interestingly, single mutations can confer some degree of resistance to pepsin digestion, particularly the mutation within the hinge region as shown in Table 1, below, summarizing the percentage of antibody recovered after digestion with pepsin. The percentage of antibody molecule remaining after digestion as well as the percentage of antibody binding to toxin A are reported after 0.5 h, 1 h, and 4 h digestion with pepsin at pH 3. The amount was measured by ELISA to detect specifically the constant domain of the wildtype and the mutants. Mutations are listed below:

TABLE 1

| | | Fc | | | | |
|---|---|---|---|---|---|---|
| | | detection | | | Binding to Toxin A | |
| BD | | 0.5 h | 1 h | 4 h | 1 h | 4 h |
| 12611 | T178S | 62% | 39% | 13% | 98% | 94% |
| 12636 | L258P | 46% | 52% | 60% | 86% | 87% |
| 12635 | L202I | 69% | 70% | 0% | 82% | 87% |
| 12632 | Y436T | 17% | 0% | 0% | 77% | 76% |
| 12629 | F427Y | 63% | 30% | 52% | 100% | 96% |
| 12623 | Y372H | 67% | 15% | 0% | 88% | 86% |
| 12613 | F264Y | 43% | 23% | 0% | 65% | 48% |

1f—Design and screening of up-mutants: Up-mutants containing multiple pepsin resistance sites were designed based on the initial 66 member library screen. Mutations were added progressively to the properly folded single point mutants that had shown high level of expression and resistance to pepsin digestion. The up-mutants were expressed in mammalian cells and tested for expression and their folding properties as addition of subsequent mutations on a single framework may alter the folding. All up-mutants expressed comparably to the wildtype gene in mammalian cells and demonstrated similar thermotolerance profiles.

The antibody molecule did not unfold until the pH was lowered below pH 3. Therefore, the pepsin digestibility of the wildtype antibody and the mutant combinations was also measured at a pH value (pH 3) where the molecule remained folded and the pepsin is still active. Examples of pepsin digestions are shown in FIG. 28. Drastic differences in digestibility were observed between the wildtype protein and two six mutant combinations at pH 3, see FIG. 28. The wildtype protein was over 80% degraded within 30 minutes of exposure to 0.005×SGF at 37° C., pH 3. SDS-page analysis of the wildtype digestion indicates the appearance of Fc fragment even at the earliest time point, 2 minutes. Several other smaller molecular weight bands were apparent after 10 minutes.

In contrast, the mutant combinations were completely undisturbed after two hours of exposure to pepsin under the same conditions. The mutants exhibited larger molecular weight bands after digestion than the wildtype protein suggesting that one or more of the mutations hindered the formation of lower molecular weight fragments. Several combinations of mutations were identified to confer resistance to pepsin digestion.

Transient exposure of antibodies to conditions of increasing acidity (pH 3 to pH 2 in the presence of pepsin) led to decreases in antibody-toxin binding, see Table 2, below, which summarizes the percentage of wildtype and mutant antibody molecules recovered after digestion with pepsin. The parent antibody molecule as well as the mutants were expressed in mammalian cells, purified, and dialyzed. 1 μg was digested for the time indicated with pepsin (×0.005) at 37° C. at pH 2, pH 2.5, and pH 3. Two separate tests were performed: one to detect the remaining constant domain and a test to assess the remaining binding activity of the antibody molecule. In all cases, antibody degradation was determined by measuring by ELISA the amount of antibody remaining after digestion. Mutations are listed in Table 2, below. The percentage of Fc remaining after digestion is reported after 0.5 h, 1 h, and 4 h digestion with pepsin at pH 3, 2.5 and 2:

TABLE 2

| | | pH3 | | | pH2 | |
|---|---|---|---|---|---|---|
| BD # | Mutation | 0.5 h | 4 h | 7 h | 5 min | 0.5 h |
| 12584 | Wildtype | 14% | 0% | 0% | 0% | 0% |
| 14079 | L258P, L332Q, F427Y, F264Y, L202I, L421Q | 91% | — | — | 0% | 0% |
| 13964 | L258P, L332Q, F427Y, F264Y, L202I, T178S | 100% | — | — | — | — |
| 13936 | L258P, L332Q, F427Y, L202I | 69% | 100% | 94% | 0% | 0% |
| | L257I, L258P, L332Q, F427Y, F264A, L202I, | | | | | |
| 14487 | L421Q, T178S | 0% | 0% | — | — | — |
| | L258P, L332Q, F427Y, F264Y, L202I, L421Q, | | | | | |
| 14357 | T178S | 100% | 100% | — | — | — |
| 12639 | L421Q, L258P | 98% | 100% | 100% | — | — |
| | L257I, L258P, L332Q, F427Y, F264A, L202I, | | | | | |
| 14568 | L421Q, T178S, L265I, Y342H | — | — | — | — | 0% |
| | L257I, L258P, L332Q, F427Y, F264A, L202I, | | | | | |
| 14563 | L421Q, T178S, L265I, L429V | — | — | — | — | 0% |

SDS-PAGE profiles demonstrate that the extent of proteolysis was considerably less when the antibody molecules were incubated with pepsin at pH 3 versus pH 2. The binding activity the wildtype and all the mutants remained stable overtime at pH 3 but decreased at pH 2.5 and pH 2. These results confirm that the pepsin cleaves the Fc domain and leaves intact the F(ab′)2 fragment that has the binding properties. However, the wildtype antibody digested with pepsin still retained toxin A binding as determined by ELISA, but did not neutralize toxin A as measured in the cell toxin neutralization assays, as illustrated in FIG. 29.

FIG. 29 illustrates pictures of cells cultured in the presence or absence of toxin and toxin-neutralizing antibody after pepsin digestion. Toxin neutralization was measured by mammalian cell proliferation in the presence of toxin (reduced cellular proliferation) or in presence of toxin plus the antibody candidate (cellular proliferation comparable to cells grown without toxin). The conditions chosen to test toxin neutralization were the following: 1×10⁴ CHO cells/well, 1 h pre-incubation of the mixture antibody to test and toxin, 48 h incubation of mixture with CHO cells. The concentration of toxin A (80 ng/well) chosen in these experiments triggered 100% cell death. Pictures illustrated in FIG. 29 represent adherent CHO cells cultured in the presence of 80 ng toxin A with the anti-toxin A antibody digested with pepsin for 0, 2, 5, 30, and 120 min.

2—Pancreatin Mutations:

2a—Relative digestibility of various classes of antibody classes in pancreatin: Various antibody classes were tested in simulated intestinal fluids. All antibody classes, IgG1, IgG2, IgG3 and IgG4 were proteolyzed by pancreatin, see FIG. 30, illustrating gels showing pancreatin digestion profiles of IgG1, IgG2, IgG3 and IgG4. Antibodies (10 ug) were digested for 0, 2, 5, 10, 20 and 30 min with pancreatin. The letter C refers to the test antibody without pancreatin. The letter P indicates the simulated intestinal fluid alone. Molecular weight markers (MW-kDa) are indicated between gels. Interestingly, the pattern of degradation appeared similar at 0 and 30 min.

2b—Identification of pancreatin cleavage sites to mutate: Trypsin and chymotrypsin are the most abundant enzymes present in pancreatin. Therefore, potential pancreatin cleavage motifs in the sequence of human IgG were determined based on known trypsin and chymotrypsin cleavage rules. Trypsin specifically recognizes Arg and Lys residues at the site where it cleaves peptide bonds. Arg and Lys residues with greater than 40% solvent exposure were identified as potential candidates for directed mutagenesis in the heavy chain and light chain constant domains. Chymotrypsin specifically recognizes Phe, Tyr or Trp. Therefore, Phe, Tyr and Trp residues with greater than 25% solvent exposure were identified as potentially candidates for directed mutagenesis in the heavy chain and light chain constant domains. The selection of residues to replace the potential cleavage sites was based on information from a database of IgG Fc sequences. Mutations were made to the next most frequently observed residue within the dataset of IgG sequences.

2c—Screening of single mutants for resistance to pancreatin: The selection of potential cleavage site replacements was designed on a database of IgG Fc sequences. Mutations were made to the next most frequently observed residue within the dataset of IgG sequences. 8 and 12 mutations were introduced in the light chain and the heavy chain respectively. Mutants were transfected into mammalian cells and the resulting supernatants were screened for expression and resistance to pancreatin digestion to determine whether mutation at each chymotrypsin and trypsin-labile position was tolerated. For thermotolerance, supernatants with recombinant antibody were heated challenged for 10 minutes at 70, 75 and 80° C. The amount of antibody remaining in the supernatant subsequent to thermal challenge was detected by ELISA assays and compared to data obtained with the wildtype protein. Most mutants demonstrate comparable thermotolerance and/or expression compared to the wildtype antibody. Interestingly, even single mutations can confer some degree of resistance to pancreatin digestion. Up-mutants containing multiple trypsin and chymotrypsin resistance sites were also tested for resistance to pancreatin. All up-mutants expressed comparably to the wildtype gene in mammalian cells and demonstrated similar thermotolerance profiles. Several combinations of mutations were identified to confer resistance to pancreatin digestion, as summarized in the table of FIG. 31.

In FIG. 31, the table summarizes the percentage of wildtype and mutant antibody molecules recovered after digestion with pancreatin. The parent antibody molecule (2934) as well as the mutants were expressed in mammalian cells, purified, and dialyzed. Antibody mutants were digested with pancreatin at 37° C. for the time indicated. ELISA assays were performed to measure the amount of the full length remaining antibody. Mutations are listed below. A score was given to each variant to describe its expression (Ex): +: Expression was greater than wildtype; : Equivalent expression compared to wildtype; −: Less material was expressed than the wildtype; −: No expression.

Each antibody variant was given a thermotolerance score (T) according to the following criteria: +: A greater percentage of folded protein remaining at 75° C. and/or 80° C. compared to wildtype; : Equivalent percentage of folded protein remaining at each temperature point compared to wildtype; −: A lesser percentage of folded protein remaining at 75° C. than wildtype; −: Thermal unfolding observed at 70° C.

3—Combination of Mutations to Confer Resistance to Pepsin and Pancreatin

In order to create an antibody molecule stable in gastric and intestinal fluids, the mutations identified to confer superior resistance to pepsin and to pancreatin were combined on one single antibody molecule. In particular, the heavy chain contained the mutations 155G, 258P, 296Q, 421Q, 143S, and 153A. The light chain contained mutations 143S and 153A. The optimized antibody exhibited comparable expression level to the control antibody without mutation when expressed in mammalian cells (HEK 293 cells). It was also tested for resistance to pepsin and pancreatin in in vitro in simulated gastric and intestinal fluids assays.

Table 4 shows the percentage of fall length antibody recovered after digestion with pepsin and with pancreatin as measured in ELISA assays. Clearly, the antibody containing the mutations exhibited higher level of resistance to digestion. In parallel, the optimized antibody was assessed for functionality. As the optimized antibody comprised the variable region of the murine antibody 543 shown to neutralize toxin A in the rat ileal loop model, it was tested for binding to toxin A by ELISA and for its activity in toxin neutralization assays. The optimized antibody was found to have the same binding affinity and toxin neutralization than the corresponding murine antibody and the control antibody without mutations.

Table 4 shows the percentage of antibody remaining after digestion with pepsin and pancreatin. The parent antibody molecule as well as the mutants were expressed in mammalian cells, purified, and dialyzed. 1 μg was digested for 2 min, 10 min or 30 min with pepsin (×0.005) at 37° C. at pH 3 and then digested with pancreatin for 120 min. The antibody degradation was determined by measuring by ELISA the amount of antibody remaining after digestion and the results are expressed as percentage of full length antibody remaining after digestion.

TABLE 4

| | Timepoint | | |
|---|---|---|---|
| Antibody | 2 min | 10 min | 30 min |
| IgG | 80 | 3 | 2 |
| 543 | 100 | 10 | 5 |
| r543 (optimized) | 97 | 66 | 26 |
| PCG4 | 91 | 4 | 2.5 |

4—Animal Studies to Evaluate Antibody Stability in the Digestive Tract

The survival of intact IgG in the cecum is relevant for the potential therapeutic use of a C difficile antibody. Pharmacokinetic studies were initiated in mice to assess the stability of the optimized chimeric antibody. Three studies were conducted sequentially:

Study 1: Test whether an acid blocker could neutralize the pH of the mouse stomach.

Study 2: Comparison of the stability of optimized vs. non-optimized antibody in mouse stomach, cecum and distal colon.

Study 3: Comparison of the stability of optimized vs. non-optimized antibody in mouse feces.

4a—Study 1: Preliminary studies were performed with a non-optimized human antibody with the goal of measuring the amount of IgG surviving passage to the mouse cecum. Additional aims were to determine whether reduced exposure to acidic gastric secretions would significantly alter IgG survival. In this first study, mice were fed orally with human IgGs and their stomach and cecal contents were collected for analysis. Because exposure of antibody to acidic gastric secretions (pH<3) resulted in rapid loss of the antibody molecule, the antibody was administered in a solution containing sodium bicarbonate. Prior to oral delivery, the antibody solution was buffered at pH 9.5 with a solution of sodium bicarbonate. Two groups of animals were fed prior to oral gavage either with a histamine H2 receptor antagonist (Cimetidine) or with the proton pump inhibitor omeprazole (40 mg, provided twice daily the day prior to and on the day of oral ingestion).

Table 5 shows the intact IgG amounts recovered from stomach and cecal content collections at the 1 hour (h) and 2 h time points, thus evaluating acid secretion inhibitors in mice. In Table 5, the pH was measured in mice stomach and cecum 1 h and 2 h after delivering cimetidine, omeprazole, bicarbonate buffer (0.1 M, pH 9.5) or a saline solution. Cimetidine and omeprazole were delivered orally 24 h prior to measurement.

TABLE 5

| | pH | pH |
|---|---|---|
| Treatment | Stomach | Cecum |
| Saline | 3 | 7 |
| Bicarbonate buffer | 3-6 | 8 |
| Cimetidine-24 h | 4-8 | 8 |
| Omeprazole-24 h | 4-5 | 8 |

The total recovery of IgG in the stomach was in the mg range when additional acid buffering capacity was provided in the form of an oral antacid. When the antibody was delivered in bicarbonate buffer (0.1 M, pH 9.5), the recovery of full length human IgG from the cecum was in the mg range. This represents 20 to 30% of the total ingested dose (5 mg). The use of additional antacid or a proton pump inhibitor did not result in any further significant increase in murine IgG survival in the cecum in this particular experiment. Thus, it appeared unnecessary to use these extra measures to protect antibody delivered orally from gastric degradation.

Table 6 summarizes data from studies that recover IgG from the stomach and cecum after oral administration of 5 mg human antibody. 5 mg of human antibody (Jackson ImmunoResearch Laboratories, Inc.) resuspended in 0.1 bicarbonate buffer (pH 9.5) was orally delivered to 25 g mice (n=2). During the study, mice had free access to food and water. Mice were then euthanized at various times (1 h and 2 h). Stomach and cecal contents were collected at indicated time points and assayed for presence of residual antibody. The antibodies were detected by using standard immunoassays to detect the full length intact antibody and Fab. The results are expressed as percentage of the total amount of antibody recovered from stomach or from cecum divided by the total amount of ingested antibody.

TABLE 6

| | | % Fc | | % Fab | |
|---|---|---|---|---|---|
| | Treatment | 1 h | 2 h | 1 h | 2 h |
| Stom- | Cimetidine | 30.2 ± | 41.5 ± 1.1 | 25.0 ± 2.9 | 39.1 ± 9.5 |
| ach | | 7.0 | | | |
| | Omeprazole | 38.9 ± | 24.8 ± 4.5 | 32.4 ± 8.5 | 28.2 ± 6.0 |
| | | 2.4 | | | |
| | Bicarbonate | 10.0 | 0.8 | 35.0 | 1.9 |
| Ce- | Cimetidine | 4.0 ± | 16.3 ± 1.7 | 8.1 ± 10.1 | 31.1 ± 10.6 |
| cum | | 5.3 | | | |
| | Omeprazole | 1.8 ± | 10.0 ± 1.1 | 9.8 ± 11.6 | 42.3 ± 19.6 |
| | | 1.4 | | | |
| | Bicarbonate | 18.1 | 20.2 | 18.1 | 38.1 |

4b—Study 2: The main study aims were to compare the optimized and the corresponding non-optimized antibody for their ability to survive passage through the mouse stomach, cecum and small intestine. Additional goals were to determine whether specific C. difficile toxin binding and neutralizing activity was preserved. In this study, mice were fed orally with IgGs and their stomach, cecal and distal colon contents were collected for analysis.

FIG. 32 illustrates data from a time course of IgG recovery from the stomach, cecum and distal colon after oral administration of antibody. The mean amounts of antibody recovered at each collection time is shown for each treatment group (n=3). The results are expressed as mean recovery of percentage of the total amount of antibody recovered in stomach or in cecum divided by the total amount of ingested antibody. CD-1 mice (Simonsen Laboratories, Inc., Gilroy Calif.) weighing about 25 g were housed in a 12-hour light/12-hour dark cycle and constant temperature environment of 22° C. A standard diet and water were supplied ad libitum during the period of acclimatization and during the study. On the day of the study, the animals were randomly divided into groups to receive the different treatments. The antibody solution was administered by oral gavage with a syringe (2.5 mg per dose). One group was administered the control human antibody (IgG), the corresponding chimeric antibody without mutations (control), and the optimized antibody (Optimized). One group was administered a saline solution only. Mice were then euthanized at various times (1 h, 2 h, 4 h and 6 h) and their stomach, cecal and distal colon contents were collected. In order to determine the recovery of antibody in the digestive tract, samples were immediately frozen and stored at −80° C. The samples were extracted in TBST buffer (TBST, 20 mM Tris, pH 7.4; 0.15M NaCl; 0.05% Tween 20; 0.05% NaN3; 0.1% BSA) supplemented with protease inhibitor (2 tablet per 10 ml of buffer, Complete EDTA-free protease inhibitor cocktail tablets, Roche #1873580). The stomach, cecal and distal colon extraction volume was recorded and an aliquot of each was dialyzed and filter-sterilized and used for toxin-neutralization experiments. The presence of residual antibody was estimated by using standard immunoassays to detect full length intact antibody.

As shown in FIG. 32, the optimized antibody showed the highest level of recovery from stomach, cecum, and distal colon with levels reaching 20% to 40%. Interestingly, very little of the control IgGs was recovered in the distal colon after 6 hours whereas the optimized antibody was still present. The small variations observed in a given mouse group can be explained by differences in transit times.

4c—Study 3: The main study aims were to quantitate the amount of IgG surviving passage through the digestive tract. In this study, mice were fed orally with IgGs and their feces were collected over time. Importantly, the antibody treatments were well tolerated. There was no evidence that any of the treatment produced any detectable effect on vital signs.

FIG. 33 illustrates data from a time course of antibody recovery from mouse feces after oral administration of 1 mg of the optimized antibody and a control antibody. 1 mg of antibody resuspended was orally delivered to 25 g mice (group size n=3). During the study, mice had free access to food and water. Feces were collected at various times (2 h, 4 h, 6 h, 8 h, 24 h and 48 h) and assayed for presence of residual antibody. The antibodies were detected by using standard immunoassays to detect the full length intact antibody. The mean amounts of antibody recovered at each collection time is shown for each treatment group (n=3). The results are expressed as mean recovery of percentage of the total amount of antibody recovered in feces divided by the total amount of ingested antibody. All animal procedures were approved by the Institutional Animal Care and Use Committee.

FIG. 33 depicts a time course of antibody recovery from feces of mice fed with 1 mg of antibody. The antibody solution was resuspended in PBS buffer at pH 7.4. IgG was first detected 2 hours after oral administration of the antibody solutions. The highest IgG recovery was observed between 2 h and 6 h. Significantly, the highest antibody recovery was obtained at all time points for the optimized antibody. The antibody concentrations were under the analytical detection limits for all groups receiving the non-optimized antibody.

FIG. 34 depicts a time course of antibody recovery from mouse feces after oral administration of 2.5 mg of the optimized antibody and a control antibody. In FIG. 34, 2.5 mg of antibody resuspended in PBS buffer pH 7.4 or in bicarbonate sodium 0.1 M pH 9.5 was orally delivered to 25 g mice. During the study, mice had free access to food and water. Feces were collected at various times (2 h, 4 h, 6 h, 8 h, 10 h, and 24 h) and assayed for presence of residual antibody. The antibodies were detected by using two different standard immunoassays to detect the full length intact antibody and the Fab fragment. All animal procedures were approved by the Institutional Animal Care and Use Committee (group size n=3).

FIG. 34 depicts the time course of antibody recovery from feces of mice fed with 2.5 mg of antibody. The antibody solutions were resuspended in PBS buffer at pH 7.4 or at pH 9.4. Similarly, the optimized antibody was detected as full length in the feces after oral administration. The highest recovery of the full length antibody was obtained at all time points for the optimized antibody whereas concentrations of the non-optimized antibody were under the analytical detection limits. Only Fab fragments of the non-optimized antibody could be recovered from feces. Interestingly, the Fab levels of the non-optimized antibody were notably increased when additional acid buffering capacity was provided in the form of bicarbonate sodium. The bicarbonate buffer protected the antibody from gastric acid degradation.

These results clearly demonstrate that using the methods of the invention pepsin and pancreatin cleavage sites can be successfully targeted within an IgG1 sequence, thus allowing the molecule to be more stable in the digestive tract. These results demonstrate that the methods of the invention can effectively generate antibodies that, because they are more stable in the digestive tract, can be used effectively in oral administration regimens.

FIG. 35 illustrates a photograph of a Western blot analysis of samples described in FIG. 34. Samples were run on a 4-12% NUPAGE™ SDS Page gel and detected with 1:1000 anti-human (Fab)′2. 400 ng was loaded per lane. Molecular weights are indicated in kDa.

### Materials and Methods

Digestibility assay of IgG1, IgG2, IgG3 and IgG4 in gastric fluids: All antibodies purchased from Calbiochem were isolated from human myelomas: IgG1 with kappa light chain (Calbiochem, Cat #400120), IgG2 with kappa light chain (Calbiochem, Cat#400122), IgG3 with lambda light chain (Calbiochem, Cat#400124), and IgG4 with lambda light chain (Calbiochem, Cat#400126). Simulated gastric fluid (SGF) was prepared fresh daily as described in the United States Pharmacopoeia. 1×SGF buffer consisted of 3.2 mg/mL pepsin (Sigma Chemical Co., St. Louis, Mo.), NaCl (2 mg/mL) at pH 1.2. Dilutions were prepared in the same buffer. A master tube was prepared in a 1.5 mL microcentrifuge tube containing 60 μg of antibody and 120 μL 0.001×SGF in a final volume of 180 μL. The reaction was incubated at 37° C. At intervals of 0, 2, 5, 10, 20, and 30 min, aliquots of 30 μL containing 10 μg of antibody were removed from the master tube and added immediately to 7 μL 4× NUPAGE™ LDS sample buffer (Invitrogen) and heated for 5 min at 100° C. Samples were subjected to SDS-PAGE using precast 4-12% Bis-Tris NUPAGE™ gels (Invitrogen, Carlsbad, Calif.). Gels were run at a 160 V for approximately 40 minutes using MES running buffer according to the manufacturer's instruction. Proteins were visualized using GELCODE™ Blue Stain Reagent (PIERCE, Rockford, Ill.). The protein MW Marker SEEBLUE PLUS2™ was purchased from Invitrogen.

Hybridoma culture: Hybridoma cell line PBA3 expressing a Clostridium difficile anti-toxin A recognizing antibody was obtained from ATCC. Cell lines were grown in DMEM (Dulbecco's Minimal Essential Medium with high glucose, Gibco/Invitrogen, Carlsbad, Calif.), 10% FBS (Sterile Fetal Bovine Serum, Sigma Chemical, St. Louis, Mo.), and 1× glutamine/penicillin/streptomycin (Gibco/Invitrogen) and cryopreserved.

Antibody gene cloning: Total RNA was isolated from 10⁷ hybridoma cells using a procedure based on the RNEASY MINI™ kit (Qiagen, Hilden, Germany). The poly-A+ RNA fraction was purified using an OLIGOTEX™ mRNA mini kit (Qiagen) and used to generate first strand cDNA (Clontech cDNA synthesis kit, Clontech Laboratories, Inc., Palo Alto, Calif.). Primers used for the amplification of the variable region from both the light chain and the heavy chains were designed as described previously (Coloma et al., 1992; Dattamajumdar et al., 1996). Primers MLALT5 and 33615 were used for amplification of the variable region from the light chain (MLALT5: 5′-CACCATGAAGTTGCCTGTTAGGCTGTTG-3′ (SEQ ID NO:10); 33615: 5′-GAAGATCTAGACTTACTATGCAGCATCAGC-3′) (SEQ ID NO:11). Primers MVG1R and MH1 were used for the amplification of the heavy chain variable region (MH1: 5′-ATATCCACCATGGRATGSAGCTGKGTMATSCTCTT-3′ (SEQ ID NO:12); MVG1R: 5′-GGCAGCACTAGTAGGGGCCAGTGGATA-3′) (SEQ ID NO:13). Sense primers (based on the FR1 region) and antisense primers (based on the 5′-end of the constant region) were then designed for both chains following sequencing of the PCR products. PCR products obtained using these primers were cloned into the modified mammalian expression vector pCEP4 (Invitrogen, Carlsbad, Calif.). The modified vector either contained the signal peptide and the constant domain region of the heavy chain or the signal peptide and the constant domain of the light chain. The constant domain of the human IgG1 was constructed by subcloning the appropriate heavy chain and light chain domains into pCEP4 from a human spleen cDNA library. The plasmid containing the light chain variable domain and its constant domain was designated BD12585. The plasmid containing the variable domain and the constant domain of heavy chain was designated BD12584.

Proteomic approach: Pepsin-digested IgG1 was submitted for proteomic analysis in an attempt to identify the pepsin cleavage sites. Because antibody fragments were still too large for analysis by tandem mass spectrometry (MS/MS) after pepsin digestion, trypsin was used to generate smaller peptides in the presence of a 1:1 mixture of 16_{O}/18_{O}, so that peptides produced with pepsin should have a normal isotopic distribution (singlet) and peptides produced from trypsin should have a modified distribution (doublet).

IgG1 mutagenesis: Site-directed mutagenesis on IgG1 was used to generate IgG1 variants in which all solvent-exposed residues in the CH1, CH2, and CH3 domains were individually altered to Ala or another residue, as specified in the list. All mutants were confirmed by DNA sequencing.

Transfection of mutant library into mammalian cells: All mutant plasmids were transformed into XL1-BLUE™ bacteria and stocked in glycerol. Plasmid DNA from every mutant was prepared as described by the manufacturer (Qiagen, endotoxin-free MAXIPREP™ kit Cat#12362). Plasmids were transfected into the adenovirus-transformed human embryonic kidney cell line 293F using 293fectin in 12-well microtiter plates and using 293F-FREESTYLE™ media for culture. Light and heavy chain plasmids were transfected at 0.5 μg/mL for each plasmid and using a 1:1 light chain plasmid versus heavy chain plasmid ratio. Supernatants were collected 7 days after transfection. Expression levels varied from approximately 0.25-1.5 μg/Ml.

Medium Scale Expression and Purification of monoclonal IgG1 from cell culture: Transfection and tissue-culture was performed as described above with the exception that 100 mL supernatants from mammalian cell cultures were collected and passed through a 0.22 μm filter. Final supernatant volumes were between 100 to 1000 mL serum-free medium. Supernatants containing antibody were applied directly to 5 mL HITRAP™ Protein G Columns (Amersham Biosciences, Piscataway, N.J., cat#17-0405-01) at 5 mL/min. Multiple passage of supernatants over the columns was unnecessary as >95% of all IgG1 material from each supernatant bound to the column on the first pass. Mobile phases consisted of 1×PBS-Tween (Sigma Aldrich, Running Buffer, cat# P-3563) and 0.1 M glycine pH 2.7 (Fisher Chemicals, Elution Buffer, cat# G48-500). Antibody collections in 0.1 M glycine were diluted 20% (v/v) with 1 M TrisHCl, pH 8.0, for neutralization. IgG1 collections were pooled and dialyzed exhaustively against 1×PBS (Pierce Slide-A-Lyzer Cassette, 3500 MWCO, cat#66110). The concentration of each IgG1 stock solution was determined by Bradford analysis (Bio-Rad protein assay, Hercules, Calif. cat#500-0006) using a commercial myeloma IgG1 stock solution (2 mg/ml—Calbiochem, cat#400120) as a standard and by UV-absorbance at 280 nm using the method of Pace and coworkers (1995).

Circular Dichroism (CD) spectroscopy: CD spectra were taken on an Aviv model 215 spectrophotometer. Far-UV scans were performed by assessing the ellipticity at every wavelength between 260 and 190 nm. A 1 nm bandwidth was used and each point was averaged for 3 seconds. The temperature was maintained at 25° C. by a Peltier cooling device coupled to a circulating water bath maintained at 20° C. All scans were performed in a 1 mm cuvette and a 1 μM IgG1 concentration. Low pH buffers were prepared by adding HCl to 10 or 100 mM phosphate solutions. The pH electrode was calibrated using pH 1.68 and 4.0 standards purchased from Fisher (pH 4, Oakton Cat#00654-00; pH 1.68, Oakton cat#00654-01).

SGF digestion stability assay: Simulated gastric fluid (SGF) was prepared fresh daily as described (Privalle et al., 2000) using 0.1×SGF buffer at pH 2 or pH 3 (3.2 mg/ml pepsin, 2 mg/ml NaCl; Sigma Chemical Co., St. Louis, Mo.). All recombinant antibodies were dialyzed into PBS and stored at 4° C. For all digestions, a master tube was prepared containing 1 μg/mL recombinant antibody and 0.0025×SGF at pH 2 and 0.005×SGF at pH 3.0. The pH of each reaction was monitored by first making appropriate dilutions of PBS with SGF and measuring the pH before and after neutralization with Tris-HCl, pH 9. Antibodies were incubated at 37° C. for intervals of 0, 2, 5, 10 and 20 min at pH 2 OR at intervals of 0, 2, 5, 10, 20, 30, 60 and 120 min at pH 3.0. The reaction was neutralized before aliquots were taken either for ELISA analysis or for SDS-Page and silver staining. SDS-Page gels were run as described above, except under reducing conditions, 10% gels provided superior separation. The amount of protein added to the gel was limited to 0.8 μg/well; therefore, protein bands were visualized using the SILVERQUEST™ Silver Staining Kit (Invitrogen cat#LC6070). 1 μg of IgG Fc and Fab standards (Pierce cat#31205 and #31203, respectively) were reduced with 100 mM DTT and added to the gel to allow for the discrimination of intact recombinant heavy chain, recombinant light chain and hinge proteolyzed recombinant Fc fragment. ELISA assays were performed as described below.

ELISA assays: Protein G (Sigma, cat# P-4689) was biotinylated using the EZ-Link Biotin-LC-ASA kit (PIERCE catalog #29982). Briefly, EZ-LINK-BIOTIN-LC-ASA™ was dissolved in DMSO and added individually to protein G at a 5:1 molar ratio. ProteinG/biotin conjugation was induced for 20 minutes under a UV lamp in a PBS buffer. Conjugated protein G was removed from unreacted biotin by application of the reaction mixture to a desalting column (PIERCE D-Salt Dextran Plastic Desalting Columns, catalog #43230). 500 μL fractions from the desalting procedure were tested for protein absorption at 280 nm to detect the presence of biotinylated protein G.

Microtiter streptavidin plates (Sigma Chemical, St. Louis, Mo., catalog #M5432) were coated with 200 ng per well of biotinylated protein G diluted into PBS buffer and incubated at 4° C. overnight. The plates were then washed 3 times with TBST buffer. All samples were diluted in Tris buffer, pH 8.0 TBST buffer (Sigma, cat#T9039). Aliquots of 100 μL of each diluted sample were transferred to the protein G-coated plates and incubated for 1-2 hours at room temperature. Following 3 washes with TBST, alkaline phosphatase-conjugated IgG heavy chain-specific mouse anti-human IgG (Zymed, cat#05-4222) was added to each well at a 1:500 dilution. The reaction was carried out for 1 hr at room temperature, the plate(s) was washed 3 times with TBST and 100 μL of p-nitrophenyl-phosphate substrate was added (Sigma, Catalog # A3469). The absorption was determined at 405 nm using a Molecular Devices vₘₐₓ kinetic microplate reader. Protein concentrations were determined using the Bradford protein assay using quantified IgG1 as the standard and/or by UV-280 absorbance.

Expression and thermotolerance analysis of constant domain mutant library: Expression of the mutant library was performed in a 12-well plate format as described above. One well of each 12-well plate was dedicated to the wildtype antibody as an internal control. The expression of each mutant variant was tested by ELISA and compared to the wildtype. The wildtype antibody begins to unfold when heated to 75° C. for 10 minutes and is completely unfolded when subjected to 80° C. for the same time period (see FIG. 29C). The unfolding is irreversible as cooling for any length of time does not result in the regeneration of signal in this ELISA format. The thermotolerance of each member of the constant domain mutant library was compared to the wildtype molecule by heating (side-by-side with the wildtype protein) to 70° C., 75° C. and 80° C. for 10 minutes. The amount of folded antibody remaining after heating was tested by ELISA.

In vitro simulated gastric and intestinal experiments: Simulated intestinal fluid (SIF) was prepared fresh daily as described in the United States Pharmacopoeia. 1×SIF buffer consisted of 10 mg/mL pancreatin, (Sigma Chemical Co., St. Louis, Mo.), and 6.8 mg/ml KH₂PO₄. A master tube was prepared in a 1.5 mL microcentrifuge tube containing 18.6 uL of sample, 70 uL of 10×SIF (10×SIF was centrifuged before use) in a final volume of 770 uL. The reaction was incubated at 37° C. At intervals of 0, 2, 10, 30, 60, 120, and 240 min, aliquots of 110 μL were removed from the master tube and 5.5 uL of PEFABLOC™ (or, 4-(2-aminoethyl)benzenesulfonylfluoride HCl) (Roche) was added immediately to halt further digestion. Antibodies were expressed in 12-well plates. The overall expression level ranged between 0.5 to 4 μg/ml. Expression varied from plate to plate, but an internal wildtype control was transfected within each plate to insure that expression level did not affect the digestion results. In general, expression was quite uniform within each plate with a standard deviation of ±26.1% of the average expression within each plate. The amount of antibody remaining after digestion was determined by quantitative ELISA. Some samples from the first round of digestion were also subjected to SDS-PAGE analysis using precast 4% to 12% Bis-Tris NUPAGE™ gels (Invitrogen, Carlsbad, Calif.) and Silver staining (SILVERQUEST™ Kit, Invitrogen). Results of the SDS-PAGE analysis correlated well with ELISA results; therefore, ELISA was used for the remaining antibody samples as it provided a more accurate quantitation of the digestion results.

ELISA detection of remaining IgG after pancreatin digestion: Microtiter streptavidin plates (Sigma Chemical, St. Louis, Mo., catalog #M5432) were coated with 200 ng per well biotinylated protein G in PBS buffer and incubated at 4° C. overnight. The plates were then washed 3 times with Tris buffered saline, pH 8.0 with Tween-20 (TBST—Sigma, cat#T9039). Aliquots of 100 μL of each antibody sample (diluted into TBST) were transferred to the protein G-coated plates and incubated for 1-2 hours at room temperature. Following 3 washes with TBST, alkaline phosphatase-conjugated goat anti-human Fab (Pierce, 31312) was added to each well at a 1:1000 dilution. The reaction was carried out for 1 hr at room temperature, the plate(s) was washed 3 times with TBST and 100 μL of p-nitrophenyl-phosphate substrate was added (Sigma, Catalog # A3469). The absorption was determined at 405 nm using a Molecular Devices uₘₐₓ kinetic microplate reader.

The contents of all documents cited above are expressly incorporated herein to the extent required to understand the invention.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. An isolated or recombinant antibody having an increased resistance to proteolysis made by a method comprising:
(a) providing an antibody having at least one protease cleavage site: and
(b) engineering at least one amino acid residue modification in the antibody, wherein the at least one amino acid residue modification(s) results in an increased resistance to proteolysis, and the at least one amino acid residue modification comprises:
(i) at least one amino acid substitution at any one or more of amino acid positions T155, L179, L235, F241, Y296, L309, Y349, L365, L398, F404, Y407 or Y436 of an IgG heavy chain;
(ii) at least one amino acid substitution at any one or more of amino acid positions L234, L242, F243, F275, Y278, Y300, L306, W313, L314, Y319, L351, L368, Y391, F405, L406, L410, F423, L432, or Y436 of an IgG heavy chain;
(iii) at least one amino acid substitution at any one or more of amino acid positions F116, K126, R143, K169 or K183 of a kappa chain;
(iv) at least one amino acid substitution at any one or more of amino acid positions K133, K205, K210, K274, K326, K340, R355, K360 or K392 of an IgG heavy chain;
(v) at least one amino acid residue modification comprising at least one amino acid substitution at a P1 or P1′ site of cleavage in a trypsin cleavage motif, wherein the substituted amino acid is K or R;
(vi) at least one amino acid substitution at a P1 or P1′ site of cleavage in a pepsin cleavage motif, wherein the substituted amino acid is L, F, Y, W, I, or T;
(vii) at least one amino acid substitution at a P1 or P1′ site of cleavage in a chymotrypsin cleavage motif, wherein the substituted amino acid is F, Y, or W;
(viii) at least one amino acid substitution selected from the group of amino acid substitutions of L235P, L398Q, F404Y, L179I, and T155S in an IgG₁ heavy chain;
(ix) at least one amino acid substitution selected from the group of amino acid substitutions of F116S and K126A in a kappa light chain;
(x) at least one amino acid substitution selected from the group of amino acid substitutions of K133G and K274Q in a IgG heavy chain; or
(xi) a combination of any of the modifications of steps (i) to (x),
wherein the numbering of the residues in the variant amino acid sequence is that of the EU index in the Kabat numbering system,
wherein optionally any one or combination of modifications of steps (i) to (x) are in a variable antibody region, a constant antibody region, or in both the variable antibody region and the constant antibody region,
and optionally the antibody comprises human antibody sequence in the constant region, human antibody sequence in the variable region or human antibody sequence in the constant and the variable region.

2. The isolated or recombinant antibody of claim 1, wherein the at least one amino acid residue modification is within a protease cleavage site in the antibody.

3. The isolated or recombinant antibody of claim 2, wherein the modification is at a P1 or P1′ residue in the protease cleavage site.

4. The isolated or recombinant isolated or recombinant antibody of claim 1, wherein the amino acid residue modification is at a site flanking a protease cleavage site in the antibody.

5. The isolated or recombinant antibody of claim 4, wherein the modification is at the P2, P3, P4, P2′, P3′, or P4′ residue of the protease cleavage site.

6. The isolated or recombinant antibody of claim 1, wherein the at least one amino acid substitution is as set forth in, or all of the combination of amino acid substitutions are as set forth in, Tables 3A or 3B, Table 4 and/or Table 5.

7. The isolated or recombinant antibody of claim 1, wherein the amino acid residue modification renders a protease cleavage site non-cleavable by the protease.

8. The isolated or recombinant antibody of claim 1, wherein the modification renders a protease cleavage site less susceptible to cleavage by the protease.

9. The isolated or recombinant antibody of claim 1, wherein at least two, three, four, five, six, seven, eight, nine, ten, eleven or more amino acid residue modifications are made.

10. The isolated or recombinant antibody of claim 9, wherein the amino acid residue modifications are in a protease cleavage site or at a site flanking the protease cleavage site, or both in a protease cleavage site and at a site flanking the protease cleavage site.

11. The isolated or recombinant antibody of claim 9, wherein the two or more amino acid residue modifications are made to the same protease cleavage site.

12. The isolated or recombinant antibody of claim 9, wherein the two or more amino acid residue modifications are made to different protease cleavage sites.

13. The isolated or recombinant antibody of claim 2, wherein the amino acid residue modification is made in a protease cleavage site that is not flanked by an amino acid residue known to inhibit or attenuate protease cleavage.

14. The isolated or recombinant antibody of claim 13, wherein the modified amino acid residue is known to inhibit or attenuate protease cleavage and is an amino acid residue selected from the group consisting of Pro, Lys, Arg and His.

15. The isolated or recombinant antibody of claim 1, wherein the antibody is an IgG, IgM, IgD, IgE, or IgA antibody.

16. The isolated or recombinant antibody of claim 15, wherein the antibody is an IgG antibody.

17. The isolated or recombinant antibody of claim 16, wherein the antibody is an IgG₁, IgG₂, IgG₃, or IgG₄ antibody.

18. The isolated or recombinant antibody of claim 1, wherein the antibody is a human antibody.

19. The isolated or recombinant antibody of claim 1, wherein the antibody is a murine, goat, rat, rabbit, camel, bovine, llama, dromedary, or simian antibody.

20. The isolated or recombinant antibody of claim 1, wherein the at least one amino acid residue modification is made in a heavy chain, a light chain or both heavy and light chains.

21. The isolated or recombinant antibody of claim 1, wherein the at least one amino acid residue modification is made in an Fc region, a hinge region, a CH_{L} domain, a CH₁ domain, a CH₂ domain, a CH₃ domain, a Fab region or a combination thereof.

22. The isolated or recombinant antibody of claim 1, wherein the at least one amino acid residue modification is made in a V_{H} or a V_{L} domain, provided the amino acid residue modification does not have a negative effect on the desired antibody function, wherein optionally the negative effect on the desired antibody function comprises a reduced affinity for antigen.

23. The isolated or recombinant antibody of claim 1, wherein the at least one amino acid residue modification comprises one mutation in the amino acid sequence of the antibody.

24. The isolated or recombinant antibody of claim 1, wherein the at least one amino acid residue modification is introduced by a modification, an addition and/or a deletion to a nucleic acid encoding the antibody.

25. The isolated or recombinant antibody of claim 24, wherein the modifications, additions or deletions to the nucleic acid encoding the antibody are introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturation Mutagenesis (GSSM), synthetic ligation reassembly (SLR) or a combination thereof.

26. The isolated or recombinant antibody of claim 24, wherein the modifications, additions or deletions to a nucleic acid encoding the antibody are introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation or a combination thereof.

27. The isolated or recombinant antibody of claim 1, wherein the at least one amino acid residue modification comprises at least one amino acid substitution at any one or more of amino acid positions T155, L179, L235, F241, Y296, L309, Y349, L365, L398, F404, Y407, and Y436 of an IgG heavy chain, wherein the amino acid position numbering is that of the EU index as in Kabat,
whereby each amino acid substitution confers to the antibody an increased resistance to pepsin proteolysis.

28. The isolated or recombinant antibody of claim 1, wherein the at least one amino acid residue modification comprises at least one amino acid substitution at any one or more of amino acid positions L234, L242, F243, F275, Y278, Y300, L306, W313, L314, Y319, L351, L368, Y391, F405, L406, L410, F423, L432, or Y436 of an IgG heavy chain, wherein the amino acid position numbering is that of the EU index as in Kabat,
whereby the amino acid substitution confers to the antibody an increased resistance to pepsin proteolysis.

29. The isolated or recombinant antibody of claim 1, wherein the at least one amino acid residue modification comprises at least one amino acid substitution at any one or more of amino acid positions F116, K126, R143, K169, K183 of a kappa chain, wherein the amino acid position numbering is that of the EU index as in Kabat,
whereby the amino acid substitution confers to the antibody an increased resistance to pancreatin proteolysis.

30. The isolated or recombinant antibody of claim 1, wherein the at least one amino acid residue modification comprises at least one amino acid substitution at any one or more of amino acid positions K133, K205, K210, K274, K326, K340, R355, K360 or K392 of an IgG heavy chain, wherein the numbering of the residues in the variant amino acid sequence is that of the EU index as in Kabat,
whereby the amino acid substitution confers to the antibody an increased resistance to pancreatin proteolysis.

31. The isolated or recombinant antibody of claim 1, wherein the at least one amino acid residue modification comprises at least one amino acid substitution at the P1 or P1′ site of cleavage in a trypsin cleavage motif, wherein the substituted amino acid is K or R,
whereby the amino acid substitution confers to the antibody an increased resistance to trypsin proteolysis.

32. The isolated or recombinant antibody of claim 1, wherein the at least one amino acid residue modification comprises at least one amino acid substitution at the P1 or P1′ site of cleavage in a pepsin cleavage motif, wherein the substituted amino acid is L, F, Y, W, I, or T,
whereby the amino acid substitution confers to the antibody an increased resistance to pepsin proteolysis.

33. The isolated or recombinant antibody of claim 1, wherein the at least one amino acid residue modification comprises at least one amino acid substitution at the P1 or P1′ site of cleavage in a chymotiypsin cleavage motif, wherein the substituted amino acid is F, Y, or W,
whereby the amino acid substitution confers to the antibody an increased resistance to chymotrypsin proteolysis.

34. The isolated or recombinant antibody of claim 1, wherein the at least one amino acid residue modification comprises at least one amino acid substitution selected from the group of amino acid substitutions of L235P, L398Q, F404Y, L179I, and T155S in an IgG₁ heavy chain, wherein the numbering of the residues in the variant amino acid sequence is that of the EU index as in Kabat,
whereby the amino acid substitution confers to the antibody an increased resistance to pepsin proteolysis.

35. The isolated or recombinant antibody of claim 1, wherein the at least one amino acid residue modification comprises at least one amino acid substitution selected from the group of amino acid substitutions of F116S and K126A in a kappa light chain, wherein the numbering of the residues in the variant amino acid sequence is that of the EU index as in Kabat,
whereby the amino acid substitution confers to the antibody an increased resistance to pepsin proteolysis.

36. The isolated or recombinant antibody of claim 1, wherein the at least one amino acid residue modification comprises at least one amino acid substitution selected from the group of amino acid substitutions of K133G and K274Q in an IgG heavy chain, wherein the numbering of the residues in the variant amino acid sequence is that of the EU index as in Kabat,
whereby the amino acid substitution confers to the antibody an increased resistance to pepsin proteolysis.

37. The isolated or recombinant antibody of the claim 1, wherein the increased resistance to proteolysis is at least 1%, 2%, 3%, 4%, 5%, 10%, 15%, 20%, 25%, 30%, 40%, 50%, 60%, 70%, 80% or 90% or more than that of the unmodified antibody.

38. The isolated or recombinant antibody of claim 1, wherein the modified antibody has greater protease-resistance than an unmodified or wildtype antibody.

39. The isolated or recombinant antibody of claim 1, wherein the modified antibody is partially or completely resistant to cleavage by more than one protease.

40. The isolated or recombinant antibody of claim 1, wherein the antibody is a humanized antibody.

41. The isolated or recombinant antibody of claim 1, wherein the antibody is a chimeric antibody.

42. The isolated or recombinant antibody of claim 1, wherein the antibody is a bispecific antibody.

43. The isolated or recombinant antibody of claim 1, wherein the antibody is a fusion protein.

44. The isolated or recombinant antibody of claim 1, wherein the antibody is a biologically active (antigen binding) fragment thereof.

45. The isolated or recombinant antibody of claim 1, wherein the modification comprises the addition of a post-translational modification site.

46. The isolated or recombinant antibody of claim 1, wherein the modification comprises the addition of an N-glycosylation site or an O-glycosylation site.

47. The isolated or recombinant antibody of claim 1, wherein the modification comprises the addition of an alkyl chain or a small molecule.

48. The isolated or recombinant antibody of claim 1, wherein the modification comprises covalent or non-covalent addition of a second molecule to the Fc chain of the antibody.

49. The isolated or recombinant antibody of claim 1, wherein the second molecule comprises an antibody secretory component.

50. The isolated or recombinant antibody of claim 1, wherein the second molecule comprises a carbohydrate.

51. The isolated or recombinant antibody of claim 1, wherein the modification comprises the addition of a disulfide bond site or a salt bridge site.

52. The isolated or recombinant antibody of claim 1, wherein the Fc region of the antibody is further modified to abrogate, diminish or enhance an Fc-mediated antibody-mediated cytotoxicity (ADCC), a complement-mediated cytotoxicity (CDC), complement activation, Fc receptor activation and/or binding or phagocytosis.

53. The isolated or recombinant antibody of claim 1, wherein the Fc region of the antibody is further modified to increase binding affinity to the Fc receptor (FcR).

54. The isolated or recombinant antibody of claim 1, wherein the antibody is further modified to have
a) an antigen binding activity comparable to or superior to the unmodified antibody;
b) a chemical stability comparable to or superior to the unmodified antibody;
c) a thermostability or thermotolerance comparable to or superior to the unmodified antibody;
d) a pH tolerance comparable to or superior to the unmodified antibody;
e) a reduced immunogenicity;
f) a reduced aggregation;
g) an increased half-life relative to the unmodified antibody;
h) an increased expression in a host cell;
i) a stability in pharmaceutical formulation comparable or superior to that of the unmodified antibody;
j) an enhanced dimerization of Fc regions;
k) an increased solubility relative to the unmodified antibody; or
l) a combination thereof.

55. The antibody of claim 1, wherein the modified antibody has
a) an antigen binding activity comparable to or superior to the unmodified antibody;
b) a chemical stability comparable to or superior to the unmodified antibody;
c) a thermostability or thermotolerance comparable to or superior to the unmodified antibody;
d) a pH tolerance comparable to or superior to the unmodified antibody;
e) a reduced immunogenicity;
f) a reduced aggregation;
g) an increased half-life relative to the unmodified antibody;
h) an increased expression in a host cell;
i) a stability in pharmaceutical formulation comparable or superior to that of the unmodified antibody;
j) an enhanced dimerization of Fc regions; or
k) a combination thereof.

56. The isolated or recombinant antibody of claim 54 or 55, wherein the antibody maintains its native conformation at about pH 3 and above.

57. The isolated or recombinant antibody of claim 54 or 55, wherein the antibody retains biological activity in conditions comprising at least pH 3, pH 3.5, pH 4, pH 4.5, pH 5 or pH 5.5.

58. The isolated or recombinant antibody of claim 1, wherein the antibody further comprises additional amino acid residue mutations that render the antibody more resistant to pH dependent unfolding.

59. The antibody of claim 1, wherein the proteolysis is mediated by proteases from the gastrointestinal track, the blood or the bile.

60. The isolated or recombinant antibody of claim 1, wherein the proteolysis is mediated by pepsin.

61. The isolated or recombinant antibody of claim 1, wherein the proteolysis is mediated by pancreatin.

62. The isolated or recombinant antibody of claim 1, wherein the proteolysis is mediated by trypsin, trypsinogen, chymo-trypsinogen, carboxy-peptidase, pro-carboxy-peptidase, elastase, pro-elastase or any combination thereof.

63. The isolated or recombinant antibody of claim 1, wherein the protease is released by an organism within the digestive tract or produced within the digestive tract.

64. The isolated or recombinant antibody of claim 1, wherein the protease is selected from a group of proteases released by an injured, an abnormal, an infected, a cancerous or otherwise diseased or abnormal tissue.

65. The isolated or recombinant antibody of claim 1, wherein the antibody specifically binds to a pathogen.

66. The isolated or recombinant antibody of claim 65, wherein the pathogen is selected from the group consisting of a bacteria, a virus and a fungus.

67. The isolated or recombinant antibody of claim 65, wherein the pathogen is an intestinal pathogen.

68. The isolated or recombinant antibody of claim 67, wherein the intestinal pathogen is selected from the group consisting of enterotoxigenic E. coli, rotavirus, Cryptosporidium parvum, Clostridium difficile, Shigella flexneri, Enterococcus faecalis, Enterococcus faecium, Campylobacter jejuni, Staphylococcus aureus, E. coli O157:H7, Helicobacter pylori, Pseudomonas aeruginosa, Shigella dysenteriae, Salmonella enteritidis, Salmonella typhi, Clostridium perfringens, Aeromonas hydrophila, and Aeromanas aerolysin.

69. The isolated or recombinant antibody of claim 65, wherein the pathogen is Streptococcus mutans.

70. The isolated or recombinant antibody of claim 1, wherein the antibody specifically binds to a toxin.

71. The isolated or recombinant antibody of claim 70, wherein the toxin is selected from the group consisting of a bacterial toxin, a chemical toxin and an environmental toxin.

72. The isolated or recombinant antibody of claim 71, wherein the bacterial toxin is selected from the group consisting of a cholera toxin, an Escherichia coli toxin, a Streptococcus toxin, a Bordetella pertussis toxin, and a Clostridium toxin.

73. The isolated or recombinant antibody of claim 72, wherein the Clostridium toxin comprises a botulinum toxin or a Clostridium difficile toxin.

74. The isolated or recombinant antibody of claim 73, wherein the botulinum toxin or Clostridium difficile toxin comprises botulinum neurotoxin, C. difficile toxin A, or C. difficile toxin B.

75. The isolated or recombinant antibody of claim 1, wherein the antibody binds a virulence factor.

76. The isolated or recombinant antibody of claim 75, wherein the virulence factor is an adherence factor, a coat protein, an invasion factor, a capsule, an exotoxin, or an endotoxin.

77. The isolated or recombinant antibody of claim 1, wherein the antibody specifically binds to a dietary enzyme.

78. The isolated or recombinant antibody of claim 77, wherein the dietary enzyme is a lipase, an esterase, a urease, a lyase, a protease, an isomerase, a ligase or a synthetase.

79. An isolated or recombinant nucleic acid comprising a sequence encoding the antibody of claim 1.

80. A vector comprising the nucleic acid of claim 79.

81. A cell comprising the nucleic acid of claim 79 or the vector of claim 80.

82. A pharmaceutical composition comprising an antibody as set forth in claim 1, and a suitable excipient.

83. The pharmaceutical composition of claim 82, wherein the composition is formulated as a suspension, a liquid, a capsule, a tablet, a gel, a microsphere, a liposome, a powder, a multiparticulate core particle or a spray.

84. The pharmaceutical composition of claim 82, wherein the antibody comprises from about 1%, 5%, 10%, 20%, 30%, 40%, 50%, 60%, 70%, 80%, 90% or 95% or more, or from about 50% to about 95%, of the batch size (weight/weight), or from about 50% to about 95% of the batch size (weight/weight).

85. The pharmaceutical composition of claim 82, wherein the composition is formulated for oral or enteric delivery or delivery as a suspension, a liquid, a capsule, a tablet, a gel, a microsphere, a liposome, a multipaiticulate core particle or a spray.

86. The pharmaceutical composition of claim 85, further comprising an enteric coating or encapsulation into gelatin capsules or liposomes, or futher comprising formulation as a pre-liposome formulation.

87. A method of ameliorating, treating or preventing gastrointestinal infections or other disorders caused by a pathogen or a toxin comprising administering orally a pharmaceutically effective amount of the antibody of claim 1, or the pharmaceutical composition of claim 82, to a subject in need thereof, whereby the infection or other disorders is treated or prevented.

88. A kit for ameliorating or preventing one or more symptoms of virulence factor-associated symptom or disease, comprising
a) the pharmaceutical composition of claim 82; and
b) instructions for administering the pharmaceutical composition.

89. A method of identifying a protease cleavage site in an antibody, which method comprises the steps of:
a) determining putative sites of protease cleavage in the antibody;
b) prioritizing the protease cleavage sites based on the likely exposure of the site to proteases; and
c) identifying a site as the protease cleavage site as one whose position results in an exposure to proteases in the three-dimensional antibody structure.

90. The method of claim 89, wherein the putative sites of protease cleavage are determined in step (a) by identifying protease cleavage motifs using N-terminal sequencing, gel electrophoresis analysis, or mass spectral analysis of peptide fragments derived from an antibody digested by protease.

91. The method of claim 89, wherein the putative sites of protease cleavage are determined in step (a) by identifying known protease motifs.

92. The method of claim 89, wherein the protease cleavage sites are prioritized in step (b) based on (i) the surface exposure on the folded form of the antibody solved by x-ray crystallography, (ii) the surface exposure on the folded form of the antibody solved by NMR spectroscopy, or (iii) the surface exposure determined using a probe of 1.4 angstroms.

93. The method of claim 89, wherein the identified protease cleavage site has 20% surface area exposure to the probe, wherein the protease cleavage site comprises hydrophobic and aromatic amino acids.

94. The method of claim 89, wherein the identified protease cleavage site has 35% surface area exposure to the probe, wherein the protease cleavage site comprises basic amino acids.

95. The method of claim 89, wherein at least one protease cleavage site is identified.

96. The method of claim 95, wherein the protease cleavage sites comprise the same protease cleavage motif.

97. The method of claim 96, wherein the protease cleavage sites comprise two or more different protease cleavage motifs.

98. The method of claim 95, wherein the at least one protease cleavage site identified is in the Fc region, the Fab region, the hinge region, CL, CH₁, CH₂, CH₃, V_{L}, V_{H}, or a combination thereof.

99. The method of claim 85, wherein the protease cleavage motif is for a protease selected from the group consisting of pepsin, pancreatin, trypsin, trypsinogen, chymotrypsin, pro-carboxy-peptidase and pro-elastase.

100. A computer implemented method for executing one or more or all of the steps of the method of claim 89.

101. A computer comprising a machine-readable medium including machine-executable instructions and systems to practice the method of claim 89, or the computer implemented method of claim 100.

102. A method of engineering a protease-resistant antibody, which method comprises the steps of:
a) providing an antibody or an amino acid sequence of the antibody;
b) identifying at least one protease cleavage site in the amino acid sequence of the antibody; and
c) introducing at least one modification in the amino acid sequence of the antibody, whereby the modification results in a variant amino acid sequence that has an increased resistance to proteolysis.

103. A method of generating an engineered antibody that is orally deliverable, which method comprises the steps of:
a) providing a nucleic acid encoding a wildtype antibody;
b) introducing at least one mutation into the coding sequence of the wildtype antibody to generate a modified antibody coding sequence, wherein the mutation of the coding sequence is in or proximate to the coding sequence of at least one protease cleavage site and the mutation results in expression of an antibody that is partially or completely resistant to digestion by the at least one protease; and
c) expressing the mutated antibody coding sequence of step b) to generate an engineered antibody,
wherein the engineered antibody retains its ability to specifically bind to antigen in the digestive system following oral administration, thereby rendering the engineered antibody orally deliverable.

104. The method of claim 102 or 103, wherein the modification is in a protease cleavage site.

105. The method of claim 104, wherein the modification is at the P1 or P1′ residue of the protease cleavage site.

106. The method of claim 102 or 103, wherein the modification is at a site flanking the protease cleavage site.

107. The method of claim 106, wherein the modification is at the P2, P3, P4, P2′, P3′, or P4′, residue of the protease cleavage site.

108. The method of claim 102 or 103, wherein the modification generates a protease resistance motif.

109. The method of claim 102 or 103, wherein the modification renders a protease cleavage site non-cleavable by the protease.

110. The method of claim 102 or 103, wherein the modification renders a protease cleavage site less susceptible to cleavage by the protease.

111. The method of claim 102 or 103, wherein the variant amino acid sequence comprises two, three, four, five, six, seven, eight nine, ten, eleven, or more amino acid residue modifications.

112. The method of claim 111, wherein the modifications are in a protease cleavage site or at a site flanking the protease cleavage site.

113. The method of claim 102 or 103, wherein the modification is made to the same protease cleavage motifs.

114. The method of claim 102 or 103, wherein the modification is made to different protease cleavage motifs.

115. The method of claim 104, wherein the modification is made in a protease cleavage site that is not flanked by an amino acid residue known to inhibit or attenuate protease cleavage.

116. The method of claim 115, wherein the amino acid residue known to inhibit or attenuate protease cleavage is an amino acid residue selected from the group consisting of Pro, Lys, Arg and His.

117. The method of claim 102 or 103, wherein the antibody is an IgG, IgM, IgD, IgE, or IgA antibody.

118. The method of claim 117, wherein the antibody is an IgG antibody.

119. The method of claim 118, wherein the antibody is an IgG₁, IgG₂, IgG₃, or IgG₄ antibody.

120. The method of claim 102 or 103, wherein the antibody is a human antibody.

121. The method of claim 102 or 103, wherein the antibody is a murine, rat, rabbit, camel, bovine, llama, dromedary, or simian antibody.

122. The method of claim 102 or 103, wherein the valiant amino acid sequence is a heavy chain, a light chain, or both chains.

123. The method of claim 102 or 103, wherein the variant amino acid sequence is in an Fc region, a hinge region, a CH_{L} domain, a CH₁ domain, a CH₂ domain, a CH₃ domain, a Fab region or a combination thereof.

124. The method of claim 102 or 103, wherein the variant amino acid sequence is a V_{H} or V_{L} domain, provided the cleavage site does not have a negative effect on the desired antibody function.

125. The method of claim 102 or 103, wherein the modification comprises at least one mutation in the amino acid sequence of the antibody.

126. The method of claim 125, wherein the mutation is introduced by modifications, additions or deletions to a nucleic acid encoding the antibody.

127. The method of claim 126, wherein the modifications, additions or deletions to a nucleic acid encoding the antibody are introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturation Mutagenesis (GSSM), synthetic ligation reassembly (SLR) or a combination thereof.

128. The method of claim 126, wherein the modifications, additions or deletions to a nucleic acid encoding the antibody are introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, or a combination thereof.

129. The method of claim 102 or 103, wherein the variant amino acid sequence comprises at least one amino acid substitution at any one or more of amino acid positions T155, L179, L235, F241, Y296, L309, Y349, L365, L398, F404, Y407, and Y436 of an IgG heavy chain, wherein the numbering of the residues in the variant amino acid sequence is that of the EU index as in Kabat,
whereby the amino acid substitution confers increased resistance to pepsin proteolysis.

130. The method of claim 102 or 103, wherein the variant amino acid sequence comprises at least one amino acid substitution at any one or more of amino acid positions L234, L242, F243, F275, Y278, Y300, L306, W313, L314, Y319, L351, L368, Y391, F405, L406, L410, F423, L432, or Y436 of an IgG heavy chain, wherein the numbering of the residues in the variant amino acid sequence is that of the EU index as in Kabat,
whereby the amino acid substitution confers increased resistance to pepsin proteolysis.

131. The method of claim 102 or 103, wherein the variant amino acid sequence comprises at least one amino acid substitution at any one or more of amino acid positions F116, K126, R143, K169 or K183 of a kappa chain, wherein the numbering of the residues in the variant amino acid sequence is that of the EU index as in Kabat,
whereby the amino acid substitution confers increased resistance to pancreatin proteolysis.

132. The method of claim 102 or 103, wherein the variant amino acid sequence comprises at least one amino acid substitution at any one or more of amino acid positions K133, K205, K210, K274, K326, K340, R355, K360 or K392 of an IgG heavy chain, wherein the numbering of the residues in the variant amino acid sequence is that of the EU index as in Kabat,
whereby the amino acid substitution confers increased resistance to pancreatin proteolysis.

133. The method of claim 102 or 103, wherein the variant amino acid sequence comprises at least one amino acid substitution at the P1 or P1′ site of cleavage in a trypsin cleavage motif, wherein the substituted amino acid is K or R,
whereby the amino acid substitution confers increased resistance to trypsin proteolysis.

134. The method of claim 102 or 103, wherein the variant amino acid sequence comprises at least one amino acid substitution,
at the P1 or P1′ site of cleavage in a pepsin cleavage motif, wherein the substituted amino acid is L, F, Y, W, I, or T,
whereby the amino acid substitution confers increased resistance to pepsin proteolysis.

135. The method of claim 102 or 103, wherein the variant amino acid sequence comprises at least one amino acid substitution at the P1 or P1′ site of cleavage in a chymotrypsin cleavage motif, wherein the substituted amino acid is F, Y, or W,
whereby the amino acid substitution confers increased resistance to chymotrypsin proteolysis.

136. The method of claim 102 or 103, wherein the variant amino acid sequence comprises at least one amino acid substitution selected from the group of amino acid substitutions of L235P, L398Q, F404Y, L179I, and T155S in an IgG₁ heavy chain, wherein the numbering of the residues in the variant amino acid sequence is that of the EU index as in Kabat,
whereby the amino acid substitution confers increased resistance to pepsin proteolysis.

137. The method of claim 102 or 103, wherein the variant amino acid sequence comprises at least one amino acid substitution selected from the group of amino acid substitutions of F116S and K126A in a kappa light chain, wherein the numbering of the residues in the variant amino acid sequence is that of the EU index as in Kabat,
whereby the amino acid substitution confers increased resistance to pepsin proteolysis.

138. The method of claim 102 or 103, wherein the variant amino acid sequence comprises at least one amino acid substitution selected from the group of amino acid substitutions of K133G and K274Q in an IgG heavy chain, wherein the numbering of the residues in the variant amino acid sequence is that of the EU index as in Kabat,
whereby the amino acid substitution confers increased resistance to pepsin proteolysis.

139. The method of the claim 102 or 103, wherein the increased resistance to proteolysis is at least 1%, 2%, 3%, 4%, 5%, 10%, 15%, 20%, 25%, 30%, 40%, 50%, 60%, 70%, 80% or more than that of the unmodified antibody.

140. The method of claim 102 or 103, wherein the modified antibody has greater protease-resistance than a wildtype antibody.

141. The method of claim 102 or 103, wherein the modified antibody is partially or completely resistant to cleavage by more than one protease.

142. The method of claim 102 or 103, wherein the antibody is a humanized antibody.

143. The method of claim 102 or 103, wherein the antibody is a chimeric antibody.

144. The method of claim 102 or 103, wherein the antibody is a bispecific antibody.

145. The method of claim 102 or 103, wherein the antibody is a fusion protein.

146. The method of claim 102 or 103, wherein the antibody is a biologically active (antigen binding) fragment thereof.

147. The method of claim 102 or 103, wherein the modification comprises the addition of a post-translational modification site.

148. The method of claim 102 or 103, wherein the modification comprises the addition of an N-glycosylation site or an O-glycosylation site.

149. The method of claim 102 or 103, wherein the modification comprises the addition of an alkyl chain or a small molecule.

150. The method of claim 102 or 103, wherein the modification comprises covalent or non-covalent addition of a second molecule to the Fc chain of the antibody.

151. The method of claim 102 or 103, wherein the second molecule comprises an antibody secretory component.

152. The method of claim 102 or 103, wherein the second molecule comprises a carbohydrate.

153. The method of claim 102 or 103, wherein the modification comprises the addition of a disulfide bond site or a salt bridge site.

154. The method of claim 102 or 103, wherein the Fc region of the antibody is further modified to abrogate, diminish or enhance an Fc-mediated antibody-mediated cytotoxicity (ADCC), a complement-mediated cytotoxicity (CDC), complement activation, Fc receptor activation and/or binding or phagocytosis.

155. The method of claim 102 or 103, wherein the Fc region of the antibody is further modified to increase binding affinity to the Fc receptor (FcR).

156. The method of claim 102 or 103, wherein the antibody is further modified to have
a) an antigen binding activity comparable to or superior to the unmodified antibody;
b) a chemical stability comparable to or superior to the unmodified antibody;
c) a thermostability or thermotolerance comparable to or superior to the unmodified antibody;
d) a pH tolerance comparable to or superior to the unmodified antibody;
e) a reduced immunogenicity;
f) a reduced aggregation;
g) an increased half-life relative to the unmodified antibody;
h) an increased expression in a host cell;
i) a stability in pharmaceutical formulation comparable or superior to that of the unmodified antibody;
j) an enhanced dimerization of Fc regions; or
k) some combination thereof.

157. The method of claim 102 or 103, wherein the modified antibody has
a) an antigen binding activity comparable to or superior to the unmodified antibody;
b) a chemical stability comparable to or superior to the unmodified antibody;
c) a thermostability or thermotolerance comparable to or superior to the unmodified antibody;
d) a pH tolerance comparable to or superior to the unmodified antibody;
e) a reduced immunogenicity;
f) a reduced aggregation;
g) an increased half-life relative to the unmodified antibody;
h) an increased expression in a host cell;
i) a stability in pharmaceutical formulation comparable or superior to that of the unmodified antibody;
j) an enhanced dimerization of Fc regions; or
k) some combination thereof.

158. The method of claim 156 or 157, wherein the antibody maintains its native conformation at about pH 3 and above.

159. The method of claim 156 or 157, wherein the antibody retains biological activity at pH 3.

160. The method of claim 102 or 103, wherein the antibody further comprises additional mutations that render the antibody more resistant to pH dependent unfolding.

161. The method of claim 102 or 103, wherein the proteolysis is the digestion mediated by proteases from the gastrointestinal track, the blood, or the bile.

162. The method of claim 102 or 103, wherein the proteolysis is mediated by pepsin.

163. The method of claim 102 or 103, wherein the proteolysis is mediated by pancreatin.

164. The method of claim 102 or 103, wherein the proteolysis is mediated by trypsin, trypsinogen, chymo-trypsinogen, carboxy-peptidase, pro-carboxy-peptidase, elastase, pro-elastase, or some combination thereof.

165. The method of claim 102 or 103, wherein the protease is released by an exogenous organism or produced within the digestive tract.

166. The method of claim 102 or 103, wherein the protease is selected from a group of proteases released by an abnormal, infected, cancerous or otherwise diseased tissue.

167. The method of claim 102 or 103, wherein the antibody specifically binds to a pathogen.

168. The method of claim 167, wherein the pathogen is selected from the group consisting of a bacteria, a virus and a fungus.

169. The method of claim 167, wherein the pathogen is an intestinal pathogen.

170. The method of claim 169, wherein the intestinal pathogen is selected from the group consisting of enterotoxigenic E. coli, rotavirus, Cryptosporidium parvum, Clostridium difficile, Shigella flexneri, Enterococcus faecalis, Enterococcus faecium, Campylobacter jejuni, Staphylococcus aureus, E. coli O157:H7, Helicobacter pylori, Pseudomonas aeruginosa, Shigella dysenteriae, Salmonella enteritidis, Salmonella typhi, Clostridium perfringens, Aeromonas hydrophila, and Aeromanas aerolysin.

171. The method of claim 167, wherein the pathogen is Streptococcus mutans.

172. The method of claim 102 or 103, wherein the antibody specifically binds to a toxin.

173. The method of claim 172, wherein the toxin is selected from the group consisting of a bacterial toxin, a chemical toxin and an environmental toxin.

174. The method of claim 173, wherein the bacterial toxin is selected from the group consisting of a cholera toxin, an Escherichia coli toxin, a Streptococcus toxin, a Bordetella pertussis toxin, and a Clostridium toxin.

175. The method of claim 174, wherein the Clostridium toxin comprises a botulinum toxin or a Clostridium difficile toxin.

176. The method of claim 175, wherein the botulinum toxin or Clostridium difficile toxin comprises botulinum neurotoxin, C. difficile toxin A, or C. difficile toxin B.

177. The method of claim 102 or 103, wherein the antibody binds a virulence factor.

178. The method of claim 177, wherein the virulence factor is an adherence factor, a coat protein, an invasion factor, a capsule, an exotoxin, or an endotoxin.

179. The method of claim 102 or 103, wherein the antibody specifically binds to a dietary enzyme.

180. The method of claim 179, wherein the dietary enzyme is a lipase, an esterase, a urease, a lyase, a protease, an isomerase, a ligase or a synthetase.

181. An isolated or recombinant nucleic acid comprising a sequence encoding an antibody made by a method as set forth in claim 102 or 103.

182. A vector comprising the nucleic acid of claim 181.

183. A cell comprising the nucleic acid of claim 181 or the vector of claim 182.

184. A pharmaceutical composition comprising the antibody produced by the method of claim 102 or 103, and a suitable excipient.

185. The pharmaceutical composition of claim 184, wherein the composition is formulated as a suspension, a liquid, a capsule, a tablet, a gel, a microsphere, a liposome, a multiparticulate core particle or a spray.

186. The pharmaceutical composition of claim 184, wherein the antibody comprises from about 1%, 5%, 10%, 20%, 30%, 40%, 50%, 60%, 70%, 80%, 90% or 95% or more of the batch size (weight/weight), or from about 50% to about 95%, of the batch size (weight/weight).

187. The pharmaceutical composition of claim 184, wherein the composition is formulated for enteric delivery or delivery as a suspension, a liquid, a capsule, a tablet, a gel, a microsphere, a liposome, a muitipaiticulate core particle or a spray.

188. The pharmaceutical composition of claim 184, further comprising an enteric coating or encapsulation into gelatin capsules or liposomes, or further comprising formulation as a pre-liposome formulation.

189. A method of ameliorating, treating or preventing gastrointestinal infections or other disorders caused by a pathogen or a toxin comprising administering orally a pharmaceutically effective amount of the antibody of claim 102 or 103, or the pharmaceutical composition of claim 184, to a subject in need thereof, whereby the infection or other disorders is treated or prevented.

190. A kit for ameliorating or preventing one or more symptoms of virulence factor-associated symptom or disease, comprising
a) the pharmaceutical composition of claim 184; and
b) instructions for administering the pharmaceutical composition.

191. A method to ameliorate or prevent toxicity associated with Clostridium difficile, comprising administering to a subject in need thereof
a) a therapeutically effective amount of a first monoclonal antibody, wherein the first monoclonal antibody comprises the heavy chain variable region sequence of SEQ ID NO:1 and the light chain variable region sequence of SEQ ID NO:2; and
b) a therapeutically effective amount of a second monoclonal antibody, wherein the second monoclonal antibody comprising the heavy chain variable region sequence of SEQ ID NO:3 and the light chain variable region sequence of SEQ ID NO:4,
whereby the antibodies ameliorate or prevent the toxicity associated with Clostridium difficile toxin A,
and optionally the first and/or the second monoclonal antibody is a chimeric, or humanized, antibody comprising human constant region sequence.

192. The method of claim 195, further comprising administering a third monoclonal antibody, wherein the third antibody is a monoclonal antibody comprising the heavy chain variable region sequence of SEQ ID NO:5 and the light chain variable region sequence of SEQ ID NO:6, whereby the antibodies ameliorate or prevent the toxicity associated with Clostridium difficile toxin B,
and optionally the monoclonal antibody is a chimeric, or humanized, antibody comprising human constant region sequence.

193. A method of ameliorating or preventing toxicity associated with Clostridium difficile, comprising administering to a subject in need thereof
a) a first antibody that partially or completely inhibits binding of a Clostridium difficile toxin A to a cell, and
b) a second antibody that partially or completely inhibits intracellular internalization of the Clostridium difficile toxin A, wherein the first antibody and the second antibody bind to the Clostridium difficile toxin A at non-overlapping epitopes.

194. The method of claim 193, further comprising administering a therapeutically effective amount of a third antibody that partially or completely neutralizes Clostridium difficile toxin B.

195. The method of claim 193, wherein the second antibody is not PCG-4.

196. The method of claim 193, wherein the first and second antibodies synergize to neutralize the virulence factor at an antibody concentration lower than the antibody concentration necessary to observe partial neutralization by each antibody alone.

197. The method of claim 193, wherein the first monoclonal antibody and the second monoclonal antibody bind to a Clostridium difficile toxin A at ToxA:1800-2710.

198. The method of claim 194, wherein the third antibody is a monoclonal antibody that binds to a Clostridium difficile toxin B at ToxB:1807-2366.

199. The method of claim 194, wherein the first monoclonal antibody and the second monoclonal antibody do not bind Clostridium difficile toxin B, and the third monoclonal antibody does not bind Clostridium difficile toxin A.

200. The method of any one of claim 191, 192, 193 or 194, wherein the monoclonal antibodies comprise recombinant or synthetic antibodies.

201. The method of any one of claim 191, 192, 193 or 194, wherein the Clostridium toxin-related toxicity in the subject comprises Clostridium-associated diarrhea, colitis or a related condition, and whereby one or more symptoms of the Clostridium-induced diarrhea, colitis, or related condition are ameliorated or prevented following administration of the monoclonal antibodies.

202. The method of any one of claim 191, 192, 193 or 194, wherein at least one of the antibodies is rendered partially or completely protease-resistant by the method of claim 102 or 103.

203. The method of any one of claim 191, 192, 193 or 194, wherein at least one of the antibodies is rendered orally deliverable by the method of claim 103.

204. The method of any one of claims 191, 192, 193 or 194, wherein at least one of the antibodies is a humanized antibody, chimeric antibody, bispecific antibody, fusion antibody, nanobody, diabody, triabody, scFv or biologically active fragment thereof.

205. The method of any one of claims 191, 192, 193 or 194, wherein at least one of the antibodies is a human, murine, rat, rabbit, camel, llama, dromedary, or simian antibody.

206. The method of any one of claims 191, 192, 193 or 194, wherein the Fc region of at least one of the antibodies is further modified to abrogate, diminish or enhance an Fc-mediated antibody-mediated cytotoxicity (ADCC), a complement-mediated cytotoxicity (CDC), complement activation, Fc receptor activation and/or binding or phagocytosis.

207. The method of any one of claims 191, 192, 193 or 194, wherein the Fc region of at least one of the antibodies is further modified to increase binding affinity to the Fc receptor (FcR).

208. The method of any one of claims 191, 192, 193 or 194, wherein at least one of the antibodies is further modified to have:
a) an antigen binding activity comparable to or superior to the unmodified antibody;
b) a chemical stability comparable to or superior to the unmodified antibody;
c) a thermostability or thermotolerance comparable to or superior to the unmodified antibody;
d) a pH tolerance comparable to or superior to the unmodified antibody;
e) a reduced immunogenicity;
f) a reduced aggregation;
g) an increased half-life relative to the unmodified antibody;
h) an increased expression in a host cell;
i) a stability in pharmaceutical formulation comparable or superior to that of the unmodified antibody;
j) an enhanced dimerization of Fc regions; or
k) some combination thereof.

209. The method of any one of claims 191, 192, 193 or 194, wherein at least one of the antibodies has:
a) an antigen binding activity comparable to or superior to the unmodified antibody;
b) a chemical stability comparable to or superior to the unmodified antibody;
c) a thermostability or thermotolerance comparable to or superior to the unmodified antibody;
d) a pH tolerance comparable to or superior to the unmodified antibody;
e) a reduced immunogenicity;
f) a reduced aggregation;
g) an increased half-life relative to the unmodified antibody;
h) an increased expression in a host cell;
i) a stability in pharmaceutical formulation comparable or superior to that of the unmodified antibody;
j) an enhanced dimerization of Fc regions; or
k) some combination thereof.

210. A monoclonal antibody, or a biologically active (antigen binding) fragment thereof, that binds to Clostridium difficile toxin A, wherein the variable region sequences of the antibody comprise SEQ ID NO:1 and SEQ ID NO:2; or, SEQ ID NO:3 and SEQ ID NO:4,
and optionally the monoclonal antibody is a chimeric, or humanized, antibody comprising human constant region sequence.

211. A monoclonal antibody, or a biologically active (antigen binding) fragment thereof, that binds to Clostridium difficile toxin B, wherein the variable region sequences of the antibody comprise SEQ ID NO:5 and SEQ ID NO:6,
and optionally the monoclonal antibody is a chimeric, or humanized, antibody comprising human constant region sequence.

212. The antibody of claim 210 or 211, wherein the antibody is an IgG antibody.

213. The antibody of claim 210 or 211, wherein the antibody is a human, murine, rat, rabbit, camel, llama, dromedary, or simian antibody.

214. The antibody of claim 210 or 211, wherein the antibody is a humanized antibody, chimeric antibody, bispecific antibody, fusion antibody, nanobody, diabody, triabody, scFv or biologically active fragment thereof.

215. The antibody of claim 210 or 211, wherein the antibody is modified to increase resistance to proteolysis.

216. The antibody of claim 215, wherein the antibody is modified by the method of claim 102.

217. The antibody of claim 210 or 211, wherein the antibody is modified to be orally deliverable.

218. The antibody of claim 217, wherein the antibody is modified by the method of claim 103.

219. The antibody of claim 210 or 211, wherein the antibody is modified to abrogate, diminish or enhance antibody-mediated cytotoxicity (ADCC), a complement-mediated cytotoxicity (CDC), complement activation, Fc receptor activation and/or binding or phagocytosis.

220. The antibody of claim 210 or 211, wherein the Fc region of the antibody is modified to abrogate, diminish or enhance (increase) binding affinity to the Fc receptor (FcR).

221. The antibody of claim 210 or 211, wherein the antibody is modified to have: a) an antigen binding activity comparable to, less than or superior to the unmodified antibody; b) a chemical stability comparable to, less than or superior to the unmodified antibody; c) a thermostability or thermotolerance comparable to, less than or superior to the unmodified antibody; d) a pH tolerance comparable to, less than or superior to the unmodified antibody; e) an abrogated, diminished or enhanced (increased) immunogenicity; f) an abrogated, diminished or enhanced (increased) ability to aggregate; g) an increased or decreased half-life relative to the unmodified antibody; h) an increased or decreased expression in a host cell; i) a stability in pharmaceutical formulation comparable or superior to that of the unmodified antibody (an abrogated, diminished or enhanced (increased) stability in pharmaceutical formulation); j) an abrogated, diminished or enhanced (increased) dimerization of Fc regions; or k) any combination thereof.

222. The antibody of claim 210 or 211, wherein the antibody has: a) an antigen binding activity comparable to, less than or superior to the unmodified antibody; b) a chemical stability comparable to, less than or superior to the unmodified antibody; c) a thermostability or thermotolerance comparable to, less than or superior to the unmodified antibody; d) a pH tolerance comparable to, less than or superior to the unmodified antibody; e) an abrogated, diminished or enhanced (increased) immunogenicity; f) an abrogated, diminished or enhanced (increased) ability to aggregate; g) an increased or decreased half-life relative to the unmodified antibody; h) an increased or decreased expression in a host cell; i) a stability in pharmaceutical formulation comparable or superior to that of the unmodified antibody (an abrogated, diminished or enhanced (increased) stability in pharmaceutical formulation); j) an abrogated, diminished or enhanced (increased) dimerization of Fc regions; or k) any combination thereof.

223. An isolated or recombinant nucleic acid comprising a sequence encoding the antibody of claim 210 or 211.

224. A vector comprising the nucleic acid of claim 223.

225. A cell comprising the nucleic acid of claim 223 or the vector of claim 224.

226. A pharmaceutical composition comprising the antibody of claim 210 or 211, and a suitable excipient.

227. The pharmaceutical composition of claim 226, wherein the composition is formulated as a suspension, a liquid, a capsule, a tablet, a gel, a microsphere, a liposome, a multiparticulate core particle or a spray.

228. The pharmaceutical composition of claim 226, wherein the antibody comprises from about 1%, 5%, 10%, 20%, 30%, 40%, 50%, 60%, 70%, 80%, 90% or 95% or more, or from about 50% to about 95%, of the batch size (weight/weight).

229. The pharmaceutical composition of claim 226, wherein the composition is formulated for enteric delivery.

230. The pharmaceutical composition of claim 226, further comprising an enteric coating.

231. A kit for ameliorating or preventing one or more symptoms of Clostridium difficile-associated toxicity, comprising
a) the pharmaceutical composition of claim 226; and
b) instructions for administering the pharmaceutical composition.

232. A method of ameliorating or preventing toxicity associated with a bacterial toxin, comprising administering to a subject in need thereof
a) a first antibody that partially or completely inhibits binding of the bacterial toxin to a cell; and
b) a second antibody that partially or completely inhibits intracellular internalization of the toxin,
wherein the first antibody and the second antibody bind to the toxin at non-overlapping epitopes.

233. The method of claim 232, wherein the bacterial toxin comprises a Clostridium difficile toxin A or a Clostridium difficile toxin B.

234. The method of claim 232, wherein the first and the second antibodies are formulated together in a pharmaceutical composition.

235. The method of claim 232, wherein the first and the second antibodies are formulated for oral administration.

236. A pharmaceutical composition comprising
a) a first antibody that partially or completely inhibits binding of the bacterial toxin to a cell; and
b) a second antibody that partially or completely inhibits intracellular internalization of the toxin,
wherein the first antibody and the second antibody bind to the toxin at non-overlapping epitopes.

237. The pharmaceutical composition of claim 236, wherein the bacterial toxin comprises a Clostridium difficile toxin A or a Clostridium difficile toxin B.

238. The pharmaceutical composition of claim 236, wherein the first and the second antibodies are formulated together in a pharmaceutical composition.

239. The pharmaceutical composition of claim 236, wherein the first and the second antibodies are formulated for oral administration.

240-246. (canceled)

247. An antibody that binds Clostridium difficile toxin B, or a fragment thereof that binds Clostridium difficile toxin B, comprising two heavy chains, each heavy chain comprising consecutive amino acids, the amino acid sequence of which is set forth in SEQ ID NO:30, and two light chains, each light chain comprising consecutive amino acids, the amino acid sequence of which is set forth in SEQ ID NO:31.

248. An antibody that binds Clostridium difficile toxin B, or a fragment thereof that binds Clostridium difficile toxin B, which comprises two heavy chain variable regions, each heavy chain variable region comprising consecutive amino acids, the amino acid sequence of which is set forth in SEQ ID NO:5, and two light chain variable regions, each light chain variable region comprising consecutive amino acids, the amino acid sequence of which is set forth in SEQ ID NO:6.

249. The antibody according to claim 248, further comprising two heavy chain constant regions, each heavy chain constant region comprising consecutive amino acids corresponding to an IgG1, IgG2, IgG2a, IgG2b, IgG3, IgG4, IgM, IgA, IgD, or IgE heavy chain constant region, and two light chain constant regions, each light chain constant region comprising consecutive amino acids corresponding to a kappa or lambda light chain constant region.

250. The antibody or antibody fragment that binds Clostridium difficile toxin B according to claim 247 or claim 248, wherein the antibody fragment is selected from an Fab antibody fragment, an Fab′ antibody fragment, an F(ab′)₂ antibody fragment, an Fv fragment, or an Fd fragment.

251. An antibody having same binding specificity for Clostridium difficile toxin B as the antibody according to claim 247.

252. An antibody having same binding specificity for Clostridium difficile toxin B as the antibody according to claim 248.

253. An antibody that binds Clostridium difficile toxin A, or a fragment thereof that binds Clostridium difficile toxin A, which comprises (i) two heavy chain variable regions, each heavy chain variable region comprising consecutive amino acids, the amino acid sequence of which is set forth in SEQ ID NO:1 and two light chain variable regions, each light chain variable region comprising consecutive amino acids, the amino acid sequence of which is set forth in SEQ ID NO:2; or (ii) two heavy chain variable regions, each heavy chain variable region comprising consecutive amino acids, the amino acid sequence of which is set forth in SEQ ID NO:3 and two light chain variable regions, each light chain variable region comprising consecutive amino acids, the amino acid sequence of which is set forth in SEQ ID NO:4.

254. The antibody that binds Clostridium difficile toxin A, or a fragment thereof that binds Clostridium difficile toxin A, according to claim 253, which comprises two heavy chain variable regions, each heavy chain variable region comprising consecutive amino acids, the amino acid sequence of which is set forth in SEQ ID NO:1, and two light chain variable regions, each light chain variable region comprising consecutive amino acids, the amino acid sequence of which is set forth in SEQ ID NO:2.

255. The antibody that binds Clostridium difficile toxin A, or a fragment thereof that binds Clostridium difficile toxin A, according to claim 253, which comprises two heavy chain variable regions, each heavy chain variable region comprising consecutive amino acids, the amino acid sequence of which is set forth in SEQ ID NO:3, and two light chain variable regions, each light chain variable region comprising consecutive amino acids, the amino acid sequence of which is set forth in SEQ ID NO:4.

256. The antibody according to any one of claims 253 to 255, further comprising two heavy chain constant regions, each heavy chain constant region comprising consecutive amino acids corresponding to an IgG1, IgG2, IgG2a, IgG2b, IgG3, IgG4, IgM, IgA, IgD, or IgE heavy chain constant region, and two light chain constant regions, each light chain constant region comprising consecutive amino acids corresponding to a kappa or a lambda light chain constant region.

257. The antibody according to claim 254, further comprising two heavy chain constant regions, each heavy chain constant region comprising consecutive amino acids corresponding to an IgG1 constant region, and two light chain constant regions, each light chain constant region comprising consecutive amino acids corresponding to a kappa light chain constant region.

258. The antibody according to claim 255, further comprising two heavy chain constant regions, each heavy chain constant region comprising consecutive amino acids corresponding to an IgG2a constant region and two light chain constant regions, each light chain constant region comprising consecutive amino acids corresponding to a kappa light chain constant region.

259. An antibody that binds Clostridium difficile toxin A, or a fragment thereof that binds Clostridium difficile toxin A, comprising (i) two heavy chains, each heavy chain comprising consecutive amino acids, the amino acid sequence of which is set forth in SEQ ID NO:26 and two light chains, each light chain comprising consecutive amino acids, the amino acid sequence of which is set forth in SEQ ID NO:27; or (ii) two heavy chains, each heavy chain comprising consecutive amino acids, the amino acid sequence of which is set forth in SEQ ID NO:28 and two light chains, each light chain comprising consecutive amino acids, the amino acid sequence of which is set forth in SEQ ID NO:29.

260. The antibody that binds Clostridium difficile toxin A, or a fragment thereof that binds Clostridium difficile toxin A, according to claim 259, which comprises two heavy chains, each heavy chain comprising consecutive amino acids, the amino acid sequence of which is set forth in SEQ ID NO:26 and two light chains, each light chain comprising consecutive amino acids, the amino acid sequence of which is set forth in SEQ ID NO:27.

261. The antibody that binds Clostridium difficile toxin A, or a fragment thereof that binds Clostridium difficile toxin A, according to claim 259, which comprises two heavy chains, each heavy chain comprising consecutive amino acids, the amino acid sequence of which is set forth in SEQ ID NO:28 and two light chains, each light chain comprising consecutive amino acids, the amino acid sequence of which is set forth in SEQ ID NO:29.

262. The antibody that binds Clostridium difficile toxin A, or a fragment thereof that binds Clostridium difficile toxin A, according to claim 260, wherein the heavy chain is an IgG1 heavy chain isotype and the light chain is a kappa light chain isotype.

263. The antibody that binds Clostridium difficile toxin A, or a fragment thereof that binds Clostridium difficile toxin A, according to claim 261, wherein the heavy chain is an IgG2a heavy chain isotype and the light chain is a kappa light chain isotype.

264. The antibody according to any one of claims 247 to 249, 254 to 256, or 259 to 261, wherein the antibody is selected from a monoclonal antibody, a chimeric antibody, a humanized antibody, or a recombinant antibody.

265. An isolated nucleic acid encoding:
(i) a polypeptide comprising consecutive amino acids, the amino acid sequence of which is set forth in SEQ ID NO:1;
(ii) a polypeptide comprising consecutive amino acids, the amino acid sequence of which is set forth in SEQ ID NO:2;
(iii) a polypeptide comprising consecutive amino acids, the amino acid sequence of which is set forth in SEQ ID NO:3;
(iv) a polypeptide comprising consecutive amino acids, the amino acid sequence of which is set forth in SEQ ID NO:4.
(v) a polypeptide comprising consecutive amino acids, the amino acid sequence of which is set forth in SEQ ID NO:5;
(vi) a polypeptide comprising consecutive amino acids, the amino acid sequence of which is set forth in SEQ ID NO:6;
(vii) a polypeptide comprising consecutive amino acids, the amino acid sequence of which is set forth in SEQ ID NO:26;
(viii) a polypeptide comprising consecutive amino acids, the amino acid sequence of which is set forth in SEQ ID NO:27;
(ix) a polypeptide comprising consecutive amino acids, the amino acid sequence of which is set forth in SEQ ID NO:28;
(x) a polypeptide comprising consecutive amino acids, the amino acid sequence of which is set forth in SEQ ID NO:29;
(xi) a polypeptide comprising consecutive amino acids, the amino acid sequence of which is set forth in SEQ ID NO:30; or
(xii) a polypeptide comprising consecutive amino acids, the amino acid sequence of which is set forth in SEQ ID NO:31.

266. An isolated nucleic acid which hybridizes to the nucleic acid according to claim 265, as determined under stringent hybridization wash conditions.

267. The isolated nucleic acid according to claim 265, wherein the stringent hybridization wash conditions comprise one or more of:
(i) washing a hybridization complex with a solution comprising a salt concentration of about 0.02 molar at pH 7 at a temperature of at least about 50° C. to about 60° C.;
(ii) washing a hybridization complex with a solution comprising a salt concentration of about 0.15 M NaCl at 72° C. for about 15 minutes;
(iii) washing a hybridization complex with a solution comprising a salt concentration of about 0.2×SSC at a temperature of at least about 50° C. to about 60° C. for about 15 to about 20 minutes; or
(iv) washing a hybridization complex at least two times with a solution comprising a salt concentration of about 2×SSC containing 0.1% SDS at room temperature for 15 minutes, followed by washing the hybridization complex at least two times with a solution comprising 0.1×SSC and 0.1% SDS at 68° C. for 15 minutes.

268. An isolated host cell comprising a vector which comprises the isolated nucleic acid according to any one of claims 264, 265, or 267.

269. An antibody having the same binding specificity for Clostridium difficile toxin A as the antibody according to any one of claims 253 to 255 or 259 to 261.

270. A composition comprising one or more of the antibodies, or the fragments thereof, according to any one of claims 247, 248, 252 to 255, or 259 to 261, and a carrier.

271. The antibody, or fragment thereof, according to any one of claims 247, 248, 252 to 255, or 259 to 261 labeled with a substance which provides for a detectable signal.

272. A method of treating a subject afflicted with Clostridium difficile toxicity or infection, comprising administering to the subject one or more of the antibodies, or a humanized version thereof, according to any one of claims 247, 248, 253 to 256, or 260 to 262, in an amount effective to neutralize one or more of toxin A and toxin B of Clostridium difficile, and, optionally, co-administering a bioactive agent or drug, so as to thereby treat the Clostridium difficile toxicity or infection in the subject.
